SEQUENCE LISTING (83 sequences)

SEQ ID NO: 1 - RNA, 422 nt, Homo sapiens
  source 1..422: mol_type = mRNA
gaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagctaggcaccggcctatgtctgggggtggctctcat

SEQ ID NO: 2 - RNA, 424 nt, Homo sapiens
  source 1..424: mol_type = mRNA
atctgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagctaggcaccggcctatgtctgggggtggctctc

SEQ ID NO: 3 - RNA, 424 nt, Homo sapiens
  source 1..424: mol_type = mRNA
gactgaggccctgcccaatgagactctagggtccagtggatgccacagcccagcttggccctttccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagctaggcaccggcctatgtctgggggtggctctt

SEQ ID NO: 4 - RNA, 337 nt, Homo sapiens
  source 1..337: mol_type = mRNA
atgtaactgcctcaaattggactttgggcataaaagaacttttttatgcttaccatcttttttttttctttaacagatttgtatttaaaaattgtttttaaaaaattttaagatttacacaatgtttctctgtaaatattgccattaaatgtaaataactttaataaaacgtttatagcagttacccagaatttcaatccaaaagtgggtgggggggggagggaggggagaaggcacctggccggggaactcagccaaggttgtgagggttgattattataacgacgaccctacaggtcggaaagcaggggaggatgcatttgaatccccgaagcaa

SEQ ID NO: 5 - RNA, 205 nt, Homo sapiens
  source 1..205: mol_type = mRNA
aaatggtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatccc

SEQ ID NO: 6 - RNA, 193 nt, Homo sapiens
  source 1..193: mol_type = mRNA
ctgcctcattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacccagaatttcaatcca

SEQ ID NO: 7 - RNA, 331 nt, Homo sapiens
  source 1..331: mol_type = mRNA
cgtggctggacctctgggcgtgaacgaacttttttatgcttaccatcttttttttttctttaacagatttgtatttaagaattgtttttaaaaaattttaagatttacacaaggtttctctgtaaatatggccattagggtaaataactttaataaaacgtttatggcagttacacggaatttcaatccacaatctgtccgtgagcccggcaatgatcgctaaaggccttctaccgacagatatagtgtctttcggcgagtagttgattattataacgacgaccctacaggtcggaaagcaggggaggaagcatttgaagcaagaaagcaa

SEQ ID NO: 8 - RNA, 381 nt, Homo sapiens
  source 1..381: mol_type = mRNA
tctattggattgtgggaataaaagaacttttttatgattaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcccaatctctgcaacccaacacttgttccctatgttctcgatcccccaaaaagactcctgtgctacaagatatcaatcattccctaatttttttaacttaggcgcactcgcattcttccttttcacatcccaaaccccccctcccgttttgcaacccatgtccactcacctctctcctaccccatagaaactctg

SEQ ID NO: 9 - RNA, 192 nt, Homo sapiens
  source 1..192: mol_type = mRNA
gcctcattggactttgggcataaaagaacttttttatgcttaccatcttttttttttctttaacagatttgtatttaagaattgtttttaaaaaattttaaaatttacacaatgtttctctgtaaatattgccattaaaggtaaataactttaataaaacgtttatagcagttacccagaatttcaatccaa

SEQ ID NO: 10 - RNA, 590 nt, Homo sapiens
  source 1..590: mol_type = mRNA
aatgtagcctcattggactgtgggcataaaagaacttttttatgcttaccatctttttttttttttaacaattggatttaaaaattggttttaaaaaattttaagaattacaccaaggttctccggaaatattgccctttaatggtaataaattttataaaacgttttttctccttaccccgaattttcatccaatatgtctttttcatatactccaatgaatgtaacacaatgtatcactgggctttggccatacactataaactttcttaccgaatcccttgcttcccaatcataaaaaattcaccaattgctataattattgcaattgaaaaattcattgattctcaatcttgaccccccctcgcccccctggtatccccagaagagcccggttgaaattttttaaattcccaacccaatcccttttcttgctaggtgcctggaatgtgggggttttggaagtcttttaattgatccgcctcccctctaggacccctggcccttttgccaccaattcggtgtgaaaattccctacgccttgggccggacaacctacataccgtttaccctttttttacttgacaaat

SEQ ID NO: 11 - RNA, 620 nt, Homo sapiens
  source 1..620: mol_type = mRNA
ggatcaattggactttgggcataaaagaacttttttatgcttaccatcttttttttttctttaacagatttgtatttaagaattgtttttaaaaaattttaagatttacccaatgtttctctgtaaatattgccattaaatgtaaataactttaataaaacgtttatagcagttacacagaatttcaatccaatgtcgtttcatatactccaatgaatgtaacacaatgtatccctggtcaaacaaccttgaaccaaaactttccaaaccgaatctctgcttccccaacctttaaaaaacccacgaaaggctataataaatgtgatttgaaaaatcccatcaatcacgaacctttgaccgaccctcgcgccccttgttttccttaggcccccccgtttaaagtcctttaaatttccccccccaccacttttccttgttgagcctggcaattgggggttttgccagttcttttatttatccttccccccctatcgaccttcgggccccttttcaaccaaattggtgtgaaactccctccccccaagaccaagacccaacatcaccttgtcccacttgcttaataaccaaataaacctcattctttccaaattctattcccc

SEQ ID NO: 12 - RNA, 504 nt, synthetic construct
  misc_feature 1..504: note = Synthetic sequence based on MYC wherein stabilizing motifs have been selectively replaced by destabilizing motifs
  source 1..504: mol_type = other RNA
gagcaatcacctatgaacttgctgctaagttatgatcaaatgcaacctcacaaccttggctgagtcttgagactgaaagatttagccataatgtaaactgcctcaaattggactttgggcataaaagaaccctcctgcatgcttaccatccctcctgccctcctttaacagcctcgtaacttataattgacctcaaaaaacctcaagatttacacaatgtttctctgtaaatattgccattaaatgtaaataactttaataaaacgtttatagcagttacacagaatttcaatcctagtatatagtacctagtattataggtactataaaccctaacctcctgcatttaagtacattttgcactgcaaagtaagttatcctctattgacctcagaaaaaataaaataactggcaaatatatcattgagccaaatcttaagttgtgaatgctgcctctgctaacttatccctcccaaccaccaccatccctgctgccctcatcaa

SEQ ID NO: 13 - RNA, 501 nt, synthetic construct
  misc_feature 1..501: note = Synthetic sequence based on ERBB2 wherein stabilizing motifs have been selectively replaced by destabilizing motifs
  source 1..501: mol_type = other RNA
caggggaacctgccatgccaggaacctgtcctaaggaacctgccttcctgcttgagttcccagatggctggaaggggtccagcctcgttggaagaggaacagcactggggagtcttcgtggattctgaggccctgcccaatgagactctagggtccagtggatgccacagcccagcttggccctctccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggtcgcgttcagagtgccctcctgcttagctgctaagttatctgcctcgctgcctcaaagatgaaataaagacccagggggagaatgggtgttgtatggggaggcaagtgtggggggtccttctccacacccactttgtccatttgcaaatatat

SEQ ID NO: 14 - RNA, 68 nt, Homo sapiens
  source 1..68: mol_type = mRNA
caggctttgtacagagtgcttttctgtttagtttttactttttttgttttgtttttttaaagatgaaa

SEQ ID NO: 15 - RNA, 71 nt, synthetic construct
  misc_feature 1..71: note = Synthetic sequence wherein stabilizing motifis have been selectively replaced by destabilizing motifs
  source 1..71: mol_type = other RNA
caggctgcgtacagagtgccctcctgcttagctgttaagttatctgcctcgctgcctcaaagatgaaataa

SEQ ID NO: 16 - DNA, 768 bp, synthetic construct
  misc_feature 1..768: note = Synthetic sequence based on ERBB2 wherein stabilizing motifs have been selectively replaced by destabilizing motifs
  source 1..768: mol_type = other DNA
gcccgcgaggacccgcccgagcttcgaaaaaaaaaagccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctcaggggaacctgccatgccaggaacctgtcctaaggaacctgccttcctgcttgagttcccagatggctggaaggggtccagcctcgttggaagaggaacagcactggggagtcttcgtggattctgaggccctgcccaatgagactctagggtccagtggatgccacagcccagcttggccctctccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggtcgcgttcagagtgccctcctgcttagctgctaagttatctgcctcgctgcctcaaagatgaaataaagacccagggggagaatgggtgttgtatggggaggcaagtgtggggggtccttctccacacccactttgtccatttgcaaatatataaaaaaaaaacctaggggccggccccgccg

SEQ ID NO: 17 - DNA, 694 bp, synthetic construct
  misc_feature 1..690: note = Synthetic sequence based on MYC wherein stabilizing motifs have been selectively replaced by destabilizing motifs
  source 1..694: mol_type = other DNA
ggacccgcccgagcttcgaaaaaaaaaagccatcgaggcaaaattcccccatctgcgtcagtcccctcaacttccgcctctacgcgggcctcgcggcagggcggtacgtcagccattctggtccgccgcgcgcatgcgtagtggagcttgcaggctggagctgagcaatcacctatgaacttgctgctaagttatgatcaaatgcaacctcacaaccttggctgagtcttgagactgaaagatttagccataatgtaaactgcctcaaattggactttgggcataaaagaaccctcctgcatgcttaccatccctcctgccctcctttaacagcctcgtaacttataattgacctcaaaaaacctcaagatttacacaatgtttctctgtaaatattgccattaaatgtaaataactttaataaaacgtttatagcagttacacagaatttcaatcctagtatatagtacctagtattataggtactataaaccctaacctcctgcatttaagtacattttgcactgcaaagtaagttatcctctattgacctcagaaaaaataaaataactggcaaatatatcattgagccaaatcttaagttgtgaatgctgcctctgctaacttatccctcccaaccaccaccatccctgctgccctcatcaaaaaaaaaacctaggggccggccccgccg

SEQ ID NO: 18 - DNA, 501 bp, synthetic construct
  misc_feature 1..501: note = Synthetic sequence based on ERBB2 wherein stabilizing motifs have been selectively replaced by destabilizing motifs
  source 1..501: mol_type = other DNA
caggggaacctgccatgccaggaacctgtcctaaggaacctgccttcctgcttgagttcccagatggctggaaggggtccagcctcgttggaagaggaacagcactggggagtcttcgtggattctgaggccctgcccaatgagactctagggtccagtggatgccacagcccagcttggccctctccttccagatcctgggtactgaaagccttagggaagctggcctgagaggggaagcggccctaagggagtgtctaagaacaaaagcgacccattcagagactgtccctgaaacctagtactgccccccatgaggaaggaacagcaatggtgtcagtatccaggtcgcgttcagagtgccctcctgcttagctgctaagttatctgcctcgctgcctcaaagatgaaataaagacccagggggagaatgggtgttgtatggggaggcaagtgtggggggtccttctccacacccactttgtccatttgcaaatatat

SEQ ID NO: 19 - DNA, 504 bp, synthetic construct
  misc_feature 1..502: note = Synthetic sequence based on MYC wherein stabilizing motifs have been selectively replaced by destabilizing motifs
  source 1..504: mol_type = other DNA
gagcaatcacctatgaacttgctgctaagttatgatcaaatgcaacctcacaaccttggctgagtcttgagactgaaagatttagccataatgtaaactgcctcaaattggactttgggcataaaagaaccctcctgcatgcttaccatccctcctgccctcctttaacagcctcgtaacttataattgacctcaaaaaacctcaagatttacacaatgtttctctgtaaatattgccattaaatgtaaataactttaataaaacgtttatagcagttacacagaatttcaatcctagtatatagtacctagtattataggtactataaaccctaacctcctgcatttaagtacattttgcactgcaaagtaagttatcctctattgacctcagaaaaaataaaataactggcaaatatatcattgagccaaatcttaagttgtgaatgctgcctctgctaacttatccctcccaaccaccaccatccctgctgccctcatcaa

SEQ ID NO: 20 - DNA, 20 bp, synthetic construct
  misc_feature 1..20: note = ERBB2 forward primer for use in PCR
  source 1..20: mol_type = other DNA
gcctcgttggaagaggaaca

SEQ ID NO: 21 - DNA, 20 bp, synthetic construct
  misc_feature 1..20: note = ERBB2 reverse primer for use in PCR
  source 1..20: mol_type = other DNA
agagccacccccagacatag

SEQ ID NO: 22 - DNA, 20 bp, synthetic construct
  misc_feature 1..20: note = MYC forward primer for use in PCR
  source 1..20: mol_type = other DNA
cctcacaaccttggctgagt

SEQ ID NO: 23 - DNA, 23 bp, synthetic construct
  misc_feature 1..23: note = MYC reverse primer for use in PCR
  source 1..23: mol_type = other DNA
ggattgaaattctgtgtaactgc

SEQ ID NO: 24 - DNA, 20 bp, synthetic construct
  misc_feature 1..20: note = DCP1A forward primer for use in PCR
  source 1..20: mol_type = other DNA
ccctcaacttccgcctctac

SEQ ID NO: 25 - DNA, 20 bp, synthetic construct
  misc_feature 1..20: note = DCP1A reverse primer for use in PCR
  source 1..20: mol_type = other DNA
cagcctgcaagctccactac

SEQ ID NO: 26 - RNA, 14 nt, synthetic construct
  misc_feature 1..14: note = digestion enhancing sequence
  source 1..14: mol_type = other RNA
ggacccgcccgagc

SEQ ID NO: 27 - RNA, 15 nt, synthetic construct
  misc_feature 1..15: note = digestion enhancing sequence
  source 1..15: mol_type = other RNA
gggccggccccgccg

SEQ ID NO: 28 - RNA, 22 nt, synthetic construct
  misc_feature 1..22: note = Digestion enhancing sequence
  source 1..22: mol_type = other RNA
gcccgcgaggacccgcccgagc

SEQ ID NO: 29 - DNA, 201 bp, synthetic construct
  misc_feature 1..201: note = DCP1A promoter sequence
  source 1..201: mol_type = other DNA
gccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagct

SEQ ID NO: 30 - DNA, 134 bp, synthetic construct
  misc_feature 1..134: note = Shortened DCP1A promoter sequence
  source 1..134: mol_type = other DNA
gccatcgaggcaaaattcccccatctgcgtcagtcccctcaacttccgcctctacgcgggcctcgcggcagggcggtacgtcagccattctggtccgccgcgcgcatgcgtagtggagcttgcaggctggagct

SEQ ID NO: 31 - DNA, 92 bp, synthetic construct
  source 1..92: mol_type = other DNA
cttttttatgcttaccatcttttttttttctttaacagatttgtatttaagaattgtttttaaaaaattttaagatttacacaaggtttctc

SEQ ID NO: 32 - DNA, 99 bp, Synthetic construct
  source 1..99: mol_type = other DNA
cttttttatgcttaccatcttttttttttctttaacagatttgtatttaaaaattgtttttaaaaaattttaagatttacacaatgtttctctgtaaat

SEQ ID NO: 33 - DNA, 91 bp, Synthetic construct
  source 1..91: mol_type = other DNA
cttttttatgcttaccatcttttttttttctttaacagatttgtatttaagaattgtttttaaaaaattttaagatttacacaatgtttct

SEQ ID NO: 34 - DNA, 95 bp, synthetic construct
  source 1..95: mol_type = other DNA
cttttttatgcttaccatcttttttttttctttaacagatttgtatttaagaattgtttttaaaaaattttaagatttacacaatgtttctctgt

SEQ ID NO: 35 - RNA, 236 nt, synthetic construct
  source 1..236: mol_type = other RNA
agctgagcaatcacctatgaacttgctgctaagttatgatcaaatgcaacctcacaaccttggctgagtcttgagactgaaagatttacccataatgtaaactgcctcaaattggactttgggcataaaagaaccctcctgcatgcttaccatccctcctgccctcctttaacagcctcgtaacttataattgacctccaaaaccctctcattaaccaatgtttatttatatattg

SEQ ID NO: 36 - RNA, 246 nt, synthetic construct
  source 1..246: mol_type = other RNA
cctcacaaccttggctgagtcttgagactgaaagatttagccataatgtaaactgcctcaaattggactttgggcataaaagaacttttttatgcttaccatcttttttttttctttaacagatttgtatttaagaattgtttttaaaaaattttaagatttacacaatgtttctctgtaaatattgccattaaatgtaaataactttaataaaacgtttatagcagttacacagaatttcaatcc

SEQ ID NO: 37 - RNA, 300 nt, synthetic construct
  source 1..300: mol_type = other RNA
agctgagcaatcacctatgaacttgctgctaagttatgatcaaatgcaacctcacaaccttggctgagtcttgagactgaaagatttagccataatgtaaactgcctcaaattggactttgggcataaaagaaccctcctgcatgcttaccatccctcctgccctcctttaacagcctcgtaacttataattgacctcaaaaaacctcaagatttacacaatgtttctctgtaaatattgccattaaatgtaaataactttaataaaacgtttatagcagttacacagaatttcaatcct

SEQ ID NO: 38 - RNA, 441 nt, synthetic construct
  source 1..441: mol_type = other RNA
caggggaacctgccatgccaggaacctgtcctaaggaacctgccttcctgcttgagttcccagatggctggaaggggtccagcctcgttggaagaggaacagcactggggagtcttcgtggattctgaggccctgcccaatgagactctagggtccagtggatgccacagcccagcttggccctctccttccagatcctgggtactgaaagccttagggaagctggcctgagaggggaagagtactgccccccatgaggaaggaacagcaatggtgtcagtatccaggtcgcgttcagagtgccctcctgcttagctgctaagttatctgcctcgctgcctcaaagatgaaataaagacccagggggagaatgggtgttgtatggggaggcaagtgtggggggtccttctccacacccactttgtccatttgcaaatatat

SEQ ID NO: 39 - RNA, 399 nt, synthetic construct
  source 1..399: mol_type = other RNA
gcctcgttggaagaggaacagcactggggagtctttgtggattctgaggccctgcccaatgagactctagggtccagtggatgccacagcccagcttggccctttccttccagatcctgggtactgaaagccttagggaagctggcctgagaggggaagagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagctaggcaccggcctatgtctgggggtg

SEQ ID NO: 40 - RNA, 358 nt, synthetic construct
  source 1..358: mol_type = other RNA
atctgaggccctgcccaatgagactctagggtccagtggatgccacagcccagcttggccctttccttccagatcctgggtactgaaagccttagggaagctggcctgagaggggaagagtactgccccccatgaggaaggaacagcaatggtgtcagtatccaggctttgtacagagtgcttttctgtttagtttttactttttttgttttgtttttttaaagatgaaataaagacccagggggagaatgggtgttgtatggggaggcaagtgtggggggtccttctccacacccactttgtccatttgcaaatatattttggaaaacagctaggcaccggcctatgtctgggggtg

SEQ ID NO: 41 - DNA, 536 bp, Escherichia coli
  source 1..536: mol_type = genomic DNA
gggnnnnaannnnnnnnncngangcctgccagtgagagtcnagggtccagtgaaggccgcatcgcaccttggccctctccttccgtatcctgggtactgaggccgtagggaagctggccnttgaggggaagcgaggactngaagtgagngaagccagcctacccggtaaaatcgggctctgagtcgnttgggcccaacctgaggaggattagcattgccgtgagcggcgggggttacctaaggtgctgaacagttccctccctgtcgcattggtngggaaccatctagctcagccacatcctcccgctacgngctttgggctgattgcaanccatgggactgacaccacttctactacatcctcnnnggcggtgactcttcnacctccacccatagacccatattnccctnattctcctcntaccannaaaanncctgttcggnnccancaccatcaattcttcnctnnnagattnntccatcattctttttctnnncaatatgtaacattgttttnnatgaannnccaaannccn

SEQ ID NO: 42 - DNA, 499 bp, Escherichia coli
  source 1..499: mol_type = genomic DNA
gnnnnnnnnntnnnnnancagaggcnngctcactgagactctatggtcagtgaaggccacaccccaacttggccctttccttccggatcctgggtactgaggccttagggaagctggcctgacatgggaagcagcccttgcggagtgtcttgaactgaaccgacccattaaaagactgtccctgaaacctcgtactgcccccctgaggaaggaactgccctgttggcagtgtcggtgctttgtacacagtgcttttcngtttatnttttactttttttgttttgcttttttaaatatgaaattcatacccatgttgacaatgggtgttgtatgggaaggcaagggactccctttggtgatgggcgcgacctttggtatcgccatatcgctggtgattatcacctggtctgatgtgtataccggacccaccccttnncacatccaanaaggctcttgctgatggcaatatctggtggtggctctcnnaaaaanctcatcc

SEQ ID NO: 43 - DNA, 547 bp, Escherichia coli
  source 1..547: mol_type = genomic DNA
nnnnnnnnnanttnnnnaacnnangccngcnnntgagactctacggtccagtggatgtaacatcccagcttggtcctttccttccatatcctgggtactgaaagccttagggaaactggcctgataggggaagcggccctacatgattggatggnacggaatcgaccctttcagagactgtccctgaaacctatcagtgcccgccacgatgaaagaacagcaacggtgacagtatcctggcattgtccaaanngcttttctgtgcttntttactttttttaatnagtttttttaaagatcaaacatccaccccaggtcccaatggggggtgcctgaggaagaacgattggcgcctgttcccgttcccctttactggactgcgtactacaccccgaggccttctttgagagagcgnnccatcctgggacagcaacctaactannccctctccccctgcggatgacaatgtctggccggggttctnataggctactcacccccttttgtttttattacaaacgattatgtttgntctgaccncannaaa

SEQ ID NO: 44 - DNA, 436 bp, Escherichia coli
  source 1..436: mol_type = genomic DNA
nnnnnnnnnnnttnaaanatatttgcaatggacaaagtgggtgtggagaaggaccccccacacttgcctccccatacaacacccattctccccctgggtctttatttcatctttaaaaaaacaaaacaaaaaaagtaaaaactaaacacaaaagcactctgtacaaagcctggatactgacaccattgctgttccttcctcatggggggcagtactaggtttcatggacagtctctgaatggttcgcttttgttcttagacactcccttagggccgcttcccctctcaggccagcttccctaaggctttcagtacccaggatctggaaggaaagggccaagctgggctgtggcatccactggaccctatagtctcattgggcagggcctcagaatccacaaagactccccagtgctgttcctcttccaaccaggca

SEQ ID NO: 45 - DNA, 468 bp, Escherichia coli
  source 1..468: mol_type = genomic DNA
nnnnnnntnnnnnnnnnnnnngnnnccntangtttctttttatatatttcgctctggaactgcgggcgtggagaaggaccccccacactggcctccccatacaacacccattctccccctgggtctttatttcgtctttgaggccgcgggggacatgacttaccacctaaccatgagggcactctcaacgcgacctggatactgacaccattgctgttccttcctcatggggggcagtactaagtttcagggacagtctctgaatgggtcgcttttgttcttangtctcccttatggcctcttcccctctcaagccaatttccntaaggatttctctaccaangatctggaaaaaaagggccaatttgggctgtggcatccccaggaccnnacattgcatgaaagctaatgccgacaaggatcaataaaaaactcttanattatttcttaatacttcccctacacttn

SEQ ID NO: 46 - DNA, 675 bp, Escherichia coli
  source 1..675: mol_type = genomic DNA
nnnnnnnnnnnnngnanntcccccatctgcgtcagtcccctcaacttccgcctctacgcgggcctcgcggcagggcggtacgtcagccattctggtccgccgcgcgcatgcgtagtggagcttgcaggctggagctgagcaatcacctatgaacttgctgctaagttatgatcaaatgcaacctcacaaccttggctgagtcttgagactgaaagatttagccataatgtaaactgcctcaaattggactttgggcataaaagaaccctcctgcatgcttaccatccctcctgccctcctttaacagcctcgtaacttataattgacctcaaaaaacctcaagatttacacaatgtttctctgtaaatattgccattaaatgtaaataactttaataaaacgtttatagcagttacacagaatttcaatcctagtatatagtacctagtattataggtactataaaccctaacctcctgcatttaagtacattttgcactgcaaagtaagttatcctctattgacctcagaaaaaataaaataactggcaaatatatcattgagccaaatcttaagttgtgaatgctgcctctgctaacttatccctcccaaccaccaccatccctgctgccctcatcaaaaaacctacgggccggtctcgccgtaatgtgagta

SEQ ID NO: 47 - DNA, 354 bp, Escherichia coli
  source 1..354: mol_type = genomic DNA
nnnnnnnnnnttnnnnaganggcacaggnntggtggtggttgggagggataagttagcacaggcagcattcacaacttaagatttggctcatgatatatttgccagttattttattttttctgaggtcaataaaggataacttactttgtagngcaaaatgaacttaaatgcaagaggtaaaggncaatagaacctttaatacaacgtatnntttacttngnaaaaaanccaangtnacggcttaaaaccctcnctaaanaancaccnntccaaaagtcaattcagacgtacnccncccncnangggccnctaaattnntactaccaacntnctgccccctccatacanaaaan

SEQ ID NO: 48 - DNA, 356 bp, Escherichia coli
  source 1..356: mol_type = genomic DNA
nnnnnnnggcttttnnngnnnnnncngggatggtggtggttgggagggataagttagcagaggcagcattcacaacttaagatttggctcaatgatacctttgccagttattttattttttctgatgtcaataaaacgataacttactttgcattgcaaaatgtncatacatgcaggangttacggcttannaaacctttaataatcaccacatatatctttcaaagaaagctaaaagttctgttataaacatcactataaaaacaataatccaagaataacttcagaactacccctcccactagnaaacgctaaattgctaataacaacatattgcnctctnnaaacaaaaaaaa

SEQ ID NO: 49 - DNA, 123 bp, Homo sapiens
  source 1..123: mol_type = genomic DNA
nnnnnnnnncnnatgtaactgcctcaattggactttgggcataaaagaacttttttatgcttaccatcttttttttttctttaacagattttgtatttaaaaattgtttttaaaaaattttaa

SEQ ID NO: 50 - DNA, 121 bp, Homo sapiens
  source 1..121: mol_type = genomic DNA
nnnnnanntnncnnaatgtaactgcctcaattggactttgggcataaaagaacttttttatgcttaccatcttttttttttctttaacagatttgtatttaagaattgtttttaaaaaatt

SEQ ID NO: 51 - DNA, 125 bp, Homo sapiens
  source 1..125: mol_type = genomic DNA
nnnnnnannnnannncnnaagcnacngncgtggctggacctctgggctgaacgaattttttatgcttaccatcttttttttttctttaacagatttgtatttaagaattgtttttaaaaaattta

SEQ ID NO: 52 - DNA, 125 bp, Homo sapiens
  source 1..125: mol_type = genomic DNA
cnnnaaannnnnncnnaangtaanctgcctcnattggactttgggcataaaagaacttttttatgcttaccatcttttttttttctttaacagatttgtatttaaagaattgtttttaaaaaatt

SEQ ID NO: 53 - DNA, 45 bp, Synthetic construct
  source 1..45: mol_type = other DNA
gcaacgcaatggacccgcccgagcttcgaaaaaaaaaagccatcg

SEQ ID NO: 54 - DNA, 28 bp, synthetic construct
  source 1..28: mol_type = other DNA
actcacattacggcggggccggccccta

SEQ ID NO: 55 - DNA, 36 bp, Synthetic construct
  source 1..36: mol_type = other DNA
ggccccgccgtaatgtgagttagctcactcattagg

SEQ ID NO: 56 - DNA, 28 bp, Synthetic construct
  source 1..28: mol_type = other DNA
gggcgggtccattgcgttgcgctcactg

SEQ ID NO: 57 - DNA, 20 bp, synthetic construct
  source 1..20: mol_type = other DNA
gtcacacccttctcccttcg

SEQ ID NO: 58 - DNA, 20 bp, Synthetic construct
  source 1..20: mol_type = other DNA
caggtacaagctggaggtgg

SEQ ID NO: 59 - DNA, 20 bp, Synthetic construct
  source 1..20: mol_type = other DNA
acgttcacgttctccacgat

SEQ ID NO: 60 - DNA, 20 bp, Synthetic construct
  source 1..20: mol_type = other DNA
tgtcaggtaagcagtgggtc

SEQ ID NO: 61 - DNA, 20 bp, Synthetic construct
  source 1..20: mol_type = other DNA
tctagtgcagcaatcccgaa

SEQ ID NO: 62 - DNA, 20 bp, Synthetic construct
  source 1..20: mol_type = other DNA
ggactggtcacttacatggc

SEQ ID NO: 63 - DNA, 21 bp, Synthetic construct
  source 1..21: mol_type = other DNA
tccataaagaagaggcagtgc

SEQ ID NO: 64 - DNA, 23 bp, Synthetic construct
  source 1..23: mol_type = other DNA
agatgattggattttgatggggt

SEQ ID NO: 65 - DNA, 10 bp, Synthetic construct
  source 1..10: mol_type = other DNA
aaaaaaaaaa

SEQ ID NO: 66 - DNA, 12 bp, synthetic construct
  source 1..12: mol_type = other DNA
aaaaaaaaaaaa

SEQ ID NO: 67 - DNA, 14 bp, synthetic construct
  source 1..14: mol_type = other DNA
ggacccgcccgagc

SEQ ID NO: 68 - DNA, 813 bp, Escherichia coli
  source 1..813: mol_type = genomic DNA
gcccgcgaggacccgcccgagcttcgaaaaaaaaaagccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctgggnnnnaannnnnnnnncngangcctgccagtgagagtcnagggtccagtgaaggccgcatcgcaccttggccctctccttccgtatcctgggtactgaggccgtagggaagctggccnttgaggggaagcgaggactngaagtgagngaagccagcctacccggtaaaatcgggctctgagtcgnttgggcccaacctgaggaggattagcattgccgtgagcggcgggggttacctaaggtgctgaacagttccctccctgtcgcattggtngggaaccatctagctcagccacatcctcccgctacgngctttgggctgattgcaanccatgggactgacaccacttctactacatcctcnnnggcggtgactcttcnacctccacccatagacccatattnccctnattctcctcntaccannaaaanncctgttcggnnccancaccatcaattcttcnctnnnagattnntccatcattctttttctnnncaatatgtaacattgttttnnatgaannnccaaannccnctaagtgttggctttaaagtcctaggggccggccccgccg

SEQ ID NO: 69 - DNA, 776 bp, Escherichia coli
  source 1..776: mol_type = genomic DNA
gcccgcgaggacccgcccgagcttcgaaaaaaaaaagccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctgnnnnnnnnntnnnnnancagaggcnngctcactgagactctatggtcagtgaaggccacaccccaacttggccctttccttccggatcctgggtactgaggccttagggaagctggcctgacatgggaagcagcccttgcggagtgtcttgaactgaaccgacccattaaaagactgtccctgaaacctcgtactgcccccctgaggaaggaactgccctgttggcagtgtcggtgctttgtacacagtgcttttcngtttatnttttactttttttgttttgcttttttaaatatgaaattcatacccatgttgacaatgggtgttgtatgggaaggcaagggactccctttggtgatgggcgcgacctttggtatcgccatatcgctggtgattatcacctggtctgatgtgtataccggacccaccccttnncacatccaanaaggctcttgctgatggcaatatctggtggtggctctcnnaaaaanctcatccctaagtgttggctttaaagtcctaggggccggccccgccg

SEQ ID NO: 70 - DNA, 824 bp, Escherichia coli
  source 1..824: mol_type = genomic DNA
gcccgcgaggacccgcccgagcttcgaaaaaaaaaagccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctnnnnnnnnnanttnnnnaacnnangccngcnnntgagactctacggtccagtggatgtaacatcccagcttggtcctttccttccatatcctgggtactgaaagccttagggaaactggcctgataggggaagcggccctacatgattggatggnacggaatcgaccctttcagagactgtccctgaaacctatcagtgcccgccacgatgaaagaacagcaacggtgacagtatcctggcattgtccaaanngcttttctgtgcttntttactttttttaatnagtttttttaaagatcaaacatccaccccaggtcccaatggggggtgcctgaggaagaacgattggcgcctgttcccgttcccctttactggactgcgtactacaccccgaggccttctttgagagagcgnnccatcctgggacagcaacctaactannccctctccccctgcggatgacaatgtctggccggggttctnataggctactcacccccttttgtttttattacaaacgattatgtttgntctgaccncannaaactaagtgttggctttaaagtcctaggggccggccccgccg

SEQ ID NO: 71 - DNA, 713 bp, Escherichia coli
  source 1..713: mol_type = genomic DNA
gcccgcgaggacccgcccgagcttcgaaaaaaaaaagccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctnnnnnnnnnnnttnaaanatatttgcaatggacaaagtgggtgtggagaaggaccccccacacttgcctccccatacaacacccattctccccctgggtctttatttcatctttaaaaaaacaaaacaaaaaaagtaaaaactaaacacaaaagcactctgtacaaagcctggatactgacaccattgctgttccttcctcatggggggcagtactaggtttcatggacagtctctgaatggttcgcttttgttcttagacactcccttagggccgcttcccctctcaggccagcttccctaaggctttcagtacccaggatctggaaggaaagggccaagctgggctgtggcatccactggaccctatagtctcattgggcagggcctcagaatccacaaagactccccagtgctgttcctcttccaaccaggcactaagtgttggctttaaagtcctaggggccggccccgccg

SEQ ID NO: 72 - DNA, 745 bp, Escherichia coli
  source 1..745: mol_type = genomic DNA
gcccgcgaggacccgcccgagcttcgaaaaaaaaaagccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctnnnnnnntnnnnnnnnnnnnngnnnccntangtttctttttatatatttcgctctggaactgcgggcgtggagaaggaccccccacactggcctccccatacaacacccattctccccctgggtctttatttcgtctttgaggccgcgggggacatgacttaccacctaaccatgagggcactctcaacgcgacctggatactgacaccattgctgttccttcctcatggggggcagtactaagtttcagggacagtctctgaatgggtcgcttttgttcttangtctcccttatggcctcttcccctctcaagccaatttccntaaggatttctctaccaangatctggaaaaaaagggccaatttgggctgtggcatccccaggaccnnacattgcatgaaagctaatgccgacaaggatcaataaaaaactcttanattatttcttaatacttcccctacacttnctaagtgttggctttaaagtcctaggggccggccccgccg

SEQ ID NO: 73 - DNA, 952 bp, Escherichia coli
  source 1..952: mol_type = genomic DNA
gcccgcgaggacccgcccgagcttcgaaaaaaaaaagccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctnnnnnnnnnnnnngnanntcccccatctgcgtcagtcccctcaacttccgcctctacgcgggcctcgcggcagggcggtacgtcagccattctggtccgccgcgcgcatgcgtagtggagcttgcaggctggagctgagcaatcacctatgaacttgctgctaagttatgatcaaatgcaacctcacaaccttggctgagtcttgagactgaaagatttagccataatgtaaactgcctcaaattggactttgggcataaaagaaccctcctgcatgcttaccatccctcctgccctcctttaacagcctcgtaacttataattgacctcaaaaaacctcaagatttacacaatgtttctctgtaaatattgccattaaatgtaaataactttaataaaacgtttatagcagttacacagaatttcaatcctagtatatagtacctagtattataggtactataaaccctaacctcctgcatttaagtacattttgcactgcaaagtaagttatcctctattgacctcagaaaaaataaaataactggcaaatatatcattgagccaaatcttaagttgtgaatgctgcctctgctaacttatccctcccaaccaccaccatccctgctgccctcatcaaaaaacctacgggccggtctcgccgtaatgtgagtactaagtgttggctttaaagtcctaggggccggccccgccg

SEQ ID NO: 74 - DNA, 631 bp, Escherichia coli
  source 1..631: mol_type = genomic DNA
gcccgcgaggacccgcccgagcttcgaaaaaaaaaagccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctnnnnnnnnnnttnnnnaganggcacaggnntggtggtggttgggagggataagttagcacaggcagcattcacaacttaagatttggctcatgatatatttgccagttattttattttttctgaggtcaataaaggataacttactttgtagngcaaaatgaacttaaatgcaagaggtaaaggncaatagaacctttaatacaacgtatnntttacttngnaaaaaanccaangtnacggcttaaaaccctcnctaaanaancaccnntccaaaagtcaattcagacgtacnccncccncnangggccnctaaattnntactaccaacntnctgccccctccatacanaaaanctaagtgttggctttaaagtcctaggggccggccccgccg

SEQ ID NO: 75 - DNA, 633 bp, Escherichia coli
  source 1..633: mol_type = genomic DNA
gcccgcgaggacccgcccgagcttcgaaaaaaaaaagccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctnnnnnnnggcttttnnngnnnnnncngggatggtggtggttgggagggataagttagcagaggcagcattcacaacttaagatttggctcaatgatacctttgccagttattttattttttctgatgtcaataaaacgataacttactttgcattgcaaaatgtncatacatgcaggangttacggcttannaaacctttaataatcaccacatatatctttcaaagaaagctaaaagttctgttataaacatcactataaaaacaataatccaagaataacttcagaactacccctcccactagnaaacgctaaattgctaataacaacatattgcnctctnnaaacaaaaaaaactaagtgttggctttaaagtcctaggggccggccccgccg

SEQ ID NO: 76 - DNA, 737 bp, Synthetic construct
  source 1..737: mol_type = other DNA
gccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctgggnnnnaannnnnnnnncngangcctgccagtgagagtcnagggtccagtgaaggccgcatcgcaccttggccctctccttccgtatcctgggtactgaggccgtagggaagctggccnttgaggggaagcgaggactngaagtgagngaagccagcctacccggtaaaatcgggctctgagtcgnttgggcccaacctgaggaggattagcattgccgtgagcggcgggggttacctaaggtgctgaacagttccctccctgtcgcattggtngggaaccatctagctcagccacatcctcccgctacgngctttgggctgattgcaanccatgggactgacaccacttctactacatcctcnnnggcggtgactcttcnacctccacccatagacccatattnccctnattctcctcntaccannaaaanncctgttcggnnccancaccatcaattcttcnctnnnagattnntccatcattctttttctnnncaatatgtaacattgttttnnatgaannnccaaannccn

SEQ ID NO: 77 - DNA, 700 bp, synthetic construct
  source 1..700: mol_type = other DNA
gccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctgnnnnnnnnntnnnnnancagaggcnngctcactgagactctatggtcagtgaaggccacaccccaacttggccctttccttccggatcctgggtactgaggccttagggaagctggcctgacatgggaagcagcccttgcggagtgtcttgaactgaaccgacccattaaaagactgtccctgaaacctcgtactgcccccctgaggaaggaactgccctgttggcagtgtcggtgctttgtacacagtgcttttcngtttatnttttactttttttgttttgcttttttaaatatgaaattcatacccatgttgacaatgggtgttgtatgggaaggcaagggactccctttggtgatgggcgcgacctttggtatcgccatatcgctggtgattatcacctggtctgatgtgtataccggacccaccccttnncacatccaanaaggctcttgctgatggcaatatctggtggtggctctcnnaaaaanctcatcc

SEQ ID NO: 78 - DNA, 748 bp, Synthetic construct
  source 1..748: mol_type = other DNA
gccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctnnnnnnnnnanttnnnnaacnnangccngcnnntgagactctacggtccagtggatgtaacatcccagcttggtcctttccttccatatcctgggtactgaaagccttagggaaactggcctgataggggaagcggccctacatgattggatggnacggaatcgaccctttcagagactgtccctgaaacctatcagtgcccgccacgatgaaagaacagcaacggtgacagtatcctggcattgtccaaanngcttttctgtgcttntttactttttttaatnagtttttttaaagatcaaacatccaccccaggtcccaatggggggtgcctgaggaagaacgattggcgcctgttcccgttcccctttactggactgcgtactacaccccgaggccttctttgagagagcgnnccatcctgggacagcaacctaactannccctctccccctgcggatgacaatgtctggccggggttctnataggctactcacccccttttgtttttattacaaacgattatgtttgntctgaccncannaaa

SEQ ID NO: 79 - DNA, 637 bp, Synthetic construct
  source 1..637: mol_type = other DNA
gccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctnnnnnnnnnnnttnaaanatatttgcaatggacaaagtgggtgtggagaaggaccccccacacttgcctccccatacaacacccattctccccctgggtctttatttcatctttaaaaaaacaaaacaaaaaaagtaaaaactaaacacaaaagcactctgtacaaagcctggatactgacaccattgctgttccttcctcatggggggcagtactaggtttcatggacagtctctgaatggttcgcttttgttcttagacactcccttagggccgcttcccctctcaggccagcttccctaaggctttcagtacccaggatctggaaggaaagggccaagctgggctgtggcatccactggaccctatagtctcattgggcagggcctcagaatccacaaagactccccagtgctgttcctcttccaaccaggca

SEQ ID NO: 80 - DNA, 669 bp, Synthetic construct
  source 1..669: mol_type = other DNA
gccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctnnnnnnntnnnnnnnnnnnnngnnnccntangtttctttttatatatttcgctctggaactgcgggcgtggagaaggaccccccacactggcctccccatacaacacccattctccccctgggtctttatttcgtctttgaggccgcgggggacatgacttaccacctaaccatgagggcactctcaacgcgacctggatactgacaccattgctgttccttcctcatggggggcagtactaagtttcagggacagtctctgaatgggtcgcttttgttcttangtctcccttatggcctcttcccctctcaagccaatttccntaaggatttctctaccaangatctggaaaaaaagggccaatttgggctgtggcatccccaggaccnnacattgcatgaaagctaatgccgacaaggatcaataaaaaactcttanattatttcttaatacttcccctacacttn

SEQ ID NO: 81 - DNA, 876 bp, Synthetic construct
  source 1..876: mol_type = other DNA
gccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctnnnnnnnnnnnnngnanntcccccatctgcgtcagtcccctcaacttccgcctctacgcgggcctcgcggcagggcggtacgtcagccattctggtccgccgcgcgcatgcgtagtggagcttgcaggctggagctgagcaatcacctatgaacttgctgctaagttatgatcaaatgcaacctcacaaccttggctgagtcttgagactgaaagatttagccataatgtaaactgcctcaaattggactttgggcataaaagaaccctcctgcatgcttaccatccctcctgccctcctttaacagcctcgtaacttataattgacctcaaaaaacctcaagatttacacaatgtttctctgtaaatattgccattaaatgtaaataactttaataaaacgtttatagcagttacacagaatttcaatcctagtatatagtacctagtattataggtactataaaccctaacctcctgcatttaagtacattttgcactgcaaagtaagttatcctctattgacctcagaaaaaataaaataactggcaaatatatcattgagccaaatcttaagttgtgaatgctgcctctgctaacttatccctcccaaccaccaccatccctgctgccctcatcaaaaaacctacgggccggtctcgccgtaatgtgagta

SEQ ID NO: 82 - DNA, 555 bp, Synthetic construct
  source 1..555: mol_type = other DNA
gccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctnnnnnnnnnnttnnnnaganggcacaggnntggtggtggttgggagggataagttagcacaggcagcattcacaacttaagatttggctcatgatatatttgccagttattttattttttctgaggtcaataaaggataacttactttgtagngcaaaatgaacttaaatgcaagaggtaaaggncaatagaacctttaatacaacgtatnntttacttngnaaaaaanccaangtnacggcttaaaaccctcnctaaanaancaccnntccaaaagtcaattcagacgtacnccncccncnangggccnctaaattnntactaccaacntnctgccccctccatacanaaaan

SEQ ID NO: 83 - DNA, 557 bp, Synthetic construct
  source 1..557: mol_type = other DNA
gccatcgaggcaaaattcccccatctgcgtcagtcccctcaacttccgcctctacgcgggcctcgcggcagggcggtacgtcagccattctggtccgccgcgcgcacgctccgggcgccgggttccggcgttgtcagggtccgcggccctacgatgtgggcggtgtccaaggctgcgtagtggagcttgcaggctggagctnnnnnnnggcttttnnngnnnnnncngggatggtggtggttgggagggataagttagcagaggcagcattcacaacttaagatttggctcaatgatacctttgccagttattttattttttctgatgtcaataaaacgataacttactttgcattgcaaaatgtncatacatgcaggangttacggcttannaaacctttaataatcaccacatatatctttcaaagaaagctaaaagttctgttataaacatcactataaaaacaataatccaagaataacttcagaactacccctcccactagnaaacgctaaattgctaataacaacatattgcnctctnnaaacaaaaaaaa

(19) United States
(12) Patent Application Publication
(10) Pub. No.: US 2026/0248965 A1
(43) Pub. Date: Aug. 27, 2026

(54) DESTABILIZED ARE 3'UTRS AS THERAPEUTICS FOR CANCER

(71) Applicants: Research Foundation of the City University of New York, New York, NY (US); President and Fellows of Harvard College, Cambridge, MA (US)

(72) Inventors: Chidiebere U. Awah, Bronx, NY (US); Kevin Struhl, Weston, MA (US); Olorunseun O. Ogunwobi, Yonkers, NY (US)

(21) Appl. No.: 19/315,085

(22) Filed: Aug. 29, 2025

Related U.S. Application Data
(63) Continuation of application No. PCT/US24/18188, filed on Mar. 1, 2024.
(63) Continuation-in-part of application No. 18/176,871, filed on Mar. 1, 2023.
(60) Provisional application No. 63/315,368, filed on Mar. 1, 2022.

(51) Int. Cl. A61K 48/00; A61K 31/7105; A61K 38/17; A61P 35/04; C12N 15/85

(52) U.S. Cl. CPC A61K 48/005; A61K 31/7105; A61K 38/1709; A61P 35/04; C12N 15/85; C12N 2830/50

(57) ABSTRACT

A nucleotide that encodes for a destabilized RNA that codes for ERBB2 (erythroblastic oncogene B) or MYC oncogenes. The destabilization occurs in an adenylate-uridylate rich element (ARE) stabilizing motif. Without wishing to be bound to any particular theory, changing the stabilizing motifs of ERBB2 and MYC to destabilization motifs is believed to lead to the rapid degradation of their transcripts. Thus, in turn, renders the composition useful as a therapeutic for cancer.

### CROSS-REFERENCE TO RELATED APPLICATIONS

This application claims priority to, and is a continuation of, International Patent Application PCT/US2024/018188 (filed Mar. 1, 2024) which claims priority to U.S. patent application Ser. No. 18/176,871 (filed Mar. 1, 2023) which is a non-provisional of U.S. Patent Application 63/315,368 (filed Mar. 1, 2022), the entirety of which is incorporated herein by reference.

### STATEMENT OF FEDERALLY SPONSORED RESEARCH OR DEVELOPMENT

This invention was made with Government support under grant number U54CA221704, awarded by the National Cancer Institute, MD007599 awarded by the National Institute of Health. The government has certain rights in the invention.

### REFERENCE TO A SEQUENCE LISTING

This application contains a Sequence Listing in computer readable form with file name 03284.0325WO01.xml (created Mar. 1, 2024, 105 kb, 106,946 bytes). The computer readable form is incorporated herein by reference.

### BACKGROUND OF THE INVENTION

The subject matter disclosed herein relates to therapeutics for cancer treatment. Almost all therapies (chemo, immuno and endocrine therapies) used in treatment of cancers, including breast cancer show resistance. This represents a significant challenge to favorable clinical outcome in breast cancer as well other cancers. One of the key factors driving tumor resistance to therapies is oncogenic signals that drives rapid proliferation of cancer tissues. ERBB2 is implicated in many cancers and it is resistant to trastuzumab and other tyrosine kinase inhibitors.

The c-MYC is a basic helix loop helix (b-HLH) master transcription factor that bind the E-box sequences and is over expressed in >74% of human cancers. Breast cancers, lung cancers, brain tumors, prostate cancers, lymphomas, pediatrics cancers, pancreatic cancers, colorectal cancers, and ovarian cancers overexpress MYC. As of today, there is no direct clinically approved inhibitors of c-MYC. The lack of direct clinically approved inhibitors of c-MYC is a significant biological and clinical question that needs to be overcome for effective cancer treatment.

While attempts have been made to address these cancers, each treatment regiment comes with its own advantages and drawbacks. Additional treatment options are therefore desirable so as to provide medical practitioners with a wider range of treatment possibilities.

The discussion above is merely provided for general background information and is not intended to be used as an aid in determining the scope of the claimed subject matter.

### SUMMARY

This disclosure provides a nucleotide that encodes for a destabilized RNA that corresponds to ERBB2 (erythroblastic oncogene B) or MYC oncogenes. The destabilization occurs in an untranslated adenylate-uridylate rich element (ARE) poly U stabilizing motif of the 3′ UTR of the RNA molecule. The destabilized RNA molecule can be used to reverse transcribe a deoxyribonucleic acid (DNA) molecule, which can then be transfected into a vector that can be formulated as a pharmaceutical composition. Upon administration (e.g., to a cell, to a subject), the DNA construct transcribes the destabilized 3′UTR of mRNA molecules that outcompete the wildtype RNA molecules that encode ERBB2 or MYC protein, resulting in the rapid degradation of the transcripts of the wildtype RNA transcripts, which are outcompeted by the destabilized RNA 3′UTR transcripts. Thus, the disclosed RNA molecules, DNA molecules, vectors, and compositions can be useful in the treatment of various cancers for which dysregulation of ERBB2 and/or MYC are implicated.

In a first embodiment, a composition of matter is provided. The composition of matter comprising a primary structure of SEQ ID NO: 19.

In a second embodiment, a composition of matter is provided. The composition of matter comprising sequential sequences, ordered from 5′ to 3′, of a first restriction sequence, a first polyA sequence, a promoter sequence, a nucleotide sequence with a primary structure of SEQ ID NO: 19, a second polyA sequence and a second restriction sequence.

In a third embodiment, a composition of matter is provided. The composition of matter comprising a primary structure of SEQ ID NO 18.

This brief description of the invention is intended only to provide a brief overview of subject matter disclosed herein according to one or more illustrative embodiments, and does not serve as a guide to interpreting the claims or to define or limit the scope of the invention, which is defined only by the appended claims. This brief description is provided to introduce an illustrative selection of concepts in a simplified form that are further described below in the detailed description. This brief description is not intended to identify key features or essential features of the claimed subject matter, nor is it intended to be used as an aid in determining the scope of the claimed subject matter. The claimed subject matter is not limited to implementations that solve any or all disadvantages noted in the background.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

So that the manner in which the features of the invention can be understood, a detailed description of the invention may be had by reference to certain embodiments, some of which are illustrated in the accompanying drawings. It is to be noted, however, that the drawings illustrate only certain embodiments of this invention and are therefore not to be considered limiting of its scope, for the scope of the invention encompasses other equally effective embodiments. The drawings are not necessarily to scale, emphasis generally being placed upon illustrating the features of certain embodiments of the invention. In the drawings, like numerals are used to indicate like parts throughout the various views. Thus, for further understanding of the invention, reference can be made to the following detailed description, read in connection with the drawings in which:

FIG. 1 is a schematic diagram of one DNA sequence for treating cancer.

FIG. 2A is a RNA extract (erythroblastic oncogene B (ERBB2)) from MCF-7 cells. Stabilizing sequences are underscored.

FIG. 2B is a RNA extract from BT474 cells (ERBB2). Stabilizing sequences are underscored.

FIG. 2C is a RNA extract from T47D cells (ERBB2). Stabilizing sequences are underscored.

FIG. 3A is a RNA extract from MCF-7 cells (MYC). Stabilizing sequences are underscored.

FIG. 3B is a RNA extract from BT474 cells (MYC). Stabilizing sequences are underscored.

FIG. 3C is a RNA extract from T47D cells (MYC). Stabilizing sequences are underscored.

FIG. 3D is a RNA extract from MDA-MB231 cells (MYC). Stabilizing sequences are underscored.

FIG. 4A is a RNA extract from LNCAP cells (MYC). Stabilizing sequences are underscored.

FIG. 4B is a RNA extract from MDA Pca-2B cells (MYC). Stabilizing sequences are underscored.

FIG. 4C is a RNA extract from C4-2B cells (MYC). Stabilizing sequences are underscored.

FIG. 4D is a RNA extract from RWPE1-ex9 cells (MYC). Stabilizing sequences are underscored.

FIG. 5A is a RNA sequence showing a destabilized MYC RNA (changes relative to wildtype underscored).

FIG. 5B is a RNA sequence showing a destabilized ERBB2 RNA (changes relative to wildtype underscored).

FIG. 6A depicts a schematic of stable ERBB2 3′UTR.

FIG. 6B depicts a schematic of destabilized ERBB2 3′UTR.

FIG. 7A depicts a DNA sequence corresponding to the destabilized ERBB2 RNA. A first digestion enhancing sequence, a first restriction sequence, a first polyA sequence and a promoter sequence are sequentially disposed 5′ of the DNA sequence. A second polyA sequence, a second restriction sequence and a second digestion enhancing sequence are sequentially disposed 3′ of the DNA sequence.

FIG. 7B depicts a DNA sequence corresponding to the destabilized MYC RNA. A first digestion enhancing sequence, a first restriction sequence, a first polyA sequence and a promoter sequence are sequentially disposed 5′ of the DNA sequence. A second polyA sequence, a second restriction sequence and a second digestion enhancing sequence are sequentially disposed 3′ of the DNA sequence.

FIG. 7C depicts a DNA sequence corresponding to the destabilized ERBB2 RNA.

FIG. 7D depicts a DNA sequence corresponding to the destabilized MYC RNA.

FIG. 7E depicts a DCP1A promoter sequence and a shortened DCP1A promotor sequence.

FIG. 8 is a schematic diagram illustrating one method of incorporating a DNA sequence into a plasmid vector.

FIG. 9A shows BT474 clone 5 breast cancer cells (trastuzumab resistant HER2+ breast cancer cells) experienced severe morphology disruption after four days. Treatment was with the plasmid vector containing destabilized 3′ UTR ERBB2. Two wild type controls (BT474 clone 5 WT and BT474 clone 5 vector (empty)) are shown for comparison.

FIG. 9B shows BT474 breast cancer cells experienced significant cell death after eight days. Treatment was with the plasmid vector containing destabilized 3′ UTR ERBB2. Two wild type controls (BT474 WT and BT474 WT vector (empty)) are shown for comparison.

FIG. 10A is a graph showing cell viability of BT474 clone 5 cells after treatment with destabilized 3′ UTR ERBB2.

FIG. 10B is a Western blot showing ERBB2 and GAPDH expression after treatment of BT474 clone 5 cells with destabilized 3′ UTR ERBB2.

FIG. 10C is a bar chart that quantifies ERBB2 expression normalized against GAPDH after treatment of BT474 clone 5 cells with destabilized 3′ UTR ERBB2.

FIG. 11A is a graph showing cell viability of MDA MB231 cells after treatment with destabilized 3′ UTR MYC.

FIG. 11B is a Western blot showing MYC and GAPDH expression after treatment of MDA MB231 cells with destabilized 3′ UTR MYC.

FIG. 11C is a bar chart that quantifies MYC expression normalized against GAPDH after treatment of MDA MB231 cells with destabilized 3′ UTR MYC.

FIG. 12A is a graph showing cell viability of C4 B2 cells after treatment with destabilized 3′ UTR MYC.

FIG. 12B is a Western blot showing MYC and GAPDH expression after treatment of C4 B2 cells with destabilized 3′ UTR MYC.

FIG. 12C is a bar chart that quantifies MYC expression normalized against GAPDH after treatment of C4-2B cells with destabilized 3′ UTR MYC.

FIG. 13A is a graph showing cell viability of NCI H1975 cells after treatment with destabilized 3′ UTR ERBB2.

FIG. 13B is a Western blot showing ERBB2 and GAPDH expression after treatment of NCI H1975 cells with destabilized 3′ UTR ERBB2.

FIG. 13C is a bar chart that quantifies ERBB2 expression normalized against GAPDH after treatment of NCI H1975 cells with destabilized 3′ UTR ERBB2.

FIG. 14A is a bar graph showing tumor volume (BT474 cells) in mice after treatment with various desARE ERBB2 clones.

FIG. 14B is a graph of the probability of survival of the mice of FIG. 14A.

FIG. 15A is a bar graph showing tumor volume (NCI H1975 cells) in mice after treatment with various desARE ERBB2 clones.

FIG. 15B is a bar graph showing number of liver metastasis sites found for the mice of FIG. 15A.

FIG. 16A is a bar graph showing number of tumor metastatic sites found in mice after implantation with MDA-MB231 cells.

FIG. 16B is a graph of the probability of survival of the mice of FIG. 16A.

FIG. 17 are Immunofluorescence pictures showING ERBB2 expression (stained in red) and nuclei (DAPI-blue) on wildtype NCI-H1975, vector, and on NCI-H1975 containing constructs desARE3′UTR ERBB2-1, -2, -3, -4, and -30 and wildtype-treated trastuzumab NCI-H1975 (n=2). Magnification is 20×.

FIG. 18 depict a bar chart showing quantification of the ERBB2 fluorescence expression on NCI-H1975 wildtype cells, vector and cells containing constructs desARE3′UTR ERBB2-1, 3, 30 (column 3, 5 7), desARE3′UTR ERBB2-2,4 and wild type trastuzumab treated NCI-H1975 cells (n=2). T-test***=0.003, WT, vector vs desARE3′UTRERBB2-1,3,30.

FIG. 19A is a bar chart showing quantification of the cleaved caspase 3 fluorescence intensity on NCI H1975 wildtype cells, vector and cells containing constructs desARE3′UTR ERBB2-1, 3, 30 (n=2). T-test, ****=0.00025 WT, vector vs desARE3′UTRERBB2-3,30.

FIG. 19B is bar charting show quantification of the caspase 9 fluorescence intensity on NCIH1975 wildtype cells, vector and cells containing constructs desARE3′UTR ERBB2-1, 3, 30 (n=2). T-test, *p=0.05 WT, vector vs desARE3′UTR ERBB2-1, ****p=0.00025 WT, vector vs desARE3′UTRERBB2-3,30 ***p=0.0022 WT, vector vs desARE3′UTR ERBB2-30.

FIG. 20 is a bar chart showing the quantification of wound opening at 0 hr, 22 and 48 hrs in NCI H1975 wildtype, vector, desARE3′UTR1,3,30. At 22 hr WT, vector vs desARE3′UTRERBB2-3,30 show significant wound opening t-test ***p=0.0014 and at 48 hr t-test ****p=0.0004 WT, vector vs desARE3′UTRERBB2-3, ***p=0.003 WT, vector vs desARE3′UTRERBB2-30. All experiment n=2.

FIG. 21 depicts immunofluorescence images show ERBB2 expression (stained in red) and nuclei (DAPI-blue) on wildtype BT474 clone 5, vector, and on BT474 clone 5 containing constructs desARE3′UTR ERBB2-1, -2, -3, -4, and -30 and BT474 clone 5 WT trastuzumab treated (n=2). Magnification is 20×.

FIG. 22 is a bar chart showing quantification of the ERBB2 fluorescence expression on BT474 clone 5 wildtype cells, vector and cells containing constructs desARE3′UTR ERBB2-1,2, 3, 4, 30 and trastuzumab treated cells (columns, 3-7) (n=2). T-test, ns=WT, vector vs desARE3′UTRERBB2-1, **=0.037 WT, vector vs desARE3′UTRERBB2-2, **=0.0178 WT, vector vs desARE3′UTRERBB2-3, **=0.0198 WT, vector vs desARE3′UTRERBB2-4, **=0.0178 WT, vector vs desARE3′UTRERBB2-30.

FIG. 23 is a bar chart showing quantification of the ERBB2 western blot expression normalized against GAPDH on BT474 clone 5 wildtype cells, vector and cells containing constructs desARE3′UTR ERBB2-1, 2, 3, 4 and 30 (columns 3-7) (n=3). T-test****=0.00012 WT, vector vs desARE3′UTRERBB2-3,30.

FIG. 24 shows immunofluorescence images show ERBB2 expression (stained in green) and nuclei (DAPI-blue) on wildtype BT474, vector, and on BT474 containing constructs desARE3′UTR ERBB2-1, -2, -3, -4, -30, and BT474 wildtype trastuzumab-treated cells (n=2). Magnification is 20×.

FIG. 25 is a bar chart showing quantification of the ERBB2 fluorescence expression on BT474 wildtype cells, vector and cells containing constructs desARE3′UTR ERBB2-1,2, 3, 4 (red bars) (n=2). T-test, **=0.0331 WT, vector vs desARE3′UTRERBB2-1, **=0.0040 WT, vector vs desARE3′UTRERBB2-2, **=0.0249 desARE3′UTRERBB2-3, **=0.0272 WT, vector vs desARE3′UTRERBB2-4, **=0.0377 WT, vector vs desARE3′UTRERBB2-30, **=0.0406 BT474 trastuzumab treated cells.

FIG. 26 is a Western blot showing ERBB2 and GAPDH expression across the wildtype BT474, vector, and on BT474 containing constructs desARE3′UTR ERBB2-1, -2, -3, and -4 (n=2).

FIG. 27 is a bar chart showing quantification of the ERBB2 western blot expression normalized against GAPDH on BT474 wildtype cells, vector and cells containing constructs desARE3′UTR ERBB2-1, 2, 3, and 4 (columns 3-6) (n=2). T-test, ***=0.0036 desARE3′UTRERBB2-1 and 3.

FIG. 28 is a bar chart showing active caspase 3/7 luminescence on BT474 wildtype cells, vector and cells containing constructs desARE3′UTR ERBB2-1, 2, 3 and 4 (n=2). T-test, *****<0.00001 WT, vector vs desARE3′UTRERBB2-1,3. ***=0.0022 WT, vector vs desARE3′UTRERBB2-2,4.

FIG. 29 is a bar chart showing cell viability of the BT474 wildtype cells, vector, and BT474 cells containing constructs desARE3′UTR ERBB2-1, -2, -3, and -4 (n=4). Two-tailed T-test (***p-value=0.0025 WT, vector vs. desARE3′UTR ERBB2-1, ****p-value=0.00029515 WT, vector vs. desARE3′UTR ERBB2-2, ***p-value=0.0045 WT, vector vs. desARE3′UTR ERBB2-3, ****p-value=0.000102 WT, vector, desARE3′UTR ERBB2-4, ***p-value=0.00120 WT, vector, vs. desARE3′UTR ERBB2-30, **p-value=0.027 WT, vector vs. BT474 WT trastuzumab treated.

FIG. 30 depicts a bar chart showing quantification of the ERBB2 expression normalized against GAPDH by qPCR on BT474 wildtype cells, vector and cells containing constructs desARE3′UTR ERBB2-1, -2, -3, and -30 (n=2). Two-tailed T-test (****p-value=0.0003 WT, vector vs. desARE3′UTR ERBB2-1, ***p-value=0.0021 WT, vector vs. desARE3′UTR ERBB2-2,3, **p-value=0.013 WT, vector vs. desARE3′UTR ERBB2-30).

FIG. 31 is a bar chart showing quantification of the ERBB2 fluorescence intensity on NCI H2030 wildtype cells, vector and cells containing constructs desARE3′UTR ERBB2-1, 3, 30 and trastuzumab treated cells (n=2). T-test, ****=0.0004 WT, vector vs desARE3′UTRERBB2-1,3,30.

FIG. 32 is a bar chart showing quantification of the ERBB2 expression normalized against GAPDH by qPCR on NCI H2030 wildtype cells, vector, and cells containing constructs desARE3′UTR ERBB2-1, -2, -3, -4, and -30 (n=2). Two-tailed T-test (*p-value=0.027 WT, vector vs. desARE3′UTR ERBB2-1, ***p-value=0.0003 WT, vector vs. desARE3′UTR ERBB2-3, ***p-value=0.0001 WT, vector vs. desARE3′UTR ERBB2-30.

FIG. 33 is a bar chart showing cell viability of the NCI H2030 wildtype cells, vector, and cells containing constructs desARE3′UTR ERBB2-1, -2, -3, -4, and -30 (n=2). Two-tailed T-test (**p-value=0.0080 WT, vector vs. desARE3′UTR ERBB2-3, ***P=0.0024 WT, vector vs. desARE3′UTR ERBB2-30).

FIG. 34 is a bar chart showing quantification of the ERBB2 expression normalized against GAPDH by qPCR on HCT116 wildtype cells, vector and HCT116 cells containing constructs desARE3′UTR ERBB2-1, 3 and 30 (n=2). T-test WT, vector vs (****=0.0003, desARE3′UTRERBB2-3 and 30).

FIG. 35 shows a volcano plot with genes downregulated (blue), and genes upregulated (red) in desARE3′UTR ERBB2-3 and -30 compared to wildtype and vector controls.

FIG. 36 depicts horizontal bar charts show the Gene Ontology (GO) theme upregulated in desARE3′UTR ERBB2-3 and -30 compared to wildtype and vector controls.

FIG. 37 is a horizontal bar chart showing UPF3B expression in wildtype NCI-H1975, vector, and desARE3′UTR ERBB2-3 and -30.

FIG. 38 is a horizontal bar chart shows Gene Ontology (GO) downregulated in desARE3′UTR ERBB2-3 and -30 compared to wildtype and vector controls.

FIG. 39 depicts a chart showing time-dependent chase of both destabilized construct transcript and WT ERBB2 mRNA in the same cells.

FIG. 40 are immunofluorescence images show ERBB2 expression (stained in red) and nuclei (DAPI-blue) on wildtype NCI H2030 cells, vector, and on NCI-H2030 containing constructs desARE3′UTR ERBB2-1, -2, -3, -4, and -30, and trastuzumab-treated cells (n=2). Magnification is 20×.

FIG. 41A is a bar chart showing mRNA expression of DCP1A and ERBB2 normalized against GAPDH in BT474 clone 5 WT, vector, desARE3′UTR ERBB2-1, -2, -3, and -4 and 30 (n=2). T-test (****p=0.0001 ERBB2 vs. DCP1A BT474 clone 5 WT), (***p=0.0012 ERBB2 vs. DCP1A vector), (*****p=0.00001 ERBB2 vs. DCP1A desARE3′UTR ERBB2-1), (*****p=0.00003 ERBB2 vs. DCP1A desARE3′UTR ERBB2-2), (*****p=0.000025 ERBB2 vs. DCP1A desARE3′UTR ERBB2-3), (****p=0.00013 ERBB2 vs. DCP1A desARE3′UTR ERBB2-4), (*****p=0.000016 ERBB2 vs. DCP1A desARE3′UTR ERBB2-30).

FIG. 41B is a bar chart showing the RNA Seq expression of DCP1A in NCI-H1975 WT, vector, desARE3′UTR ERBB2-3 and -30. T-test (****p=0.0128 ERBB2 vs. DCP1A NCI-H1975 WT), (**p=0.011 ERBB2 vs. DCP1A vector), (p=ns ERBB2 vs. DCP1A desARE3′UTR ERBB2-3), (**p=0.001 ERBB2 vs. DCP1A desARE3′UTR ERBB2-30).

FIG. 41C is a bar chart showing the mRNA expression of XRN1 normalized against GAPDH in BT474 clone 5 WT, vector vs. desARE3′UTR ERBB2-1, -2, -3, -4, and -30, **p-value=0.002. Two-tailed t-test (n=2).

FIG. 41D is a bar chart showing the mRNA expression of PARN normalized against GAPDH in BT474 clone 5 WT, vector vs. desARE3′UTR ERBB2-1, -2, -3, -4, and -30, p-value=ns, two tailed t-test (n=2).

FIG. 41E is a bar chart showing the mRNA expression of CNOT1 normalized against GAPDH in BT474 clone 5 WT, vector vs. desARE3′UTR ERBB2-1, -2, -3, -4, and -30, **p-value=0.001, two tailed t-test (n=2).

FIG. 41F is a bar chart showing the mRNA expression of XRN1 normalized against GAPDH in NCI-H1975 WT, vector vs. desARE3′UTR ERBB2-1, -2, -3, -4, and -30 and trastuzumab-treated cells **p-value=0.004, two tailed t-test (n=2).

FIG. 41G is a bar chart showing the mRNA expression of PARN normalized against GAPDH in NCI-H1975 WT, vector vs. desARE3′UTR ERBB2-1, -2, -3, -4, and -30 and trastuzumab-treated cells, p-value=ns, two-tailed t-test (n=2).

FIG. 41H is a bar chart showing the mRNA expression of CNOT1 normalized against GAPDH in NCI-H1975 WT, vector vs. desARE3′UTR ERBB2-1, -2, -3, -4, and -30 and trastuzumab-treated cells, p-value=0.0002, two tailed t-test (n=2).

FIG. 41I is a Western blot showing CNOT1 and GAPDH protein expression in NCI-H1975 WT, vector, desARE3′UTR ERBB2-1, -3, and -30.

FIG. 41J is a bar chart showing quantification of CNOT1 protein expression normalized against GAPDH in NCI-H1975 WT, vector, desARE3′UTR ERBB2-1, -3, and -30.

FIG. 42 is a dot blots diagram showing the phospho-kinase array pattern of the BT474 clone 5 wildtype, BT474 clone 5 vector, BT474 clone 5 desARE3′UTR ERBB2-3, and BT474 clone 5 desARE3′UTR c-MYC 2-3. The dot marked in the box (column 3) shows unique kinases downregulated only in BT474 clone 5 desARE3′UTR ERBB2-3 and the dot marked in boxes (column 4) shows unique kinases downregulated only in BT474 clone 5 desARE3′UTR c-MYC 2-3. Each kinase is spotted twice.

FIG. 43 is a bar chart showing the intensity of the phospho-kinase array dot blot for the BT474 clone 5 wildtype, BT474 clone 5 vector, BT474 clone 5 desARE3′UTR ERBB2-3, and BT474 clone 5 desARE3′UTR c-MYC 2-3.

FIG. 44 is a bar chart showing YES1 pT60 and WNK1 pT60 kinases specifically downregulated in BT474 clone 5 desARE3′UTR ERBB2-3. Controls BT474 clone 5 wildtype, BT474 clone 5 vector, and BT474 clone 5 desARE3′UTR c-MYC 2-3. T-test (***p-value=0.00413, WT, vector vs. desARE3′UTR ERBB2-3 for YES and WNK1).

FIG. 45 is a Venn diagram showing kinases downregulated in destabilized desARE3′UTR ERBB2-3 and -30 NCI-H1975 (p=0.02) left (from RNA Seq), and in destabilized desARE3′UTR ERBB2-3 BT474 clone 5 (right-kinase array) and the shared kinases found downregulated in destabilized ERBB2 in both resistant cell lines WNK1 and YES1.

FIG. 46A is a Western blot showing YES1 and GAPDH expression across the NCI-H1975 wildtype cells, vector, cells containing constructs desARE3′UTR ERBB2-1, -3, and -30, and wildtype trastuzumab-treated NCI-H1975 cells (n=2).

FIG. 46B is a bar chart showing quantification of the YES1 expression normalized against GAPDH on NCI-H1975 wildtype cells, vector, and cells containing constructs desARE3′UTR ERBB2-1, -3, and -30 (n=2). T-test (***p-value=0.0021, WT, vector vs. desARE3′UTR ERBB2-1, -3, and -30).

FIG. 47 is a Western blot showing WNK1 and GAPDH expression across the NCI-H1975 wildtype cells, vector, cells containing constructs desARE3′UTR ERBB2-1, -3, and -30 and wildtype trastuzumab-treated NCI-H1975 cells.

FIG. 48 is a Western blot showing YES1 and GAPDH expression across the NCI-H1975 destabilized with constructs desARE3′UTR ERBB2-3 and -30 (lanes 1 and 3) and over expressed with YES1 in lanes 2 and 4.

FIG. 49 is a bar chart showing the wound healing closing of NCI-H1975 wildtype, vector, desARE3′UTR ERBB2-1, -3, -30, and desARE3′UTR ERBB2-30,30 overexpressed with YES1 from 0 h to 72 hrs. At 24 hrs ***p=0.00025 WT, vector vs. desARE3′UTR ERBB2-3,30. At 48 hrs ****p=0.000018 WT, vector vs. desARE3′UTR ERBB2-3 and at 72 hrs ***p=0.0003 WT, vector vs. desARE3′UTR ERBB2-3 and **p=0.002 WT, vector vs. desARE3′UTR ERBB2-30.

FIG. 50 depict caspase 7 expression in vivo in tumors untreated NCI-H1975 wildtype, vector, desARE3′UTR ERBB2-1, -3, and -30 and quantified in bar charts. Magnification is 60×.

FIG. 51 is a heat map showing the ERBB2 gene expression profile in wildtype NCI-H1975, vector, desARE3′UTR ERBB2-3 and -30 compared to the BEAS-2B normal human lung epithelial cells.

FIG. 52 is a heat map showing the ERBB2 gene expression profile in wildtype NCI-H1975, vector, desARE3′UTR ERBB2-3 and -30 compared to the BEAS-2B normal human lung epithelial cell line and normal lungs from a Chinese woman non-smoker, normal lung from a Caucasian male, three normal female lungs from ENCODE, and three normal male lungs from ENCODE.

FIG. 53 is a Genome browser view of whole-genome de novo variant assembly between NCI-H1975 wildtype, vector, and desARE3′UTR ERBB2-3.

FIG. 54 is a schematic representation of the genomic alteration and their quantification in wildtype NCI-H1975, vector, desARE3′UTR ERBB2-3. Dark blue color-CNV segment gain), red-CNV segment loss), light blue-aneuploidy gain), and light orange-aneuploidy loss (n=2).

FIG. 55 depicts an outline of animal experiment. A total of 25 NSG female mice were implanted with 5 million NCI-H1975 as a flank xenograft, and after 35 days post implantation, huge tumor engrafts were present. Day 36 post implantation, the tumors were randomized for equal size distribution. Treatment groups were administered 20 μg IP 12 hrly of engineered constructs for 9 days with daily weight and tumor size measurement. Day 46 post implantation, control group tumor sizes have grown so enormous that mice were euthanized, and full blood count, electrolyte, necropsy, and histopathological analysis were performed by an expert pathologist.

FIG. 56 depicts a box plot showing tumor volumes in NCI-H1975 WT, vector, desARE3′UTR ERBB2-1, desARE3′UTR ERBB2-3, and desARE3′UTR ERBB2-30. Two-tailed T-test, p-value ns=WT vs. vector, WT vs. desARE3′UTR ERBB2-1, -3, and 30 (***0.0026, ***0.0077, and ***0.004), Vector vs. desARE3′UTR ERBB2-1, -3, and -30 (**0.0251, ***0.0088, and ***0.0031).

FIG. 57 is a line plot showing daily tumor growth by comparing NCI-H1975 WT, vector and desARE3′UTR ERBB2-1, desARE3′UTR ERBB2-3, and desARE3′UTR ERBB2-30. Two-tailed test, p-value ns=WT vs. vector, WT vs. desARE3′UTR ERBB2-1, -3, and -30 (***0.0026, ***0.0077, and ***0.004), vector vs. desARE3′UTR ERBB2-1, -3, and -30 (**0.0251, ***0.0088, and ***0.0031).

FIG. 58 is a bar chart showing weight of mice bearing tumor NCI-H1975 (WT), vector, and mice bearing tumors treated with desARE3′UTR ERBB2-1, desARE3′UTR ERBB2-3, and desARE3′UTR ERBB2-30.

FIG. 59 is a bar charts showing the red blood cell count of mice bearing tumor NCI-H1975 WT, vector, and mice bearing tumors treated with desARE3′UTR ERBB2-1, desARE3′UTR ERBB2-3, and desARE3′UTR ERBB2-30.

FIG. 60 is a bar chart showing the hemoglobin count of mice bearing tumor NCI-H1975 WT, vector, and mice bearing tumors treated with desARE3′UTR ERBB2-1, desARE3′UTR ERBB2-3, and desARE3′UTR ERBB2-30.

FIG. 61 is a bar chart showing the hematocrit level of mice bearing tumor NCI-H1975 WT, vector, and mice bearing tumors treated with desARE3′UTR ERBB2-1, desARE3′UTR ERBB2-3, and desARE3′UTR ERBB2-30.

FIG. 62 depicts a bar chart showing the white blood cell count of mice bearing tumor NCI-H1975 WT, vector and mice bearing tumors treated with desARE3′UTR ERBB2-1, desARE3′UTR ERBB2-3, and desARE3′UTR ERBB2-30 **p=0.0065 WT, vector vs. desARE3′UTR ERBB2-1, -3, and 30.

FIG. 63 is a bar chart showing the neutrophil cell count of mice bearing tumor NCI-H1975 WT, vector, and mice treated with desARE3′UTR ERBB2-1, desARE3′UTR ERBB2-3, and desARE3′UTR ERBB2-30. ***p=0.0032 WT, vector vs. desARE3′UTR ERBB2-1, 6/3 and 30.

FIG. 64 is a bar chart showing the lymphocyte cell count of mice bearing tumor NCI-H1975 WT, vector, and mice treated with desARE3′UTR ERBB2-1, desARE3′UTR ERBB2-3, and desARE3′UTR ERBB2-30. **p=0.0002 desARE3′UTR ERBB2-1.30. **p=0.0021 desARE3′UTR ERBB2-3.

FIG. 65 shows an H & E stain of the cardiac tissue of the WT (magnification 60×).

FIG. 66 shows an H& E stain of the cardiac tissue treated with the vector (magnification 60×).

FIG. 67 shows an H& E stain of the cardiac tissue treated with the desARE3′UTR ERBB2-30 (magnification 60×).

FIG. 68A is a bar chart showing the tumor volume of the control groups (IO-nanocage and IO-nanocage+Vector) and the treated groups the desARE3′UTR ERBB2-1,3 and 30 treated groups. Two tailed T-test (IO-nanocage only versus IO-nanocage+Vector p=ns; *p=0.01 Nanocage only vs desARE3′UTRERBB2-1; ****p=0.0003 IO-nanocage only vsdesARE3′UTRERBB2-3;****p=0.0003 Nanocage only vs desARE3′UTRERBB2-30), (***p=0.001 IO-nanocage+Vector vs desARE3′UTRERBB2-1; ****p=0.00033 IO-nanocage+Vector vs desARE3′UTRERBB2-3; ****p=0.00032 IO-nanocage+Vector vs desARE3′UTRERBB2-30.

FIG. 68B, FIG. 68C and FIG. 68D show images of H&E staining of the 3 tumors from the control and the treatment groups.

FIG. 68E, FIG. 68F and FIG. 68G show images of ERBB2 IHC staining (brown) and DAPI (nuclei-blue staining) of the 3 tumors each from the control and treatment groups.

FIG. 68H shows images of IO-nanocages (Prussian blue) in tumor tissues of the control and treated groups.

FIG. 68I is a bar chart showing percentage of malignant pleomorphic cells per field of the control and treated groups. Two tailed T-test (p=ns IO-nanocage only versus IO-nanocage+vector, *p=0.01 IO-nanocage only versus desARE3′UTRERBB2-1, *p= 0.003 IO-nanocage only vs desARE3′UTRERBB2-3, **p=0.00022 IO-nanocage only versus desARE3′UTRERBB2-30).

FIG. 68J is a bar chart showing the percentage of complete tumor lysis in the control and treated groups. Two tailed T-test (*p=0.01 IO-nanocage only vs desARE3′UTRERBB2-3, ****p=0.000014 Nanocage only versus desARE3′UTRERBB2-30).

FIG. 68K is a bar chart showing the percentage of partial tumor lysis in the control and the treated groups. Two tailed T-test (****p=0.00003 Nanocage only versus desARE3′UTRERBB2-1, *p=0.01 IO-nanocage only vs desARE3′UTRERBB2-3 &30).

FIG. 68L is a bar chart showing the percentage of the ERBB2 IHC negative staining in the control and treatment groups. Two tailed T-test (*p=0.02 IO-nanocage only vs desARE3′UTRERBB2-1, *p=0.013 IO-nanocage only versus desARE3′UTRERBB2-3, ****p=0.00045 Nanocage only vs desARE3′UTRERBB2-30).

FIG. 69A is a bar chart showing the number the livers with metastasis in the controls (IO-nanocage only, IO-nanocage+Vector) and the treated groups desARE3′UTRERBB2-1+nanocage, desARE3′UTRERBB2-3+IO-nanocage, desARE3′UTRERBB2-30+IO-nanocage. Two tailed T-test (p=ns IO-nanocage only vs IO-nanocage+Vector, p=ns IO-nanocage only vs desARE3′UTRERBB2-1, **p=0.002 IO-nanocage only versus desARE3′UTRERBB2-3, ***p=0.00013 IO-nanocage only vs desARE3′UTRERBB2-30).

FIG. 69B, FIG. 69C and FIG. 69D show images of H&E staining of 3 livers from the control and treated groups.

FIG. 69E and FIG. 69F show images of livers from the control and treated groups stained with Prussian blue stain for IO-nanocage detection and identification.

FIG. 69G is a bar chart showing the number of metastatic liver nodules in the control and treated groups. Two tailed T-test (p=ns Nanocage only vs Nanocage+Vector, p=ns Nanocage only vs desARE3′UTRERBB2-1, **p=0.003 Nanocage only vs desARE3′UTRERBB2-3, **p=0.00025 Nanocage only vs desARE3′UTRERBB2-30).

FIG. 69H is a bar chart showing percentages of therapeutic inhibition of liver metastasis in the control and treated groups. Two tailed T-test (p=ns IO-nanocage only versus IO-nanocage+Vector, p=ns IO-nanocage only vs desARE3′UTRERBB2-1, p=0.0013 IO-nanocage only versus desARE3′UTRERBB2-3, ***p=0.00018 IO-nanocage only versus desARE3′UTRERBB2-30).

FIG. 70A is a bar chart showing percentages of metastatic lesions within the lung parenchyma. Two tailed T-test (p=ns IO-nanocage only versus IO-nanocage+Vector, **p=0.0025 IO-nanocage only versus desARE3′UTRERBB2-1, ***p=0.00042 IO-nanocage only versus desARE3′UTRERBB2-30, and **p=0.000013 IO-nanocage only versus desARE3′UTRERBB2-30).

FIG. 70B, FIG. 70C and FIG. 70D shows images of H&E staining of 3 lungs from each experimental group, the controls, and treated groups.

FIG. 70E is a bar chart showing percentages of complete tumor lysis within the lung parenchyma. Two tailed T-test (p=ns IO-nanocage only versus IO-nanocage+Vector, **p=0.002 IO-nanocage only versus desARE3′UTRERBB2-1, **p=0.00012 IO-nanocage only versus desARE3′UTRERBB2-3, **p=0.000035 IO-nanocage only versus desARE3′UTRERBB2-30).

FIG. 71A is a PCR gel image showing the engineered destabilized 3′UTRERBB2-1, 3 and 30 in the tumors and spleens from the treated animals.

FIG. 71B is a PCR gel image showing the engineered destabilized 3′UTRERBB2-30 in the two kidneys, two lungs and two livers.

FIG. 71C is a PCR gel image showing the engineered destabilized 3′UTRERBB2-1 in the brain, lung, kidneys and livers.

FIG. 71D is a chart showing the qPCR amplification cycle of the engineered destabilized 3′UTRERBB2 constructs from tumor genomic DNA in serial dilutions of 1, 1:10 and 1:1000.

FIG. 72A is a schema depicting the in-vivo experiment which shows 15 female NSG mice were implanted with 5 million BT474clone 5 cells into their mammary fat pad, at 2 weeks tumor engrafted. Animals were randomized on day 15 and treatment with the vector, desARE3′UTRERBB2-3, and 30 at 20 μg/0.1 ml was given IP 2×/week till 57 days.

FIG. 72B shows a bar chart shows the tumor volume in the vector, desARE3′UTRERBB2-3 and 30 treated animals. Two tailed T-test (****p=3.73e-06 Vector vs desARE3′UTRERBB2-3, ****p=1.08e-05 Vector vs desARE3′UTRERBB2-30).

FIG. 72C shows a Kaplan Meier survival curve in which desARE3′UTRERBB2-3 treated mice achieved exceptional survivors. Log Rank Mantel test (**p=0.0095 Vector vs deARE3′UTRERBB2-3, p=ns Vector vs desARE3′UTRERBB2-30).

FIG. 73A is a schema depicting an in-vivo experiment which shows 15 female NSG mice were implanted with 5 million cells into their mammary fat pad, at 2 weeks tumor engrafted. Animals were randomized on day 15 and treatment with the vector+nanocage, desARE3′UTRERBB2-3+nanocage, and desARe3′UTRERBB2-30+nanocage at 20 μg/0.1 ml was given IP 2×/week till 57 days.

FIG. 73B is a bar chart showing the tumor volume in the vector+nanocage, desARE3′UTRERBB2-3+nanocage and desARE3′UTRERBB2-30+nanocage treated animals. Two tailed T-test (****p=4.4e-06 Vector+nanocage vs desARE3′UTRERBB2- 3+nanocage, ****p=5.6e-06 Vector+nanocage vs desARE3′UTRERBB2-30+nanocage).

FIG. 73C is a Kaplan Meier survival curve showing that desARE3′UTRERBB2-30+nanocage treated mice achieved significant survivors. Log Rank Mantel test (**p=0.00492 Vector+nanocage vs deARE3′UTRERBB2-30+nanocage, p=ns Vector+nanocage vs desARE3′UTRERBB2-30+nanocage).

FIG. 74A and FIG. 74B are H& E images of two tumors each from vector, desARE3′UTRERBB2-3 and 30 treated animals, with desARE3′UTRERBB2-3 and 30 showing complete tumor lysis.

FIG. 74C is a bar chart showing the percentage quantification of complete tumor lysis in the treated animals compared to the controls. Two tailed t-test (****p=0.0001 vector vs desARE3′UTRERBB2-3,30).

FIG. 74D is a bar chart showing the percentage quantification of hyperchromatic cells in the treated tumors compared with the control vector. Two tailed t-test (**p=0.001 vector vs desARE3′UTRERBB2-3,30).

FIG. 74E is a bar chart showing the percentage quantification of residual tumor cells in the vector treated compared to the constructs treated animals. Two tailed t-test (***p=0.00025 vector vs desARE3′UTRERBB2-3,30).

FIG. 74F and FIG. 74G are H&E images of two tumors each from the vector+nanocage, desARE3′UTRERBB2-3+nanocage and desARE3′UTRERBB2-30+nanocage.

FIG. 74H is a bar chart showing the percentage quantification of complete tumor lysis in the desARE3′UTRERBB2-3 and 30+nanocage treated compared to the controls. Two tailed t-test (****p=0.00045 vector+nanocage vs desARE3′UTRERBB2-3,30+nanocage).

FIG. 74I is a bar chart showing the percentage of the hyperchromatic cells in the controls compared to the desARE3′UTRERBB2-3 and 30+nanocage treated tumors.

FIG. 74J is a bar chart showing the percentage quantification of residual tumor cells in the vector+nanocage treated compared to the constructs nanocage treated animals. Two tailed t-test (****p=0.00018 vector+nanocage vs desARE3′UTRERBB2-3,30+nanocage).

FIG. 74K and FIG. 74L are IHC images showing the ERBB2 stain in two tumors each from the vector+nanocage, the desARE3′UTRERBB2-3+nanocage and desARE3′UTRERBB2-30+nanocage.

FIG. 74M is a bar chart showing the percentage quantification of ERBB2 inhibition in the vector+nanocage, the desARE3′UTRERBB2-3+nanocage and the desARE3′UTRERBB2-30+nanocage treated tumors. Two tailed t-test (****p=0.000032, vector+nanocage versus desARE3′UTRERBb2-3,30+nanocage).

FIG. 74N is an IHC image showing YES1 stain in tumors from the vector+nanocage, the desARE3′UTRERBB2-3+nanocage and the desARE3′UTRERBB2-30+nanocage.

FIG. 74O is a bar chart showing the percentage quantification of YES1 inhibition in the vector+nanocage, the desARE3′UTRERBB2-3+nanocage and the desARE3′UTRERBB2-30+nanocage treated tumors. Two tailed t-test (****p=000027 vector+nanocage vs desARE3′UTRERBB2-3, 30+nanocage).

FIG. 74P is an IHC image showing WNK1 stain in tumors from the vector+nanocage, the desARE3′UTRERBB2-3+nanocage and the desARE3′UTRERBB2-30+nanocage.

FIG. 74Q is a bar chart showing the percentage quantification of WNK1 inhibition in the vector+nanocage, the desARE3′UTRERBB2-3+nanocage and the desARE3′UTRERBB2-30+nanocage treated tumors. Two tailed t-test (****p=0.00031 vector+nanocage vs desARE3′UTRERBB2-3,30+nanocage).

FIG. 75A is a chart showing the daily tumor volume measurement of the tumor bearing mice treated with the vector, desARE3′UTERBB2-3, and desARE3′UTRERBB2-30. Two tail test (****p=3.73e-06 Vector vs desARE3′UTRERBB2-3, ****p=1.08e-05 Vector vs desARE3′UTRERBB2-30).

FIG. 75B is a chart showing the daily tumor volume measurement of the tumor bearing mice treated with the vector+nanocage, desARE3′UTERBB2-3+nanocage, and desARE3′UTRERBB2-30+nanocage. Two tailed t-test (****p=3.73e-06 Vector vs desARE3′UTRERBB2-3, ****p=1.08e-05 Vector vs desARE3′UTRERBB2-30).

FIG. 75C is a chart showing the daily weight measurement of the tumor bearing mice treated with the vector, desARE3′UTRERBB2-3 and the desARE3′UTRERBB2-30.

FIG. 75D is a chart showing the daily weight measurement of the tumor bearing mice treated with the vector+nanocage, desARE3′UTRERBB2-3+nanocage and the desARE3′UTERBB2-30+nanocage.

FIG. 76A is a bar chart showing the quantification of the red blood cell (RBC) count in the normal healthy reference compared to the exceptional survivor mice treated with the desARE3′UTRERBB2-3. Two tailed t-test (*p=0.025 control vs treatment).

FIG. 76B is a bar chart showing the quantification of the hematocrit (HCT) in the normal healthy reference compared to the exceptional survivor mice treated with the desARE3′UTRERBB2-3. Two tailed t-test (*p=0.01 control vs treatment).

FIG. 76C is a bar chart showing the quantification of the hemogloblin (Hb) in the normal healthy reference compared to the exceptional survivor mice treated with the desARE3′UTRERBB2-3. Two tailed t-test (p=ns, control vs treatment).

FIG. 76D is a chart showing the quantification of the mean corpuscular volume (MCV) in the normal healthy reference compared to the exceptional survivor mice treated with the desARE3′UTRERBB2-3.

FIG. 76E is a bar chart showing the quantification of the mean corpuscular hemoglobin count (MCHC) in the normal healthy reference compared to the exceptional survivor mice treated with the desARE3′UTRERBB2-3.

FIG. 76F is a bar chart showing the quantification of the mean corpuscular hemoglobin (MCH) in the normal healthy reference compared to the exceptional survivor mice treated with the desARE3′UTRERBB2-3.

FIG. 76G is bar chart showing the quantification of the white blood cell count in the normal healthy reference compared to the exceptional survivor mice treated with the desARE3′UTRERBB2-3. Two tailed t-test (***p=0.00024, control versus treatment).

FIG. 76H is a bar chart showing the quantification of the neutrophil count in the normal healthy reference compared to the exceptional survivor mice treated with the desARE3′UTRERBB2-3. Two tailed t-test (***p=0.00014 control versus treatment).

FIG. 76I is a bar chart showing the quantification of the lymphocyte count in the normal healthy reference compared to the exceptional survivor mice treated with the desARE3′UTRERBB2-3. Two tailed t-test (***p=0.00040 control versus treatment).

FIG. 76J is a bar chart showing the quantification of the monocyte count in the normal healthy reference compared to the exceptional survivor mice treated with the desARE3′UTRERBB2-3. Two tailed t-test (***p=0.00054 control versus treatment).

FIG. 77A is bar chart showing the quantification of the total bilirubin in the normal healthy reference compared to the exceptional survivor mice treated with the desARE3′UTRERBB2-3.

FIG. 77B is a bar chart showing the quantification of the total protein in the normal healthy reference compared to the exceptional survivor mice treated with the desARE3′UTRERBB2-3.

FIG. 77C is a bar chart showing the quantification of the globulin in the normal healthy reference compared to the exceptional survivor mice treated with the desARE3′UTRERBB2-3.

FIG. 77D is a bar chart showing the quantification of the albumin in the normal healthy reference compared to the exceptional survivor mice treated with the desARE3′UTRERBB2-3.

FIG. 77E is a bar chart showing the quantification of the triglycerides in the normal healthy reference compared to the exceptional survivor mice treated with the desARE3′UTRERBB2-3.

FIG. 77F is a bar chart showing the quantification of the glucose in the normal healthy reference compared to the exceptional survivor mice treated with the desARE3′UTRERBB2-3. Two tailed t-test (***p=0.00019 control versus treatment).

FIG. 77G is a bar chart showing the quantification of the cholesterol in the normal healthy reference compared to the exceptional survivor mice treated with the desARE3′UTRERBB2-3. Two tailed t-test (*p=0.035 control versus treatment).

FIG. 78A is a bar chart showing the quantification of the phosphate in the normal healthy reference compared to the exceptional survivor mice treated with the desARE3′UTRERBB2-3.

FIG. 78B is a bar chart showing the quantification of the calcium in the normal healthy reference compared to the exceptional survivor mice treated with the desARE3′UTRERBB2-3.

FIG. 78C is a bar chart showing the quantification of the potassium in the normal healthy reference compared to the exceptional survivor mice treated with the desARE3′UTRERBB2-3.

FIG. 78D is a bar chart showing the quantification of the sodium in the normal healthy reference compared to the exceptional survivor mice treated with the desARE3′UTRERBB2-3.

FIG. 79A is a bar chart showing the quantification of the blood urea nitrogen in the normal healthy reference compared to the exceptional survivor mice treated with the desARE3′UTRERBB2-3. Two tailed t-test (**p=0.002 control versus treatment).

FIG. 79B is a bar chart showing the quantification of the creatinine in the normal healthy reference compared to the exceptional survivor mice treated with the desARE3′UTRERBB2-3.

FIG. 79C is a bar chart showing the quantification of the alkaline phosphatase (ALP) in the normal healthy reference compared to the exceptional survivor mice treated with the desARE3′UTRERBB2-3.

FIG. 79D is a bar chart showing the quantification of the alanine transaminase (ALT) in the normal healthy reference compared to the exceptional survivor mice treated with the desARE3′UTRERBB2-3. Two tailed t-test (***p=0.00043 control versus treatment).

FIG. 79E is a bar chart showing the quantification of the aspartate aminotransferase (AST) in the normal healthy reference compared to the exceptional survivor mice treated with the desARE3′UTRERBB2-3. Two tailed t-test (***p=0.0003 control versus treatment.

FIG. 79F is a bar chart showing the quantification of the creatinine kinase in the normal healthy reference compared to the exceptional survivor mice treated with the desARE3′UTRERBB2-3. Two tailed t-test (***p=0.00013 control versus treatment).

FIG. 80A is a depiction of mice bearing tumor treated with the engineered destabilized 3′UTRERBB2 either as lenti-plasmid DNA or packaged into iron oxide nanocage shrinks the tumors with the downregulation of the ERBB2-YES1-WNK1 kinases.

FIG. 80B is a survival graph showing the therapeutic effects of exceptional survivors in the treated animals.

FIG. 81A is a Western blot showing c-MYC and GAPDH protein expression in MDAMB 231 WT (wild type or no intervention), Vector, IO-nanocage only, 3′UTRMYC1-14, 3′UTRMYC1-18 and 3′UTRMYC2-3 treated cells via IO-nanocage delivery. N=4.

FIG. 81B is a bar chart showing c-MYC mRNA expression normalized against GAPDH in MDAMB 231 WT, Vector, IO-nanocage only, 3′UTRMYC1-14, 3′UTRMYC1-18 and 3′UTRMYC2-3 treated cells via IO-nanocage delivery. Two tailed T-test ****p<0.00001 (WT, vector, IO-nanocage versus 3′UTRMYC1-14, 1-18 and 2-3). N=5.

FIG. 81C is a bar chart showing c-MYC mRNA expression normalized against GAPDH in MDAMB231 WT, Vector, 3′UTRMYC2-3, 1-18, 1-14 and BT474 cells expressing 3′UTRMYC1-14. Two tailed T-test **P<0.001 (WT vs vector, 3′UTRMYC2-3,1-14), ****P<0.00001 (WT vs 3′UTRMYC1-18), ***P<0.0001 (WT vs BT474 3′UTRMYC1-14). N=5.

FIG. 81D is a high resolution transmission electron microscopic (HR-TEM) image displaying IO-nanocages with consistent cuboidal structure and well-defined lattice structure with little to no aggregation.

FIG. 81E shows a transmission electron microscopic (TEM) image of the loaded IO-nanocages (upper arrow) in displays of DNA plasmids wrapped around the surface of IO-nanocages (lower arrow).

FIG. 81F is a diagrammatic illustration of DNA constructs wrapped around the IO nanocages.

FIG. 81G is a bar chart showing viability of cells treated with constructs. **P<0.001 (WT vs vector), ***P<0.0001 (WT vs 3′UTRMYC2-3, 1-18, 1-14). N=6.

FIG. 81H is an immunofluorescence image of c-MYC staining (red) and DAPI (nuclei blue) in WT, vector and 3′UTRMYC1-18 treated cells. N=3.

FIG. 81I is a heat map showing global gene expression pattern changes compared to controls and the 3′UTRMYC 1-18 treated cells.

FIG. 81J is a heat map showing gene expression pattern changes of MYC interacting partners compared to controls and the 3′UTRMYC 1-18 treated cells.

FIG. 81K is a Western blot showing STAT5A/5B and GAPDH protein expression MDAMB 231 WT, Vector, nanocage only, 3′UTRMYC1-14, 3′UTRMYC1-18 and 3′UTRMYC2-3 treated cells. N=3.

FIG. 82A is a Western blot showing c-MYC and GAPDH protein expression in normal epithelial RWPE1 WT, vector, 3′UTRMYC1-14, 3′UTRMYC1-18 and 3′UTRMYC2-3 treated cells for 4 days.

FIG. 82B is a bar chart showing quantification of c-MYC normalized against GAPDH for gel image from A.

FIG. 82C is a light microscopic image of normal epithelial cells RWPE1 WT, vector, 3′UTRMYC1-14, 3′UTRMYC1-18 and 3′UTRMYC2-3 treated cells for 4 days (N=4).

FIG. 82D is a Western blot showing c-MYC and GAPDH protein expression in normal epithelial RWPE1 WT, vector, 3′UTRMYC1-14, 3′UTRMYC1-18 and 3′UTRMYC2-3 treated cells for 8 days.

FIG. 82E is a bar chart showing quantification of c-MYC normalized against GAPDH for gel image from FIG. 82D.

FIG. 82F is a light image of normal epithelial cells RWPE1 WT, vector, 3′UTRMYC1-14, 3′UTRMYC1-18 and 3′UTRMYC2-3 treated cells for 8 days (N=4).

FIG. 82G is a Western blot showing c-MYC and GAPDH protein expression in NCI H1975 Vector, desERBB2-1, desARE3′UTRERBB2-1, desARE3′UTRERBB2-3, desARE3′UTRERBB2-30 and NCI H1975 trastuzumab treated cells.

FIG. 82H is a bar chart showing quantification of c-MYC normalized against GAPDH for gel image from FIG. 81G.

FIG. 82I is a bar chart showing quantification of c-MYC mRNA expression normalized against GAPDH in normal epithelial cells RWPE1 WT, vector, 3′UTRMYC2-3, 3′UTRMYC1-18, 3′UTRMYC1-14 treated cells for 8 days (N=4).

FIG. 83A is a bar chart showing weight measurement of animal bearing tumors WT, nanocage only, vector+IO-nanocage, 3′UTRMYC2-3+IO-nanocage, 3′UTRMYC1-18+IO-nanocage and 3′UTRMYC1-14+IO-nanocage treated, (N=5).

FIG. 83B shows primary tumor images sizes of different controls and treatment groups (N=5).

FIG. 83C shows immunofluorescence images of primary tumors from the control and treatment group stained with c-MYC (red), DAPI nuclei (blue) (N=5).

FIG. 83D is a bar graph showing quantification of c-MYC in primary tumors controls and treated groups. *P<0.03 (WT vs 3′UTRMYC2-3, 1-18 and 1-14) (N=5).

FIG. 83E shows immunofluorescence images of primary tumors from the control and treatment group stained with STAT5A/5B (red), DAPI nuclei (blue) (N=5).

FIG. 83F is a bar graph showing quantification of STAT5A/5B in primary tumors controls and treated groups. *P<0.03 (WT vs IO-nanocage only, 3′UTRMYC2-3). *P<0.01 (WT vs 3′UTRMYC1-18 and 1-14) (N=5).

FIG. 83G shows H&E images of primary tumors, the controls versus treated groups (N=5).

FIG. 83H shows a Kaplan Meier survival plot of animal experiment. ****P<0.0001 (WT, IO-nanocage only vs 3′UTRMYC2-3, 1-18 and 1-14) (N=5).

FIG. 84A depicts metastatic tumor images sizes of different controls and treatment groups (N=2 WT, N=3 nanocage only, N=4 IO-nanocage plus vector, N=4 3′UTRMYC2-3, N=1 3′UTRMYC1-18, N=2 3′UTRMYC1-18).

FIG. 84B shows quantification of metastatic tumors from the controls and treated groups by number.

FIG. 84C depicts immunofluorescence images of metastatic tumors from the control and treatment group stained with c-MYC (red), DAPI nuclei (blue) groups (N=2 WT, N=3 nanocage only, N=4 IO-nanocage plus vector, N=4 3′UTRMYC2-3, N=1 3′UTRMYC1-18, N=2 3′UTRMYC1-18).

FIG. 84D shows quantification of c-MYC in metastatic tumors from the controls and treated groups by immunofluorescence. *P<0.044 (WT vs 3′UTRMYC2-3), *P<0.03 (WT vs 3′UTRMYC1-18 and 1-14).

FIG. 84E shows immunofluorescence images of metastatic tumors from the control and treatment group stained with STAT5A/5B (red), DAPI nuclei (blue). groups (N=2 WT, N=3 IO-nanocage only, N=4 nanocage plus vector, N=4 3′UTRMYC2-3, N=1 3′UTRMYC1-18, N=2 3′UTRMYC1-18).

FIG. 84F shows quantification of STAT5A/5B in metastatic tumors from the controls and treated groups by immunofluorescence. P=ns.

FIG. 84G shows H&E images of primary tumors, the controls versus treated groups (N=5).

FIG. 85A shows immunohistological staining of PDL-1 in primary tumors from the control and treated groups, positive control is human tonsil, PDL-1 (brown), DAPI nuclei (blue). (N=5).

FIG. 85B is a bar graph showing quantification of PDL-1 in primary tumors from the control and treated groups, ***P<0.0005 (WT, IO-nanocage+vector vs 3′UTRMYC2-3, 1-18 and 1-14).

FIG. 85C is a bar graph showing head-to-head quantification of PDL-1 and c-MYC in the same primary tumors from the control and treated groups ***P<0.005 (WT, IO-nanocage+vector vs 3′UTRMYC2-3, 1-18 and 1-14).

FIG. 85D is a graph showing correlation analysis between PDL-1 and c-MYC in the primary tumors, R 2=0.9356, P=0.0071.

FIG. 85E is a bar graph showing head-to-head quantification of PDL-1 and STAT5A/5B in primary tumors from the control and treated groups, ***P<0.0005 (WT, Nanocage+vector vs 3′UTRMYC2-3, 1-18 and 1-14).

FIG. 85F is a graph showing correlation analysis between PDL-1 and STAT5A/5B in the primary tumors, R 2=0.97, P=0.0061.

FIG. 85G shows a table for multiple correlation analysis between c-MYC-STAT5A/5B-PDL-1 in the primary tumors from the control and treated groups.

FIG. 86A shows immunohistological staining of PDL-1 in metastatic tumors from the control and treated groups, PDL-1 (brown), DAPI nuclei (blue). (N=2 WT, N=3 nanocage only, N=4 IO-nanocage plus vector, N=4 3′UTRMYC2-3, N=1 3′UTRMYC1-18, N=2 3′UTRMYC1-18)

FIG. 86B is a bar graph showing quantification of PDL-1 in metastatic tumors from the control and treated groups, *P<0.01 (WT vs, 3′UTRMYC2-3+nanocage, 1-18 and 1-14).

FIG. 86C is a bar graph showing head-to-head quantification of PDL-1 and c-MYC in the same metastatic tumors from the control and treated groups. **P<0.001 (WT vs 3′UTRMYC2-3+nanocage, 1-18 and 1-14).

FIG. 86D is a graph showing correlation analysis between PDL-1 and c-MYC in the metastatic tumors, R 2-0.42, P=ns.

FIG. 86E is a bar graph showing head-to-head quantification of PDL-1 and STAT5A/5B in metastatic tumors from the control and treated groups, P=ns for STAT5A/5B (WT, Nanocage+vector vs 3′UTRMYC2-3+nanocage, 1-18 and 1-14).

FIG. 86F is a graph showing correlation analysis between PDL-1 and STAT5A/5B in the metastatic tumors, R 2=−0.06, P=ns.

FIG. 86G is a table for multiple correlation analysis between c-MYC-STAT5A/5B-PDL-1 in the metastatic tumors from the control and treated groups.

FIG. 87A shows WT no intervention primary tumor group, no IO-nanocage detected (N=5).

FIG. 87B shows IO-nanocage+vector treated primary tumor group, IO-nanocage detected (arrow) (N=2).

FIG. 87C shows 3′UTRMYC2-3 treated primary tumor group, IO-nanocage detected (arrow) (N=4)

FIG. 87D shows 3′UTRMYC1-18 treated primary tumor group, IO-nanocage detected (arrow) (N=2).

FIG. 87E shows 3′UTRMYC1-14 treated primary tumor group, IO-nanocage detected (arrow) (N=2)

FIG. 87F quantification of IO-nanocages detected in the primary tumor.

FIG. 87G shows WT clavicular bone metastatic tumor group, no IO-nanocage detected (N=2).

FIG. 87H shows nanocage only metastatic tumor group, no IO-nanocage detected (N=3).

FIG. 87I shows nanocage+vector metastatic tumor group, no IO-nanocage detected (N=4).

FIG. 87J depicts 3′UTRMYC2-3 treated metastatic tumor group, IO-nanocage detected (arrow) (N=4)

FIG. 87K shows 3′UTRMYC1-18 treated metastatic tumor group, IO-nanocage detected (arrow) (N=1)

FIG. 87L shows 3′UTRMYC1-14 treated metastatic tumor group, IO-nanocage detected (arrow) (N=2)

FIG. 87M is a graph showing quantification of nanocages detected in the metastatic tumor.

FIG. 88A shows a H & E lung metastasis tumor stain of MDAMB231 of no intervention group (N=3).

FIG. 88B depicts a PDL-1 stain of lung metastasis tumor of MDAMB231 of no intervention group (N=3).

FIG. 88C shows Prussian blue stain of lung metastasis tumor of MDAMB231 of no intervention group (N=3)

FIG. 88D is graph showing quantification of PDL-1 and nanocages, no IO-nanocage detected in the MDAMB231 of no intervention group.

FIG. 88E shows a H&E lung metastasis tumor stain of MDAMB231 of the vector plus IO-nanocage only intervention group (N=2).

FIG. 88F shows a PDL-1 stain of lung metastasis tumor of IO-nanocage plus vector intervention group (N=2).

FIG. 88G depicts Prussian blue stain of lung metastasis tumor of IO-nanocage plus vector intervention group, IO-nanocages marked in red arrow (N=2).

FIG. 88H is a graph showing quantification of PDL-1 and nanocages.

FIG. 88I shows a H & E lung metastasis tumor stain of MDAMB231 of 3′UTRMYC2-3 intervention group (N=4).

FIG. 88J depicts PDL-1 lung metastasis tumor stain of MDAMB231 of 3′UTRMYC2-3 intervention group (N=4).

FIG. 88K shows a Prussian blue stain of lung metastasis tumor in the 3′UTRMYC2-3 intervention group, IO-nanocages marked in red arrow (N=4).

FIG. 88L shows quantification of PDL-1 and nanocages.

FIG. 88M depicts a H & E lung metastasis tumor stain of MDAMB231 of 3′UTRMYC1-18 intervention group (N=4).

FIG. 88N shows PDL-1 lung metastasis tumor stain of MDAMB231 of 3′UTRMYC1-18 intervention group (N=4).

FIG. 88O shows Prussian blue stain of lung metastasis tumor in the 3′UTRMYC1-18 intervention group, IO-nanocages marked in red arrow (N=4).

FIG. 88P depicts quantification of PDL-1 and nanocages.

FIG. 88Q shows a H & E lung metastasis tumor stain of MDAMB231 of 3′UTRMYC1-14 intervention group (N=4).

FIG. 88R depicts a PDL-1 lung metastasis tumor stain of MDAMB231 of 3′UTRMYC1-14 intervention group (N=4).

FIG. 88S shows Prussian blue stain of lung metastasis tumor in the 3′UTRMYC1-14 intervention group, IO-nanocages marked in red arrow (N=4).

FIG. 88T depicts quantification of PDL-1 and nanocages.

FIG. 89A is an H &E stain of the MDA MB231 no intervention liver showing hyperchromatic and metastasis (N=4).

FIG. 89B shows a Prussian blue stain for IO-nanocages in MDA MB231 no intervention group, no IO-nanocage found (N=4).

FIG. 89C depicts a H &E stain of the MDA MB231 liver treated with IO-nanocage plus vector showing hyperchromatic and metastasis (N=4).

FIG. 89D depicts a Prussian blue stain for IO-nanocages in IO-nanocage plus vector treated group, IO-nanocage found marked with red arrow (N=4).

FIG. 89E shows a H&E stain of the MDA MB231 3′UTRMYC1-18 treated liver showing no hyperchromatic and no metastasis (N=4).

FIG. 89F depicts a Prussian blue stain for IO-nanocages in 3′UTRMYC1-18 plus IO-nanocages treated group, IO-nanocage were robustly found marked with red arrow (N=4).

FIG. 90A is a schematic depiction of 4SU pulse chase nascent mRNA labeling of the control cell and the cells carrying the destabilized constructs.

FIG. 90B is a graph showing time dependent mRNA expression of the endogenous and destabilized 3′UTRMYC in the same cells from 0 hr-24 hrs in the controls and destabilized cells.

FIG. 90C shows a Venn diagram with EEF2 as intercept between NCI H1975 desARE3′UTRERBB2-3, 30, MDAMB231 3′UTRMYC1-18, DAOY 3′UTRMYC1-18.

FIG. 90D depicts a Western blot of EEF2 and GAPDH protein expression in time scale 0-24 hr in wild type MDAMB231.

FIG. 90E is a Western blot of EEF2 and GAPDH protein in time scale 0-24 hr in 3′UTRMYC1-18.

FIG. 90F shows quantification of EEF2 protein expression normalized against GAPDH in time scale 0-24 hr in wild type MDAMB231.

FIG. 90G depicts quantification of EEF2 protein expression normalized against GAPDH in time scale 0-24 hr in 3′UTRMYC1-18.

FIG. 90H shows quantification of XRN1 mRNA expression normalized against ActB in WT, vector and 3′UTRMYC1-18.

FIG. 90I depicts quantification of CNOT1 mRNA expression normalized against ActB in WT, vector and 3′UTRMYC1-18.

FIG. 91A is a schematic depiction of the vector constructs containing the destabilizing constructs, the arrows indicate primers position used in the targeted sequencing.

FIG. 91B is a gel image of the constructs amplified in the plasmid vector as well in tumors treated with the constructs.

FIG. 91C is a Circos plot show with green bars shows the genomic sites of constructs integration.

FIG. 92A is a gel image of c-MYC 3′UTR amplified from cDNA of MCF7, BT474, MDAMB231 and T47D marked in black asterisks.

FIG. 92B is an electropherogram of MCF7 3′UTR with poly U sequences marked in black.

FIG. 92C is an electropherogram of BT474 3′UTR with poly U sequences marked in black.

FIG. 92D is an electropherogram of MDA MB231 3′UTR with poly U sequences marked in black.

FIG. 92E is an electropherogram of T47D 3′UTR with poly U sequences marked in black.

FIG. 92F is a bar chart showing the zeta potential (mV) of the neat IO-nanocage, neat DNA constructs and DNA-IO-nanocage.

FIG. 92G is a bar chart showing the size (nm) of the neat IO-nanocage, neat DNA constructs and DNA-IO-nanocages, determined by DLS.

FIG. 93 shows the poly U sequences are 99% conserved in 3′UTR of c-MYC across many cancers.

FIG. 94 shows alignment of the 3′UTR of WT c-MYC and engineered destabilized c-MYC with sequences that were changed.

FIG. 95A is a schematic illustration of component sequences used in the design destabilized 3′UTR c-MYC.

FIG. 95B is a schematic illustration of the plasmid vector components.

FIG. 95C is a Western blot of Caspase 7 and GAPDH in controls and cells that received the destabilized constructs.

FIG. 96A is a schematic illustration of cloned 3′UTRMYC2-3 mapped to c-MYC 3′UTR.

FIG. 96B is a schematic illustration of cloned 3′UTRMYC1-18 mapped to c-MYC 3′UTR.

FIG. 96C is a schematic illustration of cloned 3′UTRMYC1-14 mapped to c-MYC 3′UTR.

FIG. 97A depicts a bar charts showing the intensity of p38a, JNK1/2/3, STAT5A/5B, GSK3A/B, PDGFRB, LCK in BT474 clone 5 WT, BT474 clone 5 vectors, BT474 clone 5, desARE3′UTRERBB2-3 (red) and BT474 clone 5 3′UTRMYC2-3 ****P-val<0.0001, Two tailed t-test.

FIG. 97B is a Western blot showing c-MYC, STAT5A/5B and GAPDH protein expression in control and destabilized cells (N=6).

FIG. 97C illustrates bar charts showing ERBB2, EGFR, VEGFA, ABL1, RELA, RELB mRNA expression in MDA MB231 WT, vector and 3′UTRMYC1-18.

FIG. 97D is a heat map showing the expression level of c-MYC and interaction partners in MDA MB231 WT, vector, 3′UTRMYC1-18 and normal female mammary gland.

FIG. 98A is an illustration of animal experiment, tumor implantation, randomization, construct administration and daily tumor measurement and weighing.

FIG. 98B is a chart showing tumor volume recording for the control and treatment groups.

FIG. 98C is a chart showing monitoring of weight measurement of animal bearing tumors both controls and untreated group.

FIG. 99A is a bar chart showing CD274 (PDL-1) expression in different subtypes of breast cancer (ER−/HR−/HER2−), (ER+/HR+/HER2+) and (ER+/HR−/HER2−).

FIG. 99B is a bar chart showing c-MYC expression in different subtypes of breast cancer (ER−/HR−/HER2−), (ER+/HR+/HER2+) and (ER+/HR−/HER2−).

FIG. 99C is a bar chart showing STAT5A expression in different subtypes of breast cancer (ER−/HR−/HER2−), (ER+/HR+/HER2+) and (ER+/HR−/HER2−).

FIG. 99D is a bar chart showing STAT5B expression in different subtypes of breast cancer (ER−/HR−/HER2−), (ER+/HR+/HER2+) and (ER+/HR−/HER2−).

FIG. 99E shows a Kaplan Meier survival curve for c-MYC (low) and c-MYC (high) expression in TNBC.

FIG. 100A shows a PDL-1 lung metastasis tumor stain of MDAMB231 of no intervention group (brown PDL1, blue-DAPI nuclei) (N=3).

FIG. 100B shows a PDL-1 lung metastasis tumor stain of MDAMB231 of IO-nanocage plus vector intervention group (brown PDL1, blue-DAPI nuclei) (N=2).

FIG. 100C shows a PDL-1 lung metastasis tumor stain of MDAMB231 of 3′UTRMYC2-3 plus IO-nanocage intervention group (brown PDL1, blue-DAPI nuclei) (N=4).

FIG. 100D is a PDL-1 lung metastasis tumor stain of MDAMB231 of 3′UTRMYC1-18 plus IO-nanocage intervention group (brown PDL1, blue-DAPI nuclei) (N=4).

FIG. 100E is a PDL-1 lung metastasis tumor stain of MDAMB231 of 3′UTRMYC1-14 plus IO-nanocage intervention group (N=5).

FIG. 101A shows an H&E image of PDL-1, c-MYC, STAT5A/5B and Prussian blue stain of lung metastasis tumor stain of MDAMB231 of no intervention group and a graph showing quantification.

FIG. 101B shows an H&E image of PDL-1, c-MYC, STAT5A/5B and Prussian blue stain of lung metastasis tumor stain of 3′UTRMYC1-18 intervention group and a graph showing quantification.

FIG. 102A shows microscopic images of migration of the cells from the MDA MB231WT, vector, 3′UTRMYC2-3, 1-18 and 1-14 treated cells at 0 hr, 24 hr, 48 hr and 72 hrs.

FIG. 102B depicts a bar charts showing quantification of wound healing in the control and treated groups (**P<0.01, ****P<0.0001, Two tailed T-test).

FIG. 103A shows upregulated GO terms in the destabilized cells carrying 3′UTRMYC1-18.

FIG. 103B is a heat map of UPF1 mRNA expression in the WT, vector and 3′UTRMYC1-18 treated cells.

FIG. 103C is a Western blot showing DCP1A and GAPDH in the control and treated cells.

FIG. 103D is a bar chart showing head-to-head comparison of DCP1A and c-MYC protein expression normalized against GAPDH in the same cells both treated and untreated (***P<0.001, ****P<0.0001, Two tailed t-test).

FIG. 103E shows DCP1A mRNA expression from the controls and the 3′UTRMYC1-18 treated cells.

FIG. 104A shows a H &E stain of MDA MB231 no intervention brain tissue, metastasis pointed in an arrow (N=5)

FIG. 104B shows a Prussian blue stain of MDA MB231 no intervention brain tissue. No nanocages found (N=5).

FIG. 104C shows a H &E stain of MDA MB231 vector treated brain tissue, metastasis pointed in an arrow (N=5)

FIG. 104D shows a Prussian blue stain of MDA MB231 vector treated brain tissue. No nanocages found (N=5).

FIG. 104E shows a H &E stain of MDA MB231 IO-nanocages and vector treated brain tissue, metastasis pointed in an arrow (N=5).

FIG. 104F shows a Prussian blue stain of MDA MB231 IO-nanocages and vector treated brain tissue. No nanocages found (N=5).

FIG. 104G shows a H&E stain of MDA MB231 IO-nanocages 3′UTRMYC2-3 treated brain tissue. No metastasis found (N=5).

FIG. 104H shows a Prussian blue stain of MDA MB231 IO-nanocages 3′UTRMYC2-3 treated brain tissue. Nanocages found (N=5).

FIG. 104I shows a H&E stain of MDA MB231 IO-nanocages 3′UTRMYC1-18 treated brain tissue; metastasis found marked in an arrow (N=5).

FIG. 104J shows a Prussian blue stain of MDA MB231 IO-nanocages 3′UTRMYC1-18 treated brain tissue. Nanocages found (N=5).

FIG. 104K shows a H&E stain of MDA MB231 IO-nanocages 3′UTRMYC1-14 treated brain tissue; metastasis found marked in an arrow (N=5).

FIG. 104L shows a Prussian blue stain of MDA MB231 IO-nanocages 3′UTRMYC1-18 treated brain tissue. Nanocages found (N=5).

FIG. 105A is an H & E stain of MDA MB231 kidney no treatment group (N=4).

FIG. 105B is an H &E stain of MDA MB231 IO-nanocage plus the vector treated group (N=4).

FIG. 105C is an H&E stain of MDA MB231 IO-nanocage only treated group (N=4).

FIG. 105D is an H& E stain of MDA MB231 IO-nanocage plus 3′UTRMYC2-3 treated group (N=4).

FIG. 105E is an H&E stain of MDA MB231 IO-nanocage plus 3′UTRMYC1-18 treated group, tissue has normal renal tubule cellular architecture (N=4).

FIG. 105F is an H&E stain of MDA MB231 IO-nanocage plus 3′UTRMYC1-14 treated group (N=4).

FIG. 106 is a gel picture showing the cDNA amplification of ERBB2 3′UTR across different cancers band size is 450 bp marked in black asterisks.

FIG. 107 shows a sequence alignment of the HER 3′UTR sequences from a UCSC genome browser, ERBB2 3′UTR sequence from the BT474 WT and the engineered ERBB2 3′UTR sequence. The underscored sequences were replaced.

FIG. 108A-D are sequences of desARE3′UTR ERBB2-1,2,3 and 4 clones that were mapped to the ERBB2 3′UTR.

FIG. 109 is the sequence of the desARE3′UTR ERBB2-30 clone that was mapped to the ERBB2 3′UTR.

FIG. 110 is a Western blot showing active caspase 3 and GAPDH expression across the wildtype BT474 clone 5, vector and on BT474 clone 5 containing constructions desARE3′UTR ERBB2-1,3, 30 (n=2).

FIG. 111 is a bar chart showing quantification of the ERBB2 western blot expression normalized against GAPDH on NCI-H1975 wildtype cells, vector and cells containing constructs desARE3′UTR ERBB2-1, 3, 30 and wild type trastuzumab treated NCI-H1975 cells (n=2). T-test ***=0.0021 WT, vector vs desARE3′UTRERBB2-3,30, **=0.02 desARE3′UTRERBB2-1.

FIG. 112A is a bar chart showing blood urea nitrogen level of mice bearing tumor NCI H1975WT, vector, and mice treated with desARE3′UTRERBB2-1, desARE3′UTRERBB2-3 and desARE3′UTRERBB2-30.

FIG. 112B is a bar chart showing creatinine levels of mice bearing tumor NCI H1975 WT, vector, and mice treated with desARE3′UTRERBB2-1, desARE3′UTRERBB2-3 and desARE3′UTRERBB2-30.

FIG. 112C is a bar charting show alkaline phosphatase level of mice bearing tumor NCI H1975WT, vector, and mice treated with desARE3′UTRERBB2-1, desARE3′UTRERBB2-3 and desARE3′UTRERBB2-30.

FIG. 112D is a bar chart showing aspartate amino transferase of mice bearing tumor NCI H1975 WT, vector, and mice treated with desARE3′UTRERBB2-1, desARE3′UTRERBB2-3 and desARE3′UTRERBB2-30.

FIG. 112E is a bar chart showing albumin level of mice bearing tumor NCI H1975 WT, vector, and mice treated with desARE3′UTRERBB2-1, desARE3′UTRERBB2-3 and desARE3′UTRERBB2-30.

FIG. 112F is a bar chart showing globulin of mice bearing tumor NCIH1975 WT, vector, and mice treated with desARE3′UTRERBB2-1, desARE3′UTRERBB2-3 and desARE3′UTRERBB2-30.

FIG. 112G is a bar chart showing calcium level of mice bearing tumor NCI H1975 WT, vector, and mice treated with desARE3′UTRERBB2-1, desARE3′UTRERBB2-3 and desARE3′UTRERBB2-30.

FIG. 112H is a bar chart showing glucose level of mice bearing tumor NCI H1975 WT, vector, and mice treated with desARE3′UTRERBB2-1, desARE3′UTRERBB2-3 and desARE3′UTRERBB2-30.

FIG. 112I is a bar chart showing cholesterol level of mice bearing tumor NCI H1975 WT, vector, and mice treated with desARE3′UTRERBB2-1, desARE3′UTRERBB2-3 and desARE3′UTRERBB2-30.

FIG. 112J is a bar chart showing triglyceride level of mice bearing tumor NCI H1975 WT, vector, and mice treated with desARE3′UTRERBB2-1, desARE3′UTRERBB2-3 and desARE3′UTRERBB2-30.

FIG. 112K is a bar chart showing blood level of CO₂ of mice bearing tumor NCI H1975 WT, vector, and mice treated with desARE3′UTRERBB2-1, desARE3′UTRERBB2-3 and desARE3′UTRERBB2-30.

FIG. 113A is a bar chart showing blood sodium level of mice bearing tumor NCI H1975 WT, vector, and mice treated with desARE3′UTRERBB2-1, desARE3′UTRERBB2-3 and desARE3′UTRERBB2-30.

FIG. 113B is a bar chart showing blood potassium level of mice bearing tumor NCI H1975 WT, vector, and mice treated with desARE3′UTRERBB2-1, desARE3′UTRERBB2-3 and desARE3′UTRERBB2-30.

FIG. 113C is a bar chart showing blood chloride of mice bearing tumor NCI H1975 WT, vector, and mice treated with desARE3′UTRERBB2-1, desARE3′UTRERBB2-3 and desARE3′UTRERBB2-30.

FIG. 113D is a bar chart showing total bilirubin levels of mice bearing tumor NCI H1975 WT, vector, and mice treated with desARE3′UTRERBB2-1, desARE3′UTRERBB2-3 and desARE3′UTRERBB2-30.

FIG. 114A is a bar chart showing quantification of ERBB2 protein fluorescence intensity on xenograft tumors, wildtype NCI-H1975, vector and on tumors treated with constructs desARE3′UTR ERBB2-1, 3 and 30. T-test of WT vs desARE3′UTR ERBB2-1,3,30 (p=0.0074, 0.0057, 0.0094). T-test on Vector vs desARE3′UTRERBB2-1,3,30 (p=0.038, 0.0409, 0.0434).

FIG. 114B is a bar chart showing quantification of CNOT1 protein fluorescence intensity on xenograft tumors, wildtype NCI-H1975, vector and on tumors treated with constructs desARE3′UTR ERBB2-1, 3 and 30. T-T-test of desARE3′UTR ERBB2-3 vs WT, vector, desARE3′UTR ERBB2-1,30 (p=0.0040, 0.0008, 0.0027, 0.0163).

FIG. 114C is a bar chart showing quantification of UPF3B protein fluorescence intensity on xenograft tumors, wildtype NCI-H1975, vector and on tumors treated with constructs desARE3′UTR ERBB2-1, 3 and 30. T-test of WT vs desARE3′UTRERBB2-1,3,30 (p=0.023, 0.019, 0.0145). T-test of Vector vs desARE3′UTR ERBB2-1,3,30 (p=0.0242, 0.0112, 0.0148).

FIG. 114D is a bar chart showing quantification of UPF3B protein fluorescence intensity on xenograft tumors, wildtype NCI-H1975, vector and on tumors treated with constructs desARE3′UTR ERBB2-1, 3 and 30. T-test of WT vs desARE3′UTRERBB2-1, 3, 30 (p=0.027, 0.0475, 0.0054). T-test on Vector vs desARE3′UTR ERBB2-1,3,30 (p=0.028, 0.0489, 0.0055).

FIG. 115A is a diagrammatic representation of the animal experiment starting with tumor implantation, engraftment, tumor randomization and dosing schedule.

FIG. 115B is a chart showing the plot of the daily weight of the animals bearing tumors, the controls, and the treatment groups.

FIG. 115C is a chart showing the daily tumor volume of the controls and treatment groups starting from day 0 of randomization to day 32 of the end of treatment. Two tailed t-test (****p=0.000817962 Nanocage+Vector vs desARE3′UTRERBB2-1, *****p=5.94e-05 IO-nanocage+Vector versus desARE3′UTRERBB2-3, *****p=4.17182e-05 IO-nanocage+Vector versus desARE3′UTRERBB2-30).

FIG. 116 is a bar chart showing the qPCR amplification cycle of the house keeping gene ActB in comparison to the RFP in the vector in two tumors treated with the desARE3′UTRERBB2-30.

FIG. 117 is a graph showing a drug dose response of curve desARE3′UTRERBB2-30 and trastuzumab deruxtecan in NSCLC HCC827 and NCIH460.

### DETAILED DESCRIPTION OF THE INVENTION

This disclosure provides a therapeutic agent for ERBB2 (erythroblastic oncogene B) in cancers including breast cancer, and in trastuzumab resistant breast cancers, triple negative breast cancer (e.g. expressing MYC/STAT5A/5B), sarcoma (e.g., a cholangiosarcoma), a carcinoma (e.g., a hepatocellular carcinoma), a blastoma (e.g., a hepatoblastoma), a gastric adenoma, a hematological cancer, a solid tumor, breast cancer, cervical cancer, gastrointestinal cancer, colorectal cancer, brain cancer, skin cancer, prostate cancer (e.g., neuroendocrine prostate cancer), ovarian cancer, lung cancer (e.g. non-small cell lung carcinoma), thyroid cancer, testicular cancer, pancreatic cancer, liver cancer, endometrial cancer, melanoma, a glioma (e.g., astrocytoma, oligodendroglioma, oligoastrocytoma, glioblastoma), leukemia, lymphoma, chronic myeloproliferative disorder, myelodysplastic syndrome, myeloproliferative neoplasm, plasma cell neoplasm (myeloma) and osteosarcoma where ERBB2 oncogene is a key driver of cancer pathogenesis. This disclosure also provides a therapeutic agent for MYC oncogene overexpression for which there is no therapy in over sixteen major human tumors. MYC oncogene is overexpressed in breast adenocarcinoma, lung squamous carcinoma, lung adenocarcinoma, hepatocellular carcinoma, esophagus carcinoma, hepatocellular carcinoma, gastric carcinoma, colon adenocarcinoma, pancreatic adenocarcinoma, ovarian adenocarcinoma, bladder adenocarcinoma, uterine carcinosarcoma, prostate adenocarcinoma, endometrial adenocarcinoma, acute myeloid leukaemia, Diffuse B cell lymphoma and Osteosarcoma.

### A. RNA Molecules

In one aspect, disclosed are ribonucleic acid (RNA) molecules comprising a 3′-untranslated region (3′ UTR) of an mRNA encoding an ERBB2 protein, in which one or more adenylate-uridine rich element (ARE) poly(uridylic acid) (poly(U)) stabilizing motifs of the 3′ UTR are substituted with a nucleic acid sequence selected from AUUUU, CCUC, CUGC, UAAGUUAU, UAACUUAU, and GUAAAUAG.

In one aspect, disclosed are ribonucleic acid (RNA) molecules comprising a 3′-untranslated region (3′ UTR) of an mRNA encoding a MYC protein, in which one or more adenylate-uridine rich element (ARE) poly(uridylic acid) (poly(U)) stabilizing motifs of the 3′ UTR are substituted with a nucleic acid sequence selected from AUUUU, CCUC, CUGC, UAAGUUAU, UAACUUAU, and GUAAAUAG.

Disclosed herein is an innovative approach for treating various diseases and disorders associated with dysregulation of ERBB2 and/or MYC. Specifically, mRNA constructs were engineered in which the stabilization motifs corresponding to those identified in the cDNA sequences were replaced with destabilized ARE consensus motifs. Exemplary mRNA stabilization and destabilization motifs are shown in Table 1.

TABLE 1

| Construct | Sequence |
|---|---|
| Destabilized RNA sequence 1 | CUGC |
| Destabilized RNA sequence 2 | CCUC |
| Destabilized RNA sequence 3 | UAAGUUAU |
| Destabilized RNA sequence 4 | AUUUU |
| Destabilized RNA sequence 5 | UAACUUAU |
| Destabilized RNA sequence 6 | GUAAAUAG |
| PolyU (stabilization) sequence 1 | UU |
| PolyU (stabilization) sequence 2 | UUU |
| PolyU (stabilization) sequence 3 | UUUU |
| PolyU (stabilization) sequence 4 | UUUUU |
| PolyU (stabilization) sequence 5 | UUUUUU |
| PolyU (stabilization) sequence 6 | UUUUUUU |
| PolyU (stabilization) sequence | UUUUUUU and |
| more than 6 | more |
| Stabilized RNA sequence 1 | UUGCAUGG |
| Stabilized RNA sequence 2 | CCUUACAC |

As detailed herein, the 3′ UTR of ERBB2 and MYC in oncogenic cells expressing ERBB2 and/or MYC, respectively, are enriched with poly(U) sequences that are stabilizing AU rich elements. A poly(U) sequence can contain, for example, at least two consecutive U's, at least three consecutive U's, at least four consecutive U's, at least five consecutive U's, or more than five consecutive U's. Thus, the disclosed ERBB2 or MYC related RNA molecules of the invention have a 3′ UTR region in which one or more poly(U) stabilizing motifs have been replaced with a destabilizing motif, such as, for example, AUUUU, CCUC, CUGC, UAAGUUAU, UAACUUAU, or GUAAAUAG. In this way, the disclosed RNA molecules, once reverse transcribed into DNA and transfected into a vector, become integrated into the genome and outcompete the endogenous RNA molecules comprising a 3′ UTR of an mRNA encoding a ERBB2 or MYC protein, thereby degrading its transcript and protein.

Exemplary engineered destabilized ERBB2 and MYC mRNA constructs, with destabilization motifs are shown in Table 2 (SEQ ID NO: 12 and SEQ ID NO: 13).

TABLE 2

| | |
|---|---|
| MYC | GAGCAAUCACCUAUGAACUUGCUGCUAAGUUAUGAUCAA |
| (RNA) | AUGCAACCUCACAACCUUGGCUGAGUCUUGAGACUGAAAG |
| | AUUUAGCCAUAAUGUAAACUGCCUCAAAUUGGACUUUGG |
| | GCAUAAAAGAACCCUCCUGCAUGCUUACCAUCCCUCCUGC |
| | CCUCCUUUAACAGCCUCGUAACUUAUAAUUGACCUCAAAA |
| | AACCUCAAGAUUUACACAAUGUUUCUCUGUAAAUAUUGCC |
| | AUUAAAUGUAAAUAACUUUAAUAAAACGUUUAUAGCAGU |
| | UACACAGAAUUUCAAUCCUAGUAUAUAGUACCUAGUAUUA |
| | UAGGUACUAUAAACCCUAACCUCCUGCAUUUAAGUACAUU |
| | UUGCACUGCAAAGUAAGUUAUCCUCUAUUGACCUCAGAAA |
| | AAAUAAAAUAACUGGCAAAUAUAUCAUUGAGCCAAAUCUU |
| | AAGUUGUGAAUGCUGCCUCUGCUAACUUAUCCCUCCCAACC |
| | ACCACCAUCCCUGCUGCCCUCAUCAA (SEQ ID NO: 12) |
| ERBB2 | CAGGGGAACCUGCCAUGCCAGGAACCUGUCCUAAGGAACC |
| (RNA) | UGCCUUCCUGCUUGAGUUCCCAGAUGGCUGGAAGGGGUCC |
| | AGCCUCGUUGGAAGAGGAACAGCACUGGGGAGUCUUCGUG |
| | GAUUCUGAGGCCCUGCCCAAUGAGACUCUAGGGUCCAGUGG |
| | AUGCCACAGCCCAGCUUGGCCCUCUCCUUCCAGAUCCUGGG |
| | UACUGAAAGCCUUAGGGAAGCUGGCCUGAGAGGGGAAGCGG |
| | CCCUAAGGGAGUGUCUAAGAACAAAAGCGACCCAUUCAGAG |
| | ACUGUCCCUGAAACCUAGUACUGCCCCCCAUGAGGAAGGAA |
| | CAGCAAUGGUGUCAGUAUCCAGGUCGCGUUCAGAGUGCCCU |
| | CCUGCUUAGCUGCUAAGUUAUCUGCCUCGCUGCCUCAAAGA |
| | UGAAAUAAAGACCCAGGGGGAGAAUGGGUGUUGUAUGGGG |
| | AGGCAAGUGUGGGGGGUCCUUCUCCACACCCACUUUGUCCAU |
| | UUGCAAAUAUAU (SEQ ID NO: 13) |

As used herein, the term “RNA” relates to a molecule which comprises ribonucleotide residues and preferably being entirely or substantially composed of ribonucleotide residues. The term “ribonucleotide” relates to a nucleotide with a hydroxyl group at the 2′-position of a β-D-ribofuranosyl group. The term “RNA” comprises double-stranded RNA, single stranded RNA, isolated RNA such as partially or completely purified RNA, essentially pure RNA, synthetic RNA, and recombinantly generated RNA such as modified RNA, which differs from naturally occurring RNA by addition, deletion, substitution, and/or alteration of one or more nucleotides. Such alterations can include addition of non-nucleotide material, such as to the end(s) of a RNA or internally, for example, at one or more nucleotides of the RNA. Nucleotides in RNA molecules can also comprise non-standard nucleotides, such as non-naturally occurring nucleotides or chemically synthesized nucleotides. These altered RNAs can be referred to as analogs, particularly analogs of naturally-occurring RNAs. As used herein, RNA includes mRNA.

The term “mRNA” means “messenger-RNA” and relates to a transcript that is generated by using a DNA template and encodes a peptide or protein. Typically, mRNA comprises a 5′-UTR, a protein coding region, a 3′-UTR, and a poly(A) sequence. mRNA may be generated by in vitro transcription from a DNA template. The in vitro transcription methodology is known to one skilled in the art. For example, there are a variety of in vitro transcription kits commercially available. As detailed herein, mRNA can be modified by incorporating various destabilizing modifications into the 3′ UTR region (e.g., AUUUU, CCUC, CUGC, UAAGUUAU, UAACUUAU, GUAAAUAG).

The terms “3′-untranslated region” and “3′ UTR,” as used herein, relate to a region that is located at the 3′ end of an RNA molecule, preferably an mRNA molecule, downstream of the termination codon of a protein-encoding region, and that is transcribed but is not translated into an amino acid sequence. According to the invention, a first polynucleotide region is considered to be located downstream of a second polynucleotide region, if the 5′ end of said first polynucleotide region is the part of said first polynucleotide region closest to the 3′ end of said second polynucleotide region.

The 3′-untranslated region typically extends from the termination codon for a translation product to the poly(A) sequence, which is usually attached after the transcription process. The 3′-untranslated regions of mammalian mRNA typically have a homology region known as the AAUAAA hexanucleotide sequence. This sequence is presumably the poly(A) attachment signal and is frequently located from 10 to 30 bases upstream of the poly(A) attachment site. 3′-untranslated regions can contain one or more inverted repeats, which can fold to give stem-loop structures that act as barriers for exoribonucleases or interact with proteins known to increase RNA stability (e.g., RNA-binding proteins). The average length of human 3′-untranslated regions is between 800-1000 nucleotides. As would be understood by one of skill in the art, the length of the 3′ UTR plays an important role in determining both translational efficiency and the stability of an mRNA.

As would be understood by one of skill in the art, it can be determined whether a 3′-untranslated region or a nucleic acid sequence derived therefrom decreases the stability and/or translation efficiency of RNA, by incorporating the 3′-untranslated region or the nucleic acid sequence derived therefrom into the 3′-untranslated region of a mRNA and measuring whether said incorporation decreases the amount of protein synthesized.

The terms “poly(uridylic acid) sequence,” “poly(U) sequence,” and “poly(U) stabilizing motif” refer to a sequence of uridylic acid residues that are typically located at the 3′ end of an RNA molecule. Generally, the poly(A) sequence at the end of the 3′ UTR is important for the nuclear export, translation, and stability of mRNA. The sequence is shortened over time, and, when it is short enough, the mRNA is enzymatically degraded. To counteract poly(A) mediated degradation, poly(U) sequences can interact with poly(A) tails to inhibit the association of poly(A) binding protein and to confer increased stability upon introduction into ectopic transcripts. Poly(U) and poly(A) interactions can prevent negative regulation of mRNA. As detailed herein, in various aspects, a poly(U) sequence can have at least two, at least three, at least 4, at least 5, at least 6, at least 7, at least 8, at least 9, at least 10, or more than ten consecutive uridylic acid residues.

The terms “adenylate-uridylate-rich elements,” “AU-rich elements,” and “AREs” refer to sequences that are rich in adenosine and uridine bases, typically located within the 3′ UTR of a RNA molecule. These elements are binding sites for proteins, which proteins, in response to different intracellular and extracellular signals, can promote mRNA decay, effect mRNA stability, or promote translation.

As would be appreciated by one of ordinary skill, the mRNA decay rate is a key determinant of steady-state mRNA abundance and mRNA turnover. At any given time in the cell, mRNA is synthesized by polymerases and destroyed by nucleases. When these two events occur at a constant rate, they give rise to a steady-state mRNA population for each unique transcript. Variations in mRNA transcription rates are generally recognized for their central importance in regulating gene expression. UTRs, particularly the 3′ UTR, play a role in transcript expression regulation by controlling mRNA stability, decay, and translation. Transcript stability can be affected by various cis-elements, such as AREs, in the 3′ UTR. Thus, in various aspects, the stability of a RNA molecule (e.g., a RNA molecule of the invention comprising a 3′ UTR of an mRNA encoding an ERBB2 or MYC protein, in which one or more ARE poly(U) stabilizing motifs of the 3′ UTR are substituted with a nucleic acid sequence selected from AUUUU, CCUC, CUGC, UAAGUUAU, UAACUUAU, and GUAAAUAG) can be evaluated by measuring the decay rate of the RNA molecule relative to the decay rate of a wildtype RNA molecule comprising a 3′ UTR encoding a ERBB2 or MYC protein.

In various aspects, the RNA molecule comprising a 3′ UTR of an mRNA encoding an ERBB2 protein, in which one or more ARE poly(U) stabilizing motifs of the 3′ UTR are substituted with a nucleic acid sequence selected from AUUUU, CCUC, CUGC, UAAGUUAU, UAACUUAU, and GUAAAUAG, is destabilized compared to a wildtype RNA molecule comprising a 3′ UTR encoding an ERBB2 protein. For example, the disclosed RNA molecule can be at least 1%, at least 5%, at least 10%, at least 15%, at least 20%, at least 25%, at least 30%, at least 40%, at least 50%, or more than 50% destabilized compared to wildtype RNA molecules comprising a 3′ UTR encoding an ERBB2 protein. Methods of determining whether a RNA molecule is destabilized relative to a wildtype RNA molecule are well-known in the art. See, e.g., Koh et al. (2019) Scientific reports vol. 9 (1): 5976.

In various aspects, the RNA molecule comprising a 3′ UTR of an mRNA encoding an ERBB2 protein, in which one or more ARE poly(U) stabilizing motifs of the 3′ UTR are substituted with a nucleic acid sequence selected from AUUUU, CCUC, CUGC, UAAGUUAU, UAACUUAU, and GUAAAUAG, destabilizes a wildtype RNA molecule comprising a 3′ UTR encoding an ERBB2 protein, such that the destabilized wildtype RNA molecule is at least 1%, at least 5%, at least 10%, at least 15%, at least 20%, at least 25%, at least 30%, at least 40%, at least 50%, or more than 50% destabilized compared to a wildtype RNA molecule comprising a 3′ UTR encoding an ERBB2 protein that has not been destabilized by a RNA molecule of the invention.

In various aspects, the RNA molecule comprising a 3′ UTR of an mRNA encoding a MYC protein, in which one or more ARE poly(U) stabilizing motifs of the 3′ UTR are substituted with a nucleic acid sequence selected from AUUUU, CCUC, CUGC, UAAGUUAU, UAACUUAU, and GUAAAUAG, is destabilized compared to a wildtype RNA molecule comprising a 3′ UTR encoding a MYC protein. For example, the disclosed RNA molecule can be at least 1%, at least 5%, at least 10%, at least 15%, at least 20%, at least 25%, at least 30%, at least 40%, at least 50%, or more than 50% destabilized compared to wildtype RNA molecules comprising a 3′ UTR encoding a MYC protein. Methods of determining whether a RNA molecule is destabilized relative to a wildtype RNA molecule are well-known in the art. See, e.g., Koh et al. (2019).

In various aspects, the RNA molecule comprising a 3′ UTR of an mRNA encoding a MYC protein, in which one or more ARE poly(U) stabilizing motifs of the 3′ UTR are substituted with a nucleic acid sequence selected from AUUUU, CCUC, CUGC, UAAGUUAU, UAACUUAU, and GUAAAUAG, destabilizes a wildtype RNA molecule comprising a 3′ UTR encoding a MYC protein, such that the destabilized wildtype RNA molecule is at least 1%, at least 5%, at least 10%, at least 15%, at least 20%, at least 25%, at least 30%, at least 40%, at least 50%, or more than 50% destabilized compared to a wildtype RNA molecule comprising a 3′ UTR encoding a MYC protein that has not been destabilized by a RNA molecule of the invention.

In various aspects, the RNA molecule comprises a 3′ UTR of an mRNA encoding an ERBB2 or MYC protein, in which one or more ARE poly(U) stabilizing motifs of the 3′ UTR are substituted with a nucleic acid sequence selected from AUUUU, CCUC, CUGC, UAAGUUAU, UAACUUAU, and GUAAAUAG, wherein the one or more ARE poly(U) stabilizing motifs comprise at least two consecutive U's. In various further aspects, the RNA molecule comprises a 3′ UTR of an mRNA encoding an ERBB2 or MYC protein, in which one or more ARE poly(U) stabilizing motifs of the 3′ UTR are substituted with a nucleic acid sequence selected from AUUUU, CCUC, CUGC, UAAGUUAU, UAACUUAU, and GUAAAUAG, wherein the one or more ARE poly(U) stabilizing motifs comprise at least three consecutive U's. In a still further aspect, the RNA molecule comprises a 3′ UTR of an mRNA encoding an ERBB2 or MYC protein, in which one or more ARE poly(U) stabilizing motifs of the 3′ UTR are substituted with a nucleic acid sequence selected from AUUUU, CCUC, CUGC, UAAGUUAU, UAACUUAU, and GUAAAUAG, wherein the one or more ARE poly(U) stabilizing motifs comprise at least four consecutive U's.

In various aspects, the RNA molecule is destabilized in at least two ARE poly(U) stabilizing motifs. In a further aspect, the RNA molecule is destabilized in at least three ARE poly(U) stabilizing motifs. In a still further aspect, the RNA molecule is destabilized in at least four ARE poly(U) stabilizing motifs. In an even further aspect, every ARE poly(U) stabilizing motif is destabilized.

In one aspect, a ribonucleic acid (RNA) molecule is provided comprising a 3′-untranslated region (3′ UTR) of an mRNA encoding an ERBB2 protein, in which one or more adenylate-uridine rich element (ARE) poly(uridylic acid) (poly(U)) stabilizing motifs of the 3′ UTR are substituted with a nucleic acid sequence selected from AUUUU, CCUC, CUGC, UAAGUUAU, UAACUUAU, and GUAAAUAG.

One or more ARE poly(U) stabilizing motifs of the 3′ UTR may comprise UUUUU, UUGCAUGG, and CCUUACAC. The RNA molecule may be destabilized in at least two ARE poly(U) stabilizing motifs of the 3′ UTR. The RNA molecule may destabilized in at least three ARE poly(U) stabilizing motifs of the 3′ UTR. The RNA molecule may be destabilized in at least four ARE poly(U) stabilizing motifs of the 3′ UTR. In one aspect, the 3′ UTR of the mRNA encoding an ERBB2 protein is SEQ ID NO: 13. In one aspect, every ARE poly(U) stabilizing motif of the 3′ UTR is destabilized. The RNA may further comprising a polyadenyl sequence that may be located at the 3′ end of the RNA molecule.

In one aspect, the 3′ UTR of the mRNA encoding an ERBB2 protein is at least 20% identical to SEQ ID NO: 13. In one aspect, the 3′ UTR of the mRNA encoding an ERBB2 protein is at least 80% identical to SEQ ID NO: 13. In one aspect, the 3′ UTR of the mRNA encoding an ERBB2 protein is at least 85% identical to SEQ ID NO: 13. In one aspect, the 3′ UTR of the mRNA encoding an ERBB2 protein is at least 90% identical to SEQ ID NO: 13. In one aspect, the 3′ UTR of the mRNA encoding an ERBB2 protein is at least 95% identical to SEQ ID NO: 13. In one aspect, the 3′ UTR of the mRNA encoding an ERBB2 protein is at least 99% identical to SEQ ID NO: 13. In one aspect, the RNA molecule is SEQ ID NO: 13.

In another aspect, a method of making a complementary deoxyribonucleic acid (cDNA) molecule is provided, the method comprising reverse transcribing the RNA molecule of claim to produce the cDNA molecule. The reverse transcription may be carried out in vitro. In another aspect, a cDNA molecule is prepared from the RNA molecule.

In one aspect, a ribonucleic acid (RNA) molecule is provided comprising a 3′-untranslated region (3′ UTR) of an mRNA encoding a MYC protein, in which one or more adenylate-uridine rich element (ARE) poly(uridylic acid) (poly(U)) stabilizing motifs of the 3′ UTR are substituted with a nucleic acid sequence selected from AUUUU, CCUC, CUGC, UAAGUUAU, UAACUUAU, and GUAAAUAG.

One or more ARE poly(U) stabilizing motifs of the 3′ UTR may comprise UUUUU, UUGCAUGG, and CCUUACAC. The RNA molecule may be destabilized in at least two ARE poly(U) stabilizing motifs of the 3′ UTR. The RNA molecule may destabilized in at least three ARE poly(U) stabilizing motifs of the 3′ UTR. The RNA molecule may be destabilized in at least four ARE poly(U) stabilizing motifs of the 3′ UTR. In one aspect, the 3′ UTR of the mRNA encoding an ERBB2 protein is SEQ ID NO: 12. In one aspect, every ARE poly(U) stabilizing motif of the 3′ UTR is destabilized. The RNA may further comprising a polyadenyl sequence that may be located at the 3′ end of the RNA molecule.

In one aspect, the 3′ UTR of the mRNA encoding a MYC protein is at least 20% identical to SEQ ID NO: 12. In one aspect, the 3′ UTR of the mRNA encoding a MYC protein is at least 80% identical to SEQ ID NO: 12. In one aspect, the 3′ UTR of the mRNA encoding a MYC protein is at least 85% identical to SEQ ID NO: 12. In one aspect, the 3′ UTR of the mRNA encoding a MYC protein is at least 90% identical to SEQ ID NO: 12. In one aspect, the 3′ UTR of the mRNA encoding a MYC protein is at least 95% identical to SEQ ID NO: 12. In one aspect, the 3′ UTR of the mRNA encoding a MYC protein is at least 99% identical to SEQ ID NO: 12. In one aspect, the RNA molecule is SEQ ID NO: 12.

### B. DNA Molecules

In one aspect, disclosed are deoxyribonucleic acid (DNA) molecules comprising in the 5′→3′ direction of transcription relative to synthesis of a messenger RNA (mRNA) molecule comprising a 3′ UTR of an mRNA encoding an ERBB2 protein: (a) a promoter, operatively linked to (b) a nucleic acid sequence encoding a 3′ UTR of a ERBB2 gene in the mRNA molecule in which one or more ARE poly(U) stabilizing motifs of the 3′ UTR of the mRNA are substituted with a nucleic acid sequence selected from AUUUU, CCUC, CUGC, UAAGUUAU, UAACUUAU, and GUAAAUAG.

In one aspect, disclosed are deoxyribonucleic acid (DNA) molecules comprising in the 5′→3′ direction of transcription relative to synthesis of a messenger RNA (mRNA) molecule comprising a 3′ UTR of an mRNA encoding a MYC protein: (a) a promoter, operatively linked to (b) a nucleic acid sequence encoding a 3′ UTR of a MYC gene in the mRNA molecule in which one or more ARE poly(U) stabilizing motifs of the 3′ UTR of the mRNA are substituted with a nucleic acid sequence selected from AUUUU, CCUC, CUGC, UAAGUUAU, UAACUUAU, and GUAAAUAG.

As detailed herein, the disclosed DNA molecules can be cloned into a vector for use as a therapeutic agent to target ERBB2 and MYC gene (e.g. oncogene) overexpression. Such overexpression is observed in a variety of diseases and disorders for which dysregulation of ERBB2 and/or MYC is implicated including, but not limited, to various cancers (e.g., breast cancer such as trastuzumab-resistant breast cancers and triple negative breast cancer, colon cancer, lung cancer, prostate cancer, cervical squamous cell carcinomas, endometrial carcinoma, neuroblastoma, meningioma, melanoma, squamous cell carcinoma of the skin, ovarian cancer, osteosarcoma, acute myeloma leukemia, head and neck squamous cell carcinoma, and other cancers where the MYC/ERBB2 oncogene is a key driver of cancer pathogenesis).

In various aspects, the disclosed DNA molecules (e.g., a disclosed cDNA molecule) can be prepared by reverse transcribing a disclosed RNA molecule. Thus, in various aspects, disclosed are DNA (e.g., cDNA) molecules prepared from a disclosed RNA molecule. In various further aspects, reverse transcription is carried out in vitro. In this way, the ERBB2/MYC 3′ UTR are destabilized and their expression can be driven by a promoter (e.g., a de-capping promoter such as DCP1A) to specifically degrade the transcript and protein through nonsense mediated decay.

Exemplary destabilization sequences are shown in Table 3 below.

TABLE 3

| Construct | Sequence |
|---|---|
| Destabilized DNA sequence 1 | CTGC |
| Destabilized DNA sequence 2 | CCTC |
| Destabilized DNA sequence 3 | TAAGTTAT |
| Destabilized DNA sequence 4 | ATTTT |
| Destabilized DNA sequence 5 | TAACTTAT |
| Destabilized DNA sequence 6 | GAACTTAT |

Exemplary polyA sequences, exemplary promoter sequences, and exemplary restriction sequences are shown in Table 4. Exemplary destabilized ERBB2 and MYC DNA constructs are also shown in Table 5 and Table 6.

TABLE 4

| | |
|---|---|
| PolyA | AAAA |
| Sequence | |
| PolyA | AAAAAAAA |
| Sequence | |
| PolyA | AAAAAAAAAA (SEQ ID NO: 65) |
| Sequence | |
| PolyA | AAAAAAAAAAAA (SEQ ID NO: 66) |
| Sequence | |
| Promoter | GCCATCGAGGCAAAATTCCCCCATCTGCGTCAGTCCCC |
| Sequence | TCAACTTCCGCCTCTACGCGGGCCTCGCGGCAGGGCGG |
| | TACGTCAGCCATTCTGGTCCGCCGCGCGCACGCTCCGG |
| | GCGCCGGGTTCCGGCGTTGTCAGGGTCCGCGGCCCTAC |
| | GATGTGGGCGGTGTCCAAGGCTGCGTAGTGGAGCTTGC |
| | AGGCTGGAGCT (DCP1A promoter, |
| | SEQ ID NO: 29) |
| Promoter | GCCATCGAGGCAAAATTCCCCCATCTGCGTCAGTCCCC |
| Sequence | TCAACTTCCGCCTCTACGCGGGCCTCGCGGCAGGGCGG |
| | TACGTCAGCCATTCTGGTCCGCCGCGCGCATGCGTAGT |
| | GGAGCTTGCAGGCTGGAGCT (Shortened DCP1A |
| | promoter, SEQ ID NO: 30) |
| Promoter | a DCP2 promoter |
| Sequence | |
| Promoter | a ZFP36 promoter |
| Sequence | |
| Restriction | CCTAG (BamH1) |
| sequence | |
| Restriction | TTCGAA (BstB1) |
| sequence | |
| Restriction | TCTAGA (Xba1) |
| sequence | |
| Restriction | GGGCCC (Apa1) |
| sequence | |
| Restriction | GGGCCC (PspOM1) |
| sequence | |

TABLE 5

| | |
|---|---|
| SEQ ID NO: 68 | first digestion enhancing sequence is SEQ ID NO: 28; |
| ERBB2-1 | first restriction sequence is TTCGAA; |
| | first poly A sequence is AAAAAAAA; |
| | promoter is SEQ ID NO: 29; |
| | nucleic acid sequence is SEQ ID NO: 41; |
| | second poly A sequence is AAAAAAAA; |
| | second restriction sequence is CCTAG |
| | second digestion enhancing sequence is SEQ ID NO: 27. |
| SEQ ID NO: 69 | first digestion enhancing sequence is SEQ ID NO: 28; |
| ERBB2-3 | first restriction sequence is TTCGAA; |
| | first poly A sequence is AAAAAAAA; |
| | promoter is SEQ ID NO: 29; |
| | nucleic acid sequence is SEQ ID NO: 42; |
| | second poly A sequence is AAAAAAAA; |
| | second restriction sequence is CCTAG |
| | second digestion enhancing sequence is SEQ ID NO: 27. |
| SEQ ID NO: 70 | first digestion enhancing sequence is SEQ ID NO: 28; |
| ERBB2-2 | first restriction sequence is TTCGAA; |
| | first poly A sequence is AAAAAAAA; |
| | promoter is SEQ ID NO: 29; |
| | nucleic acid sequence is SEQ ID NO: 43; |
| | second poly A sequence is AAAAAAAA; |
| | second restriction sequence is CCTAG |
| | second digestion enhancing sequence is SEQ ID NO: 27. |
| SEQ ID NO: 71 | first digestion enhancing sequence is SEQ ID NO: 28; |
| ERBB2-4 | first restriction sequence is TTCGAA; |
| | first poly Asequence is AAAAAAAA; |
| | promoter is SEQ ID NO: 29; |
| | nucleic acid sequence is SEQ ID NO: 44; |
| | second poly A sequence is AAAAAAAA; |
| | second restriction sequence is CCTAG |
| | second digestion enhancing sequence is SEQ ID NO: 27. |
| SEQ ID NO: 72 | first digestion enhancing sequence is SEQ ID NO: 28; |
| ERBB2-30 | first restriction sequence is TTCGAA; |
| | first poly A sequence is AAAAAAAA; |
| | promoter is SEQ ID NO: 29; |
| | nucleic acid sequence is SEQ ID NO: 45; |
| | second poly A sequence is AAAAAAAA; |
| | second restriction sequence is CCTAG |
| | second digestion enhancing sequence is SEQ ID NO: 27. |
| SEQ ID NO: 73 | first digestion enhancing sequence is SEQ ID NO: 28; |
| MYC 2-3 | first restriction sequence is TTCGAA; |
| | first poly A sequence is AAAAAAAA; |
| | promoter is SEQ ID NO: 29; |
| | nucleic acid sequence is SEQ ID NO: 46; |
| | second poly A sequence is AAAAAAAA; |
| | second restriction sequence is CCTAG |
| | second digestion enhancing sequence is SEQ ID NO: 27. |
| SEQ ID NO: 74 | first digestion enhancing sequence is SEQ ID NO: 28; |
| MYC 1-18 | first restriction sequence is TTCGAA; |
| | first poly A sequence is AAAAAAAA |
| | promoter is SEQ ID NO: 29; |
| | nucleic acid sequence is SEQ ID NO: 47; |
| | second poly A sequence is AAAAAAAA; |
| | second restriction sequence is CCTAG |
| | second digestion enhancing sequence is SEQ ID NO: 27. |
| SEQ ID NO: 75 | first digestion enhancing sequence is SEQ ID NO: 28; |
| MYC 1-14 | first restriction sequence is TTCGAA; |
| | first poly Asequence is AAAAAAAA; |
| | promoter is SEQ ID NO: 30; |
| | nucleic acid sequence is SEQ ID NO: 48; |
| | second poly Asequence is AAAAAAAA; |
| | second restriction sequence is CCTAG |
| | second digestion enhancing sequence is SEQ ID NO: 27. |
| SEQ ID NO: 76 | promoter is SEQ ID NO: 29; |
| ERBB2-1 | Nucleotide sequence is SEQ ID NO: 41; |
| SEQ ID NO: 77 | promoter is SEQ ID NO: 29; |
| ERBB2-2 | Nucleotide sequence is SEQ ID NO: 42; |
| SEQ ID NO: 78 | promoter is SEQ ID NO: 29; |
| ERBB2-3 | Nucleotide sequence is SEQ ID NO: 43; |
| SEQ ID NO: 79 | promoter is SEQ ID NO: 29; |
| ERBB2-4 | Nucleotide sequence is SEQ ID NO: 44; |
| SEQ ID NO: 80 | promoter is SEQ ID NO: 29; |
| ERBB2-30 | Nucleotide sequence is SEQ ID NO: 45; |
| SEQ ID NO: 81 | promoter is SEQ ID NO: 29; |
| MYC 2-3 | Nucleotide sequence is SEQ ID NO: 46; |
| SEQ ID NO: 82 | promoter is SEQ ID NO: 29; |
| MYC 1-18 | Nucleotide sequence is SEQ ID NO: 47; |
| SEQ ID NO: 83 | promoter is SEQ ID NO: 29; |
| MYC 1-14 | Nucleotide sequence is SEQ ID NO: 48; |

TABLE 6
GCCCGCGAGGACCCGCCCGAGCTTCGAAAAAAAAAAGCCATCGAGGCAAA
ATTCCCCCATCTGCGTCAGTCCCCTCAACTTCCGCCTCTACGCGGGCCTCGC
GGCAGGGCGGTACGTCAGCCATTCTGGTCCGCCGCGCGCACGCTCCGGGCG
CCGGGTTCCGGCGTTGTCAGGGTCCGCGGCCCTACGATGTGGGCGGTGTCC
AAGGCTGCGTAGTGGAGCTTGCAGGCTGGAGCTGGGNNNNAANNNNNNNN
NCNGANGCCTGCCAGTGAGAGTCNAGGGTCCAGTGAAGGCCGCATCGCAC
CTTGGCCCTCTCCTTCCGTATCCTGGGTACTGAGGCCGTAGGGAAGCTGGC
CNTTGAGGGGAAGCGAGGACTNGAAGTGAGNGAAGCCAGCCTACCCGGTA
AAATCGGGCTCTGAGTCGNTTGGGCCCAACCTGAGGAGGATTAGCATTGCC
GTGAGCGGCGGGGGTTACCTAAGGTGCTGAACAGTTCCCTCCCTGTCGCAT
TGGTNGGGAACCATCTAGCTCAGCCACATCCTCCCGCTACGNGCTTTGGGC
TGATTGCAANCCATGGGACTGACACCACTTCTACTACATCCTCNNNGGCGG
TGACTCTTCNACCTCCACCCATAGACCCATATTNCCCTNATTCTCCTCNTAC
CANNAAAANNCCTGTTCGGNNCCANCACCATCAATTCTTCNCTNNNAGATT
NNTCCATCATTCTTTTTCTNNNCAATATGTAACATTGTTTTNNATGAANNNC
CAAANNCCNCTAAGTGTTGGCTTTAAAGTCCTAGGGGCCGGCCCCGCCG
(SEQ ID NO: 68, ERBB2-1)
GCCCGCGAGGACCCGCCCGAGCTTCGAAAAAAAAAAGCCATCGAGGCAAA
ATTCCCCCATCTGCGTCAGTCCCCTCAACTTCCGCCTCTACGCGGGCCTCGC
GGCAGGGCGGTACGTCAGCCATTCTGGTCCGCCGCGCGCACGCTCCGGGCG
CCGGGTTCCGGCGTTGTCAGGGTCCGCGGCCCTACGATGTGGGCGGTGTCC
AAGGCTGCGTAGTGGAGCTTGCAGGCTGGAGCTGNNNNNNNNNTNNNNNA
NCAGAGGCNNGCTCACTGAGACTCTATGGTCAGTGAAGGCCACACCCCAA
CTTGGCCCTTTCCTTCCGGATCCTGGGTACTGAGGCCTTAGGGAAGCTGGC
CTGACATGGGAAGCAGCCCTTGCGGAGTGTCTTGAACTGAACCGACCCATT
AAAAGACTGTCCCTGAAACCTCGTACTGCCCCCCTGAGGAAGGAACTGCCCT
GTTGGCAGTGTCGGTGCTTTGTACACAGTGCTTTTCNGTTTATNTTTTACTT
TTTTTGTTTTGCTTTTTTAAATATGAAATTCATACCCATGTTGACAATGGGT
GTTGTATGGGAAGGCAAGGGACTCCCTTTGGTGATGGGCGCGACCTTTGGT
ATCGCCATATCGCTGGTGATTATCACCTGGTCTGATGTGTATACCGGACCC
ACCCCTTNNCACATCCAANAAGGCTCTTGCTGATGGCAATATCTGGTGGTG
GCTCTCNNAAAAANCTCATCCCTAAGTGTTGGCTTTAAAGTCCTAGGGGCC
GGCCCCGCCG (SEQ ID NO: 69, ERBB2-3)
GCCCGCGAGGACCCGCCCGAGCTTCGAAAAAAAAAAGCCATCGAGGCAAA
ATTCCCCCATCTGCGTCAGTCCCCTCAACTTCCGCCTCTACGCGGGCCTCGC
GGCAGGGCGGTACGTCAGCCATTCTGGTCCGCCGCGCGCACGCTCCGGGCG
CCGGGTTCCGGCGTTGTCAGGGTCCGCGGCCCTACGATGTGGGCGGTGTCC
AAGGCTGCGTAGTGGAGCTTGCAGGCTGGAGCTNNNNNNNNNANTTNNNN
AACNNANGCCNGCNNNTGAGACTCTACGGTCCAGTGGATGTAACATCCCA
GCTTGGTCCTTTCCTTCCATATCCTGGGTACTGAAAGCCTTAGGGAAACTGG
CCTGATAGGGGAAGCGGCCCTACATGATTGGATGGNACGGAATCGACCCTT
TCAGAGACTGTCCCTGAAACCTATCAGTGCCCGCCACGATGAAAGAACAGC
AACGGTGACAGTATCCTGGCATTGTCCAAANNGCTTTTCTGTGCTTNTTTAC
TTTTTTTAATNAGTTTTTTTAAAGATCAAACATCCACCCCAGGTCCCAATGG
GGGGTGCCTGAGGAAGAACGATTGGCGCCTGTTCCCGTTCCCCTTTACTGG
ACTGCGTACTACACCCCGAGGCCTTCTTTGAGAGAGCGNNCCATCCTGGGA
CAGCAACCTAACTANNCCCTCTCCCCCTGCGGATGACAATGTCTGGCCGGG
GTTCTNATAGGCTACTCACCCCCTTTTGTTTTTATTACAAACGATTATGTTT
GNTCTGACCNCANNAAACTAAGTGTTGGCTTTAAAGTCCTAGGGGCCGGCC
CCGCCG (SEQ ID NO: 70, ERBB2-2)
GCCCGCGAGGACCCGCCCGAGCTTCGAAAAAAAAAAGCCATCGAGGCAAA
ATTCCCCCATCTGCGTCAGTCCCCTCAACTTCCGCCTCTACGCGGGCCTCGC
GGCAGGGCGGTACGTCAGCCATTCTGGTCCGCCGCGCGCACGCTCCGGGCG
CCGGGTTCCGGCGTTGTCAGGGTCCGCGGCCCTACGATGTGGGCGGTGTCC
AAGGCTGCGTAGTGGAGCTTGCAGGCTGGAGCTNNNNNNNNNNNTTNAAA
NATATTTGCAATGGACAAAGTGGGTGTGGAGAAGGACCCCCCACACTTGCC
TCCCCATACAACACCCATTCTCCCCCTGGGTCTTTATTTCATCTTTAAAAAA
ACAAAACAAAAAAAGTAAAAACTAAACACAAAAGCACTCTGTACAAAGCC
TGGATACTGACACCATTGCTGTTCCTTCCTCATGGGGGGCAGTACTAGGTTT
CATGGACAGTCTCTGAATGGTTCGCTTTTGTTCTTAGACACTCCCTTAGGGC
CGCTTCCCCTCTCAGGCCAGCTTCCCTAAGGCTTTCAGTACCCAGGATCTGG
AAGGAAAGGGCCAAGCTGGGCTGTGGCATCCACTGGACCCTATAGTCTCAT
TGGGCAGGGCCTCAGAATCCACAAAGACTCCCCAGTGCTGTTCCTCTTCCA
ACCAGGCACTAAGTGTTGGCTTTAAAGTCCTAGGGGCCGGCCCCGCCG
(SEQ ID NO: 71, ERBB2-4)
GCCCGCGAGGACCCGCCCGAGCTTCGAAAAAAAAAAGCCATCGAGGCAAA
ATTCCCCCATCTGCGTCAGTCCCCTCAACTTCCGCCTCTACGCGGGCCTCGC
GGCAGGGCGGTACGTCAGCCATTCTGGTCCGCCGCGCGCACGCTCCGGGCG
CCGGGTTCCGGCGTTGTCAGGGTCCGCGGCCCTACGATGTGGGCGGTGTCC
AAGGCTGCGTAGTGGAGCTTGCAGGCTGGAGCTNNNNNNNTNNNNNNNNN
NNNNGNNNCCNTANGTTTCTTTTTATATATTTCGCTCTGGAACTGCGGGCGT
GGAGAAGGACCCCCCACACTGGCCTCCCCATACAACACCCATTCTCCCCCT
GGGTCTTTATTTCGTCTTTGAGGCCGCGGGGGACATGACTTACCACCTAAC
CATGAGGGCACTCTCAACGCGACCTGGATACTGACACCATTGCTGTTCCTT
CCTCATGGGGGGCAGTACTAAGTTTCAGGGACAGTCTCTGAATGGGTCGCT
TTTGTTCTTANGTCTCCCTTATGGCCTCTTCCCCTCTCAAGCCAATTTCCNT
AAGGATTTCTCTACCAANGATCTGGAAAAAAAGGGCCAATTTGGGCTGTGGC
ATCCCCAGGACCNNACATTGCATGAAAGCTAATGCCGACAAGGATCAATA
AAAAACTCTTANATTATTTCTTAATACTTCCCCTACACTTNCTAAGTGTTGG
CTTTAAAGTCCTAGGGGCCGGCCCCGCCG
(SEQ ID NO: 72, ERBB2-30)
GCCCGCGAGGACCCGCCCGAGCTTCGAAAAAAAAAAGCCATCGAGGCAAA
ATTCCCCCATCTGCGTCAGTCCCCTCAACTTCCGCCTCTACGCGGGCCTCGC
GGCAGGGCGGTACGTCAGCCATTCTGGTCCGCCGCGCGCACGCTCCGGGCG
CCGGGTTCCGGCGTTGTCAGGGTCCGCGGCCCTACGATGTGGGCGGTGTCC
AAGGCTGCGTAGTGGAGCTTGCAGGCTGGAGCTNNNNNNNNNNNNNGNAN
NTCCCCCATCTGCGTCAGTCCCCTCAACTTCCGCCTCTACGCGGGCCTCGCG
GCAGGGCGGTACGTCAGCCATTCTGGTCCGCCGCGCGCATGCGTAGTGGAG
CTTGCAGGCTGGAGCTGAGCAATCACCTATGAACTTGCTGCTAAGTTATGA
TCAAATGCAACCTCACAACCTTGGCTGAGTCTTGAGACTGAAAGATTTAGC
CATAATGTAAACTGCCTCAAATTGGACTTTGGGCATAAAAGAACCCTCCTG
CATGCTTACCATCCCTCCTGCCCTCCTTTAACAGCCTCGTAACTTATAATTG
ACCTCAAAAAACCTCAAGATTTACACAATGTTTCTCTGTAAATATTGCCATT
AAATGTAAATAACTTTAATAAAACGTTTATAGCAGTTACACAGAATTTCAA
TCCTAGTATATAGTACCTAGTATTATAGGTACTATAAACCCTAACCTCCTGC
ATTTAAGTACATTTTGCACTGCAAAGTAAGTTATCCTCTATTGACCTCAGAA
AAAATAAAATAACTGGCAAATATATCATTGAGCCAAATCTTAAGTTGTGAA
TGCTGCCTCTGCTAACTTATCCCTCCCAACCACCACCATCCCTGCTGCCCTC
ATCAAAAAACCTACGGGCCGGTCTCGCCGTAATGTGAGTACTAAGTGTTGG
CTTTAAAGTCCTAGGGGCCGGCCCCGCCG
(SEQ ID NO: 73, MYC 2-3)
GCCCGCGAGGACCCGCCCGAGCTTCGAAAAAAAAAAGCCATCGAGGCAAA
ATTCCCCCATCTGCGTCAGTCCCCTCAACTTCCGCCTCTACGCGGGCCTCGC
GGCAGGGCGGTACGTCAGCCATTCTGGTCCGCCGCGCGCACGCTCCGGGCG
CCGGGTTCCGGCGTTGTCAGGGTCCGCGGCCCTACGATGTGGGCGGTGTCC
AAGGCTGCGTAGTGGAGCTTGCAGGCTGGAGCTNNNNNNNNNNTTNNNNA
GANGGCACAGGNNTGGTGGTGGTTGGGAGGGATAAGTTAGCACAGGCAGC
ATTCACAACTTAAGATTTGGCTCATGATATATTTGCCAGTTATTTTATTTTT
TCTGAGGTCAATAAAGGATAACTTACTTTGTAGNGCAAAATGAACTTAAATG
CAAGAGGTAAAGGNCAATAGAACCTTTAATACAACGTATNNTTTACTTNGN
AAAAAANCCAANGTNACGGCTTAAAACCCTCNCTAAANAANCACCNNTCC
AAAAGTCAATTCAGACGTACNCCNCCCNCNANGGGCCNCTAAATTNNTACT
ACCAACNTNCTGCCCCCTCCATACANAAAANCTAAGTGTTGGCTTTAAAGT
CCTAGGGGCCGGCCCCGCCG (SEQ ID NO: 74, MYC 1-18)
GCCCGCGAGGACCCGCCCGAGCTTCGAAAAAAAAAAGCCATCGAGGCAAA
ATTCCCCCATCTGCGTCAGTCCCCTCAACTTCCGCCTCTACGCGGGCCTCGC
GGCAGGGCGGTACGTCAGCCATTCTGGTCCGCCGCGCGCACGCTCCGGGCG
CCGGGTTCCGGCGTTGTCAGGGTCCGCGGCCCTACGATGTGGGCGGTGTCC
AAGGCTGCGTAGTGGAGCTTGCAGGCTGGAGCTNNNNNNNGGCTTTTNNN
GNNNNNNCNGGGATGGTGGTGGTTGGGAGGGATAAGTTAGCAGAGGCAGC
ATTCACAACTTAAGATTTGGCTCAATGATACCTTTGCCAGTTATTTTATTTT
TTCTGATGTCAATAAAACGATAACTTACTTTGCATTGCAAAATGTNCATAC
ATGCAGGANGTTACGGCTTANNAAACCTTTAATAATCACCACATATATCTT
TCAAAGAAAGCTAAAAGTTCTGTTATAAACATCACTATAAAAACAATAATC
CAAGAATAACTTCAGAACTACCCCTCCCACTAGNAAACGCTAAATTGCTAA
TAACAACATATTGCNCTCTNNAAACAAAAAAAACTAAGTGTTGGCTTTAAA
GTCCTAGGGGCCGGCCCCGCCG (SEQ ID NO: 75, MYC 1-14)
GCCATCGAGGCAAAATTCCCCCATCTGCGTCAGTCCCCTCAACTTCCGCCT
CTACGCGGGCCTCGCGGCAGGGCGGTACGTCAGCCATTCTGGTCCGCCGCG
CGCACGCTCCGGGCGCCGGGTTCCGGCGTTGTCAGGGTCCGCGGCCCTACG
ATGTGGGCGGTGTCCAAGGCTGCGTAGTGGAGCTTGCAGGCTGGAGCTGG
GNNNNAANNNNNNNNNCNGANGCCTGCCAGTGAGAGTCNAGGGTCCAGT
GAAGGCCGCATCGCACCTTGGCCCTCTCCTTCCGTATCCTGGGTACTGAGG
CCGTAGGGAAGCTGGCCNTTGAGGGGAAGCGAGGACTNGAAGTGAGNGAA
GCCAGCCTACCCGGTAAAATCGGGCTCTGAGTCGNTTGGGCCCAACCTGAG
GAGGATTAGCATTGCCGTGAGCGGCGGGGGTTACCTAAGGTGCTGAACAG
TTCCCTCCCTGTCGCATTGGTNGGGAACCATCTAGCTCAGCCACATCCTCCC
GCTACGNGCTTTGGGCTGATTGCAANCCATGGGACTGACACCACTTCTACT
ACATCCTCNNNGGCGGTGACTCTTCNACCTCCACCCATAGACCCATATTNC
CCTNATTCTCCTCNTACCANNAAAANNCCTGTTCGGNNCCANCACCATCAA
TTCTTCNCTNNNAGATTNNTCCATCATTCTTTTTCTNNNCAATATGTAACAT
TGTTTTNNATGAANNNCCAAANNCCN (SEQ ID NO: 76, ERBB2-1)
GCCATCGAGGCAAAATTCCCCCATCTGCGTCAGTCCCCTCAACTTCCGCCT
CTACGCGGGCCTCGCGGCAGGGCGGTACGTCAGCCATTCTGGTCCGCCGCG
CGCACGCTCCGGGCGCCGGGTTCCGGCGTTGTCAGGGTCCGCGGCCCTACG
ATGTGGGCGGTGTCCAAGGCTGCGTAGTGGAGCTTGCAGGCTGGAGCTGN
NNNNNNNNTNNNNNANCAGAGGCNNGCTCACTGAGACTCTATGGTCAGTG
AAGGCCACACCCCAACTTGGCCCTTTCCTTCCGGATCCTGGGTACTGAGGC
CTTAGGGAAGCTGGCCTGACATGGGAAGCAGCCCTTGCGGAGTGTCTTGAA
CTGAACCGACCCATTAAAAGACTGTCCCTGAAACCTCGTACTGCCCCCCTG
AGGAAGGAACTGCCCTGTTGGCAGTGTCGGTGCTTTGTACACAGTGCTTTT
CNGTTTATNTTTTACTTTTTTTGTTTTGCTTTTTTAAATATGAAATTCATAC
CCATGTTGACAATGGGTGTTGTATGGGAAGGCAAGGGACTCCCTTTGGTGAT
GGGCGCGACCTTTGGTATCGCCATATCGCTGGTGATTATCACCTGGTCTGAT
GTGTATACCGGACCCACCCCTTNNCACATCCAANAAGGCTCTTGCTGATGG
CAATATCTGGTGGTGGCTCTCNNAAAAANCTCATCC (SEQ ID NO: 77,
ERBB2-2)
GCCATCGAGGCAAAATTCCCCCATCTGCGTCAGTCCCCTCAACTTCCGCCT
CTACGCGGGCCTCGCGGCAGGGCGGTACGTCAGCCATTCTGGTCCGCCGCG
CGCACGCTCCGGGCGCCGGGTTCCGGCGTTGTCAGGGTCCGCGGCCCTACG
ATGTGGGCGGTGTCCAAGGCTGCGTAGTGGAGCTTGCAGGCTGGAGCTNN
NNNNNNNANTTNNNNAACNNANGCCNGCNNNTGAGACTCTACGGTCCAGT
GGATGTAACATCCCAGCTTGGTCCTTTCCTTCCATATCCTGGGTACTGAAAG
CCTTAGGGAAACTGGCCTGATAGGGGAAGCGGCCCTACATGATTGGATGG
NACGGAATCGACCCTTTCAGAGACTGTCCCTGAAACCTATCAGTGCCCGCC
ACGATGAAAGAACAGCAACGGTGACAGTATCCTGGCATTGTCCAAANNGCT
TTTCTGTGCTTNTTTACTTTTTTTAATNAGTTTTTTTAAAGATCAAACATCC
ACCCCAGGTCCCAATGGGGGGTGCCTGAGGAAGAACGATTGGCGCCTGTTC
CCGTTCCCCTTTACTGGACTGCGTACTACACCCCGAGGCCTTCTTTGAGAGA
GCGNNCCATCCTGGGACAGCAACCTAACTANNCCCTCTCCCCCTGCGGATG
ACAATGTCTGGCCGGGGTTCTNATAGGCTACTCACCCCCTTTTGTTTTTATT
ACAAACGATTATGTTTGNTCTGACCNCANNAAA
(SEQ ID NO: 78, ERBB2-3)
GCCATCGAGGCAAAATTCCCCCATCTGCGTCAGTCCCCTCAACTTCCGCCT
CTACGCGGGCCTCGCGGCAGGGCGGTACGTCAGCCATTCTGGTCCGCCGCG
CGCACGCTCCGGGCGCCGGGTTCCGGCGTTGTCAGGGTCCGCGGCCCTACG
ATGTGGGCGGTGTCCAAGGCTGCGTAGTGGAGCTTGCAGGCTGGAGCTNN
NNNNNNNNNTTNAAANATATTTGCAATGGACAAAGTGGGTGTGGAGAAGG
ACCCCCCACACTTGCCTCCCCATACAACACCCATTCTCCCCCTGGGTCTTTA
TTTCATCTTTAAAAAAACAAAACAAAAAAAGTAAAAACTAAACACAAAAG
CACTCTGTACAAAGCCTGGATACTGACACCATTGCTGTTCCTTCCTCATGGG
GGGCAGTACTAGGTTTCATGGACAGTCTCTGAATGGTTCGCTTTTGTTCTTA
GACACTCCCTTAGGGCCGCTTCCCCTCTCAGGCCAGCTTCCCTAAGGCTTTC
AGTACCCAGGATCTGGAAGGAAAGGGCCAAGCTGGGCTGTGGCATCCACT
GGACCCTATAGTCTCATTGGGCAGGGCCTCAGAATCCACAAAGACTCCCCA
GTGCTGTTCCTCTTCCAACCAGGCA (SEQ ID NO: 79, ERBB2-4)
GCCATCGAGGCAAAATTCCCCCATCTGCGTCAGTCCCCTCAACTTCCGCCT
CTACGCGGGCCTCGCGGCAGGGCGGTACGTCAGCCATTCTGGTCCGCCGCG
CGCACGCTCCGGGCGCCGGGTTCCGGCGTTGTCAGGGTCCGCGGCCCTACG
ATGTGGGCGGTGTCCAAGGCTGCGTAGTGGAGCTTGCAGGCTGGAGCTNN
NNNNNTNNNNNNNNNNNNNGNNNCCNTANGTTTCTTTTTATATATTTCGCT
CTGGAACTGCGGGCGTGGAGAAGGACCCCCCACACTGGCCTCCCCATACA
ACACCCATTCTCCCCCTGGGTCTTTATTTCGTCTTTGAGGCCGCGGGGGACA
TGACTTACCACCTAACCATGAGGGCACTCTCAACGCGACCTGGATACTGAC
ACCATTGCTGTTCCTTCCTCATGGGGGGCAGTACTAAGTTTCAGGGACAGT
CTCTGAATGGGTCGCTTTTGTTCTTANGTCTCCCTTATGGCCTCTTCCCCTC
TCAAGCCAATTTCCNTAAGGATTTCTCTACCAANGATCTGGAAAAAAAGGGC
CAATTTGGGCTGTGGCATCCCCAGGACCNNACATTGCATGAAAGCTAATGC
CGACAAGGATCAATAAAAAACTCTTANATTATTTCTTAATACTTCCCCTAC
ACTTN (SEQ ID NO: 80, ERBB2-30)
GCCATCGAGGCAAAATTCCCCCATCTGCGTCAGTCCCCTCAACTTCCGCCT
CTACGCGGGCCTCGCGGCAGGGCGGTACGTCAGCCATTCTGGTCCGCCGCG
CGCACGCTCCGGGCGCCGGGTTCCGGCGTTGTCAGGGTCCGCGGCCCTACG
ATGTGGGCGGTGTCCAAGGCTGCGTAGTGGAGCTTGCAGGCTGGAGCTNN
NNNNNNNNNNNGNANNTCCCCCATCTGCGTCAGTCCCCTCAACTTCCGCCT
CTACGCGGGCCTCGCGGCAGGGCGGTACGTCAGCCATTCTGGTCCGCCGCG
CGCATGCGTAGTGGAGCTTGCAGGCTGGAGCTGAGCAATCACCTATGAACT
TGCTGCTAAGTTATGATCAAATGCAACCTCACAACCTTGGCTGAGTCTTGA
GACTGAAAGATTTAGCCATAATGTAAACTGCCTCAAATTGGACTTTGGGCA
TAAAAGAACCCTCCTGCATGCTTACCATCCCTCCTGCCCTCCTTTAACAGCC
TCGTAACTTATAATTGACCTCAAAAAACCTCAAGATTTACACAATGTTTCTC
TGTAAATATTGCCATTAAATGTAAATAACTTTAATAAAACGTTTATAGCAG
TTACACAGAATTTCAATCCTAGTATATAGTACCTAGTATTATAGGTACTATA
AACCCTAACCTCCTGCATTTAAGTACATTTTGCACTGCAAAGTAAGTTATCC
TCTATTGACCTCAGAAAAAATAAAATAACTGGCAAATATATCATTGAGCCA
AATCTTAAGTTGTGAATGCTGCCTCTGCTAACTTATCCCTCCCAACCACCAC
CATCCCTGCTGCCCTCATCAAAAAACCTACGGGCCGGTCTCGCCGTAATGT
GAGTA (SEQ ID NO: 81, MYC 2-3)
GCCATCGAGGCAAAATTCCCCCATCTGCGTCAGTCCCCTCAACTTCCGCCT
CTACGCGGGCCTCGCGGCAGGGCGGTACGTCAGCCATTCTGGTCCGCCGCG
CGCACGCTCCGGGCGCCGGGTTCCGGCGTTGTCAGGGTCCGCGGCCCTACG
ATGTGGGCGGTGTCCAAGGCTGCGTAGTGGAGCTTGCAGGCTGGAGCTNN
NNNNNNNNTTNNNNAGANGGCACAGGNNTGGTGGTGGTTGGGAGGGATA
AGTTAGCACAGGCAGCATTCACAACTTAAGATTTGGCTCATGATATATTTG
CCAGTTATTTTATTTTTTCTGAGGTCAATAAAGGATAACTTACTTTGTAGNG
CAAAATGAACTTAAATGCAAGAGGTAAAGGNCAATAGAACCTTTAATACA
ACGTATNNTTTACTTNGNAAAAAANCCAANGTNACGGCTTAAAACCCTCNC
TAAANAANCACCNNTCCAAAAGTCAATTCAGACGTACNCCNCCCNCNANG
GGCCNCTAAATTNNTACTACCAACNTNCTGCCCCCTCCATACANAAAAN
(SEQ ID NO: 82, MYC 1-18)
GCCATCGAGGCAAAATTCCCCCATCTGCGTCAGTCCCCTCAACTTCCGCCT
CTACGCGGGCCTCGCGGCAGGGCGGTACGTCAGCCATTCTGGTCCGCCGCG
CGCACGCTCCGGGCGCCGGGTTCCGGCGTTGTCAGGGTCCGCGGCCCTACG
ATGTGGGCGGTGTCCAAGGCTGCGTAGTGGAGCTTGCAGGCTGGAGCTNN
NNNNNGGCTTTTNNNGNNNNNNCNGGGATGGTGGTGGTTGGGAGGGATAA
GTTAGCAGAGGCAGCATTCACAACTTAAGATTTGGCTCAATGATACCTTTG
CCAGTTATTTTATTTTTTCTGATGTCAATAAAACGATAACTTACTTTGCATT
GCAAAATGTNCATACATGCAGGANGTTACGGCTTANNAAACCTTTAATAAT
CACCACATATATCTTTCAAAGAAAGCTAAAAGTTCTGTTATAAACATCACT
ATAAAAACAATAATCCAAGAATAACTTCAGAACTACCCCTCCCACTAGNAA
ACGCTAAATTGCTAATAACAACATATTGCNCTCTNNAAACAAAAAAAA
(SEQ ID NO: 83, MYC 1-14)

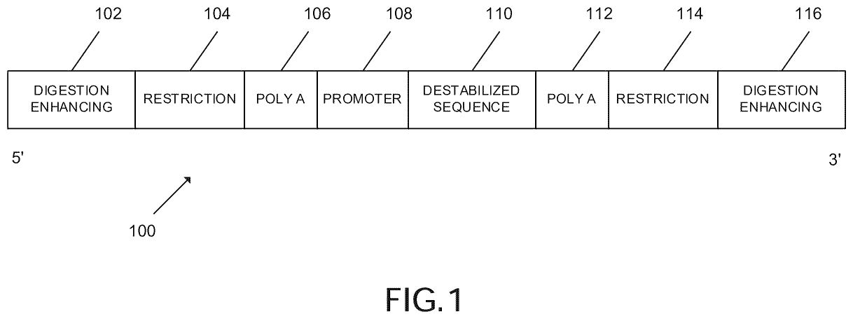

Referring to FIG. 1, a sequence 100 is shown which comprises a first digestion enhancing sequence 102, a first restriction sequence 104, a first polyA sequence 106, a promoter sequence 108, a destabilized sequence 110 (the disclosed destabilized ARE 3′UTRs), a second polyA sequence 112, a second restriction sequence 114 and a second digestion enhancing sequence 116. In one embodiment, the sequence 100 is a DNA sequence that is incorporated into a plasmid vector.

The first digestion enhancing sequence 102 is located at the 5′ end and may be selected from known sequences. In one embodiment, the first digestion enhancing sequence 102 is between five and one hundred residues. In another embodiment, the first digestion enhancing sequence 102 is between five and fifty residues. In one embodiment, both the first digestion enhancing sequence 102 and the digestion enhancing sequence 116 are the same. In one embodiment, the first digestion enhancing sequence 102 is GGACCCGCCCGAGC (SEQ ID NO: 26). In another embodiment, the first digestion enhancing sequence 102 is GGGCCGGCCCCGCCG (SEQ ID NO: 27). In yet another embodiment, the first digestion enhancing sequence 102 is GCCCGCGAGGACCCGCCCGAGC (SEQ ID NO: 28).

The first restriction sequence 104 may be selected from known sequences. Suitable examples include a BstB1 restriction site, a BamH1 restriction site, a Xba1 restriction site, a Apa1 restriction site, a PspOM1 restriction site, and the like. In one embodiment, the first restriction sequence 104 has between four and seven residues. In one embodiment, both the first restriction sequence 104 and the second restriction sequence 114 are different.

The first polyA sequence 106 generally contains between four and twenty residues. The first polyA sequence 106 and the second polyA sequence 112 may be the same or different. The first polyA sequence functions to stop the RFP transcription. In one embodiment, the first poly A sequence is AAAA, AAAAAAAA or more. In one embodiment, the second polyA sequence is AAAA or AAAAAAAA.

The promoter sequence 108 may be selected from known sequences. Suitable examples include a DCP1A promoter, a DCP2 promoter and a ZFP36 promoter. In one embodiment, the promoter sequence 108 is between one hundred forty and one hundred seventy residues. In another embodiment, promoter sequence 108 is between one hundred fifty and one hundred sixty residues.

The destabilized sequence 110 is based on RNAs that are associated with cancer but wherein the resulting RNA is less stable than the wildtype RNA found in cancer cells.

The second polyA sequence 112 contains between four and twenty residues. The first polyA sequence 106 and the second polyA sequence 112 may be the same or different. The second polyA sequence 112 functions to stop transcription.

The second restriction sequence 114 may be selected from known sequences. Suitable examples include a BstB1 restriction site and a BamH1 restriction site. In one embodiment, both the first restriction sequence 104 and the second restriction sequence 114 are different.

The second digestion enhancing sequence 116 may be selected from known sequences. In one embodiment, the first digestion enhancing sequence is between 5 and 50 residues. In one embodiment, the second digestion enhancing sequence 116 is GGACCCGCCCGAGC (SEQ ID NO: 26). In another embodiment, the second digestion enhancing sequence 116 is GGGCCGGCCCCGCCG (SEQ ID NO: 27). In yet another embodiment, the second digestion enhancing sequence 116 is GCCCGCGAGGACCCGCCCGAGC (SEQ ID NO: 28).

As detailed herein, modification, and, thereby, destabilization is driven by degrading endogenous mRNA coupled with an increase in transcription of the destabilized mRNA construct relative to the wildtype RNA (i.e., transcription of the destabilized mRNA is more efficient such that the destabilized 3′ UTR outcompetes the wildtype RNA).

Thus, in various aspects, the DNA molecules of the invention comprise a nucleic acid sequence that encodes a 3′ UTR of a MYC or ERBB2 gene in an mRNA molecule in which one or more ARE poly(U) stabilizing motifs of the 3′ UTR of the mRNA are substituted with a destabilizing motif, as further described herein. This destabilized ARE of the 3′ UTR of the mRNA will be driven by an mRNA decapping protein, e.g., DCP1A, which will specifically upregulate the mRNA decay pathway and trigger the deadenylase CNOT1 and the cleavage enzyme XRN1 to degrade the RNA transcript of interest (e.g., the MYC RNA transcript or the ERBB2 RNA transcript).

In various aspects, the RNA molecule comprises a 3′ UTR of an mRNA encoding a MYC or ERBB2 protein, in which one or more ARE poly(U) stabilizing motifs of the 3′ UTR are substituted with a nucleic acid sequence selected from SEQ ID NO: 1, SEQ ID NO: 2, and SEQ ID NO: 3, wherein the one or more ARE poly(U) stabilizing motifs comprise at least three consecutive U's. In various further aspects, the RNA molecule comprises a 3′ UTR of an mRNA encoding a MYC or ERBB2 protein, in which one or more ARE poly(U) stabilizing motifs of the 3′ UTR are substituted with a nucleic acid sequence selected from SEQ ID NO: 1, SEQ ID NO: 2, and SEQ ID NO: 3, wherein the one or more ARE poly(U) stabilizing motifs comprise at least four consecutive U's. In various further aspects, the RNA molecule comprises a 3′ UTR of an mRNA encoding a MYC or ERBB2 protein, in which one or more ARE poly(U) stabilizing motifs of the 3′ UTR are substituted with a nucleic acid sequence selected from SEQ ID NO: 1, SEQ ID NO: 2, and SEQ ID NO: 3, wherein the one or more ARE poly(U) stabilizing motifs comprise at least five consecutive U's.

In various aspects, the RNA molecule comprises a 3′ UTR of an mRNA encoding a MYC or ERBB2 protein, in which one or more ARE poly(U) stabilizing motifs of the 3′ UTR are substituted with a nucleic acid sequence selected from SEQ ID NO: 1, SEQ ID NO: 2, and SEQ ID NO: 3, wherein the one or more ARE poly(U) stabilizing motifs comprise SEQ ID NO: 4.

In various aspects, at least two ARE poly(U) stabilizing motifs of the 3′ UTR of the mRNA are substituted with a nucleic acid sequence selected from SEQ ID NO: 1, SEQ ID NO: 2, and SEQ ID NO: 3. In a further aspect, at least four ARE poly(U) stabilizing motifs of the 3′ UTR of the mRNA are substituted with a nucleic acid sequence selected from SEQ ID NO: 1, SEQ ID NO: 2, and SEQ ID NO: 3. In a still further aspect, at least four ARE poly(U) stabilizing motifs of the 3′ UTR of the mRNA are substituted with a nucleic acid sequence selected from SEQ ID NO: 1, SEQ ID NO: 2, and SEQ ID NO: 3.

In various aspects, every ARE poly(U) stabilizing motif of the 3′ UTR of the mRNA is substituted with a nucleic acid sequence selected from SEQ ID NO: 1, SEQ ID NO: 2, and SEQ ID NO: 3.

In various aspects, the DNA molecules of the invention contain various components (e.g., a promoter, a nucleic acid sequence encoding a 3′ UTR of an ERBB2 or MYC gene in a mRNA molecule, a first and/or second polyA sequence, a first and/or second restriction sequence), which components can be operatively linked to one another in order to provide a DNA construct useful as a therapeutic component as further described herein.

### C. Methods of Making cDNA Molecules

In one aspect, disclosed are methods of making a DNA molecule (e.g., a complementary deoxyribonucleic acid or cDNA molecule), the method comprising reverse transcribing a RNA molecule of the invention to produce the DNA molecule. In a further aspect, the DNA molecule is a cDNA molecule.

In one aspect, disclosed are DNA molecules (e.g., cDNA molecules) prepared by a disclosed method.

In one aspect, disclosed are DNA molecules (e.g., cDNA molecules) prepared from a disclosed RNA molecule. cDNA is distinct from genomic DNA, as the derivative template RNA transcript lacks promoters and introns. As would be understood by one of skill in the art, cDNA can be synthesized via reverse transcription, in which mRNA or miRNA is used as a template together with a reverse transcription enzyme and a thermostable primer that is complementary to the 3′ end of the RNA template to generate a cDNA product that is a complementary copy of the mRNA. This cDNA product can then be used as a template to produce a second DNA strand using polymerase chain reaction (PCR) assays.

Briefly, the RNA samples (e.g., a RNA molecule as disclosed herein) are prepared and any remaining genomic DNA is removed. The sample is then combined with the reverse transcriptase enzyme and various other components (e.g., dNTPs, DTT, buffer, RNAse inhibitors, RNase-free water), followed by primer annealing, DNA polymerization, and enzyme activation. Reverse transcription kits are well-known by those of skill (e.g., Qiagen reverse transcription kit, catalog no. 205311) and can be used to facilitate the transformation using the manufacturer's protocol. In this way, cDNA comprising, in the 5′→3′ direction of transcription, a promoter, operatively linked to a nucleic acid sequence encoding a 3′ UTR of an ERBB2 or MYC gene in the mRNA molecule in which one or more ARE poly(U) stabilizing motifs of the 3′ UTR of the mRNA are substituted with a nucleic acid sequence selected from AUUUU, CCUC, CUGC, UAAGUUAU, UAACUUAU, and GUAAAUAG can be generated.

As used herein, the term “reverse transcription” means a process wherein the genetic code in an RNA sequence (e.g., an RNA molecule as described herein) is reverse transcribed into DNA. See, e.g., Sissaoui et al. (2020) Circ. Res. 126 (7): 875-888. Subsequently, the DNA can be incorporated into a vector and then, transfected into cells. As used herein, the term “reverse transcription” includes “in vitro reverse transcription,” wherein the term “in vitro reverse transcription” relates to a process wherein DNA, in particular, cDNA, is in vitro synthesized in a cell-free system.

In various aspects, the RNA molecule used to synthesize the DNA molecule of the invention comprises a 3′ UTR of an mRNA encoding a ERBB2 protein, in which one or more ARE poly(U) stabilizing motifs of the 3′ UTR are substituted with a nucleic acid sequence selected from AUUUU, CCUC, CUGC, UAAGUUAU, UAACUUAU, and GUAAAUAG. Thus, in various aspects, the RNA molecule of the invention can include, in addition to the 3′ UTR of an mRNA encoding an ERBB2 protein, additional sequences that may or may not code for the ERBB2 protein. In various further aspects, the RNA molecule of the invention is just the 3′ UTR of an mRNA encoding an ERBB2 protein.

In various aspects, the RNA molecule used to synthesize the DNA molecule of the invention comprises a 3′ UTR of an mRNA encoding a MYC protein, in which one or more ARE poly(U) stabilizing motifs of the 3′ UTR are substituted with a nucleic acid sequence selected from AUUUU, CCUC, CUGC, UAAGUUAU, UAACUUAU, and GUAAAUAG. Thus, in various aspects, the RNA molecule of the invention can include, in addition to the 3′ UTR of an mRNA encoding a MYC protein, additional sequences that may or may not code for the MYC protein. In various further aspects, the RNA molecule of the invention is just the 3′ UTR of an mRNA encoding a MYC protein.

In various aspects, the DNA molecule of the invention is prepared by reverse transcribing a RNA molecule comprising a 3′ UTR of an mRNA encoding a ERBB2 protein, in which at least four ARE poly(U) stabilizing motifs of the 3′ UTR are substituted with a nucleic acid sequence selected from AUUUU, CCUC, CUGC, UAAGUUAU, UAACUUAU, and GUAAAUAG, and wherein the 3′ UTR of the mRNA encoding the ERBB2 protein is at least 80% identical to SEQ ID NO: 13.

In various aspects, the DNA molecule of the invention is prepared by reverse transcribing a RNA molecule consisting essentially of a 3′ UTR of an mRNA encoding a ERBB2 protein, in which at least four ARE poly(U) stabilizing motifs of the 3′ UTR are substituted with a nucleic acid sequence selected from AUUUU, CCUC, CUGC, UAAGUUAU, UAACUUAU, and GUAAAUAG, and wherein the 3′ UTR of the mRNA encoding the ERBB2 protein is at least 80% identical to SEQ ID NO: 13.

In various aspects, the DNA molecule of the invention is prepared by reverse transcribing a RNA molecule comprising a 3′ UTR of an mRNA encoding a MYC protein, in which at least four ARE poly(U) stabilizing motifs of the 3′ UTR are substituted with a nucleic acid sequence selected from AUUUU, CCUC, CUGC, UAAGUUAU, UAACUUAU, and GUAAAUAG, and wherein the 3′ UTR of the mRNA encoding the MYC protein is at least 80% identical to SEQ ID NO: 12.

In various aspects, the DNA molecule of the invention is prepared by reverse transcribing a RNA molecule consisting essentially of a 3′ UTR of an mRNA encoding a MYC protein, in which at least four ARE poly(U) stabilizing motifs of the 3′ UTR are substituted with a nucleic acid sequence selected from AUUUU, CCUC, CUGC, UAAGUUAU, UAACUUAU, and GUAAAUAG, and wherein the 3′ UTR of the mRNA encoding the MYC protein is at least 80% identical to SEQ ID NO: 12.

### D. Plasmid Vectors

In one aspect, disclosed are plasmid vectors comprising a DNA molecule of the invention. As used herein, the term “vector” means any vehicle for carrying a nucleic acid that can, for example, enable said nucleic acid to be introduced into prokaryotic and/or eukaryotic host cells and, where appropriate, to be integrated into a genome. Such vectors are preferably replicated and/or expressed in the cell. Vectors comprise plasmids, phagemids, and virus genomes. The term “plasmid,” as used herein, generally relates to a construct of extrachromosomal genetic material, usually a circular DNA duplex, which can replicate independently of chromosomal DNA.

The term “plasmid vector” means a small, single or double-stranded circular extrachromosomal DNA or RNA molecule. Plasmid vectors can consist of the transgene insert and an origin of replication, a promoter region, optionally a selectable marker, and convenient restriction sites. These features allow for semi-independent replication of the plasmid in the host (e.g., hundreds of copies can be made per cell) and convenient restriction sites. In certain embodiments as used here the term “vector” may refer to double stranded DNA plasmid vectors used to carry DNA encoding for the lentiviral plasmid vector.

Vectors derived from retroviruses such as the lentivirus (e.g., a lentiviral vector) are suitable tools to achieve long-term gene transfer since they allow long term, stable integration of a transgene and its propagation in daughter cells. These vectors have emerged as promising tools for both gene therapy and immunotherapy purposes, because they exhibit several advantages over other viral systems. For example, unlike vectors derived from onco-retroviruses, retroviral vectors can transduce non-proliferating cells, such as hepatocytes. Additionally, they are non-toxic to target cells. In in vivo applications, lentiviral vectors have the added advantage of low immunogenicity. Moreover, lentiviral vectors, in particular, are also advantageous because they can deliver genes to cell types that previous retrovirus vectors could not, such as neurons, lymphocytes, and macrophages.

Lentiviruses represent a genus of slow viruses of the Retroviridae family, which includes the human immunodeficiency viruses (HIV), the simian immunodeficiency virus (SIV), the equine infectious encephalitis virus (EIAV), the caprine arthritis encephalitis virus (CAEV), the bovine immunodeficiency virus (BIV), and the feline immunodeficiency virus (FIV). Lentiviruses can persist indefinitely in their hosts and replicate continuously at variable rates during the course of the lifelong infection. Persistent replication of the viruses in their hosts depends on their ability to circumvent host defenses.

The design of recombinant integrating lentiviral vectors is based on the separation of the cis- and trans-acting sequences of the lentivirus. Efficient transduction in non-dividing cells requires the presence of two cis-acting sequences in the lentiviral genome, the central polypurine tract (cPPT) and the central termination sequence (CTS). These lead to the formation of a triple-stranded DNA structure called the central DNA “flap”, which maximizes the efficiency of gene import into the nuclei of non-dividing cells, including dendritic cells (DCs) (Zennou et al. (2000) Cell 101 (2) 173-85; Arhel et al. (2007) EMBO J 26 (12): 3025-37).

A vital component of the integration complex of LV is the viral integrase enzyme (IN) that catalyzes viral DNA integration into the host genome, as it mediates the integration between vector and host DNA. However, this process carries predictable risks of harmful insertional mutagenesis, prompting an examination of alternatives to vector-mediated integration. To address this, non-integrating lentiviral vectors (NILVs) were established. NILVs can stably express transgenes from the extrachromosomal DNA in non-dividing cells or transiently if the target cells divide both in vitro and in vivo.

Lentiviral particles containing lentiviral vectors can be produced, by example, by recombinant technology upon transient transfection of cells (e.g., HEK 293 T human cultured cells) by a plasmid DNA such as, for example, a DNA plasmid of the invention. In this way, transient production of lentiviral particle vectors can be obtained from the transfected cells. Alternatively, lentiviral particle vectors can also be continuously produced by cells by stably inserting the packaging genes, the plasmid DNA (e.g., a DNA plasmid of the invention), and the envelope gene into the cellular genome. This allows for the continuous production of lentiviral particle vectors by the cells without the need for transient transfection. It is also possible to use a combination of these procedures, as would be understood by those of ordinary skill.

The plasmid vectors of the invention can be formulated for administration according to a variety of different techniques, which are known to those of ordinary skill in the art. For example, the disclosed plasmid vector can be formulated for administration via a lipid nanoparticle, nanodiamonds, liposomes, microspheres, polymeric micelles, GalNac-conjugation, as a dextran formulations, as a polyethylene glycol (PEG) formulation, as an exosome formulation, and any other similar formulation know to those of ordinary skill.

### 1. Nanoparticle Formulations

In various aspects, the plasmid vectors of the invention can be formulated for administration via a nanoparticle or nanoparticulate formulation.

As used herein, the term “nanoparticle” refers to any particle having a diameter making the particle suitable for systemic, in particular, parenteral, administration, of, in particular, nucleic acids, typically a diameter of less than 1000 nanometers (nm). In some aspects, a nanoparticle has a diameter of less than 600 nm. In some aspects, a nanoparticle has a diameter of less than 400 nm.

As used herein, the term “nanoparticulate formulation” or similar term refer to any substance that contain at least 0.2 μg, at least 1.0 μg, at least 5.0 μg, at least 10 μg, at least 15 μg or at least 20 μg of nanoparticles. In some aspects, a nanoparticulate composition is a uniform collection (e.g., from about 0.2 μg to about 20 μg) of nanoparticles. In some aspects, nanoparticulate compositions are dispersions or emulsions. In general, a dispersion or emulsion is formed when at least two immiscible materials are combined.

The nanoparticulate carriers such as lipid carriers contemplated for use in the present invention include any substances or vehicles with which a nucleic acid such as DNA can be associated, e.g., by forming complexes with the nucleic acid or forming vesicles in which the nucleic acid is enclosed or encapsulated. This can result in increased stability of the nucleic acid compared to a naked nucleic acid. In particular, stability of the nucleic acid in blood may be increased. In various aspects, the ratio of DNA, e.g., a DNA molecule of the invention, to lipid particle is from about 1:2 to about 2:1. In various further aspects, the ratio of DNA to lipid particle is about 1:1.

Nanoparticulate nucleic acid preparations for use in the present invention can be obtained by various protocols and from various nucleic acid complexing compounds. Lipids, polymers, oligomers, and amphipiles are typical complexing agents. In various aspects, the complexing compound comprises at least one agent selected from the group consisting protamine, polyethyleneimine, a poly-L-lysine, a poly-L-arginine, and a histone.

In addition, the nanoparticles described herein can further include a neutral lipid in view of structural stability and the like. The neutral lipid can be appropriately selected in view of the delivery efficiency of the nucleic acid-lipid complex. Examples of neutral lipids include, but are not limited to, 1,2-di-(9Z-octadecenoyl)-sn-glycero-3 -phosphoethanolamine (DOPE), 1,2-dioleoyl-sn-glycero-3-phosphocholine (DOPC), diacylphosphatidyl choline, diacylphosphatidyl ethanol amine, ceramide, sphingoemyelin, cephalin, sterol, and cerebroside. In the case where a cationic liposome includes both a cationic lipid and a neutral lipid, the molar ratio of the cationic lipid to the neutral lipid can be appropriately determined in view of stability of the liposome and the like. See, e.g., Piotrowski-Daspit et al. (2020) Advanced Drug Delivery Reviews 156:119-132; Kulkarni et al. (2021) Nature Nanotechnology 16:630-643; Zhong et al. (2023) Nature Materials 22:818-831.

According to one aspect, the nanoparticles described herein can comprise phospholipids. The phospholipids can be a glycerophospholipid. Examples of glycerophospholipid include, but are not limited to, three types of lipids: (i) zwitterionic phospholipids, which include, for example, phosphatidylcholine (PC), egg yolk phosphatidylcholine, soybean-derived PC in natural, partially hydrogenated or fully hydrogenated form, dimyristoyl phosphatidylcholine (DMPC) sphingomyelin (SM); (ii) negatively charged phospholipids: which include, for example, phosphatidylserine (PS), phosphatidylinositol (PI), phosphatidic acid (PA), phosphatidylglycerol (PG) dipalmipoyl PG, dimyristoyl phosphatidylglycerol (DMPG); synthetic derivatives in which the conjugate renders a zwitterionic phospholipid negatively charged such is the case of methoxy-polyethylene, glycol-distearoyl phosphatidylethanolamine (mPEG-DSPE); and (iii) cationic phospholipids, which include, for example, phosphatidylcholine or sphingomyelin of which the phosphomonoester was O-methylated to form the cationic lipids.

Association of nucleic acid to the lipid carrier can occur, for example, by the nucleic acid filling interstitial spaces of the carrier, such that the carrier physically entraps the nucleic acid, or by covalent, ionic, or hydrogen bonding, or by means of adsorption by non-specific bonds. Whatever the mode of association, the nucleic acid must retain its therapeutic properties.

### 2. Liposomal Formulations

Liposomes are microscopic lipidic vesicles often having one or more bilayers of a vesicle-forming lipid, such as a phospholipid, and are capable of encapsulating a drug. Different types of liposomes can be employed in the context of the present invention, including, but not limited to, multilamellar vesicles (MLV), small unilamellar vesicles (SUV), large unilamellar vesicles (LUV), sterically stabilized liposomes (SSL), multivesicular vesicles (MV), and large multivesicular vesicles (LMV), as well as other bilayered forms known in the art. The size and lamellarity of the liposome will depend on the manner of preparation and the selection of the type of vesicles to be used will depend on the preferred mode of administration. There are several other forms of supramolecular organization in which lipids can be present in an aqueous medium, comprising lamellar phases, hexagonal and inverse hexagonal phases, cubic phases, micelles, and reverse micelles composed of monolayers. These phases can also be obtained in the combination with DNA or RNA, and the interaction with RNA and DNA can substantially affect the phase state. The described phases can be present in the nanoparticulate nucleic acid formulations of the present invention. See, e.g., Piotrowski-Daspit et al. (2020) Advanced Drug Delivery Reviews 156:119-132; Kulkarni et al. (2021) Nature Nanotechnology 16:630-643; Zhong et al. (2023) Nature Materials 22:818-831.

For formation of nucleic acid lipoplexes from nucleic acid and liposomes, any suitable method of forming liposomes can be used so long as it provides the envisaged nucleic acid lipoplexes. Liposomes may be formed using standard methods such as, for example, the reverse evaporation method (REV), the ethanol injection method, the dehydration-rehydration method (DRV), sonication, and other suitable methods. After liposome formation, the liposomes can be sized to obtain a population of liposomes having a substantially homogeneous size range. See, e.g., Piotrowski-Daspit et al. (2020) Advanced Drug Delivery Reviews 156:119-132; Kulkarni et al. (2021) Nature Nanotechnology 16:630-643; Zhong et al. (2023) Nature Materials 22:818-831.

Bilayer-forming lipids typically have two hydrocarbon chains, particularly acyl chains, and a head group, either polar or nonpolar. Bilayer-forming lipids are either composed of naturally-occurring lipids or of synthetic origin, including the phospholipids, such as phosphatidylcholine, phosphatidylethanolamine, phosphatide acid, phosphatidylinositol, and sphingomyelin, where the two hydrocarbon chains are typically between about 14-22 carbon atoms in length, and have varying degrees of unsaturation. Other suitable lipids for use in the composition of the present invention include glycolipids and sterols such as cholesterol and its various analogs which can also be used in the liposomes.

The terms “lipoplex” and “nucleic acid lipoplex,” in particular, “DNA lipoplex,” means a complex of lipids and nucleic acids, in particular RNA. Lipoplexes are formed spontaneously when cationic liposomes, which often also include a neutral “helper” lipid, are mixed with nucleic acids.

Cationic lipids typically have a lipophilic moiety, such as a sterol, an acyl or diacyl chain, and have an overall net positive charge. The head group of the lipid typically carries the positive charge. The cationic lipid preferably has a positive charge of 1 to 10 valences, more preferably a positive charge of 1 to 3 valences, and more preferably a positive charge of 1 valence. Examples of cationic lipids include, but are not limited to, 1,2-di-O-octadecenyl-3-trimethylammonium propane (DOTMA); dimethyldioctadecylammonium (DDAB); 1,2-dioleoyl-3-trimethylammonium-propane (DOTAP); 1,2-dioleoyl-3-dimethylammonium-propane (DODAP); 1,2-diacyloxy-3-dimethylammonium propanes; 1,2-dialkyloxy-3-dimethylammonium propanes; dioctadecyldimethyl ammonium chloride (DODAC), 1,2-dimyristoyloxypropyl-1,3-dimethylhydroxyethyl ammonium (DMRIE), and 2,3-dioleoyloxy-N-[2 (spermine carboxamide)ethyl]-N,N-dimethyl-1-propanamium trifluoroacetate (DOSPA).

Cationic lipids, cationic polymers, and other substances with positive charges can form complexes with negatively charged nucleic acids. These cationic molecules can be used to complex nucleic acids, thereby forming, e.g., so-called lipoplexes or polyplexes, respectively. These complexes have been shown to deliver nucleic acids into cells.

### E. Drug Delivery Systems

In one aspect, disclosed are drug delivery system comprising: (a) an inorganic nanocage comprising palladium, iron oxide, or gold, wherein the inorganic nanocage has a diameter of 15 nm or less, loaded with (b) a plasmid vector comprising a therapeutically effective amount of a DNA molecule, wherein the DNA molecule comprises in the 5′→3′ direction of transcription relative to synthesis of a messenger RNA (mRNA) molecule comprising a 3′ UTR of an mRNA encoding an ERBB2 protein: (i) a promoter, operatively linked to (ii) a nucleic acid sequence encoding a 3′ UTR of an ERBB2 gene in the mRNA molecule in which one or more ARE poly(U) stabilizing motifs of the 3′ UTR of the mRNA are substituted with a nucleic acid sequence selected from AUUUU, CCUC, CUGC, UAAGUUAU, UAACUUAU, and GUAAAUAG.

In one aspect, disclosed are drug delivery system comprising: (a) an inorganic nanocage comprising palladium, iron oxide, or gold, wherein the inorganic nanocage has a diameter of 15 nm or less, loaded with (b) a plasmid vector comprising a therapeutically effective amount of a DNA molecule, wherein the DNA molecule comprises in the 5′→3′ direction of transcription relative to synthesis of a messenger RNA (mRNA) molecule comprising a 3′ UTR of an mRNA encoding a MYC protein: (i) a promoter, operatively linked to (ii) a nucleic acid sequence encoding a 3′ UTR of a MYC gene in the mRNA molecule in which one or more ARE poly(U) stabilizing motifs of the 3′ UTR of the mRNA are substituted with a nucleic acid sequence selected from AUUUU, CCUC, CUGC, UAAGUUAU, UAACUUAU, and GUAAAUAG.

DHCA-coated IO-nanocage synthesis: The IO-nanocages were synthesized with oleic acid by a modified version of a previously published method (Nano lett. 2016, 16, 7357-7363, (Nano Lett., 2016, 16, 7357-7363; J. Am. Chem. Soc., 2014, 136, 12552-5; Oncol. Rep., 2020, 43, 169-176; Nat. Commun., 2021, 12, 2903). Manganese (II) acetate (0.17 g), oleylamine (0.82 mL), and oleic acid (0.16 mL) were added to p-xylene (15 mL) in a three-necked 50 mL flask with a reflux condenser and sonicated for 10 mins. The flask was heated to 90° C. in air under magnetic stirring, then 1 mL of deionized water was rapidly injected into the flask. The reaction mixture was heated at 90° C. for 1.5 hrs, producing Mn₃O₄ nanoparticles. 1 mL of 2.4 M aqueous iron (II) perchlorate solution was added and the mixture maintained at 90° C. for an additional 1.5 hours to produce IO-nanocages by galvanic replacement. After cooling, IO-nanocages were collected by centrifugation, rinsed with ethanol, and dispersed in THF.

These hydrophobic IO-nanocages were coated with DHCA and transferred to the aqueous phase using a modified version of a previously published method. (Liu, Y.; Chen, T.; Wu, C.; Qiu, L.; Hu, R.; Li, J.; Cansiz, S.; Zhang, L.; Cui, C.; Zhu, G.; You, M.; Zhang, T.; Tan, W., Facile surface functionalization of hydrophobic magnetic nanoparticles. J. Am. Chem. Soc. 2014, 136, 12552-5). First, 100 mg of DHCA was dissolved in 5 mL of THE in a three-neck flask (25 mL). The resulting solution was heated to 50° C. after bubbling for 30 seconds with flowing nitrogen gas. Then, 20 mg of hydrophobic IO-nanocages capped by oleic acid were dispersed in 1 mL of THF which was added to the solution. The solution was heated to 50° C. for 3 hours, then cooled to room temperature, and 500 μL NaOH (0.5 M) was introduced to precipitate the magnetic nanoparticles. The precipitate was collected by centrifugation and redispersed in 2 mL water, then dialyzed overnight.

DHCA is a base coating directly in nanocages. The end group of DHCA is carboxylic acid, which can be conveniently conjugated with dextran or polyethylene glycol (PEG) as amine group of PEG/dextran can be covalently conjugated with the carboxylic acid with the established protocol.

In one aspect, a drug delivery system is provided comprising: an inorganic nanocage comprising palladium, iron oxide, or gold, wherein the inorganic nanocage has a diameter of 15 nm or less, loaded with a plasmid vector comprising a therapeutically effective amount of a DNA molecule, wherein the DNA molecule comprises in the 5′→ 3′ direction of transcription relative to synthesis of a messenger RNA (mRNA) molecule comprising a 3′ UTR of an mRNA encoding an ERBB2 protein: a promoter, operatively linked to a nucleic acid sequence encoding a 3′ UTR of an ERBB2 gene in the mRNA molecule in which one or more ARE poly(U) stabilizing motifs of the 3′ UTR of the mRNA are substituted with a nucleic acid sequence selected from AUUUU, CCUC, CUGC, UAAGUUAU, UAACUUAU, and GUAAAUAG.

In one aspect, the inorganic nanocage comprises iron oxide. In another aspect, the inorganic nanocage is an iron oxide nanocage. The drug delivery system may further comprises a polymer shell that surrounds the inorganic nanocage. The polymer shell may further comprise one or more biocompatible polymers. The one or more biocompatible polymers may be dextran (e.g. the polymer shell is a dextran polymer shell). The polymer shell may be capped with a functional organic molecule. The functionalized organic molecule may be a catechol or 3-(3,4-dihydroxyphenyl) propionic acid (DHCA). The inorganic nanocage may be covalently attached to the polymer shell. The polymer shell may have a diameter of 50 nm or less. A pharmaceutical composition comprising the drug delivery system of claim 1 and a pharmaceutically effective carrier.

In one aspect, a method of treating cancer in a subject in need thereof is provided, the method comprising administering to the subject a therapeutically effective amount of a pharmaceutical composition. The subject may be a mammal, including a human.

### F. Compositions

In one aspect, disclosed are pharmaceutical compositions comprising a plasmid vector of the invention and a pharmaceutically acceptable carrier.

In one aspect, disclosed are pharmaceutical compositions comprising a drug delivery system of the invention and a pharmaceutically effective carrier.

Pharmaceutical compositions are preferably sterile and contain an effective amount (e.g., a therapeutically effective amount) of the nucleic acid (e.g., the DNA molecule of the invention encoding for a destabilizing ERBB2 or MYC 3′ UTR). Pharmaceutical compositions are usually provided in a uniform dosage form and can be prepared in a manner known in the art. The pharmaceutical composition can, for example, be in the form of a solution or suspension.

The term “pharmaceutically acceptable” describes a material that is not biologically or otherwise undesirable, i.e., without causing an unacceptable level of undesirable biological effects or interacting in a deleterious manner.

As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. The injectable formulations can be sterilized, for example, by filtration through a bacterial-retaining filter or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

The pharmaceutical composition can comprise salts, buffer substances, preservatives, carriers, diluents and/or excipients, all of which are preferably pharmaceutically acceptable. Salts that are not pharmaceutically acceptable can yet be used for preparing pharmaceutically acceptable salts and are included in the invention. Pharmaceutically acceptable salts of this kind comprise, in a non-limiting way, those prepared from the following acids: hydrochloric, hydrobromic, sulfuric, nitric, phosphoric, maleic, acetic, salicylic, citric, formic, malonic, succinic acids, and the like. Pharmaceutically acceptable salts can also be prepared as alkali metal salts or alkaline earth metal salts, including, but not limited to, sodium salts, potassium salts, and calcium salts.

Suitable buffer substances for use in the disclosed pharmaceutical composition include, but are not limited to, acetic acid in a salt, citric acid in a salt, boric acid in a salt, and phosphoric acid in a salt.

Suitable preservatives for use in the disclosed pharmaceutical composition include, but are not limited to, benzalkonium chloride, chlorobutanol, paraben, and thimerosal.

The term “excipient,” as used herein, refers to an organic or inorganic component, of a natural or non-natural (synthetic) nature, with which the active component is combined in order to facilitate, enhance, or enable application. As used herein, the term “excipient” also includes one or more compatible solid or liquid fillers, diluents, or encapsulating substances, which are suitable for administration to a patient.

Possible carrier substances for parenteral administration include, but are not limited to, sterile water, glucose solutions, Ringer, Ringer lactate, sterile sodium chloride solution, polyalkylene glycols, hydrogenated naphthalenes and, in particular, biocompatible lactide polymers, lactide/glycolide copolymers, and polyoxyethylene/polyoxy-propylene copolymers.

The term “excipient” means all substances that can be present in a pharmaceutical composition and which are not active ingredients. Exemplary excipients include, but are not limited to, carriers, binders, lubricants, thickeners, surface active agents, preservatives, emulsifiers, buffers, flavoring agents, and colorants.

The pharmaceutical compositions described herein can be administered via any conventional route including, but not limited to, parenteral administration including by injection or infusion. Administration is preferably parenterally, e.g., intravenously, intraarterially, subcutaneously, in the lymph node, intradermally, or intramuscularly, although alternative routes of administration (e.g., oral administration, intraperitoneal, subcutaneous, transuretheral, transperineal, transrectal) are also envisioned.

The molecules, vectors, and compositions disclosed herein are preferably administered in effective amounts. An “effective amount” means the amount that achieves a desired reaction or a desired effect alone or together with further doses. In the case of treatment of a particular disease or of a particular condition, the desired reaction preferably relates to inhibition of the course of the disease. This comprises slowing down the progress of the disease and, in particular, interrupting or reversing the progress of the disease. The desired reaction in a treatment of a disease or of a condition can also be delay of the onset or prevention of the onset of said disease or said condition.

As used herein, the terms “effective amount” and “amount effective” refer to an amount that is sufficient to achieve the desired result or to have an effect on an undesired condition. For example, a “therapeutically effective amount” refers to an amount that is sufficient to achieve the desired therapeutic result (e.g., CS or complete response, PR or partial response, PFS or progression free survival, OS or overall survival) or to have an effect on undesired symptoms, but is generally insufficient to cause adverse side effects. The specific therapeutically effective dose level for any particular patient will depend upon a variety of factors including the disorder being treated and the severity of the disorder; the specific composition employed; the age, body weight, general health, sex and diet of the patient; the time of administration; the route of administration; the rate of excretion of the specific compound employed; the duration of the treatment; drugs used in combination or coincidental with the specific compound employed and like factors well known in the medical arts. For example, it is well within the skill of the art to start doses of a compound at levels lower than those required to achieve the desired therapeutic effect and to gradually increase the dosage until the desired effect is achieved. If desired, the effective daily dose can be divided into multiple doses for purposes of administration.

An effective amount of an agent or composition described herein will depend on a variety of factors including, but not limited to, the condition to be treated, the severeness of the disease, the individual parameters of the patient, including age, physiological condition, size, and weight, the duration of treatment, the type of an accompanying therapy (if present), the specific route of administration, and other similar factors. Accordingly, the doses administered of the agents, molecules, vectors, and compositions described herein may depend on several of these parameters. In the case that a reaction in a patient is insufficient with an initial dose, higher doses (or effectively higher doses achieved by a different, more localized route of administration) can be used.

In various aspects, the plasmid vector comprises a disclosed DNA molecule of the invention. Plasmid DNA vectors can be used as either a preventative or therapeutic DNA vaccine for a wide range of indications, from viral, bacterial, and parasitic disease to cancer and as gene therapy products. See, e.g., Williams et al. (2009) Biotechnol. Adv. 27 (4): 353-370. Processes for manufacturing plasmid DNA (pDNA) are well-known by those of skill in the art. See, e.g., Williams et al. (2009). Briefly, E. coli cells expressing the plasmid (e.g., a plasmid vector of the invention) are fermented before being harvested by, for example centrifugation or microfiltration tangential flow filtration (MF-TFF). The cell membrane is broken down (e.g., via cell lysis) to reveal a mixture of cellular contents including the plasmid DNA (e.g., a DNA molecule of the invention), genomic DNA, proteins, RNA, and other cell debris. Contaminants are removed (e.g., via precipitation or flocculation) as is any solid content that arose during the chemical lysis and neutralization of the feed stream. The plasmid is further purified using, for example, anion exchange chromatography, hydrophobic ineraction chromatography, and/or size exclusion chromatography, before being separated (e.g., via ultrafiltration or diafiltration), concentrated, washed, and resuspended in an appropriate buffer. Finally, sterile filtration is used to remove any microbial contaminants that may have been introduced via processing. Once the pDNA satisfies quality specifications set by regulatory agencies, it can be packaged into a vaccine (e.g., as a pharmaceutical composition of the invention) and subsequently introduced into a patient by, for example, intramuscular injection or particle bombardment. See, e.g., Mor (1998) Biochemical Pharmacology 55 (8): 1151-1153.

### G. Methods of Reducing ERBB2 or MYC Expression in a Cell

In one aspect, disclosed are methods of reducing ERBB2 expression in a cell expressing ERBB2, the method comprising transfecting the cell with an amount of a vector of the invention to cause a reduction of ERBB2 expression.

In one aspect, the methods of the invention include reducing MYC expression in a cell expressing MYC, the method comprising transfecting the cell with an amount of a vector according to the invention to cause a reduction of MYC expression.

As used herein, the term “cell” means any cell that can be transformed or transfected with an exogenous nucleic acid. Particular preference is given to mammalian cells including, but not limited to, cells from humans, mice, hamsters, pigs, goats, and primates. The cells can be derived from a multiplicity of tissue types and include primary cells and cell lines. Exemplary cells include, but are not limited to, keratinocytes, peripheral blood leukocytes, bone marrow stem cells, and embryonic stem cells. A nucleic acid (e.g., a DNA molecule) can be present in the cell in a single or in several copies and, in various aspects, is expressed in the cell.

In various aspects, the disclosed DNA molecules can be administered to a patient by ex vivo methods, i.e., by removing cells from a patient, genetically modifying said cells, and reintroducing the modified cells into the patient. Transfection and transduction methods are known to those of ordinary skill.

The term “transfection” means the introduction of nucleic acids, in particular, DNA, into a cell. As used herein, the term “transfection” also includes the introduction of a nucleic acid into a cell or the uptake of a nucleic acid by such cell, wherein the cell can be present in a subject, e.g., a patient. Thus, according to the invention, a cell for transfection of a nucleic acid according to the invention described herein can be present in vitro or in vivo, e.g., the cell can form part of an organ, a tissue, and/or an organism of a patient. As used herein, transfection can be transient or stable. For some applications of transfection, it is sufficient if the transfected genetic material is only transiently expressed. Cells allowing episomal amplification of nucleic acids greatly reduce the rate of dilution. If it is desired that the transfected nucleic acid actually remains in the genome of the cell and its daughter cells, a stable transfection must occur. Any technique useful for introducing, i.e., transferring or transfecting, nucleic acids into cells can be used. Preferably, DNA is transfected into cells by standard techniques. Such techniques include, but are not limited to, electroporation, lipofection, and microinjection. In various aspects, DNA is introduced into cells by electroporation. Electroporation or electropermeabilization relates to a significant increase in the electrical conductivity and permeability of the cell plasma membrane caused by an externally applied electrical field. It is usually used in molecular biology as a way of introducing some substance into a cell. Introduction of nucleic acid encoding a 3′ UTR encoding an ERBB2 or MYC gene in a mRNA molecule in which one or more ARE poly(U) stabilizing motifs are destabilized, as detailed further herein, results in transcription of a mRNA molecule that is destabilized compared to a wildtype mRNA molecule expressing a 3′ UTR encoding an ERBB2 or MYC protein.

In various aspects, the cell is mammalian. In a further aspect, the cell is human.

In various aspects, the cell has been isolated from a mammal prior to the transfecting step.

In various aspects, the cell is a cancer cell. In a further aspect, the cancer cell is a breast cancer cell, an ovarian cancer cell, a pancreatic cancer cell, or a prostate cancer cell. In a still further aspect, the cancer cell is trastuzumab-resistant. In yet an even further aspect, the cancer cell is a triple negative breast cancer cell. In an even further aspect, the cancer cell is a colon cancer cell, a lung cancer cell, a cervical cancer cell, an endometrial cancer cell, a neuroblastoma cell, a meningioma cell, a melanoma cell, a squamous cell carcinoma cell, a bone cancer cell, white blood cell cancer, or a throat cancer cell. In a still further aspect, the cell is a cancer cell where ERBB2/MYC oncogenes are key drivers of pathogenesis. In a still further aspect, the cancer cell is resistant to a chemotherapeutic drug. In yet a further aspect, the chemotherapeutic drug to which resistance has developed is selected from paclitaxel, cisplatin, olaparib, doxorubicin, bevacizumab, trastuzumab, trastuzumab dexrutecan, fluorouracil, methotrexate, capecitabine, carboplatin, cyclophosphamide, oxaliplatin, altretamine, bendamustine, busulfan, chlorambucil, daunorubicin, gemcitabine, idarubicin, ifosfamide, mitoxantrone, cabazitaxel, ceritinib, cladribine, dacarbazine, and docetaxel. In an even further aspect, the cancer cell is resistant to an immunotherapeutic. In a still further aspect, the immunotherapeutic is an anti-PD-1 monoclonal antibody, an anti-PD-L1 monoclonal antibody, or a checkpoint inhibitor. In yet a further aspect, the immunotherapeutic is pembrolizumab, nivolumab, brexucabtagene autoleucel, ado-trastuzumab emtansine, aldesleukin, amivantamab-vmjw, atezolizumab, axicabtagene ciloleucel, bevacizumab, blinatumomab, cetuximab, daratumumab, durvalumab, elotuzumab, gemtuzumab ozogamicin, ipilimumab, mogamulizumab, naxitamab, obinutuzumab, ramucirumab, siltuximab, and trastuzumab.

In various aspects, transfecting is via administration to a mammal. For example, as detailed herein, a DNA molecule of the invention can be cloned into a vector (e.g., a vector of the invention) and, thereafter, transfected into a cell. In various aspects, the cell is in a mammal, in which case transfection can be accomplished by formulating the vector as a pharmaceutical composition (e.g., a pharmaceutical composition of the invention) and thereafter administering the pharmaceutical composition to the mammal. Thus, transfection of the cell is accomplished by virtue of administering the pharmaceutical composition comprising the plasmid DNA.

### H. Methods of Reducing ERBB2 or MYC Expression in a Subject

In one aspect, disclosed are methods of reducing ERBB2 expression in a subject in need thereof, the method comprising transfecting the cell with an amount of a disclosed vector to cause a reduction of ERBB2 expression.

In one aspects, disclosed are methods of reducing MYC expression in a subject thereof, the method comprising transfecting the cell with an amount of a disclosed vector to cause a reduction of MYC expression.

As used herein, the term “subject” can be a vertebrate, such as a mammal, a fish, a bird, a reptile, or an amphibian. Thus, the subject of the herein disclosed methods can be a human, non-human primate, horse, pig, rabbit, dog, sheep, goat, cow, cat, guinea pig or rodent. The term does not denote a particular age or sex. Thus, adult and newborn subjects, as well as fetuses, whether male or female, are intended to be covered. In one aspect, the subject is a mammal. A patient refers to a subject afflicted with a disease or disorder. The term “patient” includes human and veterinary subjects.

As used herein, the terms “administering” and “administration” refer to any method of providing a pharmaceutical preparation to a subject. Such methods are well known to those skilled in the art and include, but are not limited to, oral administration, transdermal administration, administration by inhalation, nasal administration, topical administration, intravaginal administration, ophthalmic administration, intraaural administration, intracerebral administration, rectal administration, transuretheral administration, transperineal administration, transrectal administration, sublingual administration, buccal administration, and parenteral administration, including injectable such as intravenous administration, intra-arterial administration, intramuscular administration, and subcutaneous intratumoral administration. Administration can be continuous or intermittent. In various aspects, a preparation can be administered therapeutically; that is, administered to treat an existing disease or condition. In further various aspects, a preparation can be administered prophylactically; that is, administered for prevention of a disease or condition.

Consequently, single dose compositions can contain such amounts or submultiples thereof to make up the daily dose. The dosage can be adjusted by the individual physician in the event of any contraindications. Dosage can vary, and can be administered in one or more dose administrations daily, for one or several days. Guidance can be found in the literature for appropriate dosages for given classes of pharmaceutical products. In further various aspects, a preparation can be administered in a “prophylactically effective amount”; that is, an amount effective for prevention of a disease or condition.

In various aspects, the DNA molecule comprises in the 5′→3′ direction of transcription relative to synthesis of a messenger RNA (mRNA) molecule comprising a 3′ UTR encoding an ERBB2 protein: (a) a promoter, operatively linked to (b) a nucleic acid sequence encoding a 3′ UTR encoding an ERBB2 gene in the mRNA molecule in which one or more ARE poly(U) stabilizing motifs are substituted with a nucleic acid sequence selected from AUUUU, CCUC, CUGC, UAAGUUAU, UAACUUAU, and GUAAAUAG, wherein the mRNA molecule encoded by the DNA molecule is destabilized compared to a wildtype mRNA molecule comprising a 3′ UTR encoding an ERBB2 protein.

In various aspects, the DNA molecule comprises in the 5′→3′ direction of transcription relative to synthesis of a messenger RNA (mRNA) molecule comprising a 3′ UTR encoding a MYC protein: (a) a promoter, operatively linked to (b) a nucleic acid sequence encoding a 3′ UTR encoding a MYC gene in the mRNA molecule in which one or more ARE poly(U) stabilizing motifs are substituted with a nucleic acid sequence selected from AUUUU, CCUC, CUGC, UAAGUUAU, UAACUUAU, and GUAAAUAG, wherein the mRNA molecule encoded by the DNA molecule is destabilized compared to a wildtype mRNA molecule comprising a 3′ UTR encoding a MYC protein.

In various aspects, the subject is a mammal. In further various aspects, the subject is a human.

In various aspects, administering is via oral, intraveneous, intraperitoneal, subcutaneous, or intramuscular, intracranial or intraspinal or intrarectal, transperineal, trans urethral administration.

In various aspects, the subject has been diagnosed with a need for inhibition of ERBB2 expression prior to the administering step.

In various aspects, the subject has been diagnosed with a need for inhibition of MYC expression prior to the administering step.

### I. Methods of Treating a Disease or Disorder

In one aspect, disclosed are methods of treating cancer in a subject in need thereof, the method comprising administering to the subject a therapeutically effective amount of a disclosed pharmaceutical composition. Examples of cancers include, but are not limited to, a sarcoma (e.g., a cholangiosarcoma), a carcinoma (e.g., a hepatocellular carcinoma), a blastoma (e.g., a hepatoblastoma), a gastric adenoma, a hematological cancer, a solid tumor, breast cancer, cervical cancer, gastrointestinal cancer, colorectal cancer, brain cancer, skin cancer, prostate cancer (e.g., neuroendocrine prostate cancer), ovarian cancer, non-small cell lung carcinoma, thyroid cancer, testicular cancer, pancreatic cancer, liver cancer, endometrial cancer, melanoma, a glioma (e.g., astrocytoma, oligodendroglioma, oligoastrocytoma, glioblastoma), leukemia, lymphoma, chronic myeloproliferative disorder, myelodysplastic syndrome, myeloproliferative neoplasm, and plasma cell neoplasm (myeloma).

In one aspect, disclosed are methods of treating cancer in a subject in need thereof, the method comprising administering to the subject a pharmaceutical composition comprising a plasmid vector comprising a therapeutically effective amount of a DNA molecule, wherein the DNA molecule comprises in the 5′→3′ direction of transcription relative to synthesis of a messenger RNA (mRNA) molecule comprising a 3′ UTR of an mRNA encoding an ERBB2 protein: (a) a promoter, operatively linked to (b) a nucleic acid sequence encoding a 3′ UTR of an ERBB2 gene in the mRNA molecule in which one or more ARE poly(U) stabilizing motifs of the 3′ UTR of the mRNA are substituted with a nucleic acid sequence selected from AUUUU, CCUC, CUGC, UAAGUUAU, UAACUUAU, and GUAAAUAG, wherein the cancer is liver cancer or colorectal cancer.

In one aspect, disclosed are methods of treating cancer in a subject in need thereof, the method comprising administering to the subject a pharmaceutical composition comprising a plasmid vector comprising a therapeutically effective amount of a DNA molecule, wherein the DNA molecule comprises in the 5′→3′ direction of transcription relative to synthesis of a messenger RNA (mRNA) molecule comprising a 3′ UTR of an mRNA encoding a MYC protein: (a) a promoter, operatively linked to (b) a nucleic acid sequence encoding a 3′ UTR of a MYC gene in the mRNA molecule in which one or more ARE poly(U) stabilizing motifs of the 3′ UTR of the mRNA are substituted with a nucleic acid sequence selected from AUUUU, CCUC, CUGC, UAAGUUAU, UAACUUAU, and GUAAAUAG, wherein the cancer is liver cancer or colorectal cancer.

In one aspect, disclosed are methods of treating a cancer in a subject having a metastatic tumor, the method comprising administering to the subject a pharmaceutical composition comprising a plasmid vector comprising a therapeutically effective amount of a DNA molecule, wherein the DNA molecule comprises in the 5′→3′ direction of transcription relative to synthesis of a messenger RNA (mRNA) molecule comprising a 3′ UTR of an mRNA encoding an ERBB2 protein: (a) a promoter, operatively linked to (b) a nucleic acid sequence encoding a 3′ UTR of an ERBB2 gene in the mRNA molecule in which one or more ARE poly(U) stabilizing motifs of the 3′ UTR of the mRNA are substituted with a nucleic acid sequence selected from AUUUU, CCUC, CUGC, UAAGUUAU, UAACUUAU, and GUAAAUAG.

In one aspect, disclosed are methods of treating a cancer in a subject having a metastatic tumor, the method comprising administering to the subject a pharmaceutical composition comprising a plasmid vector comprising a therapeutically effective amount of a DNA molecule, wherein the DNA molecule comprises in the 5′→3′ direction of transcription relative to synthesis of a messenger RNA (mRNA) molecule comprising a 3′ UTR of an mRNA encoding a MYC protein: (a) a promoter, operatively linked to (b) a nucleic acid sequence encoding a 3′ UTR of a MYC gene in the mRNA molecule in which one or more ARE poly(U) stabilizing motifs of the 3′ UTR of the mRNA are substituted with a nucleic acid sequence selected from AUUUU, CCUC, CUGC, UAAGUUAU, UAACUUAU, and GUAAAUAG.

In one aspect, disclosed are methods of treating cancer in a subject having disease progression after osimertinib- or trastuzumab-based chemotherapy, the method comprising administering to the subject a pharmaceutical composition comprising a plasmid vector comprising a therapeutically effective amount of a DNA molecule, wherein the DNA molecule comprises in the 5′->3′ direction of transcription relative to synthesis of a messenger RNA (mRNA) molecule comprising a 3′ UTR of an mRNA encoding an ERBB2 protein: (a) a promoter, operatively linked to (b) a nucleic acid sequence encoding a 3′ UTR of an ERBB2 gene in the mRNA molecule in which one or more ARE poly(U) stabilizing motifs of the 3′ UTR of the mRNA are substituted with a nucleic acid sequence selected from AUUUU, CCUC, CUGC, UAAGUUAU, UAACUUAU, and GUAAAUAG.

In one aspect, disclosed are methods of treating cancer in a subject having disease progression after osimertinib- or trastuzumab-based chemotherapy, the method comprising administering to the subject a pharmaceutical composition comprising a plasmid vector comprising a therapeutically effective amount of a DNA molecule, wherein the DNA molecule comprises in the 5′→3′ direction of transcription relative to synthesis of a messenger RNA (mRNA) molecule comprising a 3′ UTR of an mRNA encoding a MYC protein: (a) a promoter, operatively linked to (b) a nucleic acid sequence encoding a 3′ UTR of a MYC gene in the mRNA molecule in which one or more ARE poly(U) stabilizing motifs of the 3′ UTR of the mRNA are substituted with a nucleic acid sequence selected from AUUUU, CCUC, CUGC, UAAGUUAU, UAACUUAU, and GUAAAUAG.

As disclosed herein, the terms “cancer disease” and “cancer” (medical term: malignant neoplasm) refers to a class of diseases in which a group of cells display uncontrolled growth (division beyond the normal limits), invasion (intrusion on and destruction of adjacent tissues), and sometimes metastasis (spread to other locations in the body via lymph or blood). These three malignant properties of cancers differentiate them from benign tumors, which are self-limited, and do not invade or metastasize. Most cancers form a tumor, i.e., a swelling or lesion formed by an abnormal growth of cells (called neoplastic cells or tumor cells), but some, like leukemia, do not. Examples of cancers include, but are not limited to, carcinoma, lymphoma, blastoma, sarcoma, glioma and leukemia. More particularly, examples of such cancers include bone cancer, blood cancer, lung cancer, liver cancer, pancreatic cancer, skin cancer, cancer of the head or neck, cutaneous or intraocular malignant melanoma, uterine cancer, ovarian cancer, rectal cancer, cancer of the anal region, stomach cancer, colon cancer, breast cancer, prostate cancer, uterine cancer, carcinoma of the sexual and reproductive organs, Hodgkin's disease, cancer of the esophagus, cancer of the small intestine, cancer of the endocrine system, cancer of the thyroid gland, cancer of the parathyroid gland, cancer of the adrenal gland, sarcoma of soft tissue, cancer of the bladder, cancer of the kidney, renal cell carcinoma, carcinoma of the renal pelvis, neoplasms of the central nervous system (CNS), neuroectodermal cancer, spinal axis tumors, glioma, meningioma, and pituitary adenoma. The term “cancer” also includes cancer metastases.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

As used herein, the term “diagnosed” means having been subjected to a physical examination by a person of skill, for example, a physician, and found to have a condition that can be diagnosed or treated by the molecules, vectors, compositions, or methods disclosed herein.

As used herein, “dosage form” means a pharmacologically active material in a medium, carrier, vehicle, or device suitable for administration to a subject. A dosage forms can comprise inventive a disclosed compound, a product of a disclosed method of making, or a salt, solvate, or polymorph thereof, in combination with a pharmaceutically acceptable excipient, such as a preservative, buffer, saline, or phosphate buffered saline. Dosage forms can be made using conventional pharmaceutical manufacturing and compounding techniques. Dosage forms can comprise inorganic or organic buffers (e.g., sodium or potassium salts of phosphate, carbonate, acetate, or citrate) and pH adjustment agents (e.g., hydrochloric acid, sodium or potassium hydroxide, salts of citrate or acetate, amino acids and their salts) antioxidants (e.g., ascorbic acid, alpha-tocopherol), surfactants (e.g., polysorbate 20, polysorbate 80, polyoxyethylene 9-10 nonyl phenol, sodium desoxycholate), solution and/or cryo/lyo stabilizers (e.g., sucrose, lactose, mannitol, trehalose), osmotic adjustment agents (e.g., salts or sugars), antibacterial agents (e.g., benzoic acid, phenol, gentamicin), antifoaming agents (e.g., polydimethylsilozone), preservatives (e.g., thimerosal, 2-phenoxyethanol, EDTA), polymeric stabilizers and viscosity-adjustment agents (e.g., polyvinylpyrrolidone, poloxamer 488, carboxymethylcellulose) and co-solvents (e.g., glycerol, polyethylene glycol, ethanol). A dosage form formulated for injectable use can have a disclosed compound, a product of a disclosed method of making, or a salt, solvate, or polymorph thereof, suspended in sterile saline solution for injection together with a preservative.

In various aspects, the subject is a mammal. In further various aspects, the subject is a human.

In one aspect, a method of treating cancer in a subject in need thereof is provided, the method comprising administering to the subject a pharmaceutical composition comprising a plasmid vector comprising a therapeutically effective amount of a DNA molecule, wherein the DNA molecule comprises in the 5′→3′ direction of transcription relative to synthesis of a messenger RNA (mRNA) molecule comprising a 3′ UTR of an mRNA encoding an ERBB2 protein: a promoter, operatively linked to a nucleic acid sequence encoding a 3′ UTR of an ERBB2 gene in the mRNA molecule in which one or more ARE poly(U) stabilizing motifs of the 3′ UTR of the mRNA are substituted with a nucleic acid sequence selected from AUUUU, CCUC, CUGC, UAAGUUAU, UAACUUAU, and GUAAAUAG, wherein the cancer is liver cancer or colorectal cancer. In one aspect, the DNA molecule is SEQ ID NO: 68, SEQ ID NO: 69, SEQ ID NO: 70; SEQ ID NO: 71 or SEQ ID NO: 72.

In one aspect, a method of treating cancer in a subject having disease progression after osimertinib- or trastuzumab-based chemotherapy is provided, the method comprising administering to the subject a pharmaceutical composition comprising a plasmid vector comprising a therapeutically effective amount of a DNA molecule, wherein the DNA molecule comprises in the 5′→3′ direction of transcription relative to synthesis of a messenger RNA (mRNA) molecule comprising a 3′ UTR of an mRNA encoding an ERBB2 protein: a promoter, operatively linked to a nucleic acid sequence encoding a 3′ UTR of an ERBB2 gene in the mRNA molecule in which one or more ARE poly(U) stabilizing motifs of the 3′ UTR of the mRNA are substituted with a nucleic acid sequence selected from AUUUU, CCUC, CUGC, UAAGUUAU, UAACUUAU, and GUAAAUAG. In one aspect, the DNA molecule is SEQ ID NO: 68, SEQ ID NO: 69, SEQ ID NO: 70; SEQ ID NO: 71 or SEQ ID NO: 72.

In one aspect, a method of treating cancer in a subject in need thereof is provided, the method comprising administering to the subject a pharmaceutical composition comprising a plasmid vector comprising a therapeutically effective amount of a DNA molecule, wherein the DNA molecule comprises in the 5′->3′ direction of transcription relative to synthesis of a messenger RNA (mRNA) molecule comprising a 3′ UTR of an mRNA encoding a MYC protein: a promoter, operatively linked to a nucleic acid sequence encoding a 3′ UTR of a MYC gene in the mRNA molecule in which one or more ARE poly(U) stabilizing motifs of the 3′ UTR of the mRNA are substituted with a nucleic acid sequence selected from AUUUU, CCUC, CUGC, UAAGUUAU, UAACUUAU, and GUAAAUAG, wherein the cancer is liver cancer or colorectal cancer. In one aspect, the DNA molecule is SEQ ID NO: 73, SEQ ID NO: 74 or SEQ ID NO: 75.

In another aspect, a method of treating a cancer in a subject having a metastatic tumor is provided, the method comprising administering to the subject a pharmaceutical composition comprising a plasmid vector comprising a therapeutically effective amount of a DNA molecule, wherein the DNA molecule comprises in the 5′→3′ direction of transcription relative to synthesis of a messenger RNA (mRNA) molecule comprising a 3′ UTR of an mRNA encoding a MYC protein: a promoter, operatively linked to a nucleic acid sequence encoding a 3′ UTR of a MYC gene in the mRNA molecule in which one or more ARE poly(U) stabilizing motifs of the 3′ UTR of the mRNA are substituted with a nucleic acid sequence selected from AUUUU, CCUC, CUGC, UAAGUUAU, UAACUUAU, and GUAAAUAG. In one aspect, the DNA molecule is SEQ ID NO: 73, SEQ ID NO: 74 or SEQ ID NO: 75.

In one aspect, a method of treating cancer in a subject having disease progression after osimertinib- or trastuzumab-based chemotherapy is provided, the method comprising administering to the subject a pharmaceutical composition comprising a plasmid vector comprising a therapeutically effective amount of a DNA molecule, wherein the DNA molecule comprises in the 5′→3′ direction of transcription relative to synthesis of a messenger RNA (mRNA) molecule comprising a 3′ UTR of an mRNA encoding a MYC protein: a promoter, operatively linked to a nucleic acid sequence encoding a 3′ UTR of a MYC gene in the mRNA molecule in which one or more ARE poly(U) stabilizing motifs of the 3′ UTR of the mRNA are substituted with a nucleic acid sequence selected from AUUUU, CCUC, CUGC, UAAGUUAU, UAACUUAU, and GUAAAUAG. In one aspect, the DNA molecule is SEQ ID NO: 73, SEQ ID NO: 74 or SEQ ID NO: 75.

Also provided are the uses of the disclosed molecules, vectors, pharmaceutical compositions, and products. In one aspect, the invention relates to use of at least one disclosed molecule or at least one disclosed vector. In a further aspect, the molecule or vector used is a product of a disclosed method of making.

In various aspects, the use relates to a treatment of disease or disorder associated with dysregulation of ERBB2 and/or MYC. In one aspect, the use is characterized in that the subject is a human. In one aspect, the use is characterized in that the disease or disorder is cancer.

In a further aspect, the use relates to a process for preparing a pharmaceutical composition comprising a therapeutically effective amount of a disclosed molecule or vector or a product of a disclosed method of making, for use as a medicament.

In a further aspect, the use relates to a process for preparing a pharmaceutical composition comprising a therapeutically effective amount of a disclosed molecule or vector or a product of a disclosed method of making, wherein a pharmaceutically acceptable carrier is intimately mixed with a therapeutically effective amount of the molecule or vector or the product of a disclosed method of making.

In a further aspect, the invention relates to the use of a disclosed molecule, a disclosed vector, a disclosed pharmaceutical composition, or a disclosed product in the manufacture of a medicament for the treatment of disease or disorder associated with dysregulation of ERBB2 and/or MYC signaling in a mammal. In a further aspect, the disease or disorder is cancer.

### J. EXAMPLES

The Examples are provided herein to illustrate the invention, and should not be construed as limiting the invention in any way. Examples are provided herein to illustrate the invention and should not be construed as limiting the invention in any way.

Briefly, as detailed herein, it has been found that the 3′ UTR of the oncogenes ERBB2 and MYC are enriched with poly(U) sequences in various cancer cell lines, including, but not limited to, breast, prostate, ovarian, and pancreatic cancer cell lines. These poly(U) sequences act as a mRNA stabilizing motif. It is, therefore, hypothesized that nonsense mediated decay of the transcript can be achieved by engineering unstable forms driven by mRNA decapping promoters. As detailed herein, ERBB2 and MYC constructs were engineered in which poly(U) stabilizing motifs were converted to destabilizing motifs. Using these constructs, it has been demonstrated in vivo in multiple cancer types that destabilization of the MYC and ERBB2 3′ UTR poly(U) motif leads to degradation of their oncogenic transcript and impairs their growth, proliferation, and metastasis, offering an improved survival outcome.

By way of illustration, total RNA was extracted from breast cancer cell lines: BT474, MDA MB231, T47D and MCF7. Total RNA was also extracted from the prostate cancer cell lines: LNCAP, MDA PCa2B, C4-2B and RWPE1 ex9. Samples of cDNA were made from these total RNA using the Qiagen reverse transcription kit (Catalog no: 205311). RT-PCR was performed using the cDNA according to the Qiagen manufacturers protocol using the following primer sequences:

ERBB2 forward:
(SEQ ID NO: 20)
GCCTCGTTGGAAGAGGAACA
ERBB2 reverse:
(SEQ ID NO: 21)
AGAGCCACCCCCAGACATAG
MYC Forward
(SEQ ID NO: 22)
CCTCACAACCTTGGCTGAGT
MYC reverse:
(SEQ ID NO: 23)
GGATTGAAATTCTGTGTAACTGC
DCP1A Forward
(SEQ ID NO: 24)
CCCTCAACTTCCGCCTCTAC promoter
DCP1A Reverse
(SEQ ID NO: 25)
CAGCCTGCAAGCTCCACTAC promoter

The PCR cycles were as follows: (Expected ERBB2 3′UTR amplicon size=464 bp, Expected MYC 3′UTR amplicon size=246 bp). PCR Cycle for cDNA synthesis: 94 for 2 mins, 94° C. for 15 sec, 55° C. for 30 sec, 68-72° C. for 1 min, Step 2-4 for 40 cycles, 4° C. hold. PCR set up: 38.1 μL of H₂O, 1.5 μL of 50 mM Mgcl, 1 μL of 10 mM DNTP, 1 μL ERBB2/MYC Forward primers, 1 μL ERBB2/MYC Reverse primers, 2 μL of cDNA (200 ng), 1 μL of Taq polymerase. Amplicon was separated on 2% agarose gel. The band sizes of ERBB2 464 bp and 264 bp for MYC were excised under UV light and then extracted with Qiagen gel extraction kit (Catalog no: 28706X4) according to manufacturer's protocol. The amplicon was sequenced by Sanger Sequencing method.

To find the poly U stabilizing ARE motif on the 3′UTR of ERBB2 and MYC genes, from the cDNA sequences after sequencing, the DNA nucleotide sequences were converted to mRNA sequences using conventional web software.

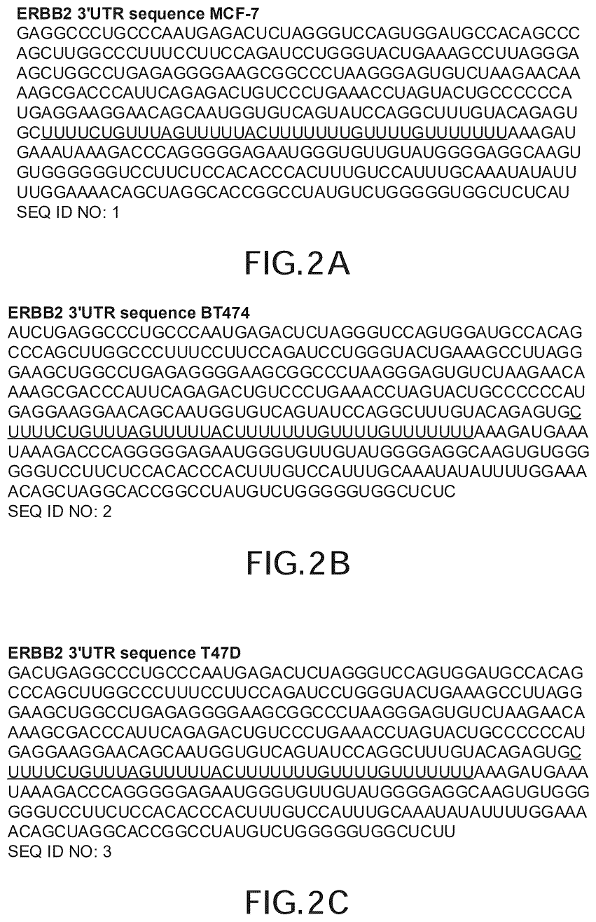

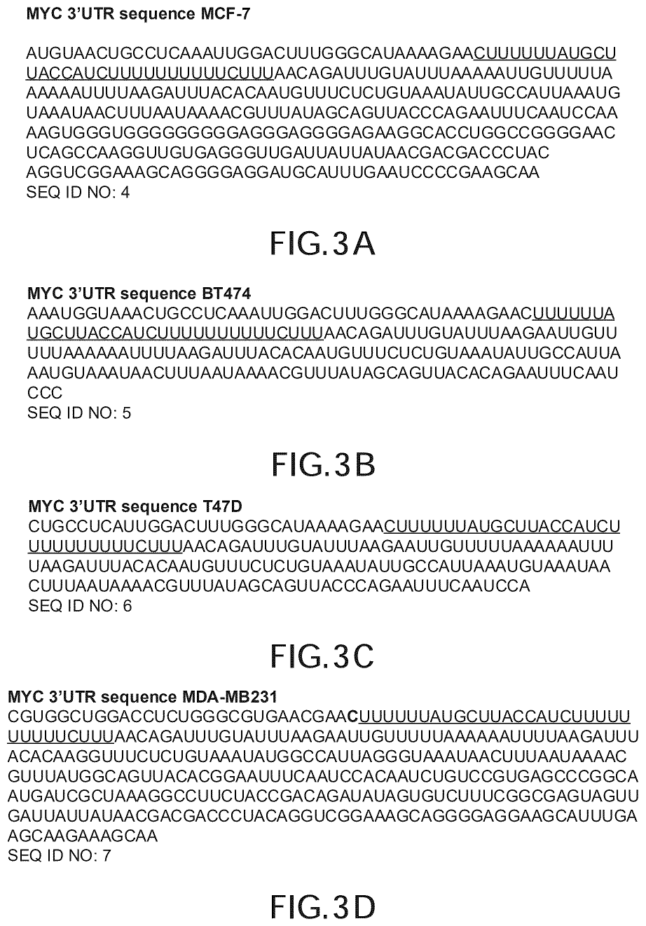

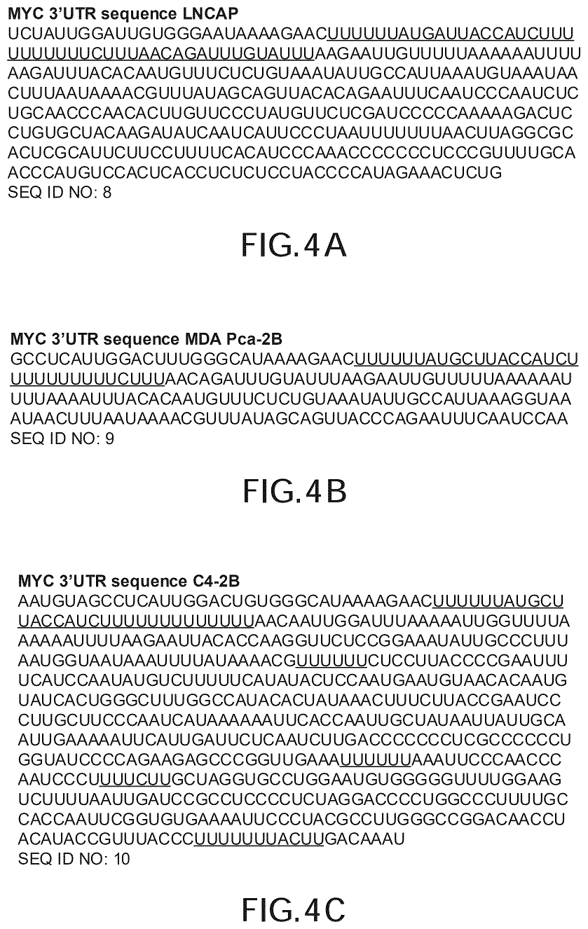

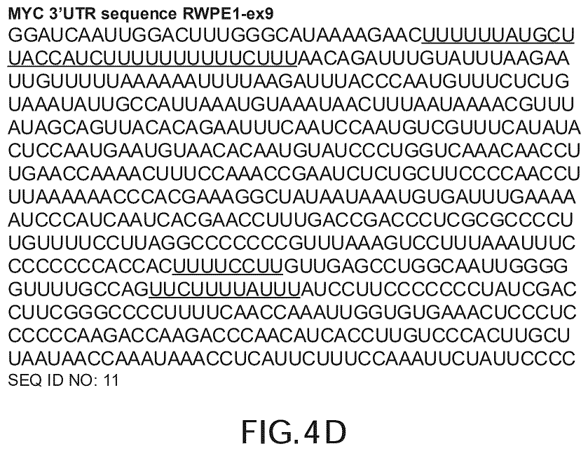

TABLE 7

| Cell line | Cancer | Oncogene | FIG. |
|---|---|---|---|
| MCF7 | Breast | ERBB2 | FIG. 2A (SEQ ID NO: 1) |
| BT474 | Breast | ERBB2 | FIG. 2B (SEQ ID NO: 2) |
| T47D | Breast | ERBB2 | FIG. 2C (SEQ ID NO: 3) |
| MCF7 | Breast | MYC | FIG. 3A (SEQ ID NO: 4) |
| BT474 | Breast | MYC | FIG. 3B (SEQ ID NO: 5) |
| T47D | Breast | MYC | FIG. 3C (SEQ ID NO: 6) |
| MDA MB231 | Breast | MYC | FIG. 3D (SEQ ID NO: 7) |
| LNCAP | Prostate | MYC | FIG. 4A (SEQ ID NO: 8) |
| MDA PCa2B | Prostate | MYC | FIG. 4B (SEQ ID NO: 9) |
| C4-2B | Prostate | MYC | FIG. 4C (SEQ ID NO: 10) |
| RWPE1 ex9 | Prostate | MYC | FIG. 4D (SEQ ID NO: 11) |

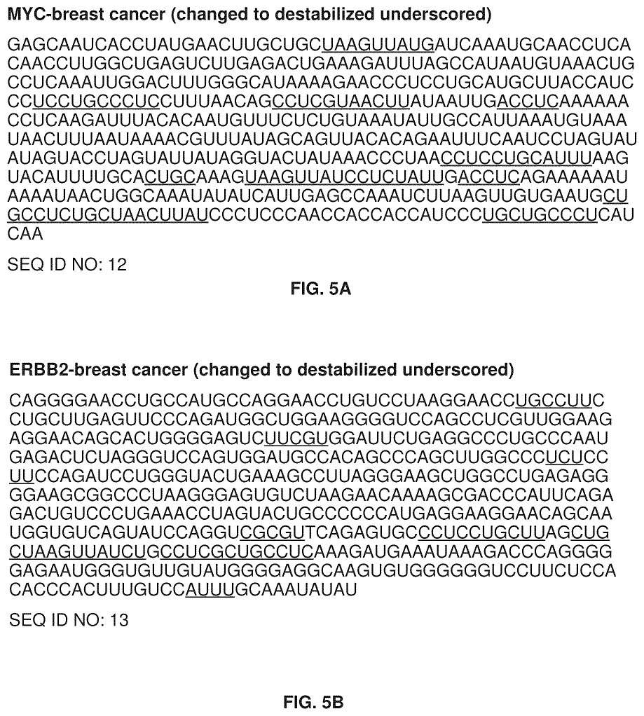

To design the destabilized 3′UTR of ERBB2 and MYC, the consensus stabilized motifs (e.g., UUUUU, UUGCAUGG, CCUUACAC) in the mRNA were selectively replaced with destabilized ARE consensus motifs. The resulting RNA sequences are shown in FIG. 5A (MYC) and FIG. 5B (ERBB2), corresponding to SEQ ID NO: 12 and SEQ ID NO: 13 respectively.

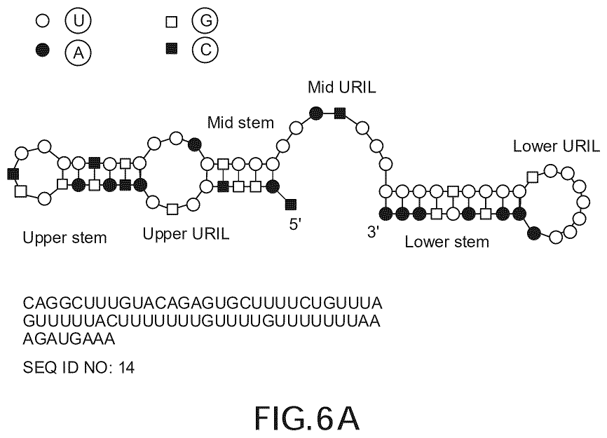

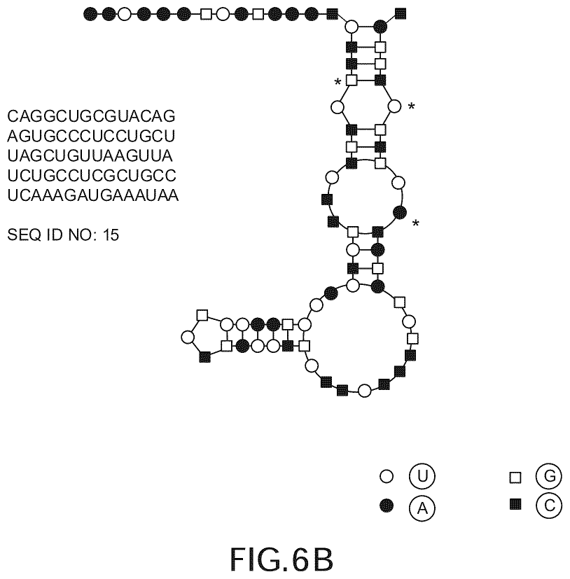

Some residues on the 3′ end of the 3′UTR were modified to increase stability. Torabi (Science, 371:6529 Jan. 7, 2021), used structural biology and biochemical assays to delineate residues of helices of the nucleic acids on the poly A tail of 3′UTR mRNA that determines their strong, moderate stability as well as rapid degradation. Based on this, the loop structures of the stabilized (FIG. 6A, SEQ ID NO: 14) and destabilized (FIG. 6B, SEQ ID NO: 15) 3′UTR of ERBB2 and MYC (BMC Bioinformatics, 11:129, 2010) were analyzed. The stabilized ERBB2 ARE structures have stability determining U-A on the lower loop stem as well as on the lower URIL. Using the mutated residues from Torabi, that confers moderate degree stability as an example. In designing the destabilizing ERBB2 and MYC 3′UTR (the M5 (C-U), M6 (U-C) and M11 (A-U) were changed with all residues marked with asterisks in FIG. 6B). Changes in these residues have a remarkable impact on the stability of transcript up to 120 mins.

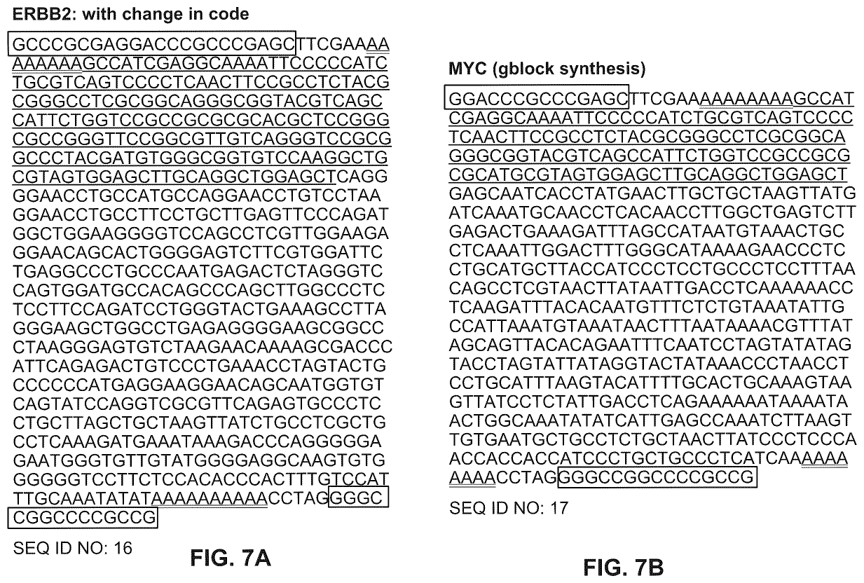

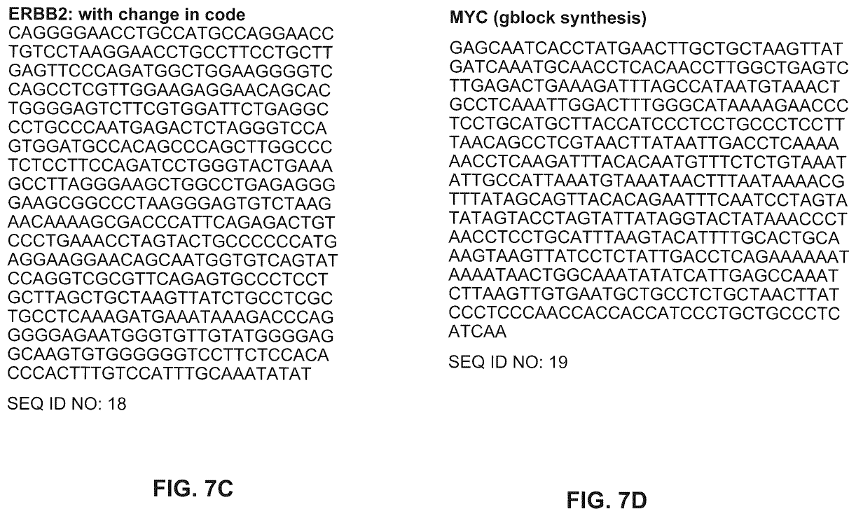

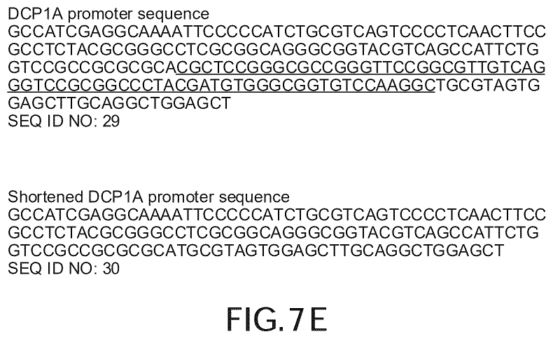

Having established the structure of stabilized ERBB2 and MYC 3′UTR and mutated residue of destabilized ERBB2 and MYC 3′UTR that increased their stability, these destabilized constructs were incorporated with an upstream 5′ BstB1 restriction site followed by a polyA sequence to stop the RFP transcription and then followed by a DCP1A promoter. At the 3′ end of the DNA sequences, a poly A sequence was added followed by BamH1 restriction site as shown in FIG. 7A (ERBB2) and FIG. 7B (MYC), corresponding to SEQ ID NO: 16 and SEQ ID NO: 17, respectively.

In FIG. 7A, the 5′ sequence contains a first digestion enhancing sequence (boxed), a Bstb1 sequence (TTCGAA) followed by a polyA sequence (AAAAAAAA, double underscored), a DCP1A promoter sequence (single underscore in FIG. 7A, SEQ ID NO: 29 in FIG. 7E) the destabilized 3′UTR sequence (no underscoring or boxing), followed by a polyA sequence (double underscored), a BamH1 restriction sequence (CCTAG) and a second digestion site (boxed). The 5′ Bstb1 site as well as the 3′ BamH1 sites enables the cloning into the sp6 vector. The upstream polyA sequence is to stop the transcription of the RFP from the Sp6 vector. The polyA tail at the 3′end is to stop the transcription of DCP1A promoter. FIG. 7C depicts the same DNA sequence with the first digestion enhancing sequence, the first restriction sequence, the polyA sequence, the promoter sequence, the second restriction sequence, and the second digestion sequence removed, corresponding to SEQ ID NO: 18.

In FIG. 7B, the 5′ sequences contain first digestion enhancing sequence (boxed) which is GGACCCGCCCGAGC (SEQ ID NO: 67), a BstB1 restriction site (TTCGAA), a polyA sequence (double underscore), a shortened DCP1A promoter sequence (single underscore in FIG. 7B, SEQ ID NO: 30 in FIG. 7E), the destabilized 3′UTR sequence (no underscore), followed by a polyA sequence and then a BamH1 restriction sequence (double underscored). FIG. 7D depicts the same DNA sequence with the first enhancing digestion sequence, the first restriction sequence, the polyA sequence, the promoter sequence, the second restriction sequence, and the second digestion sequence, removed, corresponding to SEQ ID NO: 19.

Referring to FIG. 7E, the DCP1A promoter sequence (SEQ ID NO: 29) was shortened by removing the underscored residues, thereby resulting in the shortened DCP1A promoter sequence (SEQ ID NO: 30).

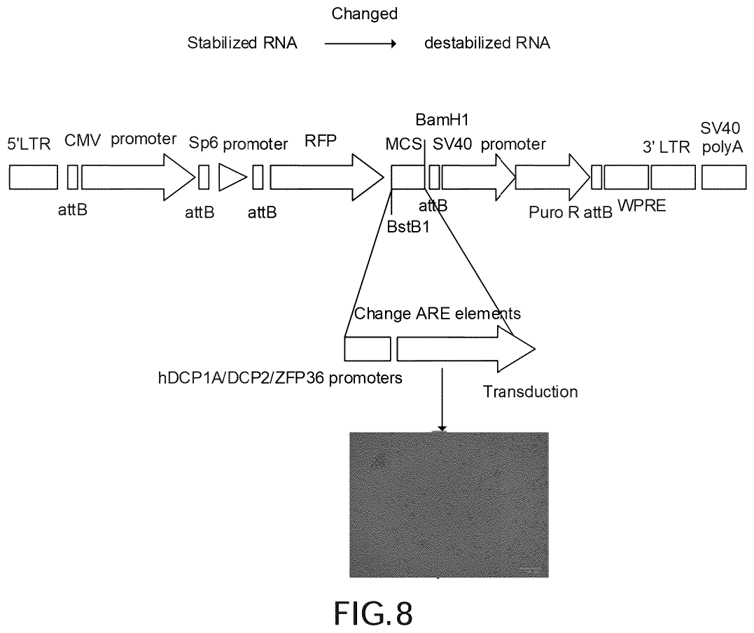

The fully destabilized 3′UTR of ERBB2 and MYC were synthesized as gblock from IDT Cloning of the destabilized 3′UTR of ERBB2 and MYC into Sp6 vector. See FIG. 8.

The destabilized 3′UTR of ERBB2 and MYC were cloned into pLenti-CMVSP6-nEGFP-SV40-PURO (Addgene: #138364) as described below.

Miniprep of vector: A stab of the vector was inoculated into LB media and grown over night shaking at 250 rpm at 37° C. The plasmid vector gDNA was extracted using the Qiagen midikit (Cat no: 12943).

Digest of vector with BstBi and BamH1: The vector was digested in BstBi and BamH1 at 37° C. overnight using NEB buffer 2.1 1 μl (400 ng) of vector gDNA, 1 μl of Bstb1 and Bamh1 respectively 3 μl of buffer, 25 μl of H₂O.

Gel extraction of vector: The digested vector was gel extracted using Qiagen gel extraction kit (Cat no28704).

Digest insert with BstB1 and BamH1: The synthetic gblock containing the destabilized ERBB2 and MYC 3′UTR were digested with BstB1 and BamH1 in NEB buffer 2.1 for 1 hr at 37° C., 1 μl (200 ng) of gblock DNA, 1 ul of Bstb1 and Bamh1 respectively 3 μl of buffer, 25 μl of H₂O.

Gel extraction of insert: The digested synthetic gblock containing the destabilized ERBB2 and MYC 3′UTR was gel extracted using Qiagen gel extraction kit (Cat no: 28704).

Ligation: T4 ligase buffer 2 μl, Plasmid vector 0.5 μl, Insert (digested synthetic gblock) 4 μl Nuclease free water 20 μl, T4 ligase 1 μl (NEB), At 22° C. for 3 hrs.

Transformation: competent E-coli (NEB 5 alpha Cat no: C298H) were transformed using SOC media, 25 ul of each cells was incubated on ice with 5 ul of the ligation mix for 30 mins. After 30 mins was heat shocked in water bath at 42° C. for 30 sec. Tubes were placed back on ice for 2 mins and 900 ul of SOC media was added and then incubated at 37° C. shaking for 1 hr at 250 rpm. After which they were plated on LB Agar plate containing ampicillin and the plate was incubated overnight at 37° C.

Colony picking and Miniprep: Colonies were picked with pipette tips and inoculated into 5 ml LB media containing Ampicillin and grown overnight at 37° C. shaking at 250 rpm. The pellets were spun down and gDNA was extracted using the Qiagen midikit (Cat no: 12943) and using a nanodrop machine the gDNA were quantified.

Colony PCR with ERBB2 3′UTR primers and DCP1A promoter primers: PCR was performed with ERBB2 primers and DCP1A primers using the PCR cycle described elsewhere in this specification.

Gel extraction: The colony PCR products of ERBB2 3′UTR and DCP1a promoters was gel extracted using Qiagen gel extraction kit (Cat no: 28704).

Sanger sequencing: The cloned synthetic gblock amplicons of ERBB2 3′UTR were sequenced using the primers described elsewhere in this specification by Sanger Sequencing.

Gibson Assembly: While the destabilized ERBB2 3′UTR was successfully incorporated into the vector by ligation (sticky end cloning), a GIBSON Assembly was used with MYC 3′UTR. The GIBSON Assembly was successful in cloning MYC and ERBB2 destabilized 3′UTR synthetic constructs into the plasmid vector. Each of the ERBB2 clones includes random variations that naturally occurs during cloning which impacts their performance. Likewise, similar natural variations are present in each of the MYC clones.

TABLE 8

| Clone | Vector incorporation method |
|---|---|
| desARE 3′UTR ERBB2-1 | Ligation |
| desARE 3′UTR ERBB2-2 | Ligation |
| desARE 3′UTR ERBB2-3 | Ligation |
| desARE 3′UTR ERBB2-4 | Ligation |
| desARE 3′UTR ERBB2-30 | Gibson Assembly |
| desARE 3′UTR MYC1-14 | Gibson Assembly |
| desARE 3′UTR MYC1-18 | Gibson Assembly |
| desARE 3′UTR MYC2-3 | Gibson Assembly |

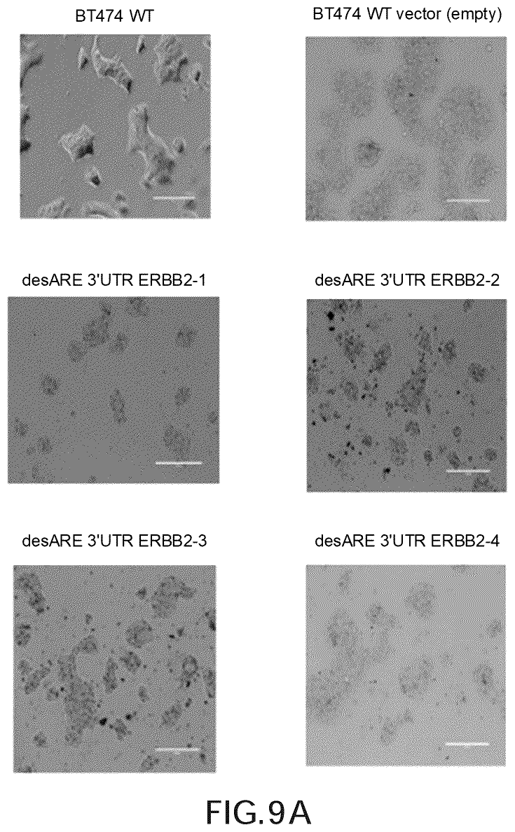

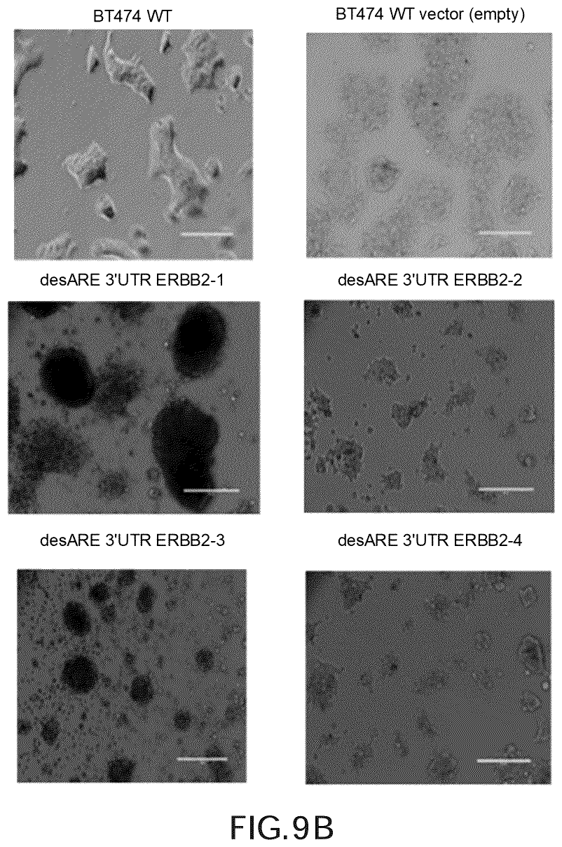

To introduce the destabilized 3′UTR into the cancer cells, constructs were electroporated into 200,000 to 500,000 cells with 50 ng of plasmid containing the constructs using the BioRad electroporation system. The preset mammalian protocol set was used for 293T cells and pulsed the cells in a cuvette 2×. Cells were then seeded into six well plates and viewed for morphology and red fluorescent protein (RFP) expression in 24 hrs. After 24 hrs the cells expressed RFP. Cell morphology of BT474 clone 5 cells is depicted in FIG. 9A and FIG. 9B.

FIG. 9A depicts two controls (BT474 clone 5 and BT474 clone 5 vector (empty)). Four different images are shown after treatment with destabilized ERBB2 showing degradation of the tumor after four days. In FIG. 9B four additional images are shown showing the desARE3′UTR ERBB2 killed up to 90% of the cancer cells after eight days.

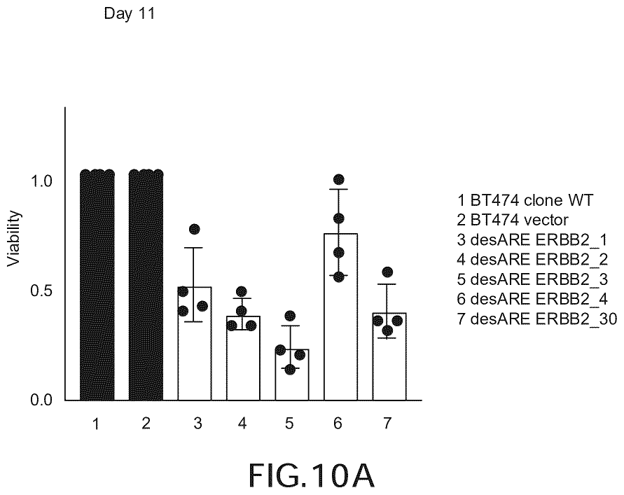

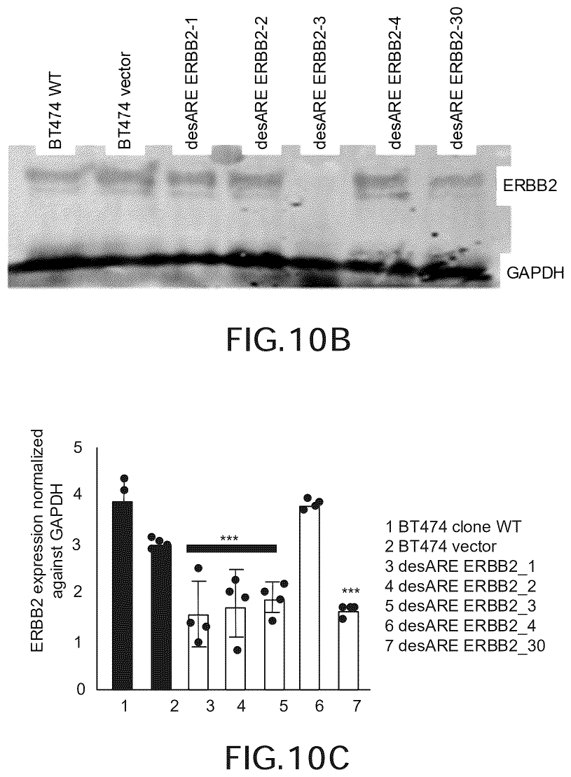

FIG. 10A depicts cell viability data of BT474 cell lines after eleven days of treating with 10 μg per μL of destabilized 3′ UTR ERBB2 plasmid vector (n=2, paired two tailed T-test (**P<0.01, ****P<0.0001, ns=0.084504)). FIG. 10B is a Western blot showing ERBB2 and GAPDH expression after treatment with destabilized 3′ UTR ERBB2 (n=3). FIG. 10C is a bar chart that quantifies the ERBB2 expression using qPCR normalized against GAPDH (Paired two tailed T-test (**P<0.01, ****P<0.0001, ns=0.084504, n=2)).

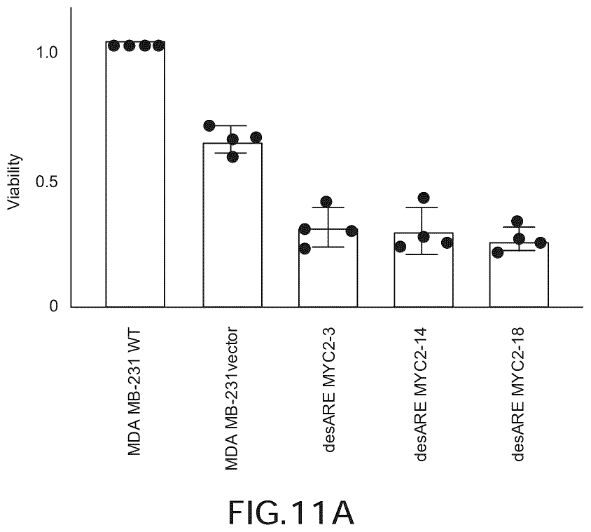

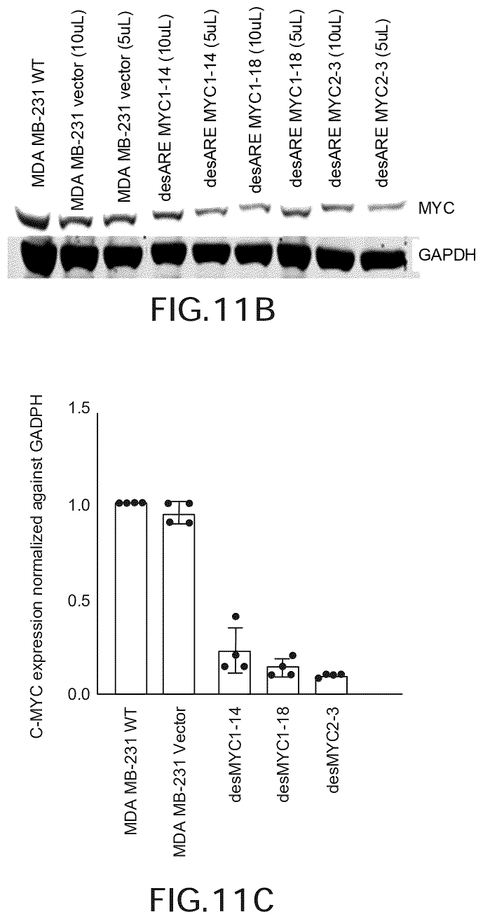

FIG. 11A depicts cell viability data of MDA MB231 cell lines after treating with 450 ng per μL of destabilized 3′ UTR MYC plasmid vector (n=6, **P<0.001 (WT vs vector), ***P<0.0001 (WT vs 3′UTRMYC2-3, 1-18, 1-14)). FIG. 11B is a Western blot showing MYC and GAPDH expression after treatment with destabilized 3′ UTR MYC (n=4). FIG. 11C is a bar chart that quantifies the MYC expression using qPCR normalized against GAPDH (****P<0.00001 (WT vs 3′UTR MYC1-18), ***P<0.0001 (WT vs BT474 3′UTRMYC1-14, n=5).

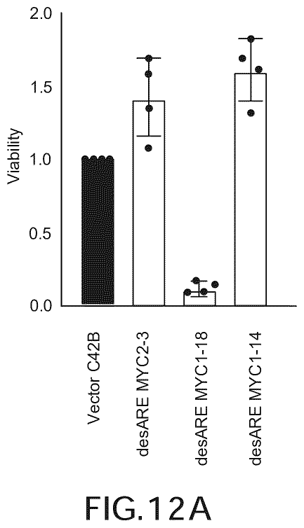

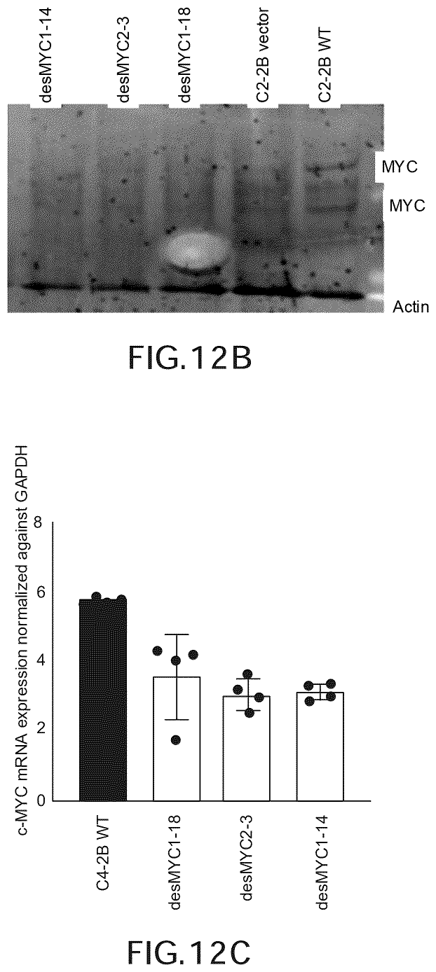

FIG. 12A depicts cell viability data of the C4-B cell line after treating with 10 μg per μL of destabilized 3′ UTR MYC plasmid vector. FIG. 12B is a Western blot showing MYC and GAPDH expression after treatment with destabilized 3′ UTR MYC of C4 2B cell lines. FIG. 12C is a bar chart that quantifies the MYC expression using qPCR normalized against GAPDH.

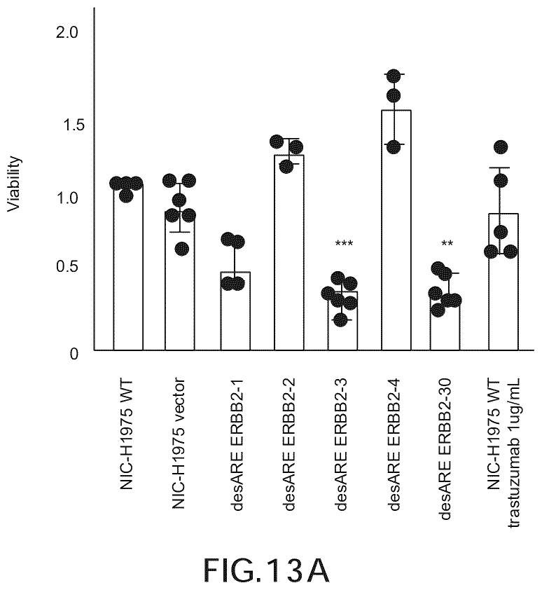

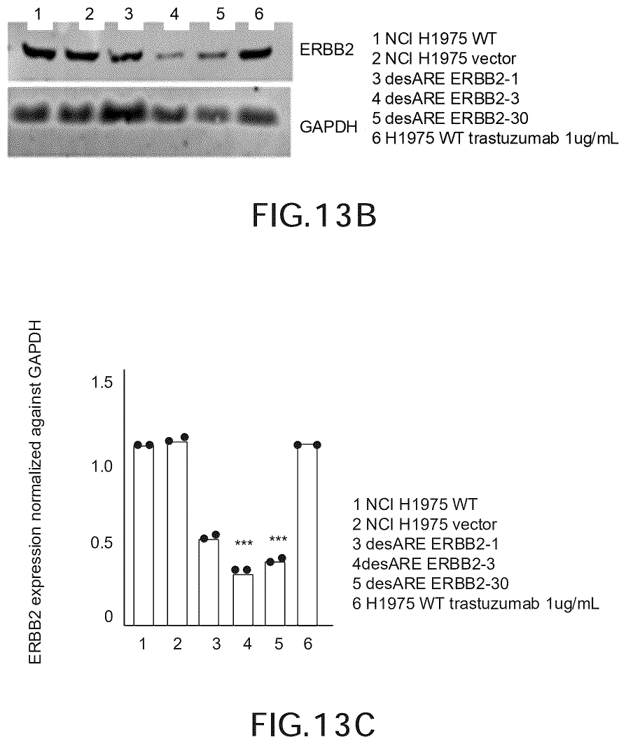

FIG. 13A depicts cell viability data of NCI H1975 cell lines (EGFRT790M HER2+ Osimertinib resistant non-small cell lung cancer) after treating with 10 μg per μL of destabilized 3′ UTR ERBB2 plasmid vector (n=2, Paired two tailed T-test (***P value-0.0013 desARE3′UTR ERBB2-3, **P value=0.03 desARE3′UTR ERBB2-30)). FIG. 13B is a Western blot showing ERBB2 and GAPDH expression after treatment with destabilized 3′ UTR ERBB2 (n=2). FIG. 13C is a bar chart that quantifies the ERBB2 expression using qPCR normalized against GAPDH (***P value=0.007569 desARE3′UTR ERBB2-3, ***P value=0.001123 desARE3′UTR ERBB2-30, n=2).

### Animal Studies

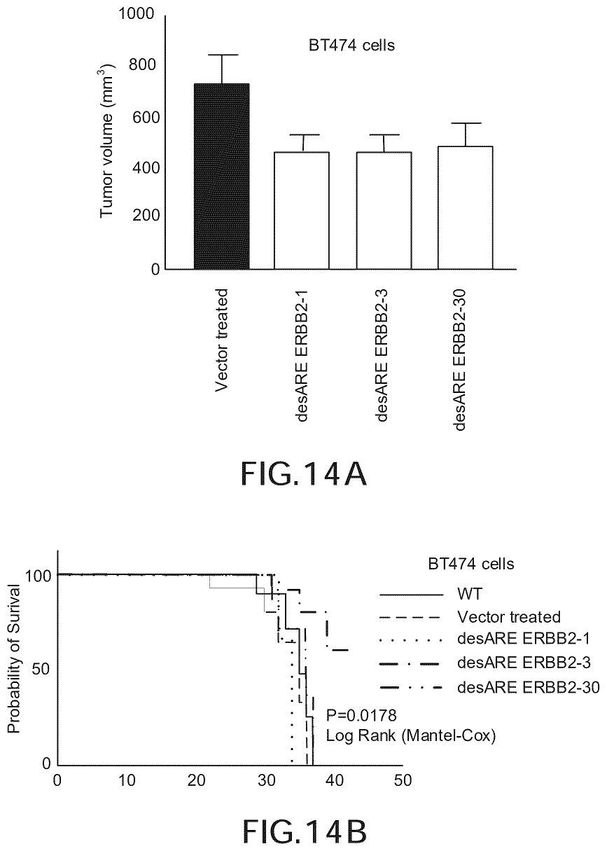

Referring to FIG. 14A and FIG. 14B, animal studies were conducted on the ERBB2 clones using twenty-five NSG mice. Mammary fat pads were implanted with five million BT474 clone 5 cells (ERBB2+ trastuzumab resistant breast cancer). On day fourteen, huge tumors were observed. On day fifteen, the mice were randomized. From day sixteen 20 μg of desARE ERBB2-1, desARE ERBB2-2 or desARE ERBB2-30 was administered 2× every week (6 μm first dose, 6 am the second dose) till 57 days. The tumor volumes were measured at the conclusion of the experiment at fifty-seven days (FIG. 14A). In FIG. 14B, the survival data of the mice is shown. Mice treated with desARE ERBB2-3 had exceptional survivors.

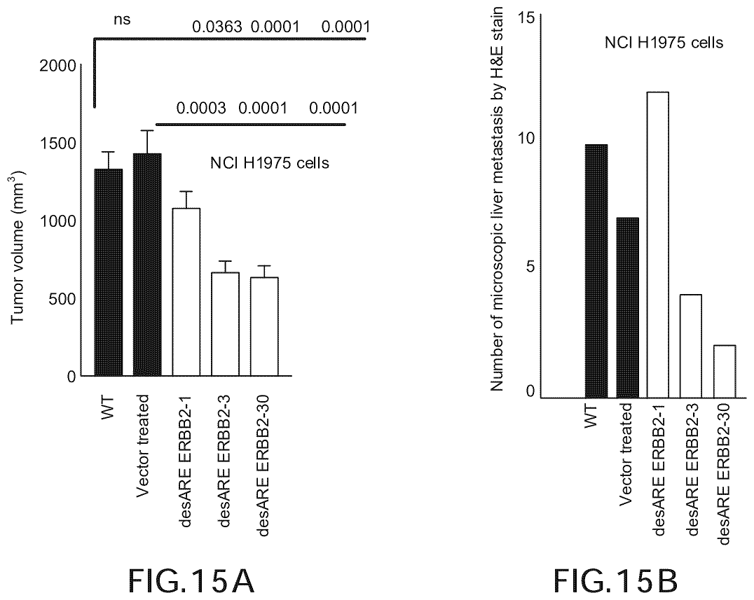

Referring to FIG. 15A and FIG. 15B, five million NCI H1975 cells (ERBB2+ osimertinib resistant EGFRT790M non-small cell lung cancer) were flank xenograft implanted into twenty five mice. On day thirty-five, huge tumors were observed. On day thirty-six the mice were randomized. From days thirty-seven to fifty-seven 2× per every 12 hrly (8 μm first dose, 8 am second dose), the mice received daily injections of 20 μg of desARE ERBB2-1, desARE ERBB2-2 or desARE ERBB2-30. Dosing break was observed on day fifty. The tumor volumes were measured at the conclusion of the experiment at seventy-seven days (FIG. 15A). In FIG. 15B, the number of liver metastatsis tumors is displayed in a bar chart as determined by H&E staining.

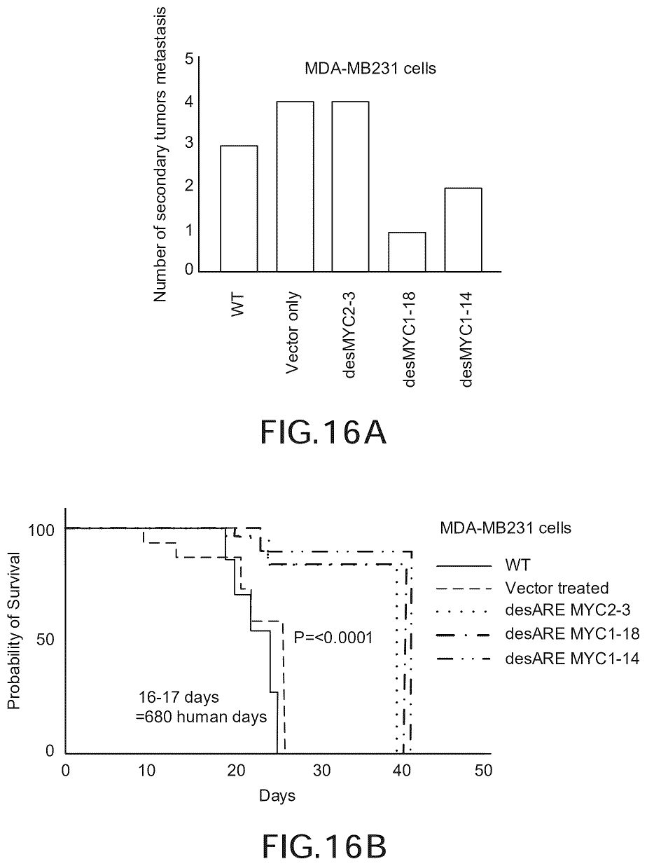

Referring to FIG. 16A and FIG. 16B, mammary fat pads of thirty mice were implanted with ten million MDA-MB231 cells. By day twenty-seven huge tumors were observed. On day twenty-eight, the mice were randomized. From day thirty to thirty-one, the mice received daily injections of 20 μg of desARE MYC2-3, desARE MYC2-18, desARE MYC2-14. On day thirty-two, a dosing break was permitted. From day thirty to day sixty-eight, a dosing break was observed on day 32 The experiment concluded at day sixty-eight. FIG. 16A depicts the number of secondary tumor metastatic sites while FIG. 16B depicts the probability of survival of the mice.

Degradation of ERBB2 mRNA in Drug-Resistant ERBB2+ Cells

Many ERBB2+ cancers are resistant to ERBB2-targeted therapy. ERBB2+ cancer cells are enriched with poly U sequences on their 3′UTR which are mRNA-stabilizing sequences. This disclosure provides sequences that successfully overwrote and outcompeted the endogenous ERBB2 mRNA-encoded message and degraded ERBB2 transcripts which led to the loss of the protein across multiple cancer cell types both in the wildtype and drug-resistance settings in vitro and in vivo, offering a unique safe novel modality to control ERBB2 mRNA and other pervasive oncogenic signals where current targeted therapies fail.

ERBB2/HER2 is a member of the subclass I receptor tyrosine kinase superfamily of ERBB/EGFR (epidermal growth factor receptor family) which has four members, namely, EGFR/ERBB1, ERBB2, ERBB3, and ERBB4. ERBB2 is activated upon binding of the neuregulin ligand onto the ERBB receptor which leads to its homo and heterodimerization triggering the activation of tyrosine kinases which have docking sites on the ERBB receptors and from there control large-scale signaling proteins, transcription factors, and kinases which mediates ERBB functions. ERBB2/ERBB2 is overexpressed in many human cancers including breast, lung, and colorectal cancers. The overexpression of ERBB2 is associated with very aggressive breast and drug-resistant lung cancer because ERBB2 is a membrane protein that signals and amplifies for proliferation, pro-survival, and prometastatic signals of the cancer leading to poor clinical outcomes.

Tyrosine kinase inhibitors such as osimertinib, erlotinib, and gefitinib target the ERRB/EGFR, and antibodies such as trastuzumab, which target the ecto-domain of ERBB2, have been developed as therapeutics such as ERBB2-overexpressing cancers. Trastuzumab in various forms in combination with chemotherapy has been successful in targeting ERBB2 overexpression in ERBB2+ breast cancer and improving survival as a standard first-line therapy for more than a decade. However, there are groups of ERBB2+ breast cancer, lung cancer, esophageal cancers, and gastrointestinal cancers that are resistant to trastuzumab and tyrosine kinase inhibitors. It is well known that 25% of early-stage and 75% of late-stage ERBB2+ breast cancer is resistant to trastuzumab. The same is true for EGFR T790M non-small-cell lung cancer that is osimertinib-resistant with 6% of patients bearing ERBB2 amplification.

For these patients who have treatment-resistant cancers with an overexpression of ERBB2, there is limited clinical intervention for them. More so, for these trastuzumab-resistant ERBB2-driven cancer patients, regional and distant organ metastasis of the tumor is a significant obstacle to good clinical outcomes. Resistance to trastuzumab and other tyrosine kinase inhibitors in ERBB2+ breast and lung cancer is a grand challenge that needs to be overcome to improve clinical outcomes and to prevent drug resistance and help sufferers of these deadly cancers.

To address this grand challenge of ERBB2+ drug resistance in breast and lung cancer, this disclosure provides a composition based on genome engineering of the genetic codes on the 3′UTR (3′ untranslated region) of the ERBB2 gene to destabilize, overwrite/outcompete, and degrade ERBB2 transcript, protein expression, ERBB2-dependent kinases, and interactome. The structure of a typical gene has various parts, namely, 5′UTR (untranslated region), the CDS (coding sequence), and the end of the gene called 3′UTR. The 3′UTR of genes serves as a hub for translation, stabilizing, and destabilizing of transcript and polyadenylation. Various motifs have been discovered in the 3′UTR region of genes to determine which transcripts are stabilized or destabilized and degraded. The AU-rich elements (AREs) found on the 3′UTR have been demonstrated to be involved in the nonsense-mediated decay of transcript via a complex process that involves decapping enzymes, deadenylation proteins, and cleaving enzymes.

Nonsense-mediated decay is an eukaryotic universally conserved mRNA surveillance mechanism that targets mRNA transcripts with premature termination codons for targeted degradation. This process is used for mRNA quality control to remove the toxic transcript and for lengthening or shortening the transcript to create mRNA isoforms. This is a tightly regulated process orchestrated by four distinct steps, namely, mRNA decapping, deadenylation, 5′-3′ and 3′-5′ exonuclease activity, and endonuclease activity. The UPF1-UPF2-UPF3 complex is the master regulator of the nonsense-mediated decay. Proteins such as the human DCP1A, DCP2 and ZFP36, XRN1 (5′-3 exonuclease), and CNOT1 (3′-5′ exonuclease) are involved in this process of ARE-mediated decay. Motifs on the 3′UTR of the gene were found to control mRNA transcript stabilization or destabilization. The consensus stabilization motifs are UUUUU, UUGCAUGG, and CCUUACAC, (corresponding to DNA motifs TTTTT, TTGCATGG and CCTTACAC) whereas the destabilization motifs are AUUUU, CCUC, CUGC, UAAGUUAU, UAACUUAU, and GUAAAUAG (corresponding to DNA motifs ATTTT, CCTC, CTGC, TAAGTTAT, TAACTTAT and GTAAATAG).

Without wishing to be bound to any particular theory, it is believed that the 3′UTR of oncogenes, such as ERBB2, are enriched with poly U mRNA-stabilizing elements, which stabilize their transcripts and drive tumor aggressiveness. Therefore, if these stabilizing elements were changed to destabilizing elements by motif engineering and drive them by mRNA decapping promoter DCP1A, one can control the oncogene by overwriting/outcompeting the endogenous ERBB2 mRNA and degrade the transcript, which will lead to the loss of proteins and signaling cascade. This disclosure provides data that demonstrate that, by destabilizing the stable 3′UTR elements of ERBB2 across multiple recalcitrant drug-resistant cancer models, pervasive ERBB2 oncogenic transcript are controlled that specifically degraded the ERBB2 protein and its associated kinases and interactome which triggered apoptosis and killed the tumors and is validated in vivo. This disclosure therefore provides technology to target and control oncogenes where drugs failed and for therapeutic gene targeting in diseases and control of any transcript of interest.

### Materials and Methods

Cell culture of BT474, BT474 clone 5, NCI-H1975, NCI H2030, HCT116, and MCF10A: BT474 was grown in DMEM supplemented with 10% FBS and 1% penicillin-streptomycin. The BT474 clone 5 (trastuzumab-resistant) and NCI-H1975 EGFR T790M and NCI H2030 were grown in RPMI-1640 supplemented with 10% FBS and 1% penicillin-streptomycin. HCT116 were grown in McCoy's 5A modified medium with 10% FBS and 1% penicillin-streptomycin. MCF10A was grown in MEBM with additives as listed in ATCC. The cells were grown to 80% confluency before use.

RNA extraction from breast cancers: Total RNA was extracted from the BT474 using the RNA Easy kit from the Qiagen (cat no: 74104). The RNA was stored at −80° C. until use.

QPCR with ERBB2 3′UTR primers: To determine the mRNA-stabilizing poly U-rich elements on the 3′UTR of ERBB2, cDNA was made from the total RNA of BT474, MCF7, T47D, and MDA MB231 using the Qiagen Reverse Transcription kit (Catalog no: 205311). RT-PCR was performed using the cDNA according to the Qiagen manufacturer's protocol and the primers used are in Table 4.

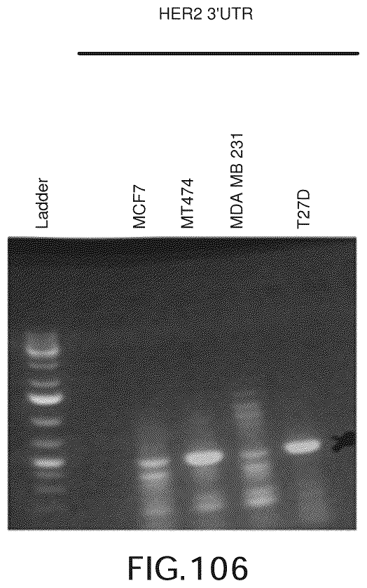

Sanger sequencing of 3′UTR of ERBB2 cDNA amplicon: To confirm the ERBB2-stabilizing 3′UTR poly U sequences, the amplified bands were excised under UV light and then extracted with the Qiagen Gel Extraction kit (Catalog no. 28706X4) according to manufacturer's protocol. The amplicon was then sequenced with the ERBB2 3′UTR PCR primers using the Sanger Sequencing method by the commercial company Psomagen Inc., Brooklyn, New York, United States (FIG. 106). Subsequently, an online software DNA to the mRNA translator was used to identify the stable ARE motifs of ERBB2 3′UTR.

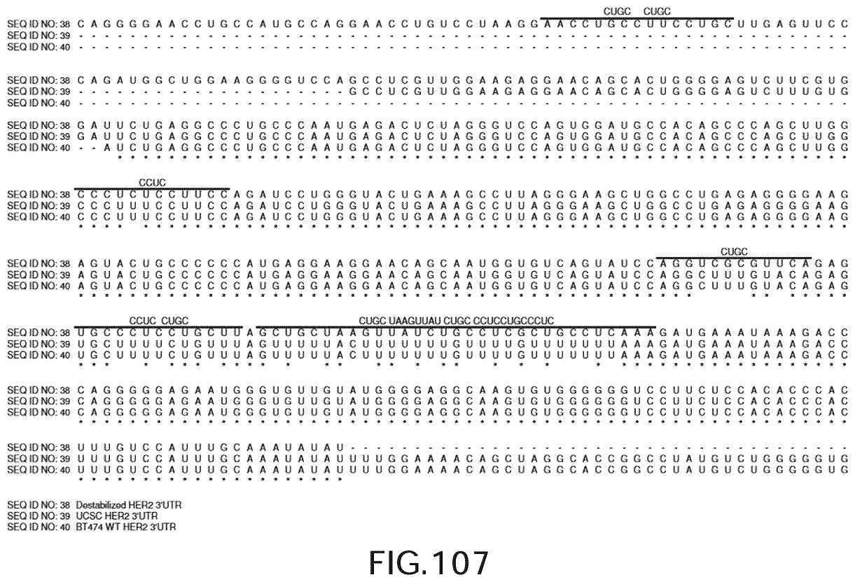

Design of stable destabilized 3′UTR of ERBB2: To design the destabilized 3′UTR of ERBB2, the consensus-stabilized UUUUU, UUGCAUGG, and CCUUACAC motifs in the mRNA were replaced with destabilized ARE consensus motifs of CCUC, CUGC, and UAAGUUAU, respectively (FIG. 107). Some residues on the 3′ end of the 3′UTR were modified to increase stability. Torabi et al. (Science, 371:6529 Jan. 7, 2021) used structural biology and biochemical assays to delineate residues of helices of the nucleic acids on the poly A tail of 3′UTR mRNA that determines their strong, moderate stability as well as rapid degradation. With this information, the loop structures of the stabilized and destabilized 3′UTR of ERBB2 were analyzed (FIG. 6A and FIG. 6B). The stabilized ERBB2 structures have stability determining U-A on the lower loop stem as well as on the lower URIL. The mutated residues from Torabi et al that confer moderate-degree stability were used as an example. In designing the destabilizing ERBB2 3′UTR, M5 (C-U), M6 (U-C), and M11 (A-U) were changed, and all residues are marked with an asterisk (FIG. 6B). The changes in these residues have a remarkable impact on the stability of the transcript for up to 120 mins. Having established the structure of stabilized ERBB2 3′UTR and mutated residue of destabilized ERBB2 3′UTR that increased their stability, these destabilized constructs were incorporated with an upstream 5′ BstB1 restriction site followed by a poly A sequence to stop the RFP transcription of the vector followed by a DCP1A promoter. At the 3′ end, a poly A sequence was added followed by the BamH1 restriction site (FIG. 7A).

Synthesis of destabilized 3′UTR of ERBB2 as gBlocks: The destabilized 3′UTR of ERBB2 were synthesized as gBlocks.

Cloning of destabilized 3′UTR of ERBB2 into the Sp6 vector both by sticky end cloning and Gibson assembly: The destabilized 3′UTR ARE of ERBB2 were cloned into pLenti-CMVSP6-nEGFP-SV40-PURO (Addgene: #138364).

Transfection/electroporation of destabilized 3′UTR of ERBB2 into BT474 wildtype, BT474 clone 5 (trastuzumab resistance), and NCI-H1975, NCI-H2030, and HCT116 cancer cells: To introduce the destabilized 3′UTR into the cancer cells, constructs were electroporated into 200,000-500,000 cells with 50 ng of plasmid containing the constructs using the BioRad electroporation system. The preset mammalian protocol was used set for 293 T cells and pulsed the cells in a cuvette 2×. The cells were then seeded into six-well plates and viewed for morphology and red fluorescent protein (RFP) expression in 24 hrs. After 24 hrs, the cells expressed RFP indicating that the constructs were successfully integrated into the cells. The constructs were also injected intraperitoneally to the animals as a plasmid.

YES1 overexpression: The YES1 Y537F overexpression vector (Addgene: 51299) was transfected into desARE3′UTR ERBB2-3 and -30 cells that have lost ERBB2 and YES1 by introduction of destabilized desARE3′UTR ERBB2. Overexpression of YES1 was confirmed by the YES1 Western blot.

Cell microscopy: The cells were observed regularly under the light microscope at 20×. Within day 4, the BT474 wildtype containing the destabilized elements showed a distorted and ruptured membrane compared to the control. For the trastuzumab-resistant cells, the changes in cellular morphology were observed from the starting of day 9 for the cells containing destabilized constructs. The cell size was drastically reduced compared to controls.

Immunofluorescence: To determine the ERBB2, caspases 3 and 9 and cleaved caspase 3 protein expression changes in the destabilized breast and lung cancer compared to the wildtype. The cells were seeded on a tissue culture slide or six-well plate at 2,000-3,000 per well. The cells were allowed to grow for 1 day, and after they were fixed with 200 μl of 4% paraformaldehyde, they were added to the cell media. The cells were placed at 4° C. for 5 mins, after which the paraformaldehyde was decanted. Anti-ERBB2 (Human ErbB2/Her2 Mab, Clone 191924 R&D systems) 1:1000 in BSA was added to the slide wells and sealed with an aluminum foil and kept at 4° C. overnight. After that, the antibody is removed and secondary antibodies Alexa 488 (green) or Alexa 610 (red) were added at 1:10,000 and covered with the aluminum foil and kept at room temperature for 1 h. After 1 h, the secondary dye was washed with PBS (phosphate-buffered saline) 3×, and then, the water from edges were wiped off with Kim wipes. In addition, a drop of DAPI (NucBlue nuclear stain) was added and then sealed with cover slips, and the slides were viewed under a Nikon confocal microscope. For the tissue slides, slides returned from the expert pathologists were washed in 1×PBS by dipping and, subsequently, dripped off and cleaned with Kim wipes. Anti-ERBB2, CNOT1, XRN1, and UPF3B (1:1000) were dropped on the slides and covered overnight at 4° C. to avoid desiccation. The slides were washed in 1×PBS and Alexa 610 was added and incubated at room temp for 1 h. After that, the slides were washed and a drop of DAPI was added and covered with a coverslip, and finally, the image was acquired under a Nikon confocal microscope.

Western blot: To quantify the ERBB2, YES1, WNK1, and CNOT1 protein expression changes in wildtype cells compared to the cells containing the destabilized 3′UTR ARE. The cells were harvested and lysed them in a cocktail of the protease inhibitor in an MPER buffer. The protein extract was stored in −80° C. until use. The protein was separated in 12% stacked SDS page gels and separated by initial run at 75 V and after 15 mins at 120 V until complete separation. The proteins were transferred onto a PVDF membrane already charged with methanol at 25 V for 1 h. The membrane was blocked with BSA and subsequently incubated with anti-ERBB2 (Human ErbB2/Her2 MAb Clone 191924) or anti-YES1 and WNK1 (Cell Signaling #65890 and #4979), CNOT1 (14276-1-AP) overnight in BSA. The primary antibody was removed, and secondary anti-mouse IR 800CW dye (Li-COR) was added at 1:1000 incubated with BSA covered from light for 2 hrs. After incubation, the membrane was washed with TBST 3×, and the image was taken on an Li-COR Oddysey chemiluminescent imager.

Cell viability: To quantify if the destabilized constructs affect the cell survival, cell viability of wildtype cells were compared with the cells carrying destabilized constructs with CellTiter-Glo (Promega cat: G7570).

Caspase 3/7 assay, caspase 3, cleaved caspase 3, and caspase 9 protein analysis: To assay for increased caspase activity, the caspase 3/7 Glo kit (Promega cat: G8090) was used as well anti-caspases 3 and 9 (Abcam #4051, #25758) and cleaved caspase 3 (Cell Signaling #9664). The kit was used according to the manufacturer's instructions. Immunofluorescence was performed with antibodies against cleaved caspase 3 and caspase 9 on wildtype cells and the engineered destabilized cells.

Phosphorylation kinase array: To determine how kinases are affected by the destabilization of the ERBB2 protein, the Human Phospho-Kinase Array kit (Cat no. ARY003C) was used.

Migration assay: Wounds were made on the cell monolayer using a sterile 200 μL pipette tip. The cells were washed with 1×PBS, and fresh media were added to each well following the wound. The images of scratched areas were taken at 10× magnification using an AE30 inverted microscope (Motic, Richmond, BC, Canada).

RNA seq: To confirm the significant downregulation of ERBB2 on the genome scale following the destabilization of the 3′UTR ARE as well as to ascertain to what level the ERBB2 interactome is affected by the significant downregulation of ERBB2, its specificity is compared to the wildtype controls. RNA Seq was performed on the RNA from desARE3′UTR ERBB2-3 and desARE3′UTR ERBB2-30 compared to the wildtype and vector controls. RNA Seq analysis was performed using established pipelines and BioJupies.

Quantitative reverse transcript PCR: To quantify the ERBB2 expression changes upon destabilization of the ERBB2 3′UTR comparing the wildtype and the vector controls, quantitative PCR was performed on ERBB2, deadenylases CNOT1, XRN1, and PARN, and decapping enzyme DCP1A using their exon primers and GAPDH as control. All primers used are listed in Table 4.

Optical genome mapping: To understand if the engineering method introduced copy number variation, indel, etc, optical genome mapping provided by Bionano was used. The experiments were performed in two independent replicates.

Animal study: A total of 25 female NSG mice were purchased from the Jackson laboratory. The animals were received and allowed to acclimate according to the institutional protocol. NCI-H1975 was expanded as described previously, and on the day of implantation, the cells were harvested, washed, and resuspended in phosphate-buffered saline (PBS). The confluency of the cells was 80%. Five million cells in PBS were implanted on the flank of each mouse. After 35 days, huge tumors engrafted, and on 36 days, the mice were randomized into five mice per cage into five groups and ensured equal distribution of tumor size within each group. The vector and desARE3′UTR ERBB2-1, -3, and -30 were administered as plasmids at 20 μg/0.1 m per mice intraperitoneally 12 hrly for 9 days with a 1-day dosing break. The group of wildtype NCI-H1975 mice received no treatment. Daily measurements of tumor size (length and width), weight, and body condition score were obtained. At day 46, due to the enormous size of the wildtype and vector tumor sizes, the control groups were euthanized and, subsequently, the mice receiving the constructs were also euthanized to perform complete necropsy, full blood count, and blood chemistry analysis. The necropsy and complete blood count and histopathology were performed by an expert pathologist from the Memorial Sloan Kettering Hospital, New York. They were blinded to the experimental details.

Full blood count and electrolyte analysis: The full blood count and necropsy and electrolyte analysis were performed by the expert pathologist at the Memorial Sloan Kettering Cancer Center, New York City. See FIG. 106, FIG. 6A and FIG. 6B.

Statistical analysis: All experiments were performed in multiple replicates. The cell-based experiments were performed more than 3× at the minimum as indicated on the legends, for those n=2 that show the best results from more than 3× experiments. A total of 25 female NSG mice were used at five mice per group per cage. The groups were WT, vector, and desARE3′UTR ERBB2-1, -3, and -30. All animal data presented were performed as five mice in a cage per group. A two-tailed T-test was used to determine the statistical significance difference between the different treatment groups.

ImageJ: We used the software ImageJ to quantify the immunofluorescence signal of ERBB2, cleaved caspase 3, and caspase 9.

GraphPad prism: The GraphPad Prism software was used to performed all statistics with the software.

4SU mRNA labeling pulse-chase experiment: To understand how the destabilized exogenous constructs destabilize the endogenous ERBB2 mRNA, 1 million cells of the NCI-H1975 WT, NCI-H1975 vector, and desARE3′UTR ERBB2-3 and -30 were seeded in a six-well plate and treated with 4SU at lug/ml and collected cells at 0 h, 30 mins, 1 h, 3 h, 6 h, and 24 h. RNA was collected, cDNA was made, and RT-qPCR was performed with primers targeting the endogenous ERBB2 as well as primers targeting the vector as well as RFP and the cloned destabilized ERBB2, and the control primer is ACTB. Delta Ct (Ct target-Ct destabilized ERBB2) and fold enrichment were calculated as 0.5 Ct. The destabilized ERBB2 transcript was 2.5×10⁴ overexpressed than the endogenous ERBB2.

Identification of mRNA poly U stabilizing motifs and design of the engineered destabilized 3′UTR ERBB2 constructs: Without wishing to be bound to any particular theory, the oncogenic transcript of ERBB2 is believed to be stabilized on the 3′UTR with poly U sequence. If the stabilizing element is destabilized, one could overwrite/outcompete the endogenous ERBB2-encoded message, degrade the ERBB2 transcript and protein expression, and thus control the aggressiveness of ERBB2+-driven cancers both in wildtype and drug-resistant settings. For this purpose, RNA from ERBB2+ and non-ERBB2+ breast cancer cells (BT474, MCF7, MDA MB231, and T47D) were extracted, cDNA synthesis was performed, PCR amplification of the cDNA were performed, and then sequenced with ERBB2 3′UTR primers by Sanger sequencing (see FIG. 106). The 3′UTR of ERBB2 were found in only ERBB2-expressing cancer cells MCF7, BT474, and T47D is enriched with UUUUUU sequences which are mRNA-stabilizing elements. However, these stabilizing elements are not enriched in triple-negative breast cancer MDA MB231. A synthetic gBlock of this 3′UTR region was designed, wherein the various stabilizing elements were engineered into destabilized elements and cloned them into a vector. To design the destabilized 3′UTR, all the stable elements were replaced with the destabilized ARE elements which are CCUC, CUGC, and UAAGUUAU. If the ERBB2-3′UTR were destabilized and its expression driven by the mRNA decapping enzyme promoter DCP1A, one could overwrite the endogenous ERBB2 mRNA and degrade the transcript, specifically through nonsense-mediated decay, resulting in loss of protein expression. Therefore, the destabilizing ERBB2 synthetic gBlock was designed to contain the BstB1 site, a poly A sequence to stop RFP transcription, and then a DCP1A promoter element to drive the destabilized ERBB2 3′UTR and then on the 3′ end with the Bamh1 site and a poly A to stop DCP1A.

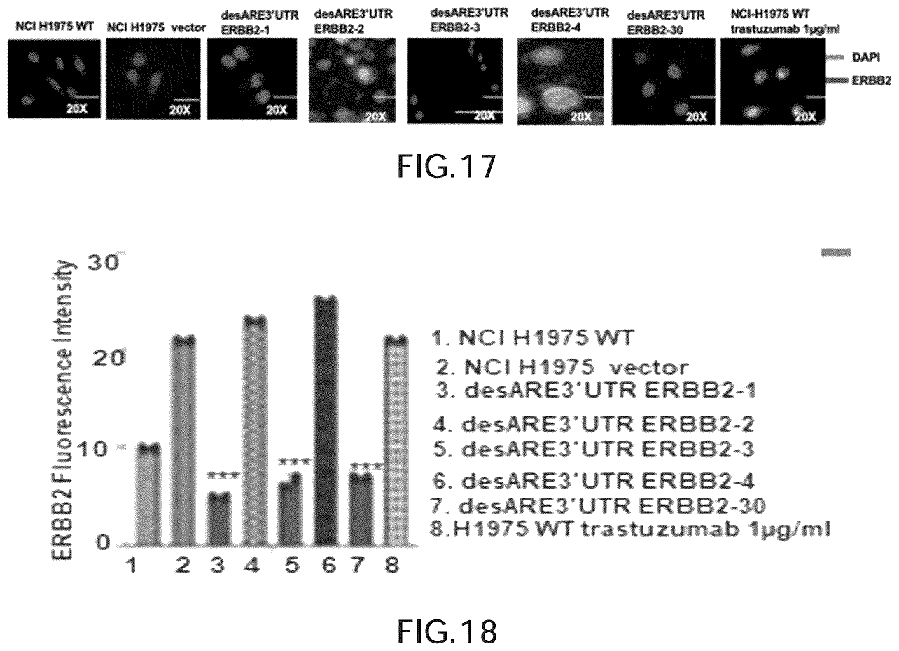

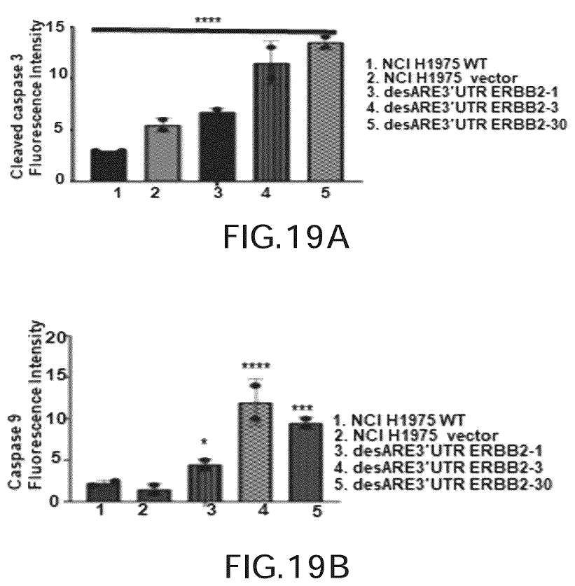

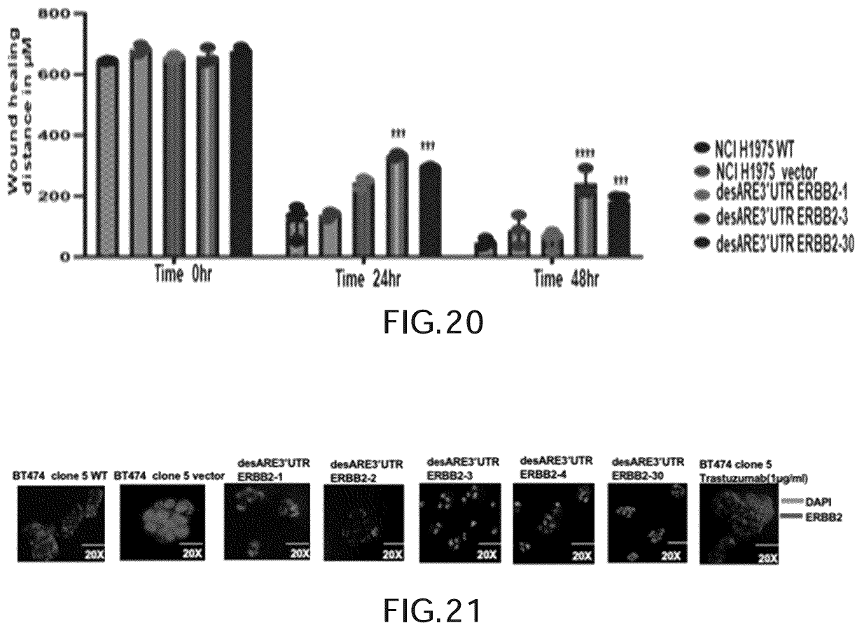

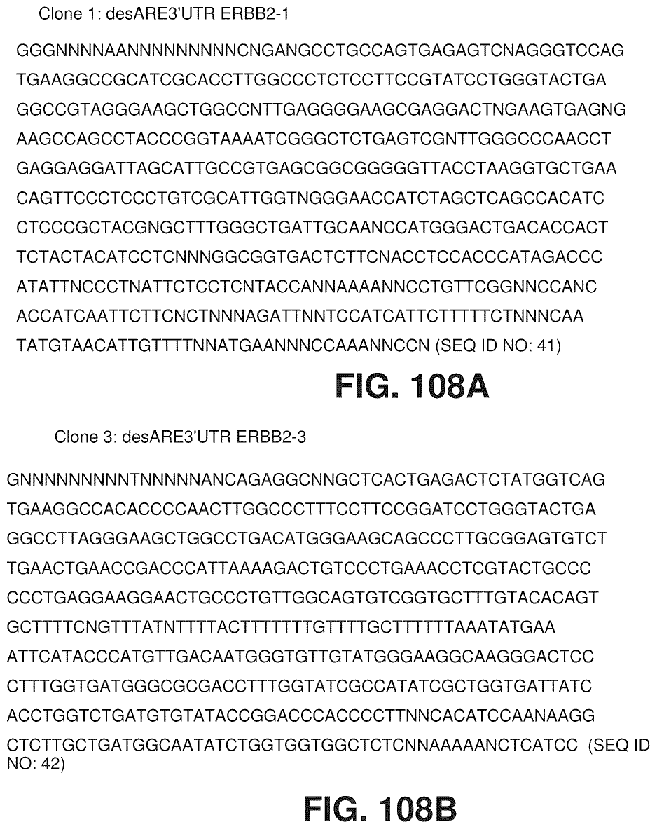

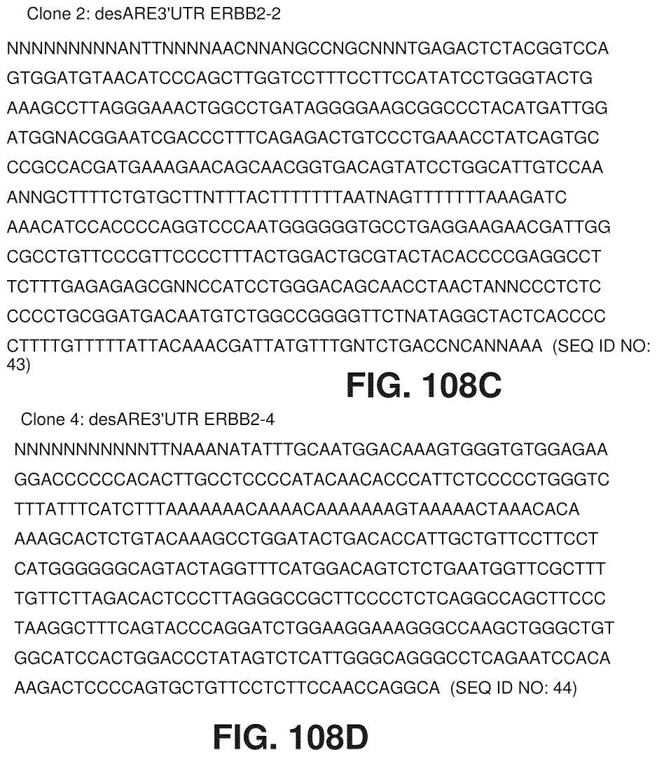

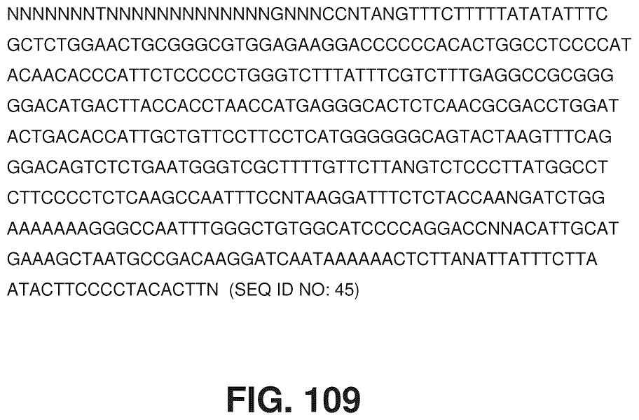

Engineered destabilized 3′UTR of ERBB2 degrades ERBB2 in ERBB2-expressing EGFR T790M lung cancer cells, ERBB2+ trastuzumab-resistant breast cancer cells, in wildtype ERBB2+ breast cancer cells, in ERBB2-mutated lung cancer cells, and in ERBB2-expressing colorectal cancer cells: The cloning strategy yielded four destabilizing 3′UTR ERBB2 clones by sticky end cloning in recombinant-competent E. coli, namely, desARE3′UTR ERBB2-1, desARE3′UTR ERBB2-2, desARE3′UTR ERBB2-3, and desARE3′UTR ERBB2-4 (FIGS. 108A-D) and a clone by Gibson assembly in recombinant-deficient E. coli: desARE3′UTR ERBB2-30 (FIG. 109). The four destabilized (des) clones (desARE3′UTR ERBB2-1, desARE3′UTR ERBB2-2, desARE3′UTR ERBB2-3, desARE3′UTR ERBB2-4) and desARE3′UTR ERBB2-30 were transfected into non-small-cell lung cancer cells that carry mutation in the EGFR T790M but expresses ERBB2. Within 4 days (FIG. 17), by immunofluorescence staining for ERBB2 (ERBB2 stained in red, and nuclei stained blue with DAPI) and Western blotting detection of ERBB2 (FIG. 13B), ERBB2 protein expression was lost in desARE3′UTR ERBB2-1, -3, and -30 compared to the wildtype cells, vector control, and trastuzumab-treated NCI-H1975 cells. The NCI-H1975 cells transfected with desARE3′UTR ERBB2-1, -3, and -30 show significant loss of viability compared to the controls (FIG. 13A). In Western blot, the desARE3′UTR ERBB2-3 and -30 (FIG. 13B) caused significant downregulation of ERBB2 protein expression compared to controls. The desARE3′UTR ERBB2-1, -3, and -30 also caused a significant decrease in ERBB2 transcript expression compared to controls (FIG. 13C). The morphology of the destabilized cells shows a ruptured membrane compared to controls. The significant downregulation of ERBB2 at the protein level as determined by immunofluorescence and Western blot was quantified and displayed in FIG. 18, FIG. 111. Increased expression of cleaved caspases 3 and 9 was found in the desARE3′UTR ERBB2-1, -3, -30-treated cells (FIG. 19A and FIG. 19B), and treated cells show significant inhibition of migration as demonstrated by significantly impaired wound healing (FIG. 20). Taken together, these results demonstrate that the engineered destabilized 3′UTR ARE of ERBB2 targeted, destabilized, and degraded the ERBB2 transcript and protein expression and induced increased apoptosis and reduced migration and outperformed trastuzumab in the NCI-H1975 model of the deadly EGFR T790M non-small-cell lung cancer.

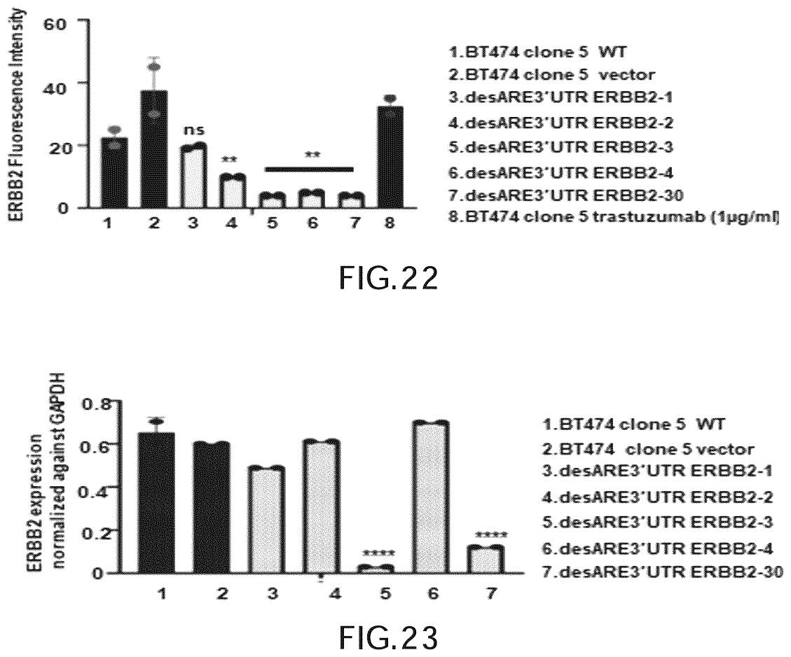

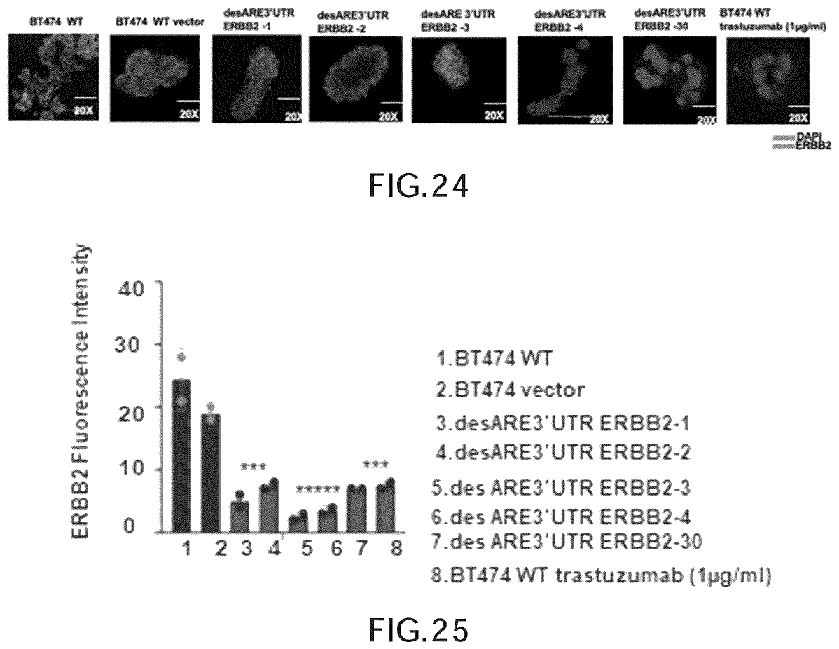

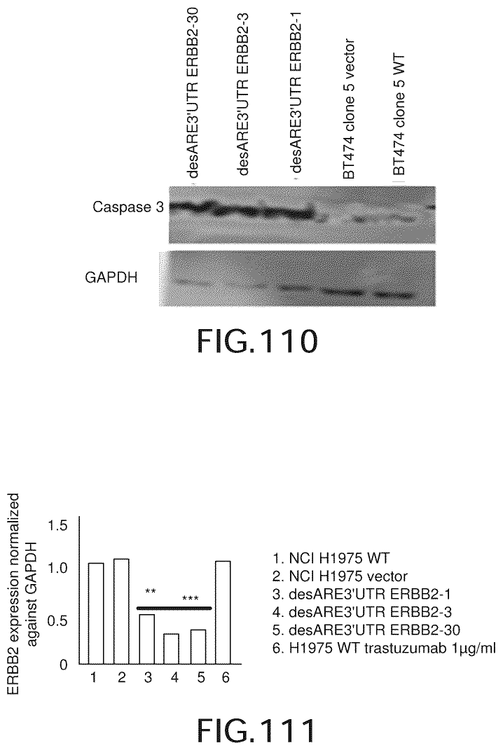

Trastuzumab-resistant cancers: Trastuzumab has improved overall survival outcomes for ERBB2+ breast cancer. However, more than 25% of early-stage ERBB2+ breast cancer patients develop resistance to trastuzumab and more than 75% late-stage ERBB2+ breast cancers are resistant to trastuzumab even if given in combination with anthracyclines. More so, there is no effective treatment for drug resistance in ERBB2+ breast cancer tumor relapse. To ascertain if the novel engineered destabilized 3′UTR of ERBB2 will successfully target ERBB2 in trastuzumab-resistant breast cancer cells and control its aggressiveness, the BT474 clone 5 was used (Torabi et al 2021). The ERBB2+ trastuzumab-resistant breast cancer cell line was developed by prolonged exposure to trastuzumab. These cells were transfected with the ERBB2 3′UTR destabilizing constructs, and within 9 days (FIG. 21). Immunofluorescence analysis showed that ERBB2 (stained in red) in the treated cells is diminished compared to the wildtype cells and vector controls (FIG. 21; FIG. 22). This was confirmed by Western blotting (FIG. 10B) and showed that desARE3′UTR ERBB2-3 significantly degraded ERBB2 expression and desARE3′UTR ERBB2-30 led to more than 70% significant downregulation of ERBB2 protein expression in the ERBB2+ trastuzumab-resistant breast cancer cells compared to wildtype and vector control cells (FIG. 10B, FIG. 24). The treated cells (except for those treated with desARE3′UTR ERBB2-4) also showed loss of viability compared to the controls (FIG. 10A). The morphology of the trastuzumab-resistant ERBB2+ cancer cells containing the destabilized elements shows shrinking and reduction in cell size compared to the controls (FIG. 21). Transcript analysis by the quantitative reverse transcriptase polymerase chain reaction (qPCR) showed that all the ERBB2 3′UTR-destabilizing constructs (FIG. 10C) significantly reduced the ERBB2 transcript, except for desARE3′UTR ERBB2-4 compared to the controls. ERBB2 3′UTR-destabilizing constructs induced increased caspase 3 in the trastuzumab-resistant ERBB2+ cancer cells (FIG. 110). Again, this result demonstrates that the engineered destabilizing ERBB2 3′UTR outperformed trastuzumab in the drug-resistant setting.

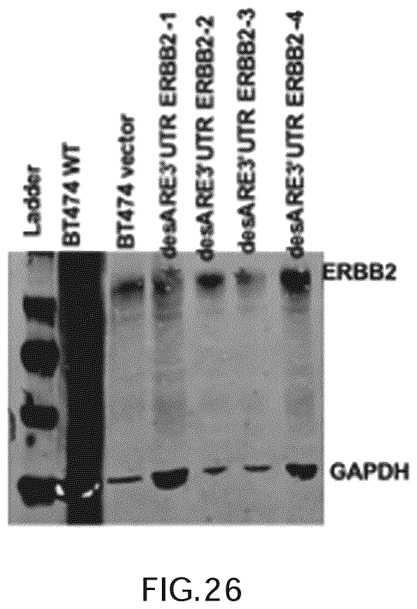

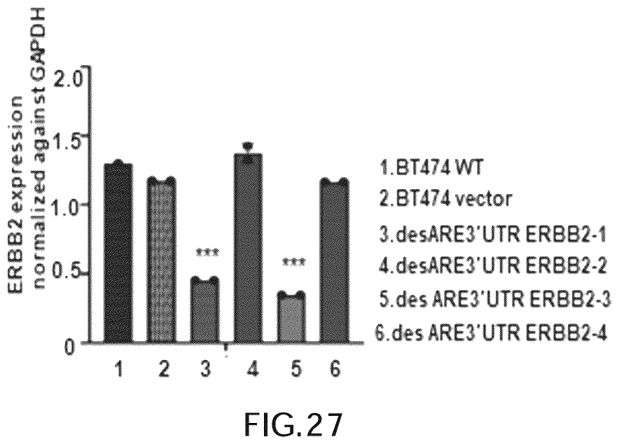

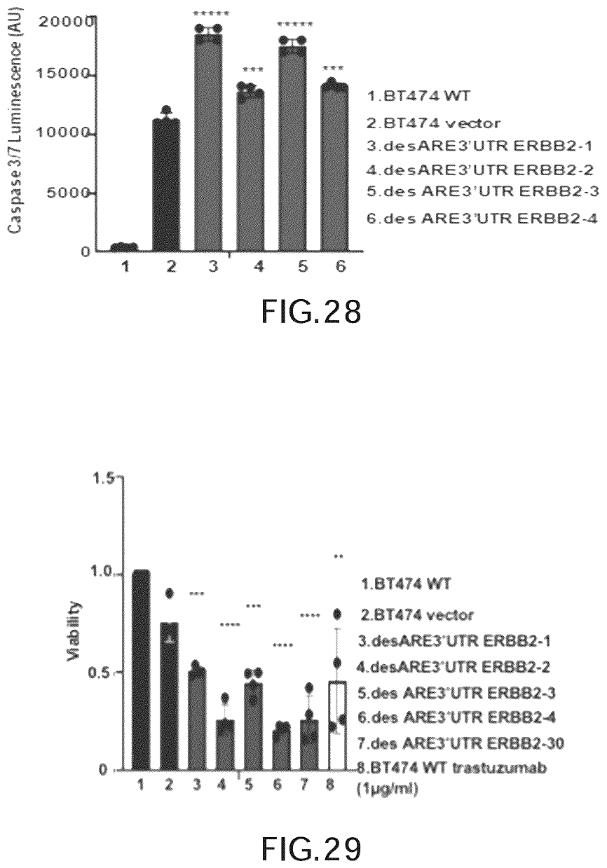

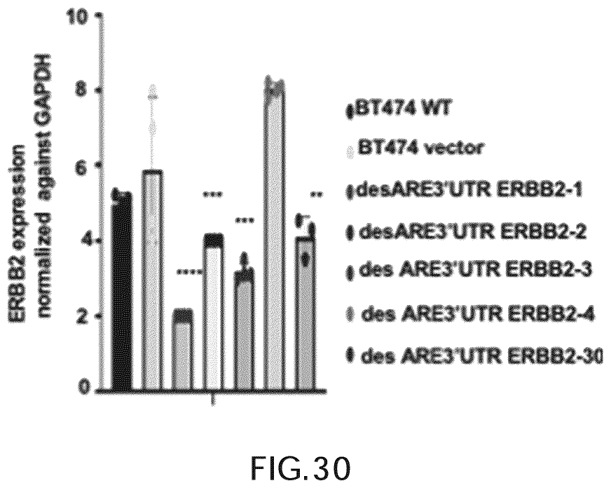

To further validate this, the four clones (desARE3′UTR ERBB2-1, desARE3′UTR ERBB2-2, desARE3′UTR ERBB2-3, and desARE3′UTR ERBB2-4) of the engineered destabilized 3′UTR ARE of ERBB2 were transfected into ERBB2+BT474 cells. Within 4 days (FIG. 24; FIG. 25), immunofluorescence showed that ERBB2 protein (stained green) expression has been significantly decreased in the cancer cells containing the destabilizing elements compared to the wildtype and vector control. The morphology of the destabilized cells under a microscope shows a distorted and ruptured membrane compared to the wildtype cells and the vector control. To confirm the significant downregulation of the ERBB2 protein, Western blotting was performed on the wildtype, vector controls cells, and the treated cells (FIG. 26; FIG. 27). The desARE3′UTR ERBB2-1 and desARE3′UTR ERBB2-3 degraded the ERBB2 protein and transcript (FIG. 30) expression. Within 8 days, all the cancer cells transfected with ERBB2 3′UTR-destabilizing elements showed loss of viability and increased caspase 3/7 expression (FIG. 28) compared to the wildtype cells and the vector control cells (FIG. 29).

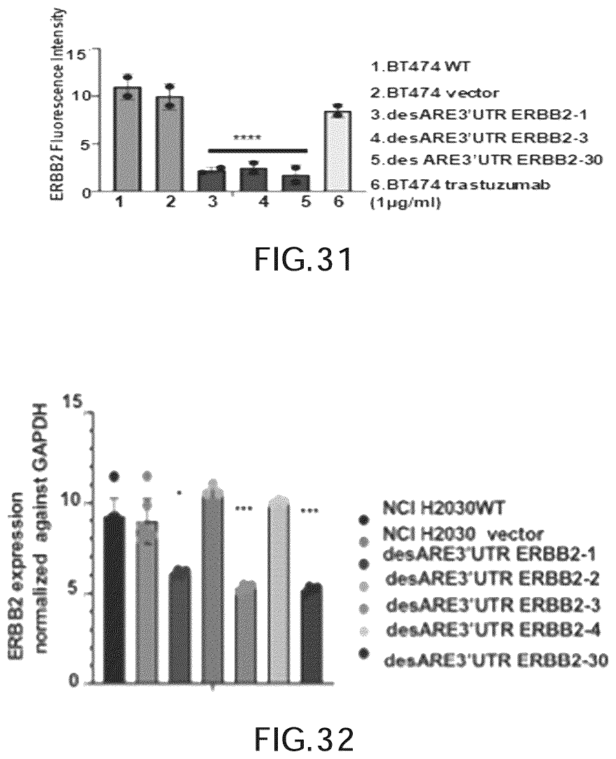

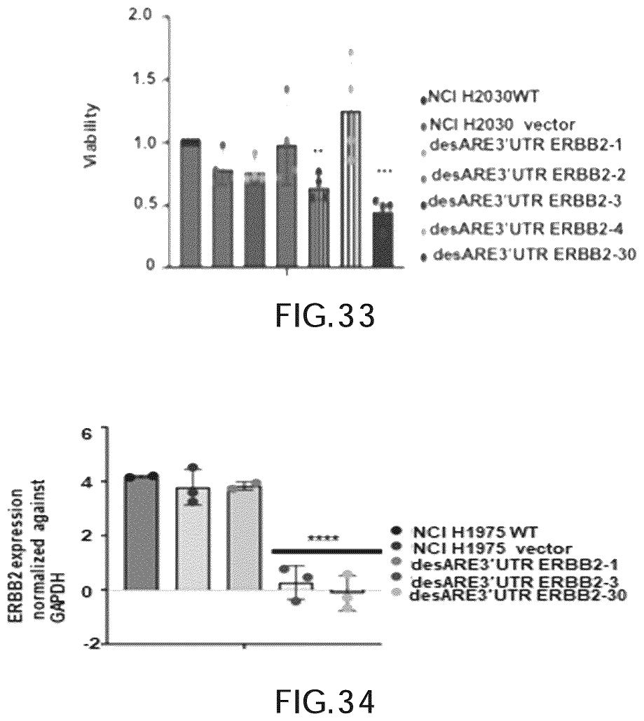

To further demonstrate the generalizability of the engineered 3′UTR destabilization of ERBB2 oncogene, this work was extended into the NCIH2030 ERBB2-mutated non-small-cell lung cancer cell line and the HCT116 colorectal cancer cell line. The cells containing ERBB2 3′UTR-destabilizing constructs show significant downregulation of the ERBB2 protein (FIG. 30; FIG. 31) and ERBB2 transcript (FIG. 32) and loss of viability (FIG. 33) compared to the wildtype and controls. The morphology of the cells containing the destabilizing constructs desARE3′UTR ERBB2-3 and -30 is different compared to the controls in NCI-H2030, while desARE3′UTR ERBB2-1, -3, and -30 show morphological changes compared to controls in HCT116. The desARE3′UTR ERBB2-3 and desARE3′UTR ERBB2-30 degraded the ERBB2 transcript in HCT116 cells (FIG. 34).

Taken together, the findings demonstrate that the engineered destabilized 3′UTR of ERBB2 effectively degraded the ERBB2 transcript and protein expression to kill cancer cells in a clinically relevant model of ERBB2+-overexpressing osimertinib-resistant EGFR T790M non-small-cell lung cancer, in ERBB2-mutated non-small-cell lung cancer, in ERBB2+-expressing colorectal cancer cell line, in difficult-to-treat ERBB2+ trastuzumab-resistant breast cancer cell line, and in wildtype ERBB2+-expressing breast cancer.

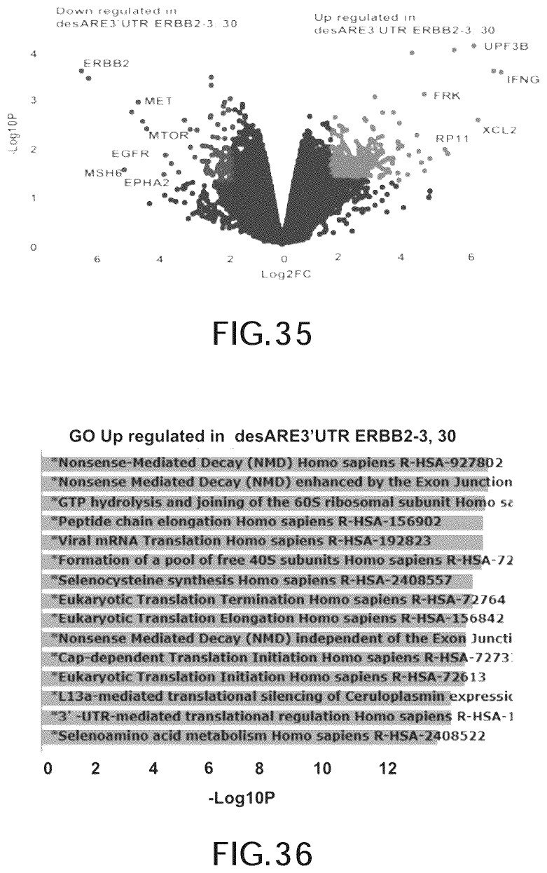

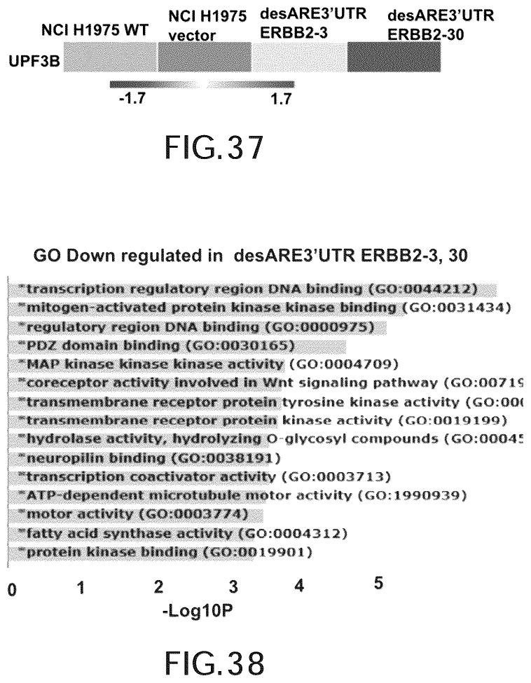

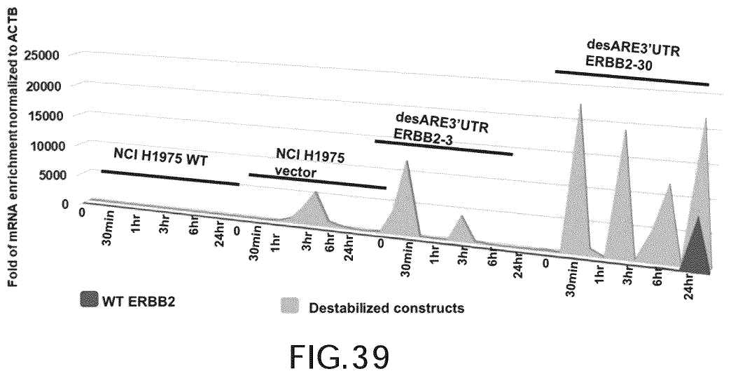

Transcriptome analysis shows that engineered destabilized 3′UTR ERBB2 degraded ERBB2 and its interactome through the designed nonsense-mediated decay pathway: To validate at the transcriptome level that the engineered destabilized 3′UTR of ERBB2 degraded ERBB2 and its interactome through the designed mechanism incorporated in the ERBB2 3′UTR-destabilizing constructs, RNA sequencing was performed on the ERBB2-expressing EGFR T790M lung cancer cells NCI-H1975 WT, NCI-H1975 vector, and in NCI-H1975 cancer cells containing desARE3′UTR ERBB2-3 and desARE3′UTR ERBB2-30. The desARE3′UTR ERBB2-3 and -30 show strong reduction in ERBB2 and EGFR, respectively (FIG. 35). The constructs were engineered to degrade mRNA once the transcript ARE is destabilized through mRNA decay machinery. By gene ontology analysis, we investigated if the mRNA decay mechanism is triggered once the transcript is destabilized and driven by the mRNA cap deadenylating DCP1A promoter. As designed in the constructs, the gene ontology theme of nonsense-mediated decay is most significantly enriched GO biological and molecular function with a p-value=5.2e⁻¹³ and FDR=5.3e⁻¹⁰, and the genes UPF3B and UPF3A, which are known regulators of nonsense-mediated decay, were implicated (FIG. 35, FIG. 36 and FIG. 37). Again, the ontology theme also identified upregulated 3′UTR-mediated translation regulation (p-value=1.6e⁻¹¹ and FDR=8.7e₋₁₀) as part of the mechanism implicated in corroborating the engineering design which specifically targeted the ARE on the 3′UTR of ERBB2 (FIG. 36). The GO-downregulated theme implicated transmembrane receptor tyrosine kinase (p=0.0008, FDR=0.048) to which ERBB2, which is an EGFR family receptor, belongs (FIG. 38). To determine how the destabilizing constructs degrade the endogenous ERBB2 mRNA, a 4SU mRNA labeling pulse-chase experiment was performed on the NCI-H1975 WT, NCI-H1975 vector, and desARE3′UTR ERBB2-3 and -30 at 0 h, 30 mins, 1 h, 3 h, 6 h, and 24 h. RNA was collected, made into cDNA, and RT-qPCR was performed with primers targeting the endogenous ERBB2 (forward and reverse primers for exon 6) as well as primers targeting the vector and RFP (forward) and cloned destabilizing ERBB2 constructs (reverse). ACTB served as a control housekeeping gene. The destabilized construct transcript is 2.5×10⁴ overexpressed more than the endogenous ERBB2. These data indicate that the destabilizing ERBB2 transcript is made at such a high amount that the transcription machinery overwrites/outcompetes the endogenous ERBB2 message with the destabilizing one using this approach (FIG. 39). Based on the thorough 4SU mRNA labeling data shown in FIG. 39, the minimum destabilized transcript level required to outcompete/overwrite the endogenous ERBB2 mRNA is approximately 6×10³. Using the data from the mRNA pulse-chase time-dependent 4SU experiment, the half-life of the wildtype ERBB2 and destabilized ERBB2 was calculated using one phase decay equation. The half-life of WT ERBB2 approximately 1.5 hrs and the destabilized construct transcript approximately 6 hrs (i.e. greater than 3 hours or greater than 5 hours). Indeed, the transcriptome analysis confirmed that these engineered constructs reliably targeted ERBB2 and worked as the engineered constructs were designed to function through mRNA-mediated decay driven by the decapping DCP1A promoter to trigger nonsense-mediated mRNA decay and exonucleases, deadenylases to overwrite/outcompete, and degrade the ERBB2 transcript.

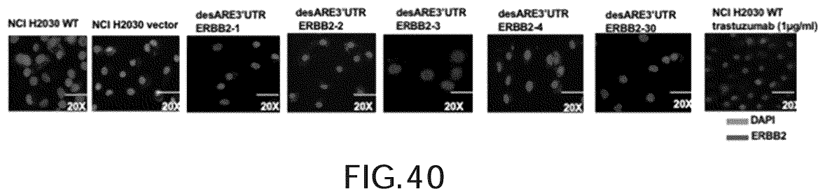

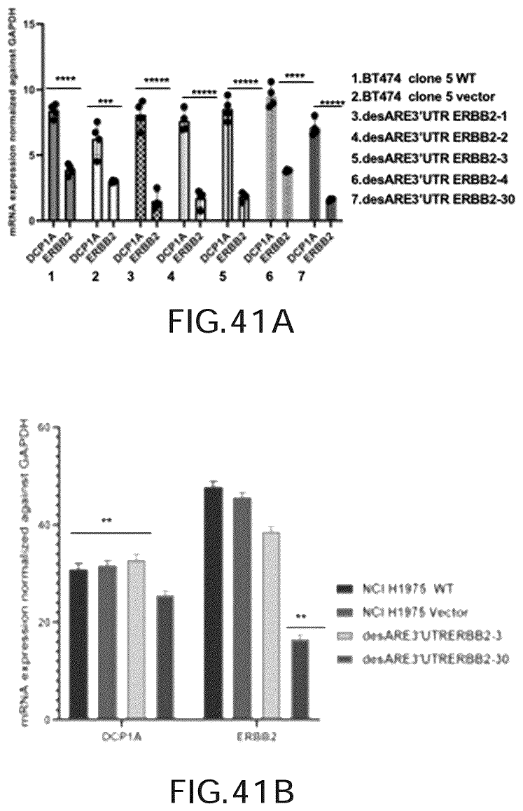

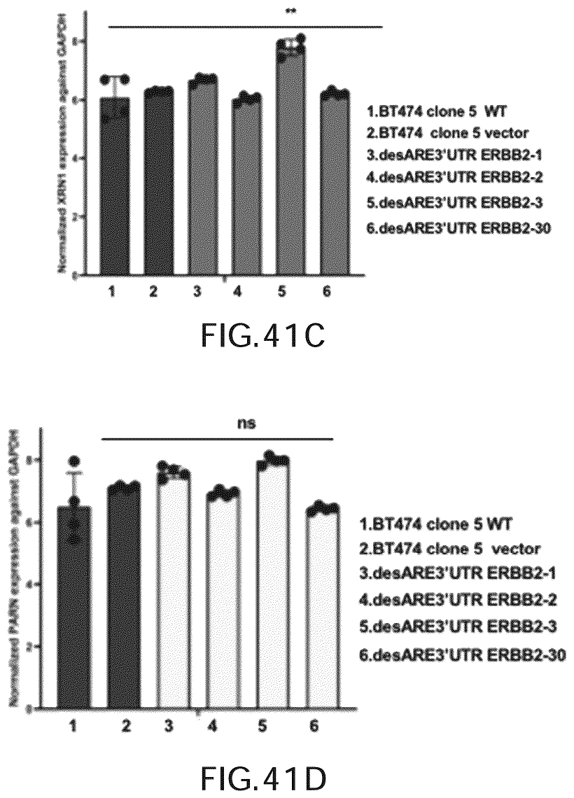

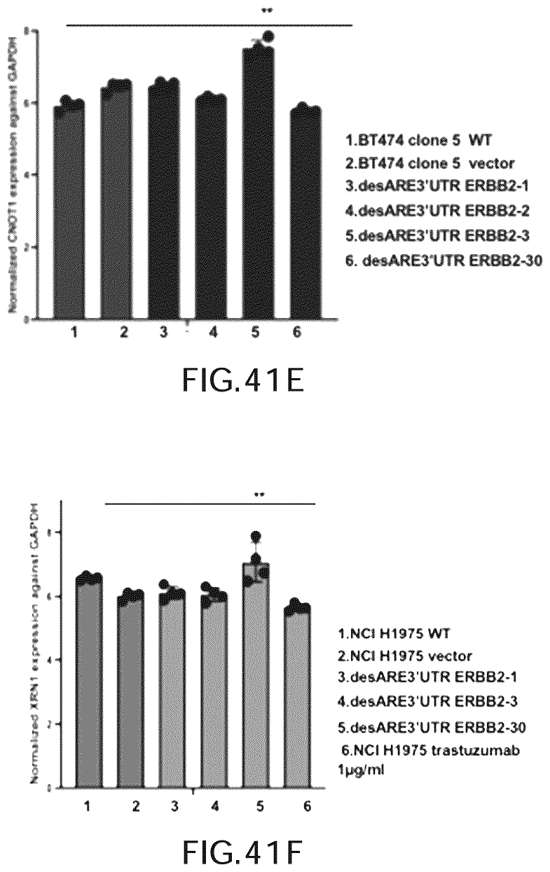

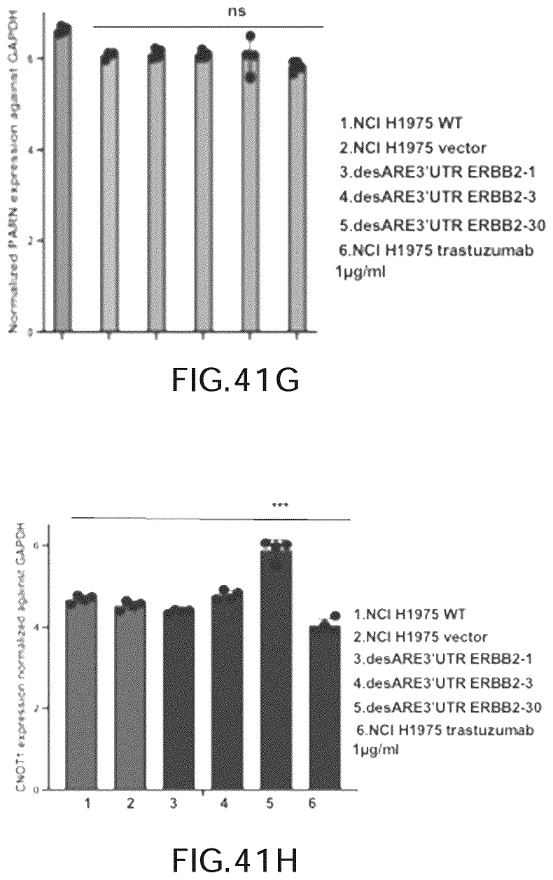

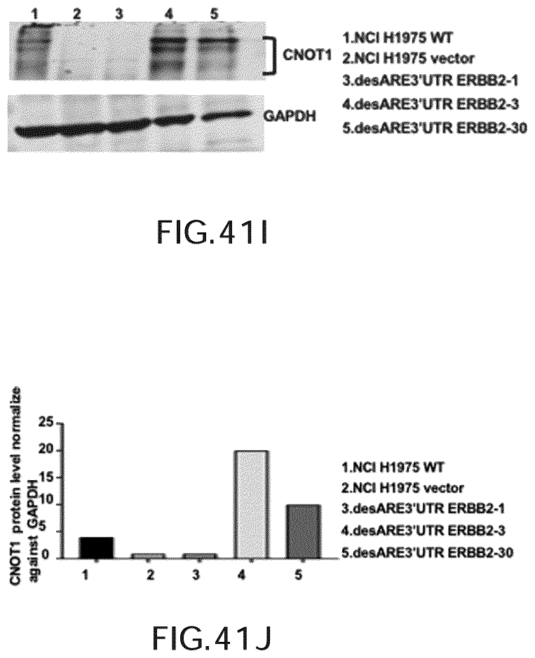

Engineered destabilized 3′UTR significant downregulation of ERBB2 is sequence specific and triggers exonucleases XRN1 and CNOT1 to degrade the ERBB2 transcript: To ascertain the molecular details of how the degradation of ERBB2 is achieved (FIG. 17, FIG. 13B, FIG. 21, FIG. 10B; FIG. 24, FIG. 26, FIG. 40), we designed the engineered destabilized 3′UTR of ERBB2 to be driven by the mRNA decapping enzyme DCP1A promoter. To understand if the DCP1A promoter contributed to ERBB2 degradation through mRNA decapping upon the introduction of the engineered destabilizing 3′UTR ARE of ERBB2 into the breast and lung cancer cells, qRT-PCR was performed which showed that DCP1A promoter expression did not change upon destabilization of the ERBB2 transcript compared to the controls (FIG. 41A). More so, DCP1A expression from RNA seq (FIG. 41B) shows that DCP1A does not increase in the destabilized constructs compared to control. This finding suggests that the engineered destabilized 3′UTR ARE sequence only explains the degradation of ERBB2. To understand the deadenylation and cleaving proteins involved in this process and if there is a preference for 5′-3′ or 3′-5′ deadenylation and cleavage, a qRT-PCR was performed on the 5′-3′ cleaving enzyme XRN1 and 3′-5′ deadenylase PARN and CNOT1 both on the BT474 clone 5 and the NCI-H1975 containing the destabilized constructs compared to the controls. In the BT474 clone 5 containing the destabilized 3′UTR ARE of ERBB2, the XRN1 expression is significantly elevated in the desARE3′UTR ERBB2-3 (FIG. 41C), which completely degraded the ERBB2 protein in FIG. 41F; FIG. 41H. The PARN expression is not elevated (FIG. 41D). The CNOT1 expression is significantly elevated in desARE3′UTR ERBB2-3 (FIG. 41E). Similar results were found in NCI-H1975 transduced with the destabilizing constructs of the 3′UTR of ERBB2. XRN1 is significantly elevated in desARE3′UTR ERBB2-30 (FIG. 41F); also, CNOT1 is significantly elevated in the desARE3′UTR ERBB2-30 (FIG. 41H). desARE3′UTR ERBB2-30 showed the most significant downregulation of ERBB2 in FIG. 17. Again, the PARN expression is not elevated in NCI-H1975 (FIG. 41G) as seen in the BT474 clone 5 (FIG. 41D). Finally, to prove the increased CNOT1 protein expression in the destabilized cells, a Western blot was performed which showed that CNOT1 is highly expressed in the cells carrying desARE3′UTR ERBB2-3 and -30 (FIG. 41I, FIG. 41J). In vivo, a significant downregulation of ERBB2 was found in animals bearing tumors treated with desARE3′UTR ERBB2-1, -3, and -30 and increased expression of UPF3B, XRN1, and CNOT1. These results suggest a general mechanism that both 5′-3′ and 3′-5′ deadenylases, cleaving enzymes, and nonsense-mediated decay are involved in the degradation of the ERBB2 once the 3′UTR of ERBB2 is destabilized.

Destabilized 3′UTR degradation of ERBB2 in the osimertinib- and trastuzumab-resistant cancer cell lines leads to loss of kinases implicated in breast and lung cancer drug resistance: The signaling cascade of ERBB2 is mediated by kinases and as such the resistance to anti-ERBB2 is concomitant with the increased activity of kinase that helps the drug-resistant cancers to proliferate and metastasize. Osimertinib EGFR T790M resistance arises due to upregulation of EGFR-dependent kinases and EGFR-independent pathway. Destabilization of ERBB2 in this non-small-cell lung cancer line does, indeed, control these implicated kinases and proteins. EGFR, MET, and MAPK kinases, implicated in the EGFR dependent pathway of osimertinib resistance, were all downregulated. More so, the EGFR-independent pathways such as MTOR, CNNTB1, EPHA2, and NOTCH were downregulated upon destabilization of ERBB2. This finding points to the superiority of this approach in controlling drug resistance driven by ERBB2 and its widespread interactome, such that when we control ERBB2, we controlled all the pathways that are dependent on it.

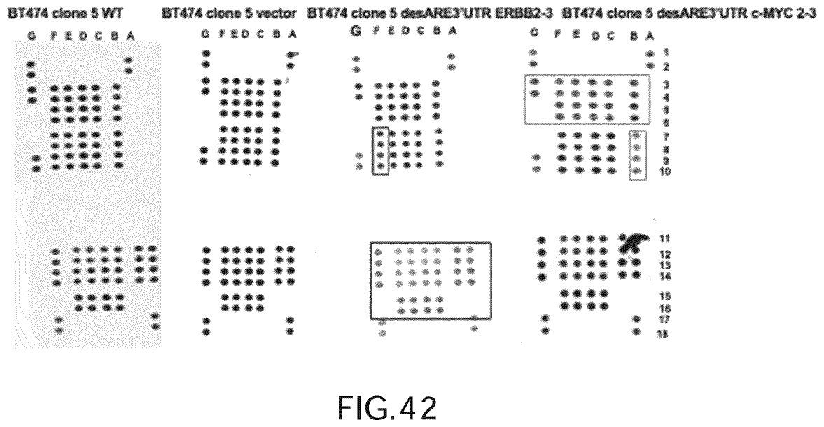

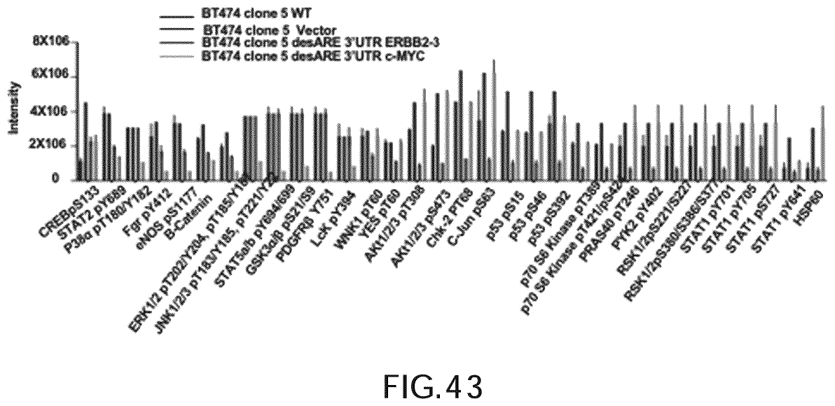

To ascertain if the destabilized 3′UTR of ERBB2 degradation of ERBB2 led to loss of kinases known to contribute to ERBB2+ trastuzumab resistance, a phosphorylation kinase array assay was performed with protein extracts from BT474 clone 5 WT, BT474 clone 5 vector, BT474 clone 5 desARE3′UTR ERBB2-3, and desARE3′UTR c-MYC 2-3 (a specificity control for ERBB2 destabilization) (FIG. 21, FIG. 10A, FIG. 10B, FIG. 42, FIG. 43). Kinases such as AKT1/2/3 pT308, AKT1/2/3 pS473, Chk-2 pT68, c-JUN pS63, P53 pS46, P53pS15, P53 pS392, P70 S6 kinase pT389, P70 S6 kinase pT421/pS424, PRAS 40 pT246, PYK2 pY402, RSK1/2 pS221/pS227, RSK1/2/3 pS380/pS386, pS377, STAT1pY701, STAT2 pY705, STAT2 pS727, STAT3 pS727, STAT6 pY641, and HSP60 marked with a box in the lower panel (FIG. 42) of desARE3′UTR ERBB2-3 were all downregulated upon the degradation of ERBB2 by 3′UTR destabilization compared to controls (FIG. 42, FIG. 43).

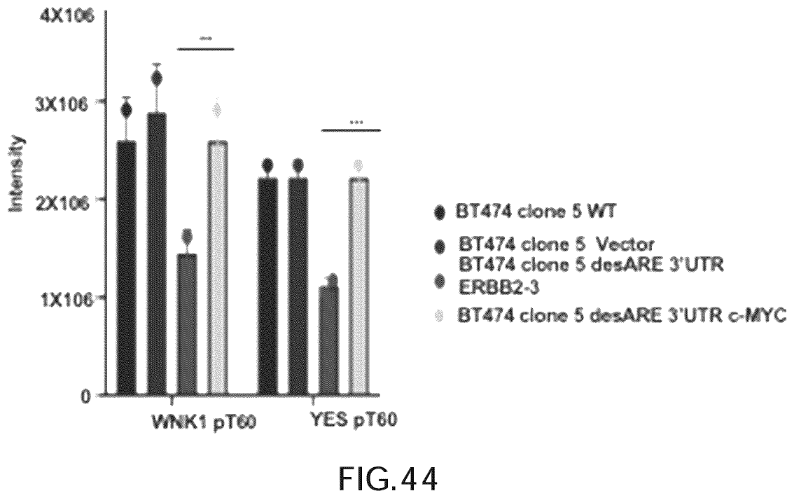

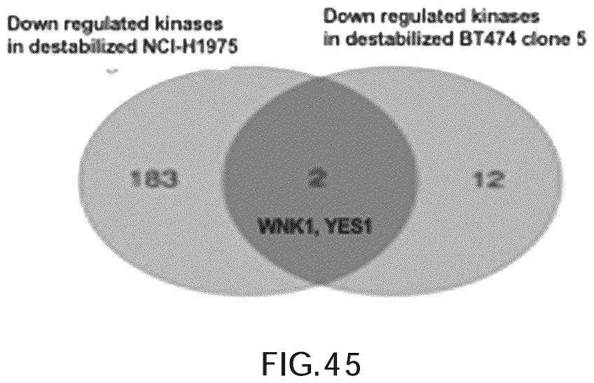

These kinases downregulated have been implicated in mediation of trastuzumab resistance in breast cancer, both from clinical trials and in cancer cell lines. The result revealed ERBB2 is a master regulator controlling a vast array of kinases and transcription factors and that the disclosed technology can control not only the ERBB2 but as well all the signaling cascade and interactome associated with it. Kinases were also explored that were lost in desARE3′UTR ERBB2-3 samples but whose expression is relatively similar both in wildtype, vector control, and desARE3′UTR c-MYC 2-3; a specificity control for ERBB2 destabilization, the kinases YES pT60 and WNK1 pT60 (FIG. 42, FIG. 43 and FIG. 44) marked with a box in the upper panel of desARE3′UTR ERBB2-3. To find the universal common kinases that mediate drug resistance both in osimertinib-resistant EGFR T790M lung cancer and trastuzumab-resistant breast cancer that are downregulated upon our destabilization of ERBB2, we triaged all the kinases downregulated in ERBB2 destabilized NCI-H1975 found by RNA Seq with kinases downregulated in ERBB2 destabilized BT474 clone 5 (FIG. 42, FIG. 43 and FIG. 44) found by the kinase array. WNK1 and YES1 is the common shared kinase by these two cancer cell lines that mediates drug resistance in them amongst other (FIG. 45).

To assess the specificity of the kinases downregulated through the destabilization of the 3′UTR of ERBB2 is unique to ERBB2 and not to other oncogenes such as c-MYC, a phosphorylation kinase array assay was performed on the protein extract of the BT474 clone 5 destabilized with desARE3′UTR MYC 2-3. In FIG. 42, in the last panel marked with a box, the kinases lost by the destabilization of c-MYC is unique to c-MYC and that kinases lost by destabilization of ERBB2 is unique to ERBB2, proving surprisingly that the destabilization of the ARE 3′UTR of oncogenes is unique and specific and a targeted molecular therapy.

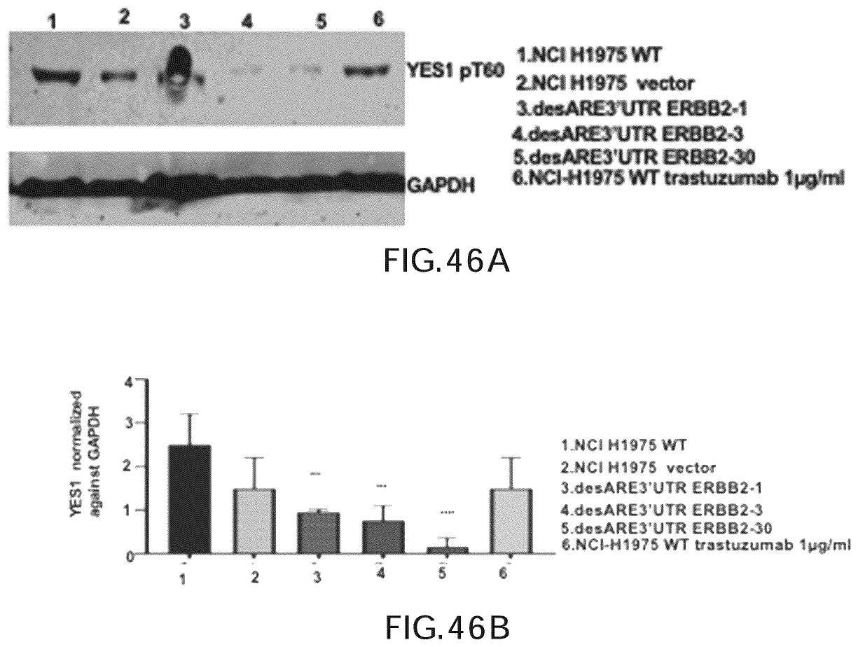

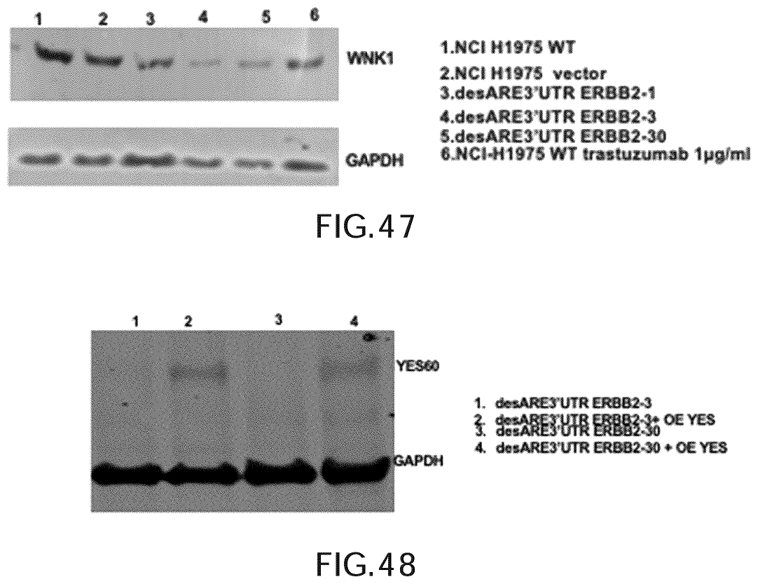

To validate the loss of YES1 pT60 and WNK1 upon the destabilization of the 3′UTR of ERBB2 (FIGS. 42-45), a Western blot was performed on desARE3′UTR ERBB2 samples compared to the controls. YES1 pT60 protein expression was decreased in the desARE3′UTR ERBB2-3 and -30 compared to the controls across multiple ERBB2-driven cancer types such as NC-H1975 (FIG. 46A, FIG. 46B). The engineered destabilized desARE3′UTR ERBB2-1, -3, -30 degraded WNK1 (FIG. 47). Taken together, this demonstrates that by destabilizing ERBB2, one can control and downregulate YES1 and WNK1 that are well-known causes of drug resistance in ERBB2+ trastuzumab drug-resistant breast cancer and in osimertinib-resistant EGFR T790M lung cancer, respectively.

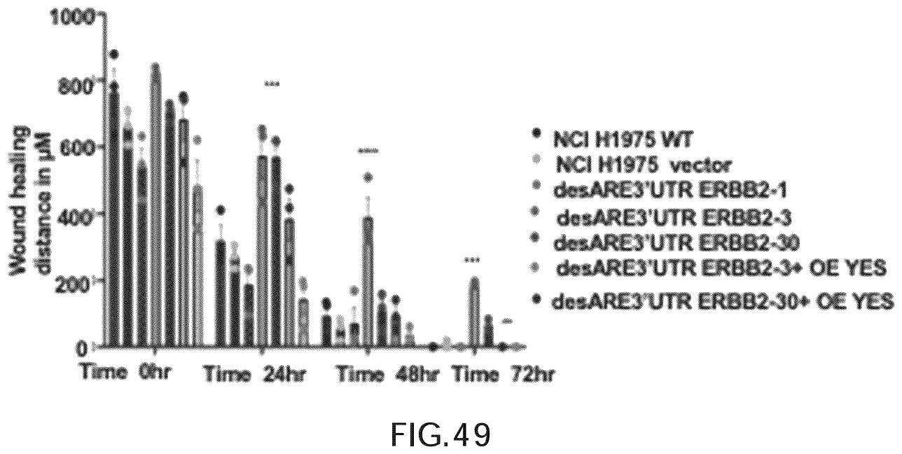

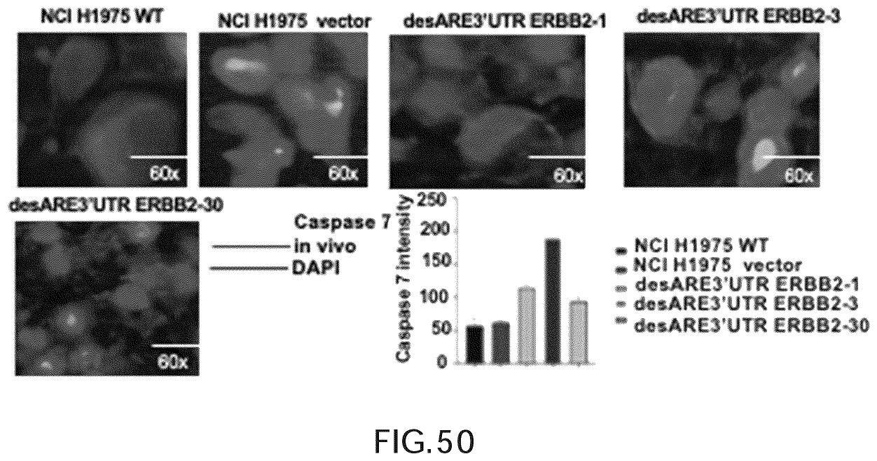

In a gain of function experiment, YES1 was overexpressed in NCI-H1975 desARE3′UTR ERBB2-3 and -30 cells that were degraded and reduced ERBB2 and YES1. The overexpression of YES1 was confirmed by the Western blot (FIG. 48). Next, a wound healing assay was performed comparing the wildtype control, vector, desARE3′UTR ERBB2-1, -3, and -30 and desARE3′UTR ERBB2-3 and -30 with overexpressed YES. As shown in FIG. 49, desARE3′UTR ERBB2-3 and -30 OE YES closed their wound at similar time points as the controls. The cells bearing the destabilized desARE3′UTR ERBB2-3 and -30 were unable to close their wounds due to lack of ERBB2 and YES and this triggered caspase 7 (FIG. 50). This finding confirms ERBB2 and its downstream YES kinase control resistance in NCI-H1975 and that the disclosed approach is superior in controlling ERBB2 pervasive oncogene signal and their downstream YES kinases that are involved in drug resistance which Osimertinib and trastuzumab cannot control.

The engineered destabilized 3′UTR of ERBB2 outperforms ShRNAi and genome-scale CRISPR in controlling the ERBB2 transcript in ERBB2-expressing colorectal cancer HCT116: To evaluate the performance of the engineered destabilized ARE 3′UTR of ERBB2 in controlling the ERBB2 transcript and downstream kinases in comparison to ShRNAi and genome-scale CRISPR, the publicly available database were analyzed for ShRNAi and CRISPR (Vizeacoumar et al., 2013; Hart et al., 2015; Martin et al., 2017) in HCT116, a colorectal carcinoma cell, an ERBB2-expressing cancer. Table 9 shows that ShRNAi and CRISPR across multiple studies were unable to downregulate the ERBB2 transcript in HCT116 and its associated kinases, whereas desARE3′UTR ERBB2 degraded the ERBB2 transcript in this disclosure as well as YES1. Hart et al. (2015) suggested that lack of TP53 S241F EGFR dependency in HCT116 prevented erlotinib and ERBB2 and EGFR sgRNA from controlling ERBB2 in the HCT116 KRAS G13D cell line. The engineered destabilized ARE 3′UTR of ERBB2 is agnostic to these mutations and reliably controlled and degraded the ERBB2 transcript in this cancer. This finding strongly suggests that destabilization of stable oncogene 3′UTR ARE is superior to RNAi and CRISPR in certain genetic mutation settings.

TABLE 9
Comparison of ShRNAi, CRISPR, and mRNA 3′UTR destabilization in
targeting HER2 in colorectal carcinoma HCT116

| Transcript | | | | | |
|---|---|---|---|---|---|
| Perturbation | | | | | Associated |
| method | Publication | Target | Cell line | Outcome | kinases outcome |
| Genome-wide | Vizeacoumar, | EGFR, | HCT116 | ERBB2 not | YES1 not |
| ShRNA | F. et al. | ERBB2 | | downregulated | downregulated |
| | (2013). Mol. | | | | |
| | Syst. Biol. 8, | | | | |
| | 696. | | | | |
| | doi:10.1038/ | | | | |
| | msb.2013.54 | | | | |
| Genome-wide | Hart, T., et al. | EGFR, | HCT116 | EGFR and | |
| CRISPR | (2015) Cell | ERBB2 | | ERBB2 not | |
| KO | 163, 1515- | | | downregulated | |
| | 1526. | | | | |
| | doi:10.1016/j. | | | | |
| | cell.2015.11.0 | | | | |
| | 15 | | | | |
| Genome-wide | | EGFR, | HCT116 | EGFR and | YES1 not |
| CRISPR | | ERBB2 | | ERBB2 not | downregulated |
| KO plus | | | | downregulated | |
| shRNA | | | | | |
| desARE3′ | This | EGFR, | HCT116 | ERBB2 | YES1 |
| UTR | disclosure | ERBB2 | | downregulated | downregulated |
| ERBB2-1 | | | | | |
| and -3 | | | | | |

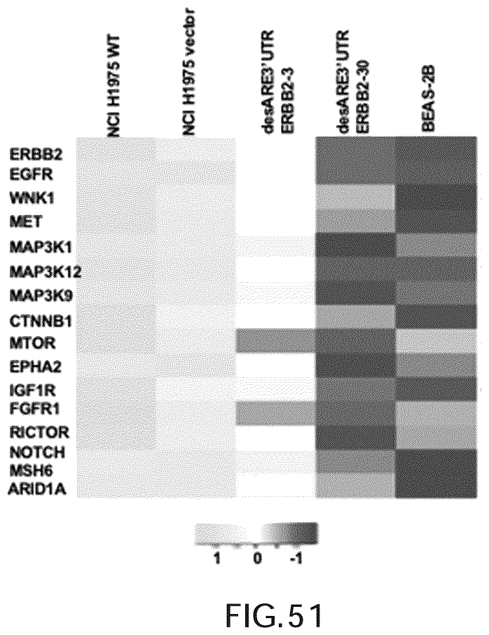

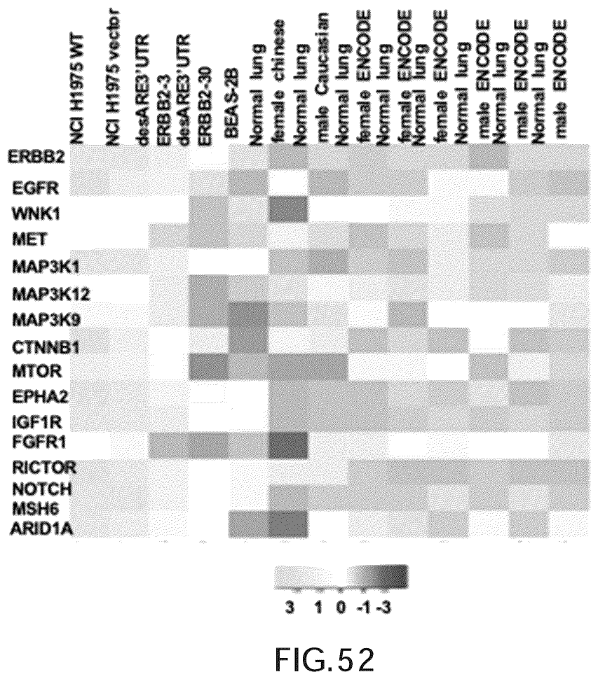

Engineered destabilized 3′UTR of ERBB2 reprogrammed lung cancer ERBB2 gene expression toward the normal lung epithelial gene expression pattern: To understand if destabilization of the ERBB2 transcript by 3′UTR engineering restored the lung genetic program toward normal lung epithelial cells, the ERBB2 gene expression profile of wildtype NCI-H1975, vector, desARE3′UTR ERBB2-3 and -30, and BEAS-2B, a normal human lung epithelial cell line (E-MTAB-4729) were compared. The degrading of the ERBB2 transcript by destabilized 3′UTR changed the cancer ERBB2 gene expression almost to near normal lung epithelial cell gene expression program (FIG. 51). These data were extended to compare if destabilization of the ERBB2 transcript and protein in cancers does reprogram the gene expression of cancers toward the normal human lung gene expression pattern. For this purpose, the RNA Seq of diverse normal human lung individuals (normal Chinese female non-smokers, SRR1797221, normal Caucasian male, normal female (3), and normal male (3) ENCODE data were obtained. Indeed, in FIG. 52, desARE3′UTR ERBB2-30 reprogrammed the cancer ERBB2 gene expression to the normal pattern as in healthy humans. These data strongly suggest that 3′UTR destabilization of the oncogene transcript is safe and achieves the restoration of normal gene expression patterns.

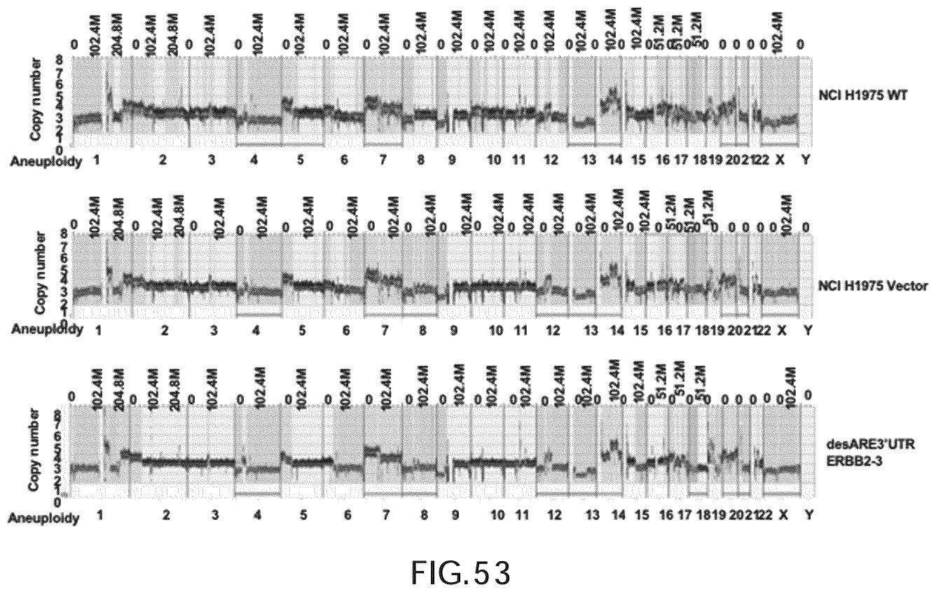

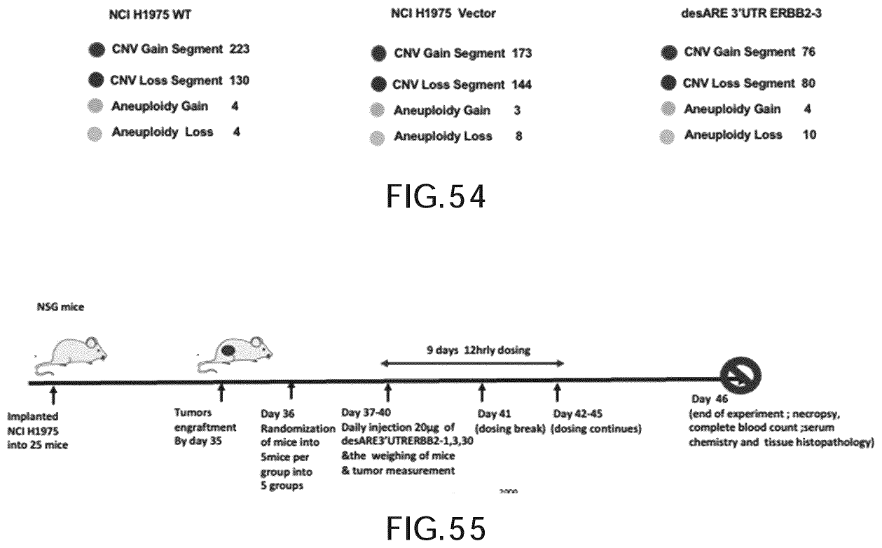

Genome-wide optical genome mapping shows that engineered destabilized 3′UTR ERBB2 de novo and rare variant assembly are similar with the parental cell line: To rule out that the engineered destabilization of the 3′UTR of ERBB2 did not cause severe genome alterations and genome rearrangements in the engineered cells compared to the wildtype and control cells, the ultrasensitive technique of optical genome mapping was used to find found that the cells carrying the constructs desARE3′ERBB2-3 showed almost the same de novo and rare variant assembly compared to the wildtype cells and the vector (FIG. 53). The desARE3′UTR ERBB2-3 has lesser copy number variation segment gain, lesser copy number variation segment loss, and more aneuploidy loss than the wildtype and vector. This result can be interpreted as the destabilizing construct desARE3′UTR ERBB2 is safe for the cells and had lesser deleterious changes than the wildtype and vector control (FIG. 54). Aneuploidy is a major cause of chromosomal instability and cancer. It is surprising to find that in these results, the engineered constructs destabilized ERBB2 and led to a significant loss of aneuploidy in the cancers carrying the constructs compared to the controls. These results suggest that destabilized 3′UTR degradation of the ERBB2 transcript has anti-cancer activity amongst which is reduction in cancer cell aneuploidy.

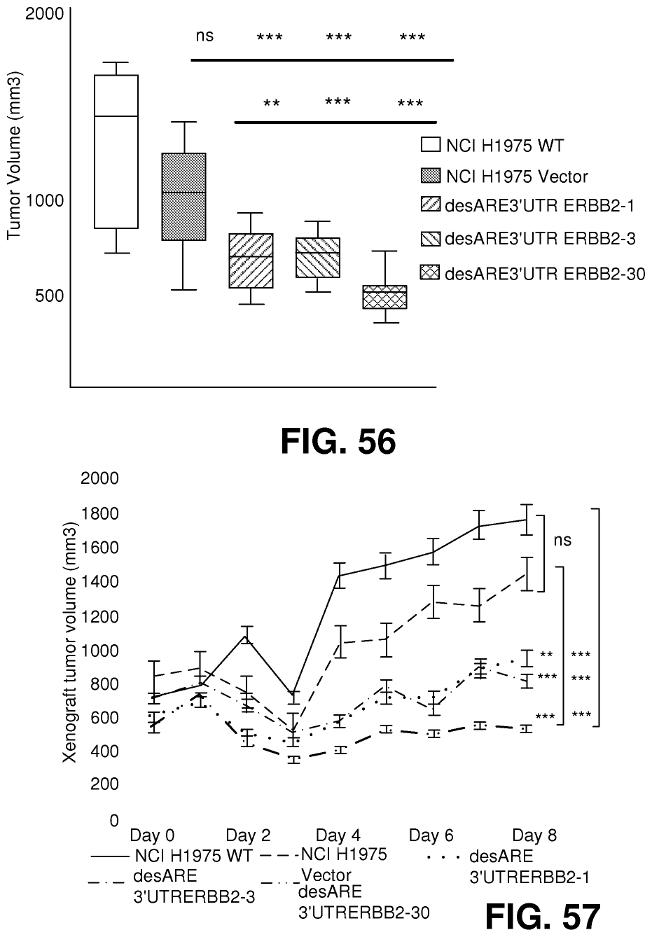

Engineered destabilized 3′UTR of ERBB2 significantly decreased tumor volume in vivo with no toxicity or adverse effects: To validate the findings that the engineered destabilized 3′UTR of ERBB2 degrades ERBB2 in vivo and reduces ERBB2-driven tumor growth and volume in a mouse tumor-bearing model, 25 female NSG mice were implanted with 5 million NCI-H1975 cells on the flank and after 35 days, huge tumors engrafted (FIG. 55). On day 36, the mice were randomized into five different groups, namely, wildtype, vector, desARE3′UTR ERBB2-1, desARE3′UTR ERBB2-3, and desARE3′UTR ERBB2-30 with the groups having equal representation of tumor size. The vector and the constructs desARE3′UTR ERBB2-1, -3, and -30 were administered at 20 μg per dose given intraperitoneal 12 hrly for 9 days. Upon each administration, the mice were weighed, tumor size was measured with calipers, and both length and width were recorded. In addition, the body condition score of the mice was recorded too. Tumor volumes were plotted comparing the wildtype and the mice that received the vector, desARE3′UTR ERBB2-1, -3, and -30. Referring to FIG. 56 and FIG. 57, the tumor volumes were significantly reduced in the mice bearing tumors that received the engineered constructs desARE3′UTR ERBB2-1, -3 and -30 compared to the wildtype and vector and that there were no significant differences between tumor volumes in the wildtype and the vector. This finding strongly validates in vivo the already established findings here in which the engineered destabilized 3′UTR of ERBB2 degraded ERBB2 and its interactome and impaired cancer cell growth, size, and migration.

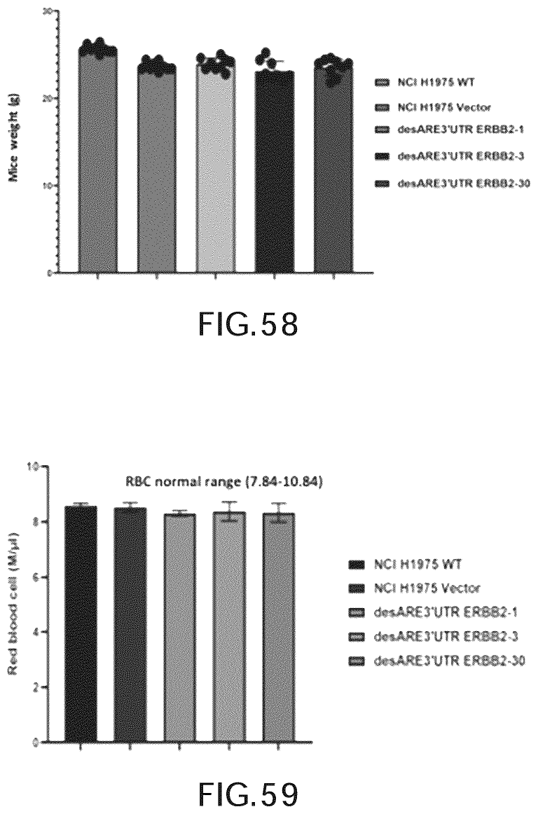

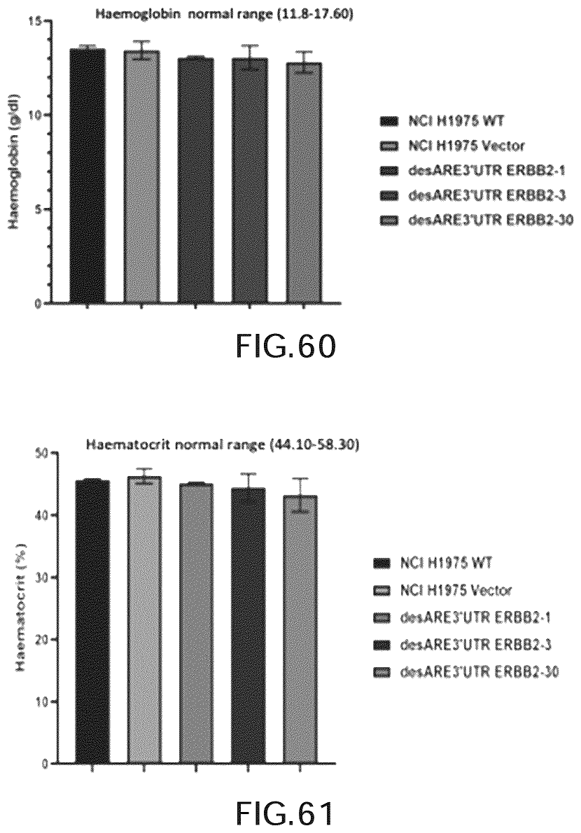

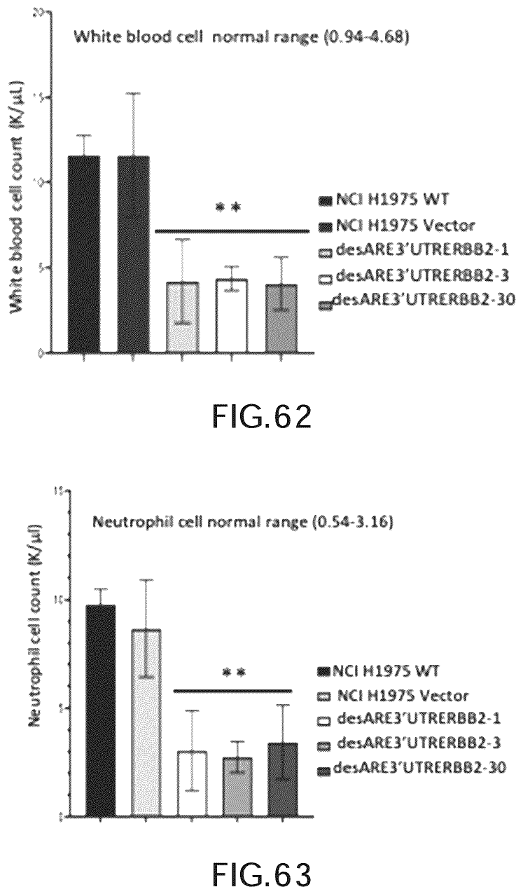

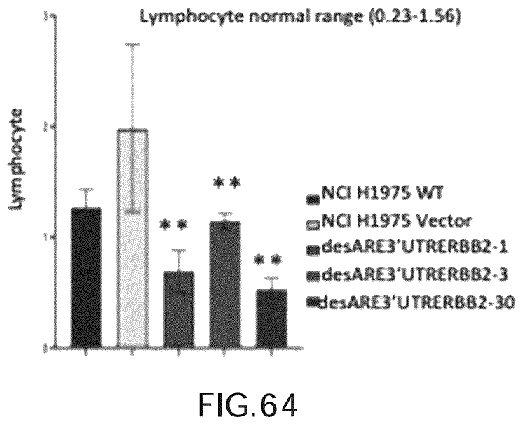

Next, we analyzed the weight of the mice as an early pointer of toxicity. We found that there is no difference between the wildtype, vector, and the mice receiving the engineered desARE3′UTR ERBB2-1, -3, and -30 (FIG. 58). This finding shows that while the tumor volumes in the controls were increasing, the mice that received the engineered constructs had their tumor volume reduced and they gained weight by feeding well which points to the absence of toxicity. To prove at the molecular level that the disclosed technology had no adverse effects or toxicity on the mice that received them, a complete blood count and complete blood chemistry analysis was performed on the controls (wildtype and vector tumors) and desARE3′UTR ERBB2-1, -3, and -30 mice. There is no change in the red blood cell count (FIG. 59), hemoglobin (FIG. 60), and hematocrit (FIG. 61), and this points to the fact that the engineered constructs are safe and do not cause red blood cell dyscrasias. The white blood cells, neutrophils, and lymphocytes were analyzed. White blood cells, neutrophils, and lymphocytes (FIG. 62, FIG. 63, FIG. 64) were significantly elevated in the wildtype and vector mice, whereas desARE3′UTR ERBB2-1, -3, and -30 restored the white blood cells, neutrophils, and lymphocytes to the normal levels. This finding confirms that the engineered constructs while reducing the tumor volume by degrading ERBB2 restores the white blood cells, neutrophils, and lymphocytes to the normal level. The high level of the white blood cells, neutrophils, and lymphocytes in the controls shows that the immune cells in those mice are elevated due to the tumor size increase.

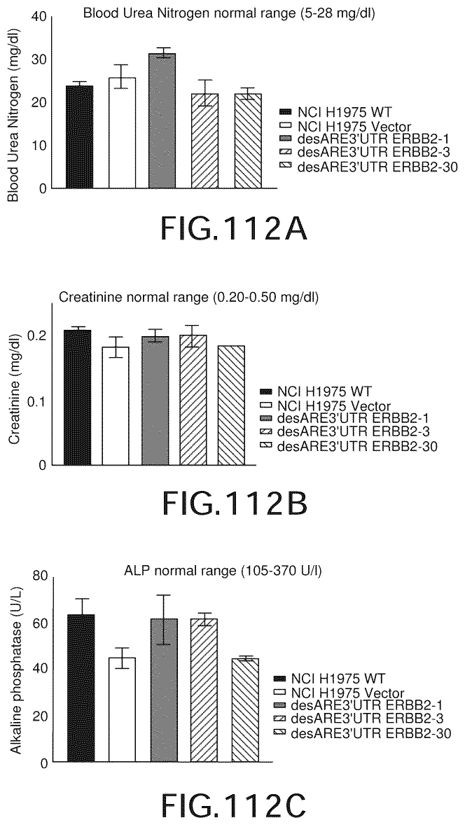

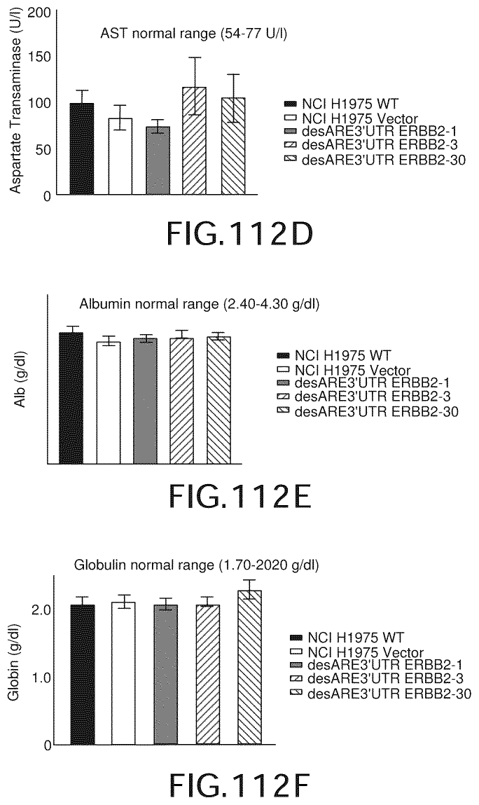

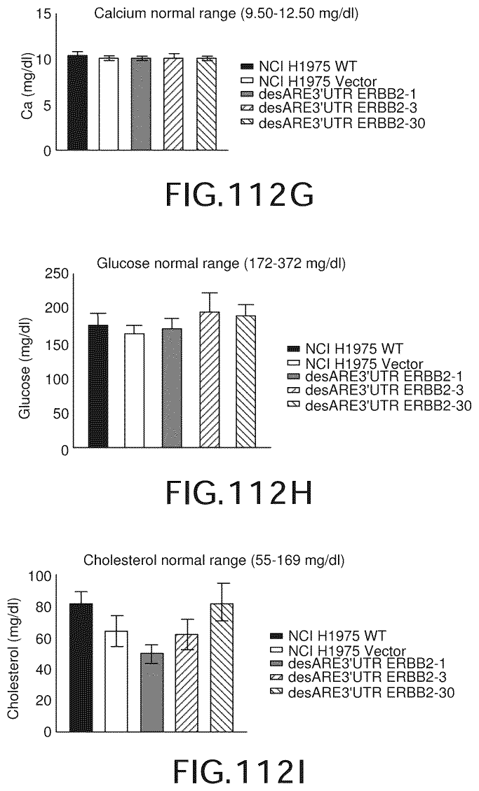

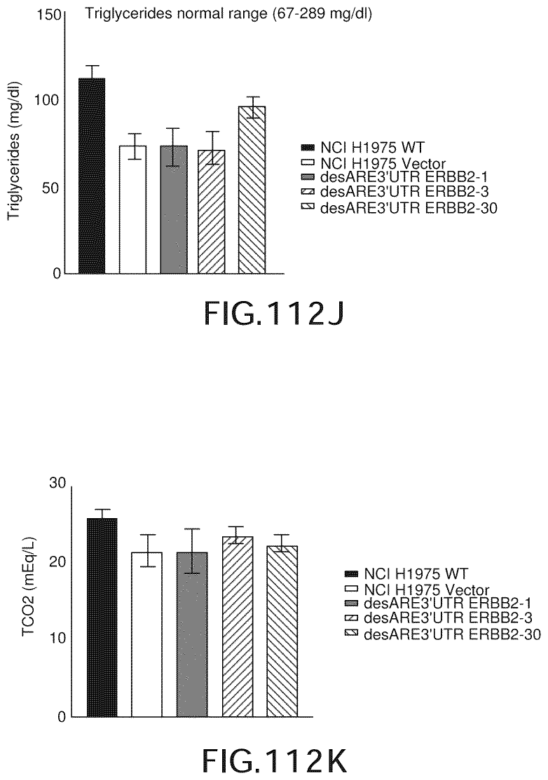

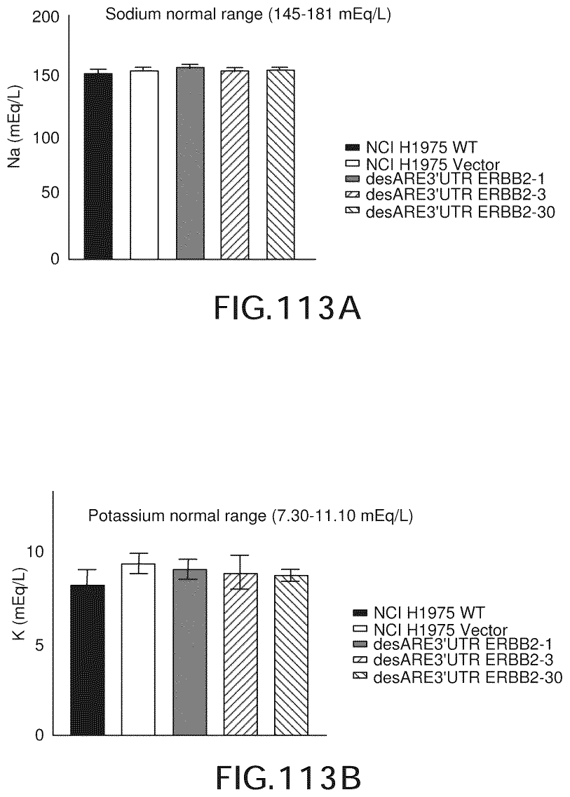

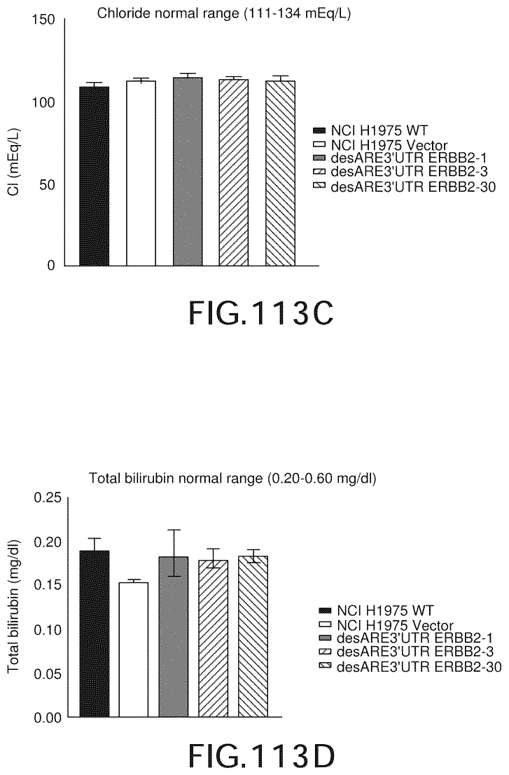

Next, the complete blood chemistry of the tumor-bearing control mice and those receiving desARE3′UTR ERBB2-1, -3, and -30 were studied. In the kidney function analysis, no increase in blood urea nitrogen and creatinine levels were found (FIG. 112A, FIG. 112B). The liver function test showed no increase in albumin (FIG. 112E) and globulin (FIG. 112F) in controls and tumor-bearing constructs. The alkaline phosphatase levels were low across the control and tumor-bearing mice treated with the engineered destabilized constructs (FIG. 112C). The aspartate amino transferase level (FIG. 112D) was elevated across all mice bearing tumors except for the mice receiving desARE3′UTR ERBB2-1. The blood glucose (FIG. 112H), cholesterol (FIG. 112I), triglycerides (FIG. 112J), and total bilirubin (FIG. 113D) levels are normal between the controls and the tumor-bearing mice receiving the engineered constructs. These results strongly show that there is no abnormal liver, bone, pancreas, and gall bladder function in the mice receiving the engineered destabilized constructs.

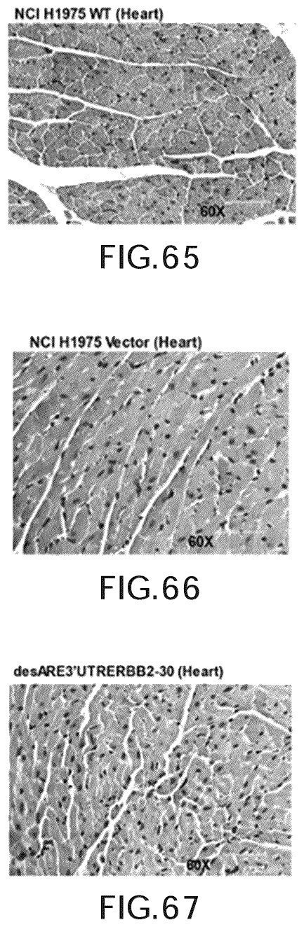

Furthermore, the blood electrolyte levels were examined by comparing the controls and the tumor-bearing mice receiving the constructs. The calcium (FIG. 112G), blood CO₂ (FIG. 112K), sodium (FIG. 113A), potassium (FIG. 113B), and chloride (FIG. 113C) levels were all normal. Trastuzumab is known to cause cardiac toxicity. H &E staining of the cardiac tissues was used to evaluate if the constructs affected cardiac tissue morphology between the treated and untreated animals bearing tumors. No observable histological changes were found in the cardiac tissues (FIG. 65, FIG. 66 and FIG. 67).

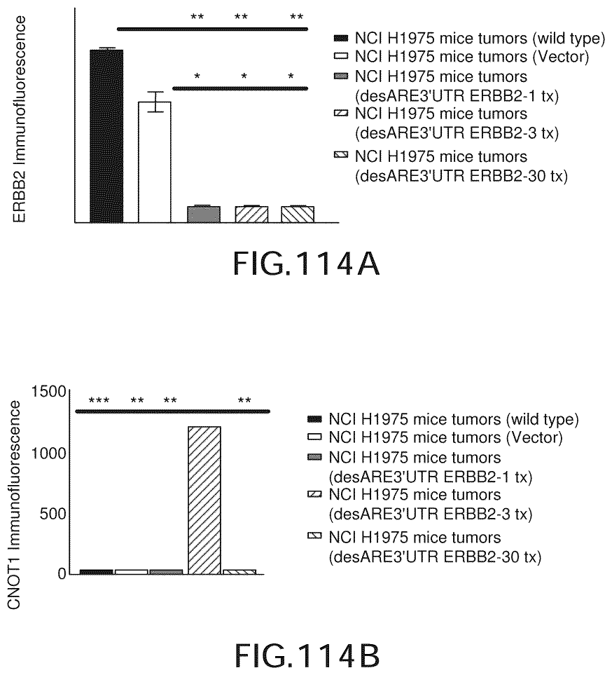

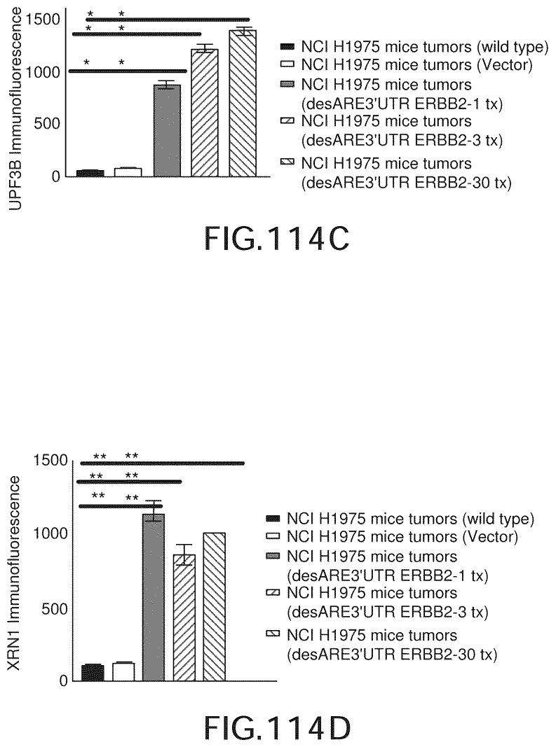

Taken together, these data validate in vivo that the engineered destabilized 3′UTR of ERBB2 significantly reduced the tumor volume in a deadly drug-resistant tumor model with no abnormal blood, liver, kidney, bone, pancreas, gall bladder, and electrolyte imbalance. These findings strongly suggest that the therapy is safe and can be rapidly translated to human cancers. Finally, the xenograft slides of WT mice tumors, vector, and tumors treated with desARE3′UTR ERBB2-1, -3, and -30 were stained with antibodies against ERBB2, CNOT1, UPF3B, and XRN1 (FIG. 114A, FIG. 114B, FIG. 114C). The results show that ERBB2 protein expression is significantly lost in mice that received the constructs compared to the controls. CNOT1 expression is significantly upregulated in desARE3′UTR ERBB2-3, and UPF3B, XRN1, and caspase 7 are significantly upregulated in desARE3′UTR ERBB2-1, -3, and -30 compared to the controls (FIG. 50). This finding strongly validates the in vitro results across multiple cancers and validated the biology of design and the mechanistic of the engineered constructs.

### Discussion

This disclosure provides a demonstration of effective targeting of an oncogene by introducing destabilizing motifs into its 3′UTR resulting in destabilization of its transcript and degradation of its protein and consequent death of the cancer cells in vitro and inhibition of in vivo tumor growth. The technology was developed by engineering the destabilized 3′UTR ARE that reliably degrades the oncogenic transcript and protein expression and their downstream kinases that cause cancer drug resistance. The findings provided in this study show that the deadly cancer drug resistance driven by pervasive oncogenic signals can, indeed, be controlled by this technology. This technology demonstrates the ability to modulate levels of oncogenic transcripts by replacing the stabilizing with the destabilizing elements in the 3′ UTR in difficult-to-treat cancers. This work represents a technical advancement and a strategy for reducing oncogene expression and their interactome. The disclosure provides proof-of-principle evidence in this study with findings from diverse cancer models and established a paradigm which has opened the doors to applying this technique to control any transcript of interest in the temporal and spatial scale.

This technology is versatile and generally applicable to controlling diverse oncogenes and cellular transcripts across any tissue of interest. This disclosure shows that one can control ERBB2 across a wide range of cancers, namely, NCI-H1975 ERBB2-expressing osimertinib-resistant EGFR T790M non-small-cell lung cancer, wildtype ERBB2+ breast cancer BT474, BT474 clone 5 ERBB2+ trastuzumab-resistant breast cancer, NCI-H2030 ERBB2-mutated non-small-cell lung cancer, and ERBB2-expressing HCT116 colorectal cancer. The technology is tissue-agnostic, but oncogene-targeted.

The constructs were designed to have the destabilized sequences of the ARE in which stabilizing elements were changed, and this destabilized ARE is driven by mRNA decapping protein DCP1A which specifically upregulates the mRNA decay pathway and trigger the deadenylases CNOT1 and cleavage enzyme XRN1 to degrade the transcript of interest. Also integrated in the design is the sustained slow decay rate in the constructs. Some nucleotides were modified as described in Torabi et al (2021) to achieve a slow decay rate destabilizing construct. The results show that the constructs worked as designed in destabilizing the ERBB2 transcript, degrading the transcript through mRNA deadenylases CNOT1 and XRN1, and by nonsense-mediated decay. The destabilizing constructs integrated into the genome and outcompeted the endogenous ERBB2 and degraded its transcript and protein.

The destabilization of the oncogene transcript described herein is versatile. This disclosure showed that the approach works in the oncogene of interest regardless of the tissue. The disclosure demonstrated that ERBB2 can be targeted and controlled with tumor death across many tissues and in diverse biological backgrounds, namely, in EGFR T790M lung cancer, in ERBB2+ wildtype breast cancer, in ERBB2+ trastuzumab-resistant breast, and in ERBB2-mutated lung cancer and ERBB2-expressing colorectal cancer. The results suggest that this technology has the potential to be superior in controlling cancer chemoresistance driven by oncogene and its kinases. In osimertinib- and trastuzumab-resistant cancers, once one controls the oncogenes, one controlled the kinase signaling pathways and interactome through which they function to become resistant. For example, in ERBB2-overexpressing EGFR T790M lung cancer, one controlled and degraded ERBB2 and as well degraded EGFR, MET, and YES1 kinases that are known to drive this tumor resistance to drugs. The same finding was made in ERBB2+ trastuzumab-resistant breast cancer, in that the control of ERBB2 leads to loss of kinases such as WNK1 and YES1 implicated in this cancer mechanism of resistance.

The optimal constructs show the difference in the rate of degradation of ERBB2. This is explained by the method of cloning the constructs and the evolutionary pressure introduced in the destabilizing constructs by the E. coli in which they were cloned into. desARE3′UTR ERBB2-1, -2, -3, and -4 were obtained by cloning the constructs into recombinant-efficient E. coli. Of these four constructs, desARE3′UTR ERBB2-3 and desARE3′UTR ERBB2-1 degraded both the transcript and protein in most ERBB2 cellular settings both in wildtype and drug-resistant settings. desARE3′UTR ERBB2-2 downregulated the transcript and protein in the native ERBB2 WT setting but did not perform optimally in the ERBB2 drug-resistant setting. desARE3′UTR ERBB2-4 was the least performing. This work was extended by using Gibson assembly to clone the constructs in a recombinant-deficient E. coli to obtain desARE3′UTR ERBB2-30. desARE3′UTR ERBB2-30 is effective as desARE3′UTR ERBB2-1 and -3 in degrading ERBB2 across multiple ERBB2 cancer types. desARE3′UTR ERBB2-30 has little to no mutation in the engineered destabilized ARE. deARE3′UTR ERBB2-1 and -3 contain some point mutation introduced by the recombinant efficient E. coli. Pointing to the fact that bacterial evolutionary pressure further selected and enhanced the construct's function, it is worthwhile to mention that the destabilizing constructs were engineered based on the BT474 wildtype ERBB2 3′UTR poly U mRNA-stabilizing elements. Yet, they work across various ERBB2-expressing cancers from diverse biological organs. The constructs showed anti-cancer activity such as impaired cancer cell viability, reduced migration and induction of apoptosis, and increased caspase 3/7, cleaved caspase 3, and caspase 9 expressions.

The disclosed constructs do not kill normal breast epithelial cell MCF10A. These proof-of-principle findings strongly suggest that the destabilizing constructs do not affect normal cells but are cancer-specific and on target, which is very promising and only works in cancers carrying the stabilizing ERBB2 elements. The data show that destabilizing ERBB2 does not affect c-MYC and its kinases (FIG. 42). The results and finding are now validated in an animal tumor-bearing model, where the tumor volume is significantly reduced by the engineered destabilized constructs, and no abnormal vital organ functions or electrolyte imbalance were found (FIGS. 55-64). Technologies such as CRISPR, RNAi, the degron system, and PROTAC are competitors to the findings presented in this study. CRISPR has been used to knockout gene expression with high efficiency of up to 90% loss of protein. However, CRISPR has remarkably high off-target effects and can cause unwanted chromosomal rearrangements as has been shown. Regarding RNAi, even though it can interfere and silence the expression of a protein, it also has off-target effects in order of magnitude greater than CRISPR. The degron system has been effective in mediating degradation of proteins; however, the system relies on inducibility by auxin or similar molecules to initiate its functions. The degron degradation is leaky and requires high levels of auxin. Last, for PROTAC which targets proteins that have already been made, the limitation of PROTAC is highly toxic, costly to make, targets only intracellular proteins, and cannot target membrane proteins. This engineered destabilized 3′UTR-mediated degradation of the transcript offers a unique and novel approach to target oncogenic transcripts at their untranslated region which has been difficult to study. The engineered destabilized 3′UTR AREs are easy to synthesize as gBlocks, cheap and easy to clone, and they are very specific to target oncogenes in cancer cells but do not degrade proteins in normal cells, and the therapeutic degradation of transcript can be achieved at a very low dose of a nanogram of vectors containing the destabilizing constructs. This makes the technology very attractive. This disclosure shows that in certain genetic settings of cancer as in ERBB2 HCT116, the disclosed methodology is superior to CRISPR, RNAi, and shRNA in a head-to-head comparison. This is because these technologies show genetic dependencies, whereas the disclosed technology is agnostic to these dependencies and degrades the transcript once the stabilizing elements are destabilized (Table 9). This disclosure shows that the constructs reprogram the ERBB2 genetic programs toward the normal individual ERBB2 gene program from diverse ethnicity (FIG. 51, FIG. 52). By optical genome mapping, the de novo rare variants and genomic alterations are not different between the wildtype, vector, and ERBB2 mRNA-destabilized cancer (FIG. 53, FIG. 54). This evidence strongly suggests that the constructs are safe for the cells. In fact, the data in FIG. 54 show that desARE3′UTR ERBB2-3 also leads to loss of aneuploidy in cancer cells, and aneuploidy is a major cause of chromosomal instability, a key hall mark of cancer cells. Taken together, the data demonstrate that one can control ERBB2 across many cancer types and as well control the major drivers of cancers, which is aneuploidy without causing genome rearrangement.

### Delivery Agents

### Non-Small Cell Lung Cancer (NSCLC)

To target the recalcitrant ERBB2 driver oncogene, engineered destabilizing 3′UTR ERBB2 constructs were developed that degrade the endogenous ERBB2 transcript and proteins by overwriting the encoded endogenous ERBB2 mRNA with the destabilizing message. When a delivery agent, such as an iron oxide nanocage (IO-nanocages), was used as vehicles to deliver them to tumors and whole tissues in mice bearing tumors, it was well tolerated and safe and caused no genome rearrangement while they were integrated into genome deserts (non-coding regions). In those embodiments, where a nanocage is utilized the nanocage is a hollow metal oxide cage with a diameter of less than 50 nm or less than 15 nm. See, for example, U.S. Pat. No. 10,688,556, the content of which is incorporated by reference.

The nanocages are generally formed of palladium, gold or an iron oxide and are hollow. In some embodiment, a biocompatible polymer shell (e.g. dextran) is covalently bound to the nanocage. In one embodiment, the nanocage has a diameter of 15 nm or less. In one embodiment, the polymer-coated nanocage has a diameter of 50 nm or less. The nanocages may be functionalized with, for example, a catechol or dopamine or treated with 3-(3,4-dihydroxyphenyl) propionic acid (DHCA) prior to use.

A significant reduction of the primary tumor volume was achieved with desARE3′UTRERBB2-30, achieving 50% complete tumor lysis and inhibited 60-80% of liver metastasis, hepatomegaly, and 90% of lung metastasis, respectively, through the ERBB2 downregulation. These constructs were distributed robustly into tumors, livers, lungs, kidneys, spleen and mildly in the brain and not in the heart. They caused no abnormality both in short- and long-term administrations as well as in healthy mice. In summary, we accomplished significant breakthrough for the therapeutics of intractable lung cancer patients whose cancers become resistant and metastasize. Other suitable delivery agents include, but are not limited to, lipid nanoparticles, gold nanoparticles, nanodiamonds, dextran formulations, PEG formulations, exosome formulations, liposomes, microspheres, polymeric micelles, GalNac-conjugates and combination thereof.

According to NCI SEER statistics, 53.1 per 100,000 men and women will develop lung and bronchial cancer per year, with 5-year survival rate at 21.7% (1-3). Lung cancer caused more deaths in 2017 than breast cancer, prostate, and brain cancers combined. The prevalence of lung cancer and its related mortality arises from the oncogenes that drives the malignant lung tissues to excessively proliferate and then metastasize to distant lymph nodes and onwards to organs such as brain, liver, bones, and adrenal glands leading to death of the patient.

HER2/ERBB2 is a member of the subclass I receptor tyrosine kinase superfamily of ERBB/EGFR (epidermal growth factor receptor family) which consists of four members namely: EGFR/ERBB1, ERBB2, ERBB3 and ERBB4. Non-small cell lung cancer (NSCLC) with EGFR T790M is moderately responsive to first- and second-generation EGFR tyrosine kinase inhibitor. However, osimertinib which is a third generation EGFR-TKI has been shown to be superior to erlotinib and gefitinib in a clinical trial AURA and FLAURA in lung cancer. Osimertinib is particularly effective for 60% of lung cancer patients who have EGFR mutation (T790M), but for the remaining 40% there is no benefit and resistance start arising 10-18 months after the therapy. Resistance to Osimertinib can arise in two ways, EGFR-dependent and EGFR-independent mechanisms. For the EGFR-dependent mechanism, it was found that mutation arises due to; MET amplification (15% patients), K-RAS & N-RAS (8% of patients), PI3KCA (7% patients), HER2 amp (5-6% of patients) and MAPK1 and EGFR and BRAF. Moreso studies have shown that the treatment with Osimertinib leads to increased HER2 surface expression and HER2 amplification. The EGFR-independent mechanism described the Osimertinib resistance to be under the control of PTEN, TSC2, YES1, CTNNB1, FGF2 etc.

To address these problems, this disclosure provides engineered stable poly U rich elements on the 3′UTR of ERBB2 to unstable forms and destabilized the transcript and degraded the expression of ERBB2 protein. This technology controls ERBB2 in EGFR T790M HER2-overexpressing osimertinib and trastuzumab-resistant NSCLC. This treatment significantly reduced the primary tumors and inhibited 80% of liver and lung metastasis in mice that received our constructs. This finding has provided a potential therapy to address this intractable end stage drug-resistant NSCLC.

Without wishing to be bound to any particular theory, it is believed that 3′UTR of oncogenes, such as HER2, are enriched with ARE stabilizing elements which stabilizes their oncogenic transcript and drives tumor aggressiveness and therefore, if these stable elements were changed to destabilizing elements by motif engineering and driven by mRNA de-capping promoter DCP1A that one can control the oncogene by degrading the oncogenic transcript and protein. There are unique sequences on the poly U rich elements (ARE) of the 3′UTR of mRNA that makes oncogenic HER2 transcript stable, and thus these elements were engineered to unstable forms and degraded HER2 across many HER2 driven cancer cell types both in wildtype and drug resistant cancer models which reduced primary tumor growth and inhibited metastasis (liver and lungs) in vivo in EGFRT790M HER2 positive osimertinib/trastuzumab resistant NSCLC.

This disclosure provides two constructs, desARE3′UTRERBB2-3 and 30, effective for destabilizing the ERBB2 transcript and degrading the protein, and ERBB2-dependent kinases WNK1, and YES1 in EGFR T790M NSCLC. The loss of ERBB2 led to the loss of cell viability. The degradation of ERBB2 is through upregulated nonsense mediated decay proteins UPF3B, CNOT1 and XRN1. In two independent in vivo studies, inhibition of primary tumor, inhibition of liver and lung metastasis, and control of hepatomegaly were achieved. The desARE3′UTRERBB2-3 and 30 are specific to ERBB2 and do not affect normal cells. No weight changes, blood dyscrasias, electrolyte, renal or liver malfunctions were found. The desARE3′UTRERBB2-3 and 30 were systemically biodistributed.

### Materials and Methods

Cell Culture: The NCI H1975 cells were cultured in RPMI supplemented with the 10% FBS and 5% antibiotics. Cells were grown until they are 80% confluent before use.

Engineering the destabilized 3′UTR ERBB2 constructs: Briefly, the poly U mRNA stabilizing sequences on the 3′UTR of ERBB2 across different ERBB2 driven cancer was identified. A modular designed synthetic construct was designed driven by DCP1A promoter in which all the stable poly U sequences were change to unstable forms. The synthetic construct was cloned either by sticky end ligation using BamH1 and BstB1 site or by Gibson assembly to the receiving vector (pLenti-CMVSP6-nEGFP-SV40-PURO, Addgene #138364). They were used to transform competent E. coli and the clones picked were miniprepped and confirmed by Sanger Sequencing. For generation of high yield plasmid DNA containing the constructs for animal experiment, the Maxi Prep kit of Zymo Research (Cat no: D4203) was used.

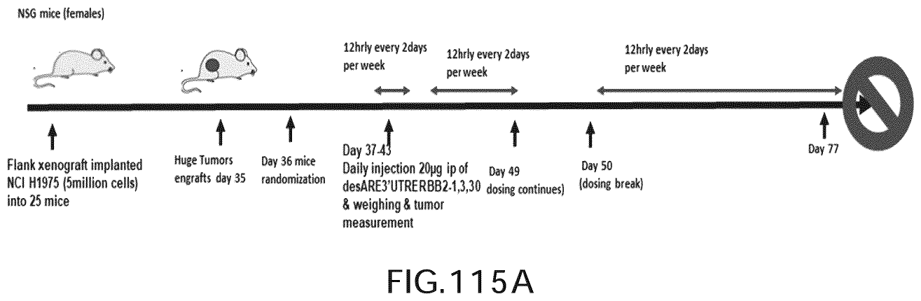

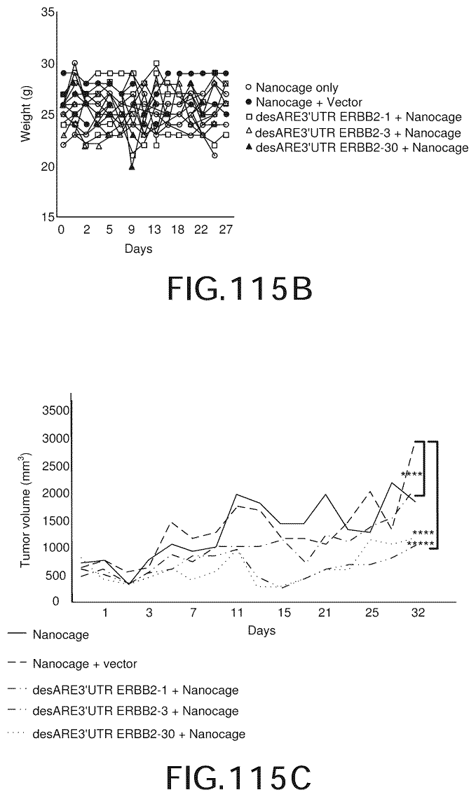

Animal Study: 25 NSG female mice were ordered from the Jackson Laboratory. The mice were received and allowed to acclimatize according to institutional protocol. NCI-H1975 cells were cultured in RPM1, when at 80% confluency, the cells were harvested and washed in PBS and counted. 5 million cells in 0.1 ml were implanted on the flank of the animals. After 35 days, huge tumors engrafted and on the 36 day, the animals were randomized into 5 mice per cage into 5 cages with equal distribution of tumor size (FIG. 115C). On day 37, intraperitoneal administration of the iron oxide plus the engineered destabilized construct was started at 20 μg 12 hrly every 2 days per week between days 37-49. On day 50, a dosing break was observed and resumed from day 51-77 at 12 hrly every 2 days per week. At 77 days, the experiment ended. A complete necropsy was performed and the organs were sent to the core pathology group of the Memorial Sloan Kettering Cancer Institute for histological analysis and H& E staining. The expert pathologist was blinded to the experimental details.

IO-nanocage packaging of the engineered destabilized construct: 3,4-dihydroxyhydrocinnamic acid (DHCA) by Alfa Aesar, Manganese (II) acetate, oleylamine, oleic acid, and iron (II) perchlorate) were purchased from Sigma-Aldrich. p-Xylene, 1-ethyl-3-[3-(dimethylamino) propyl]carbodiimide (EDC), and N-hydroxysuccinimide (NHS) were purchased from Thermo Scientific. Tetrahydrofuran (THF), dimethylsulfoxide (DMSO), ferric chloride hexahydrate, and phosphate buffer saline (PBS) was purchased from Fisher Scientific. Cy7.5 dye was purchased from Lumiprobe.

Generation and structure analysis of DNA plasmid-IO-nanocage complexes: The IO-nanocages were synthesized by a galvanic reaction as manganese ions of template Mn₃O₄ nanocubes are replaced by iron ions to form a cage-structure with a hollow center, as shown in previous publication. Water-soluble IO-nanocages were engineered by capping with 3-(3,4-Dihydroxyphenyl) propionic acid (DHCA) before loading the DNA plasmids containing the destabilized ERBB2 constructs as well as vector controls only. DNA plasmids were mixed with IO-nanocages with the mass ratio of 1:1 for the complexation based on the concentration of DNA plasmids needed for the sufficient efficacy with respect to the optimized dosage of IO-nanocage concentration for in vivo experiments.

Haematoxylin and Eosin (H&E) Staining and Immunohistochemistry (IHC): The lung, liver H&E was done by experts pathologist of the Memorial Sloan Kettering Cancer Center, Core pathology laboratory New York. ERBB2 IHC and primary tumor H&E according to standard protocol. After sacrifice, tumor, spleen, liver, kidney, lung, hearth and brain samples obtained from mice via dissection were fixed in formalin and embedded in paraffin. Sections of 4 μm thickness was stained with hematoxylin & eosin (HE) and HIC antibodies.

HE and IHC staining were performed. Briefly, samples of the tumors, livers, brains, spleens, kidneys, and lungs were fixed, stained with HE, and visualized under an optical microscope. HE staining enabled determination of the morphological features of the tumors. For IHC staining, slides were deparaffinized, dehydrated, blocked. The slides were blocked with mouse serum for 30 min and incubated with HER 2 antibody (1:500) (Proteintech, 18299-1-AP), were performed at 4 C overnight. All the buffer and reagents used in IHC was used from IHC Prep & Detect Kit for Mouse Primary Antibody (Proteintech, PK10018) according to manufacturer's instructions.

Next, the slides were incubated with the secondary antibody, stained with diaminobenzidine substrate and counterstained with hematoxylin. Lastly, all samples were analyzed via optical microscope.

Prussian blue staining and detection of IO-nanocage in tissues: The Prussian Blue Staining on the tissue slides with and without the treatment of ERBB2-incorporated IO-nanocages was performed as follows. First, the tissue slides were deparaffinized with xylene for 10 min, 100% ethanol, for 9 min, 95% ethanol for 6 min, 70% ethanol for 3 min, and lastly hydrated with MilliQ water 5 min. Then, the tissue slides were placed in the Working Iron Staining Solution (Sigma Aldrich) for 30 min as the Working Iron Stain Solution was prepared by mixing equal volumes (100 mL) of potassium ferrocyanide solution and hydrochloric acid solution. The tissue slides were then collected and rinsed thoroughly in MilliQ water. The tissue slides were then placed in the Working pararosamiline solution (Sigma Aldrich) for 3-5 min as the Working Pararosamiline solution was prepared by mixing 1 mL of Pararosamiline Solution with 50 mL of MilliQ water. After the tissue slides were collected and rinsed thoroughly in MilliQ water, the tissue slides were then rapidly dehydrated through a series of alcohol treatment and cleaning with xylene as stated above before mounting. One drop of Permount Mounting Media (Fisher Scientific) was placed on the tissue slide and a cover slip was placed on top of the tissue slide for imaging.

Pathological scoring: Reporting of cancer cell lysis, mitotic counts, histological subtyping and ERBB2 grading were done. Cell lysis was reported using high resolution microscopy as well as observing the appearance of disrupted cell membranes and naked nuclei under high power resolution. Nottingham's prognostic index was used to grade the no of mitosis in breast cancer 1. While histological typing was done according to the degree of differentiation, with poorly differentiated cells forming no glands and growing in sheets.

ERBB2 grading was considered positive if at least 30% of tumor cells exhibited 3+ cell membrane staining and a borderline result was given when at least 10% of cells showed 2+ cytoplasmic membrane staining.

Quantitative PCR: To validate the sensitivity and specificity of detecting the engineered destabilized 3′UTR of ERBB2 from the genomic DNA of the tumors from treated animals. The gDNA of the tumors was extracted according to standard protocol using Qiagen kit. The concentration of the DNA was measured by nanodrop. To determine the sensitivity, specificity, and limit of detecting the constructs, quantitative PCR was performed from the extracted gDNA from the tumor using a serial dilution of 1, 1:10 and 1:1000 assaying for the expression of the construct as well as the house keeping gene GAPDH with primers that recognize only the engineered destabilized 3′UTR of ERBB2 and primers targeting the exons of GAPDH.

Targeted Sequencing: To detect the integration site of the engineered destabilized constructs. A targeted sequencing of the gDNA obtained from the tumors of the treated animals was performed. A set of primers that recognizes the engineered destabilized 3′UTR ERBB2 constructs and the RFP marker on the vector in the genomic DNA was used. Thus, validating that reliably detected the construct and the vector.

Biodistribution analysis: To detect the biodistribution of the constructs across the tissues and organs of the treated mice. Genomic DNA was extracted from the tumors, spleens, livers, lungs, hearts and brains of the treated animals. PCR was performed with primers that only detect the engineered destabilized 3′UTR ERBB2 constructs. The amplified products were resolved on 2% agarose gels and imaged.

Statistical analysis: All the experiments done were performed in replicates. Starting with the animal experiment. A total of 25 NSG female mice were used at 5 mice per group per cage. The groups were IO-nanocage only, IO-nanocage+vector, desARE3′UTRERBB2-1+IO-nanocage, desARE3′UTRERBB2-3+Nanocage and desARE3′UTRERBB2-30+IO-nanocage. Two-tailed t-test were used to determine statistically significant between the controls and the different treatment groups.

GraphPad Prism: All graphs plotted were drawn with the GraphPad Prism software. And all statistical significance analysis was done with the same tool.

### Results

### IO-Nanocage Delivered Engineered Destabilized ERBB2 Constructs Inhibits Primary EGFR T790M HER2+ Osimertinib-Resistant NSCLC:

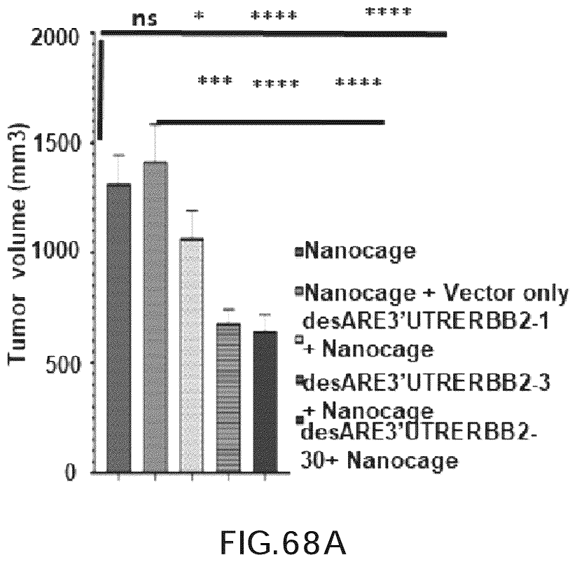

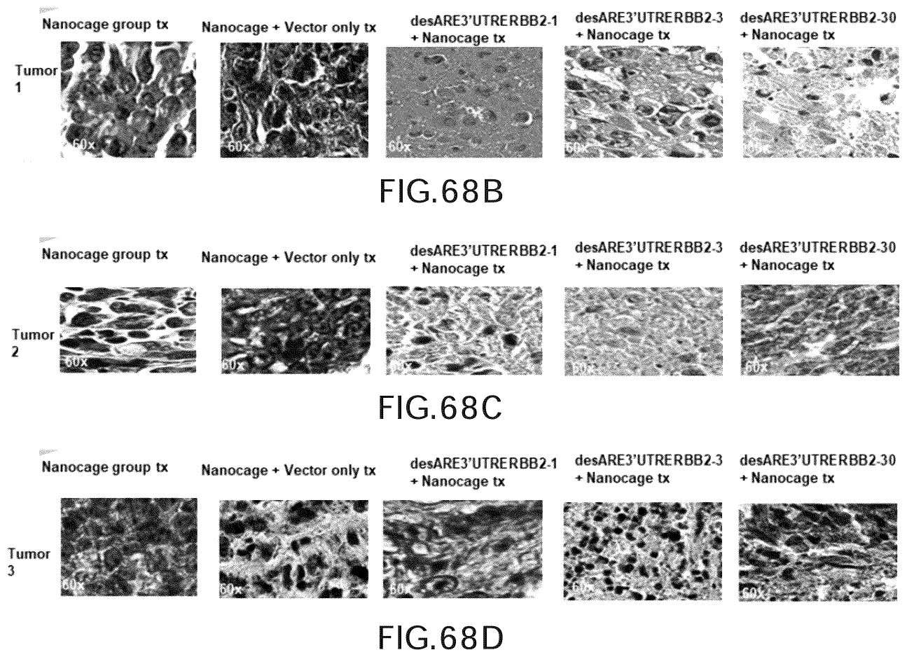

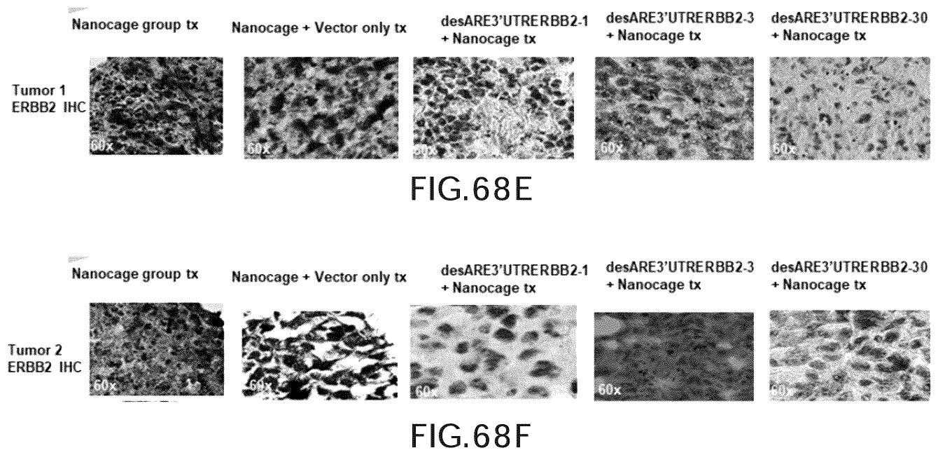

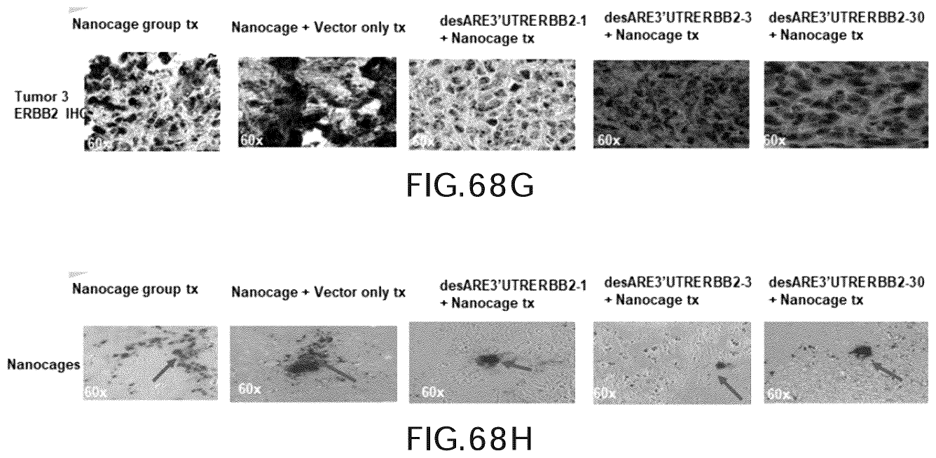

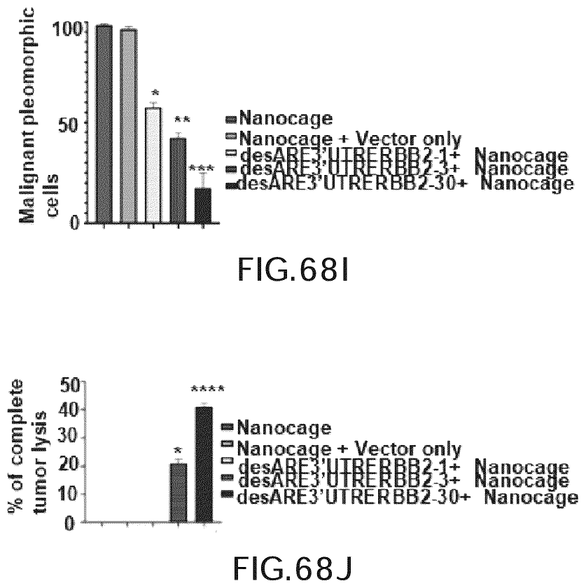

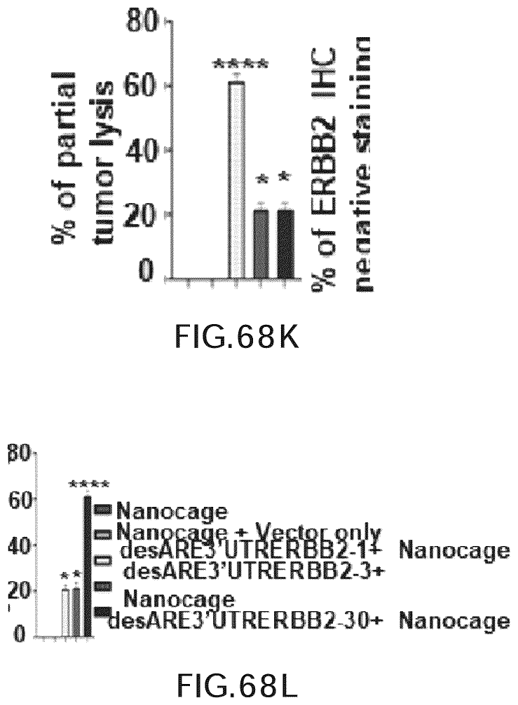

To determine if the engineered destabilized 3′UTR ERBB2 constructs packaged into IO-nanocage inhibit the primary tumor, 25 NSG mice were implanted with 5 million NCI-H1975 cells into the flank, after 35 days the tumor engrafted, we randomized the mice into 5 mice per cage on the day 36 (FIG. 115A). Starting from the day 37-43, the desARE3′UTRERBB2-1,3, 30 was administered as IO-nanocage complexed intraperitoneally at 20 μg/0.1 ml every 12 hrs for every 2 days per week till the day 77. There were dosing breaks at the day 49-50. The animals were weighed daily and the tumor size was measured with calipers and body conditions were noted. No change in body weight in the treated animals was found, meaning no toxicity (FIG. 115B). The constructs desARE3′UTRERBB2-3,30 significantly inhibited tumors (FIG. 68A to FIG. 68H), ****P=0.0001). The construct desARE3′UTRERBB2-30 achieved a very significant reduction of malignant pleomorphic cells, an 80% reduction as compared to the controls (FIG. 68B, FIG. 68C, FIG. 68D, FIG. 68I). The desARE3′UTRERBB2-30 achieved 40% complete tumor lysis, a 20% partial tumor lysis and 60% ERBB2 negative staining by IHC (FIG. 68E, FIG. 68F, FIG. 68G, FIG. 68J, FIG. 68K, FIG. 68L). The desARE3′UTRERBB2-1 and 3 achieved variable complete (20% desARE3′UTRERBB2-3) and partial (60% and 20%) tumor lysis respectively, with 20% ERBB2 negative IHC staining (FIG. 68E, FIG. 68F, FIG. 68G, FIG. 68J, FIG. 68K, FIG. 68L). Both empty IO-nanocages and construct-nanocage complexes were detected across all tumors by Prussian blue stain (FIG. 68G).

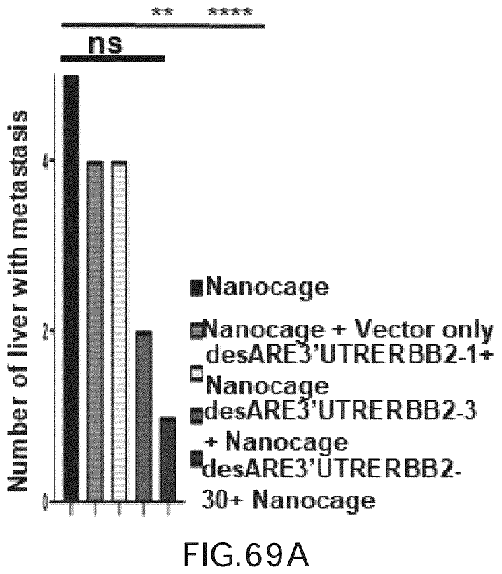

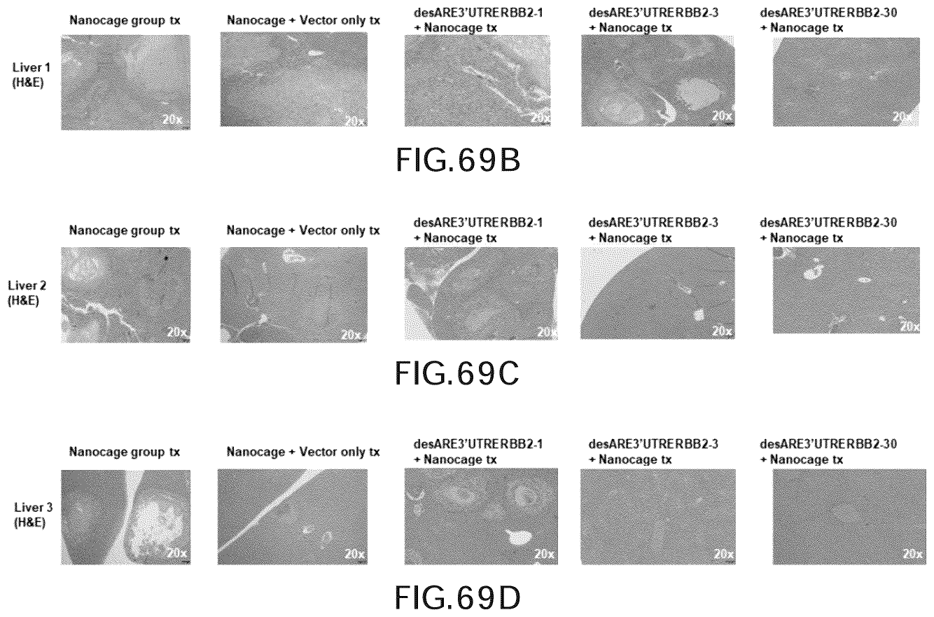

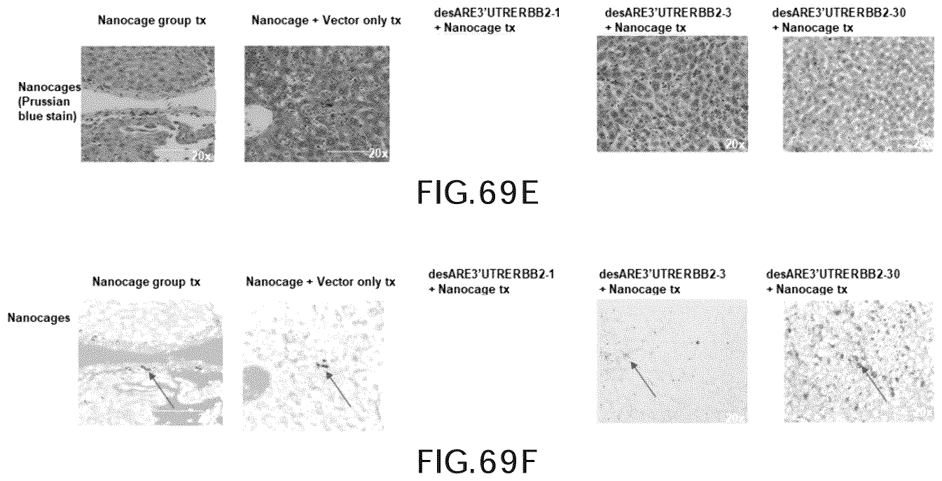

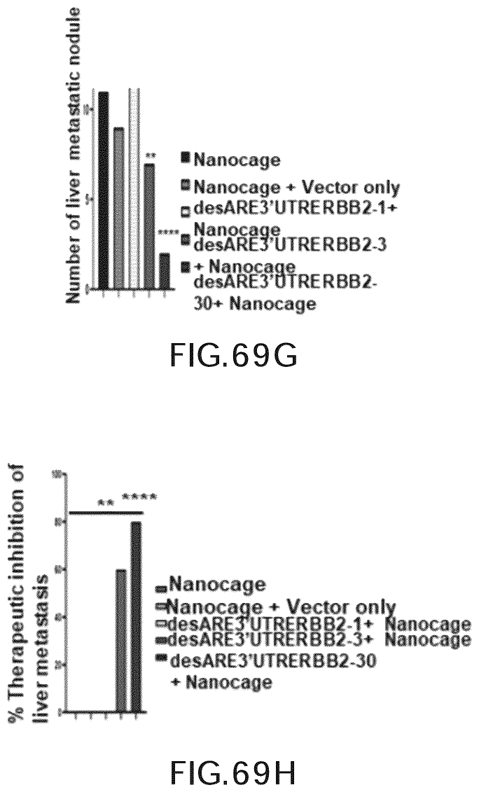

The destabilized construct inhibited 80% liver metastasis in EGFR T790M HER2+ Osimertinib-resistant NSCLC: Metastasis is the number one killer of cancer patients and there are no therapies targeting it. To determine that the constructs inhibited liver metastasis, necropsy was performed and first the gross pathology of the liver was assessed to ascertain visible liver metastasis and then the livers were sectioned and stain the tissues with H& E. The desARE3′UTRERBB2-30 treated group had significantly lower number of livers with metastasis, lower number of livers with metastatic nodules and achieved 80% therapeutic inhibition of liver metastasis (FIG. 69A to FIG. 69H). The desARE3′UTRERBB2-3 achieved 60% therapeutic inhibition of liver metastasis (FIG. 69H). Taken together, this demonstrates that the therapeutic efficacy of the constructs in inhibiting the primary tumor and metastatic liver nodules.

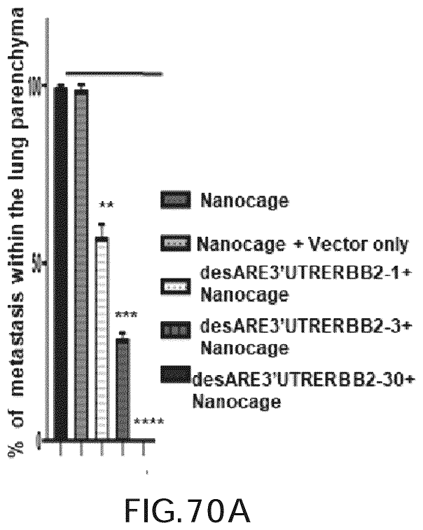

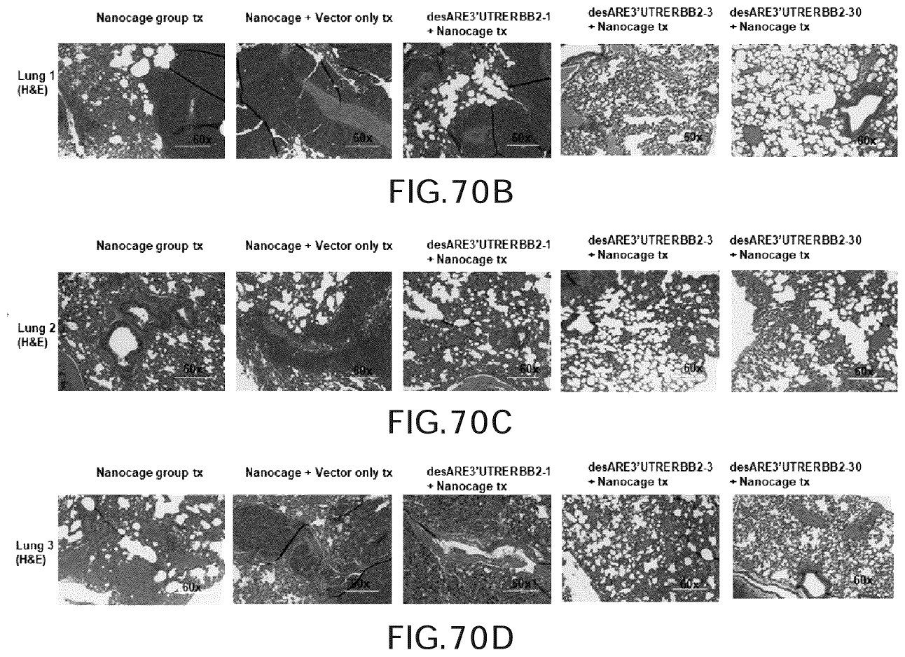

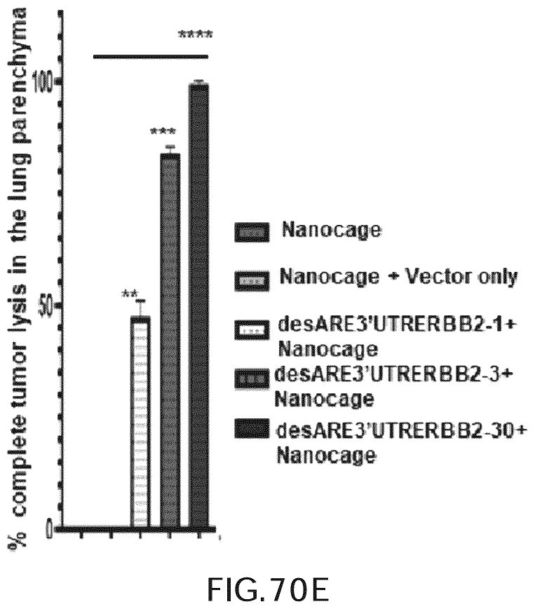

Engineered destabilized 3′UTR of HER2 inhibits lung metastasis: The NCI-H1975 EGFR T790M non-small cell lung cancer is highly metastatic. The treatment with the novel engineered destabilized 3′UTR of ERBB2 was explored to determine if the complex inhibited the lung cancer metastasis. The desARE3′UTRERBB2-30 constructs completely inhibited the lung metastasis (FIG. 70A to FIG. 70D). The inhibition of the lung metastasis by the desARE3′UTRERBB2-30 is followed by the desARE3′UTRERBB2-3. The desARE3′UTRERBB2-30 achieved complete tumor lysis of the metastatic tumors in the lung parenchyma, and this is followed by the parenchyma (FIG. 70E). Taken together, we show that the desARE3′UTRERBB2-30 is very effective in inhibiting and destroying metastatic tumors in the lung parenchyma of the treated animals.

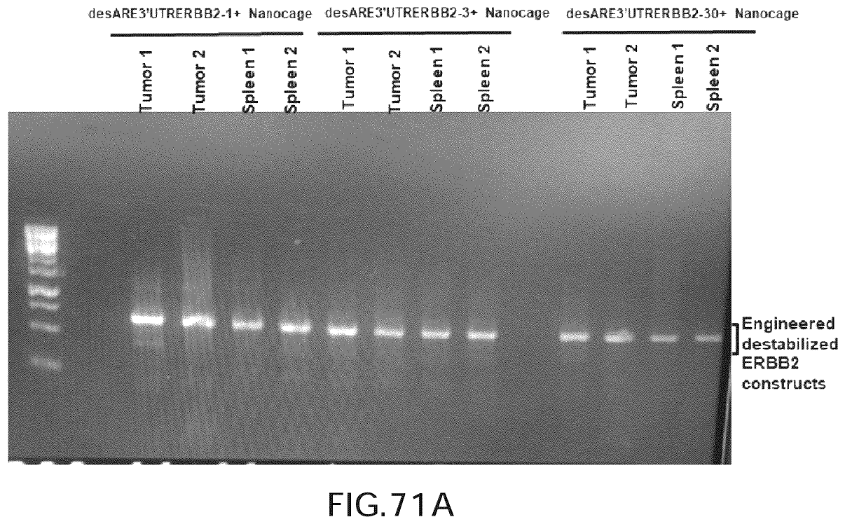

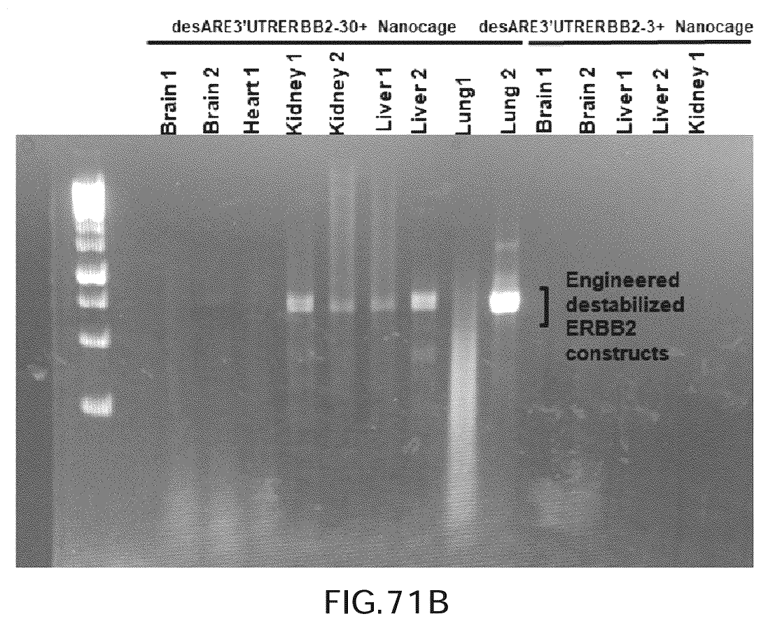

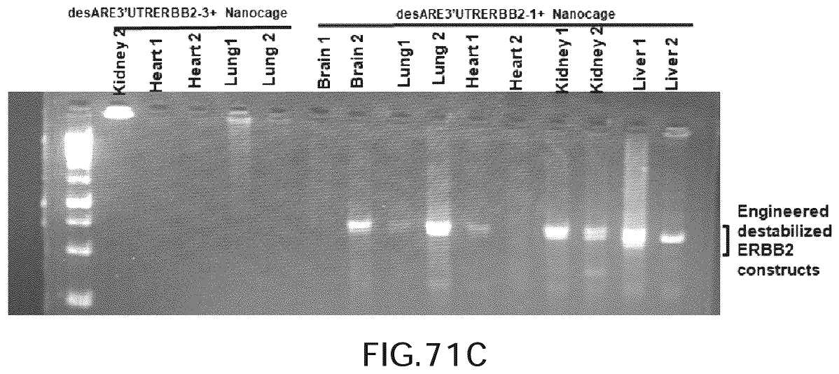

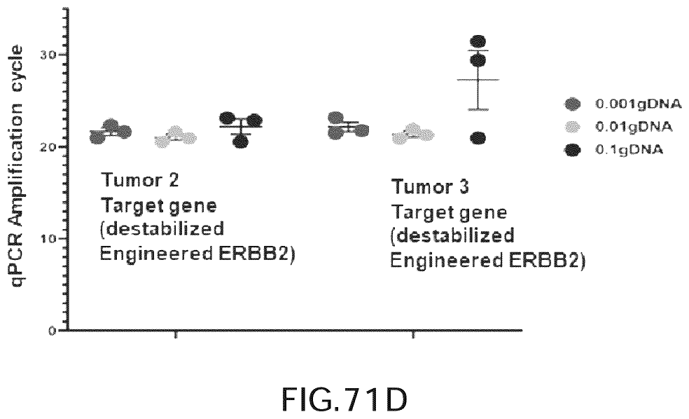

IO-nanocages delivered the constructs to the tumors, liver, lungs, spleen, and kidney of the treated animals: To understand the biodistribution of the engineered destabilized ERBB2 constructs in the treated animals. Primers that are designed to specifically amplify the synthetic engineered destabilized ERBB2 were used. The engineered destabilized 3′UTR ERBB2 constructs were identified in the tumors, spleens, kidneys, livers, and lungs of the treated animals (FIG. 71A-C). The constructs were not found in the brain and heart of the treated animals (FIG. 71B-C). Only the destabilized 3′UTRERBB2-1 is expressed minimally expressed in the heart (FIG. 71C). Next, the transcript level of the engineered destabilized ERBB2 constructs was examined. Quantitative polymerase chase reaction was performed for the constructs on the tumor samples of the treated animals, and desARE3′UTRERBB2 transcript level was found to be high (FIG. 71D).

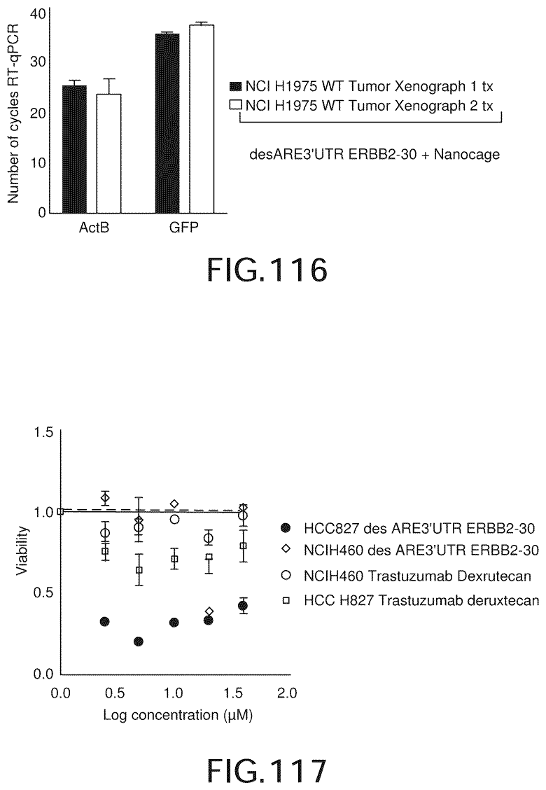

The destabilized ERBB2 constructs integrated safely into non-coding regions: To ascertain the safety and genome integration site of the constructs in the treated animals, targeted sequencing of the tumors was performed using primers that identify only the engineered destabilized ERBB2 constructs and the RFP sequences in the vector. The constructs were integrated in the non-coding regions of the chromosome 1, 7 and 10. This finding supports the safety profile as well as the ability of constructs not to cause genome rearrangements as described previously. To validate the house keeping gene expression level is not perturbed by the constructs. A head-to-head qPCR was performed for ActB and RFP in the vector in the tumor samples and the ActB expression was found to be higher than the RFP, indicating that the house keeping genes for normal cellular functions are optimal (FIG. 116).

IO-nanocage-construct complexes delivered to the healthy animal with no tumors caused no vital organ damage: To prove the safety of the constructs to healthy non tumor bearing animals, the constructs were administered to animals intraperitoneally for 3 months. No gross organ abnormality was found across all the organs of the animals including the muscle tissues. The animals had no weight loss. Thus, the constructs are safe and well tolerated.

Discussion: Lung cancers have remained the leading cause of cancer death worldwide. Resistance to tyrosine kinase inhibitor is rampant. The recalcitrant ERBB2 signal driving the EGFR T790M non-small cell lung cancer can be degraded by the engineered destabilized 3′UTR of ERBB2. The desARE3′UTRERBB2-30 constructs packaged into an IO-nanocages achieved 50% therapeutic inhibition of the tumor, 80% malignant cells reduction, 50% complete tumor lysis and 60% complete reduction of ERBB2 IHC (FIG. 68A to FIG. 68L).

Furthermore, the construct desARE3′UTRERBB2-30 achieved 80% therapeutic inhibition of liver metastasis (FIGS. 69A-G) and 90% inhibition of lung metastasis with complete tumor lysis in the lung's parenchyma (FIGS. 70A-E). The IO-nanocages delivered the constructs into the tumors, spleen, lung, livers, and kidneys (FIGS. 71A-D) and the released constructs were integrated into the non-coding regions of the genome. No evidence of weight loss was found, both in the treated tumor and non-tumor bearing animals which strongly suggests that it is well tolerated and safe.

Referring to FIG. 117, dose response curve was constructed that showed the viability dose dependent titration of desARE3′UTRERBB2-30 in Osimertinib resistant NSCLC HCC827 and NCIH460 in comparison with the standard of care trastuzumab deruxtecan.

The engineered destabilized 3′UTR ERBB2-30 delivered by IO-nanocages inhibited the drug resistant EGFR T790M NSCLC with 50% complete tumor lysis, 60% inhibition of ERBB2 protein, 50% therapeutic inhibition of tumor, and 80% reduction of malignant cells. These IO-nanocage-construct complexes were well tolerated, systemically distributed, and caused no organ damage both to the tumor bearing and healthy mice. This novel drug and the delivery package represent a new paradigm to target drug resistant cancers that are recalcitrant to available therapy.

### Trastuzumab Resistant Breast Cancer

Metastatic drug resistant breast cancer is the most aggressive form of ERBB2 positive breast cancer. The American Cancer Society statistics show that the overall survival rate for the metastatic breast cancer is 28% for 5 years, this is in contrast with all other stages having 90% 5-year survival rate. In England, since 2019 about 19% of the new breast cancer deaths recorded were from metastatic breast cancer. In the US, according to SEER, about 154794 women are estimated to be living with metastatic breast cancer. The incidence of metastatic breast cancer is as high as 27% in Africa. According to Austrian metastatic breast cancer study, about 20% of these cancers express high ERBB2, whereas about 35% express low ERBB2

Trastuzumab, tyrosine kinase inhibitor and anthracyclines are standard of care therapies for these ERBB2 positive metastatic breast cancer. Yet, the recalcitrant ERBB2 signal drives these cancers aggressiveness and resistance to the drugs. In this disclosure, the 3′UTR of ERBB2 is stabilized with the mRNA poly U sequences. These mRNA stabilizing sequences were engineered into unstable forms and drove their expression through mRNA de-capping promoter DCP1A. This triggered nonsense mediated decay of the ERBB2 which led to the degradation of the ERBB2 transcript, protein, and the kinases WNK1 and YES1 implicated in the metastatic trastuzumab resistant breast cancer.

The 3′UTR of genes are hubs for integrating diverse signals and they host both mRNA stabilizing, destabilizing elements, and polyadenylation sites. These elements play critical roles in the spatial and temporal control of transcripts. Also, they are sites for precise targeted control of transcripts. Nonsense mediated decay are universally conserved RNA quality control machineries that the genome uses in the precise regulation and fidelity of transcripts.

By integrating the knowledge domain in 3′UTR mRNA stabilizing and destabilizing elements and nonsense mediated decay. This disclosure provides two ERBB2 destabilizing constructs named desARE3′UTRERBB2-3 and 30 and administered them intraperitoneally to the tumor bearing mice of trastuzumab resistant breast cancer cells either as plasmid DNA or packaged into iron oxide nanocages delivery vehicle. Complete tumor lysis was achieved with the inhibition of the ERBB2, WNK1 and YES1 with 40% of treated animals being exceptional survivors. Toxicity profile assay shows that constructs were well tolerated and caused no excessive abnormal blood dyscrasia or changes in electrolyte or liver and pancreatic function. This provides therapeutics for the deadly ERBB2 positive drug resistant metastatic breast cancer, thereby offering a new means to target these recalcitrant cancers with therapeutic efficacy.

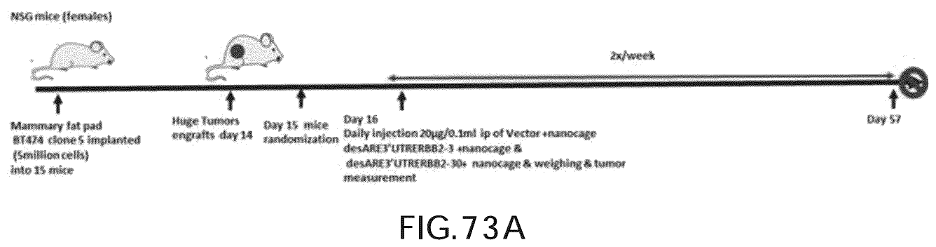

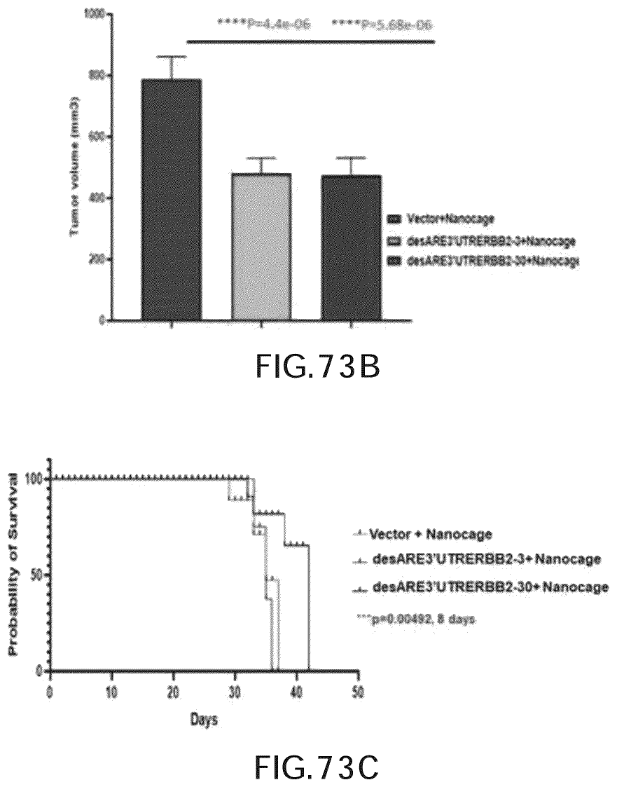

Results: Two ERBB2 destabilizing constructs desARE3′UTRERBB2-3 and 30, were developed. In-vitro, they destabilize and degrade ERBB2 transcript and protein in the drug resistant breast cancer cells BT474 clone 5 leading to loss of cancer cell viability. To prove their therapeutic efficacy in-vivo, two independent animal experiments were performed. In the first experimental cohort, BT474 clone 5 cells were implanted into the mammary fat pad of female NSG mice. After 14 days the tumors engrafted and were randomized into 5 mice per cage into 3 groups: the vector treated, the desARE3′UTRERBB2-3 treated and the desARE3′UTRERBB2-30 treated (FIG. 71A). In the second experimental cohort, BT474 clone 5 cells were implanted into the mammary fat pad of female NSG mice. After 14 days the tumors engrafted and were randomized into 5 mice per cage into 3 groups: the vector+nanocage treated, the desARE3′UTRERBB2-3+nanocage treated and the desARE3′UTRERBB2-30+nanocage treated (FIG. 73A).

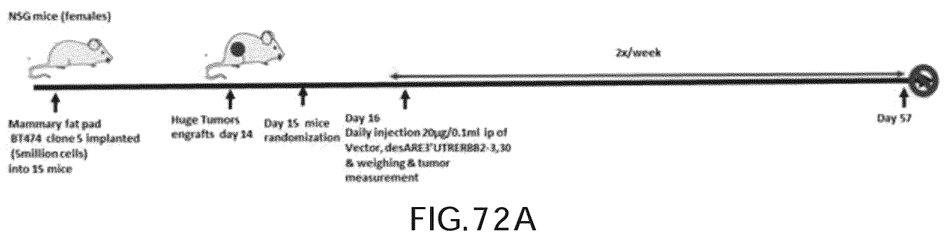

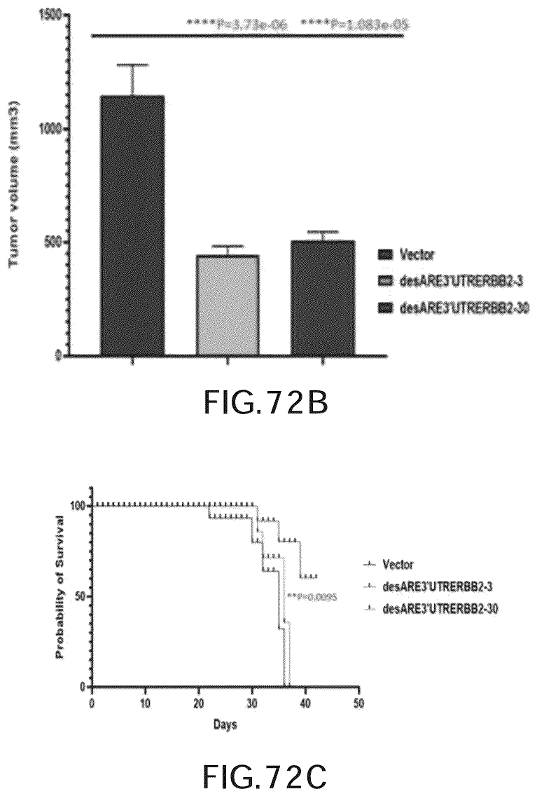

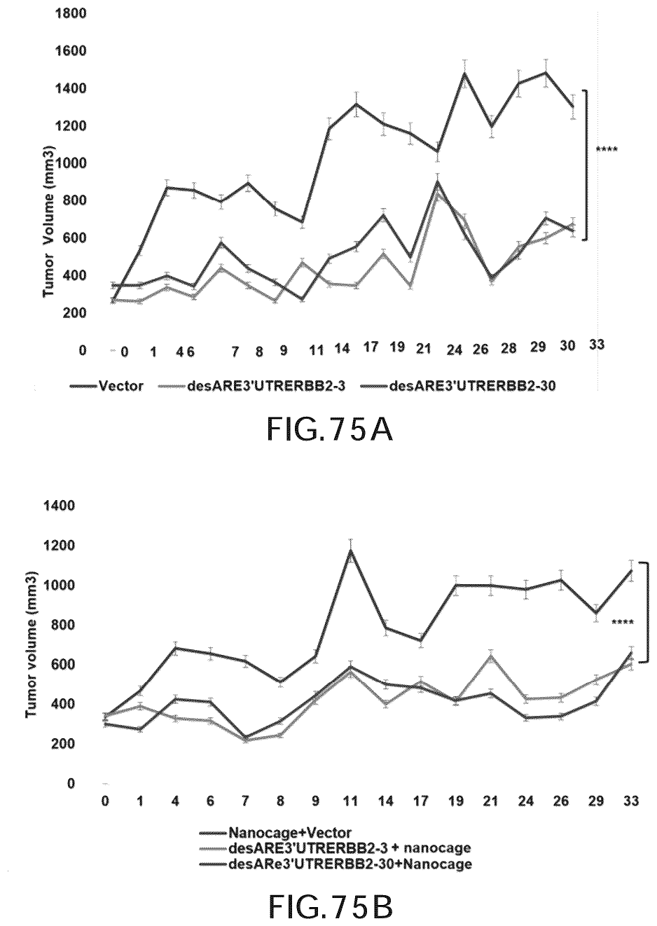

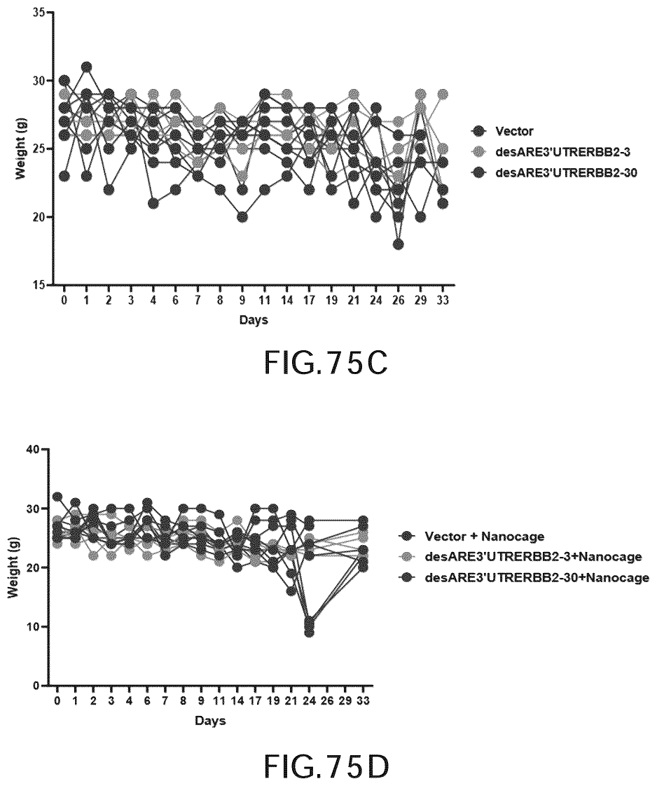

The constructs were administered intraperitoneally as plasmid DNA in cohort 1 (FIG. 72A) and as complexed with iron oxide nanocage in cohort 2 (FIG. 73A) at 20 μg/0.1 ml 2× per week for 41 days. The daily tumor volume measurement shows that the engineered destabilized 3′UTRERBB2-3 and 30 achieved very significant reduction of tumor volume (****p=3.73e-06, ****1.08e-05) respectively (FIG. 72B, FIG. 75A). The desARE3′UTRERBB2-3 and 30+nanocage achieved very significant reduction of tumor volume (****p=4.4e-06, ****p=5.68e-06, FIG. 73B, FIG. 75B) respectively. In the desARE3′UTRERBB2-3 treated tumor bearing mice, about 40% were exceptional survivors (**p=0.0095, FIG. 72C). In the nanocage treated cohort, the desARE3′UTRERBB2-30+nanocage achieved significant survival (***p=0.00495, 120 FIG. 73C).

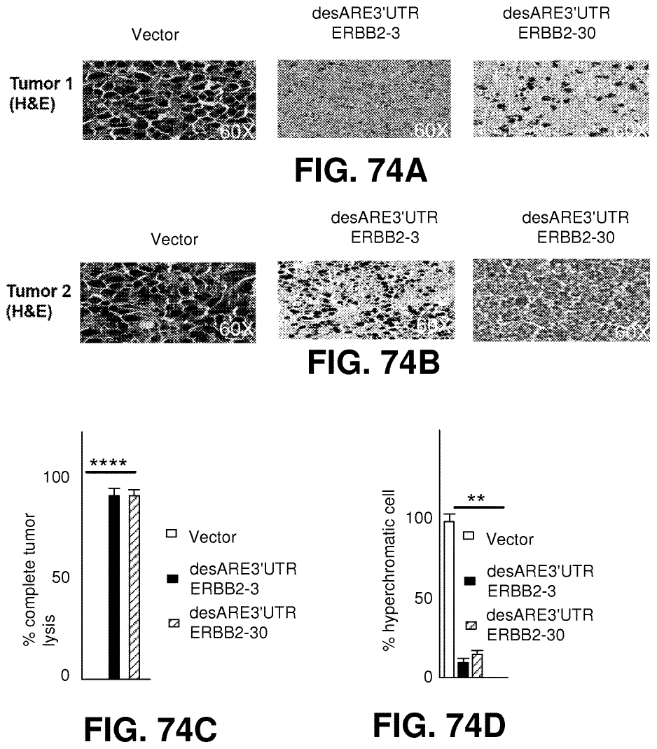

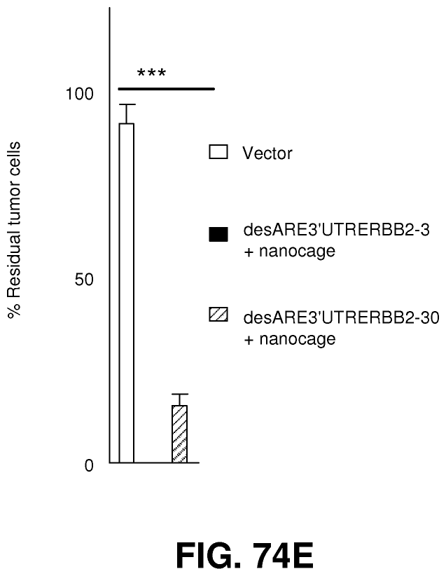

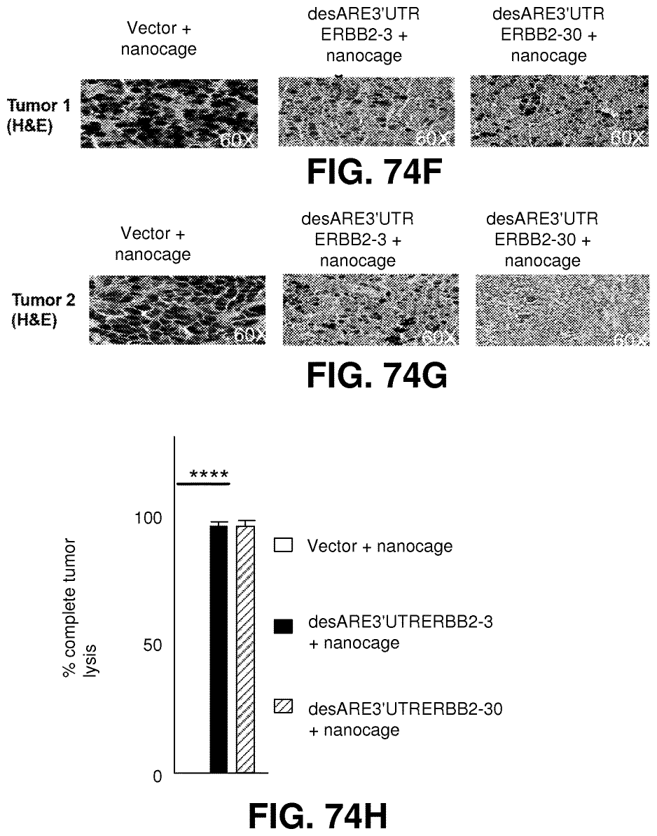

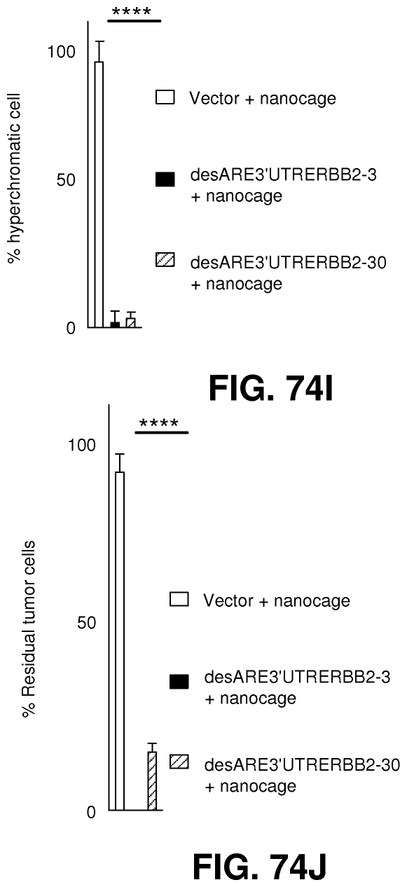

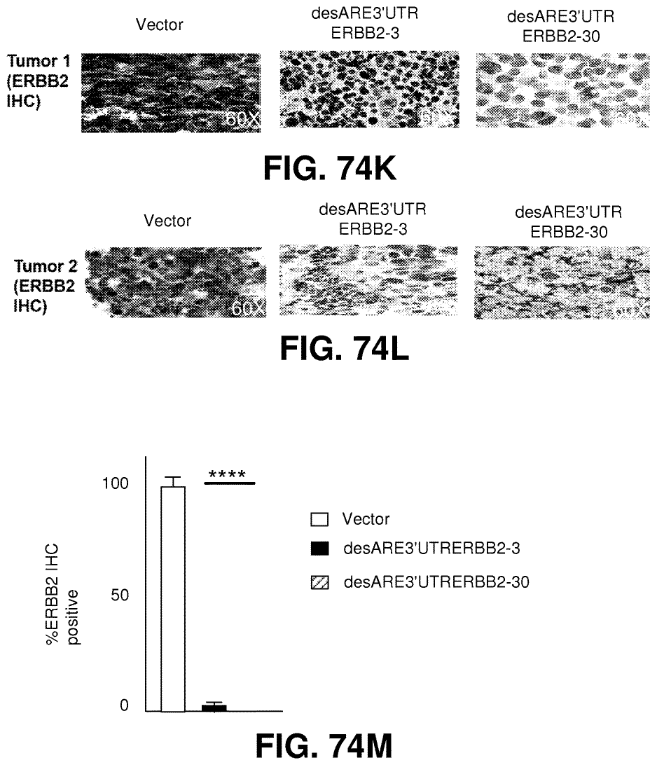

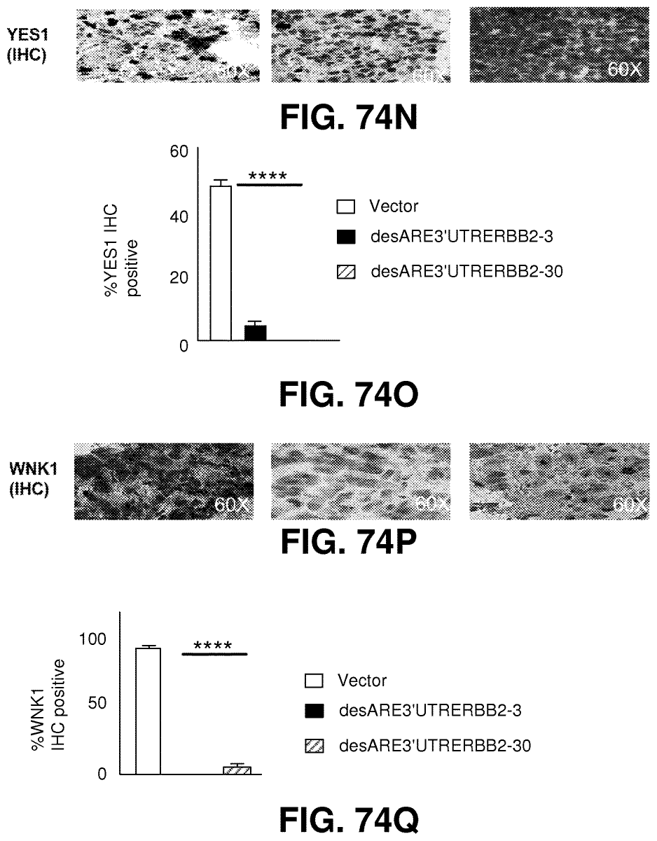

To validate the therapeutic efficacy of the constructs, H&E analysis of the tumors treated with the desARE3′UTRERBB2-3 and 30 was performed. The constructs desARE3′UTRERBB2-3 and 30 achieved complete tumor lysis (FIG. 74A, FIG. 74B, FIG. 74C) with near complete eradication of the large hyperchromatic cancer cells (FIG. 74D) and few residual hyperchromatic nuclei in the treated animals (FIG. 74A, FIG. 74B, FIG. 74E). Similarly, to assess the therapeutic efficacy of the constructs complexed with iron oxide nanocages. H&E analysis of the tumors treated with the vector plus nanocages, desARE3′UTRERBB2-3 and 30 plus nanocages, was performed. The constructs again achieved complete tumor lysis (FIG. 74F, FIG. 74G, FIG. 74H), with the desARE3′UTRERBB2-3 and 30+nanocages achieving near complete eradication of the large hyperchromatic nuclei and residual cells (FIG. 74I, FIG. 74J).

To confirm the molecular on target of ERBB2 degradation by the constructs in the treated tumors. ERBB2 IHC staining of the tumors from the controls and the treated animals was performed. The desARE3′UTRERBB2-3 and 30 achieved complete reduction of ERBB2 signal (FIG. 74K, FIG. 74L, FIG. 74M). Drug resistance in ERRB2 positive metastatic breast cancer is driven by kinases YES1 and WNK115,16. To assess if these ERBB2 dependent kinases expression were reduced by the degradation of ERBB2. The vector and the construct treated tumors was stained with antibodies against WNK1 and YES1 by IHC. The construct treated tumors achieved loss of YES1 and WNK1 (FIG. 74N, FIG. 74P, FIG. 74O, FIG. 74Q).

Taken together, these findings demonstrate that the constructs were specific and achieved therapeutic inhibition of tumors and the molecular target ERBB2 and its dependent kinases YES1 and WNK1 that drive chemoresistance.

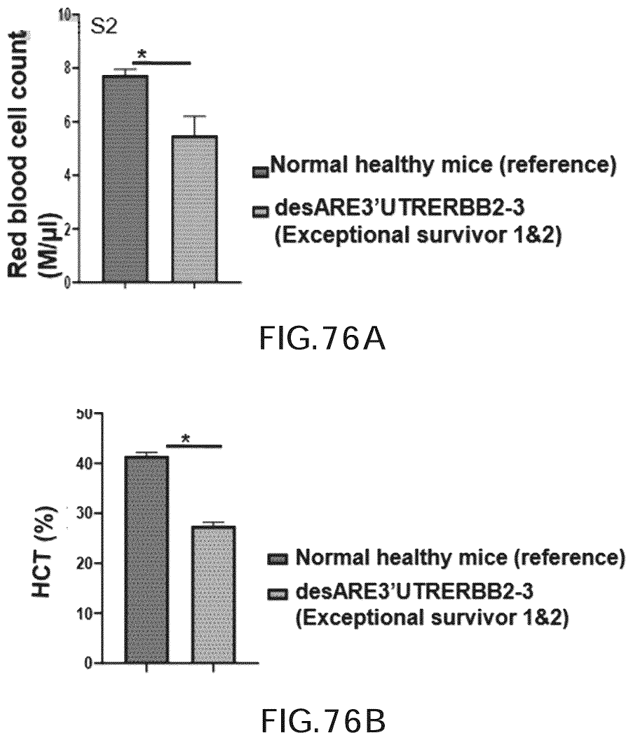

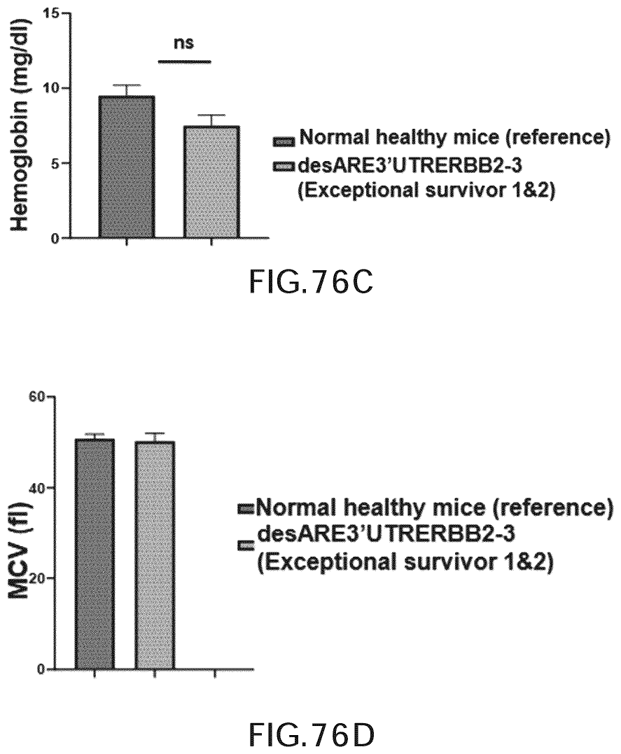

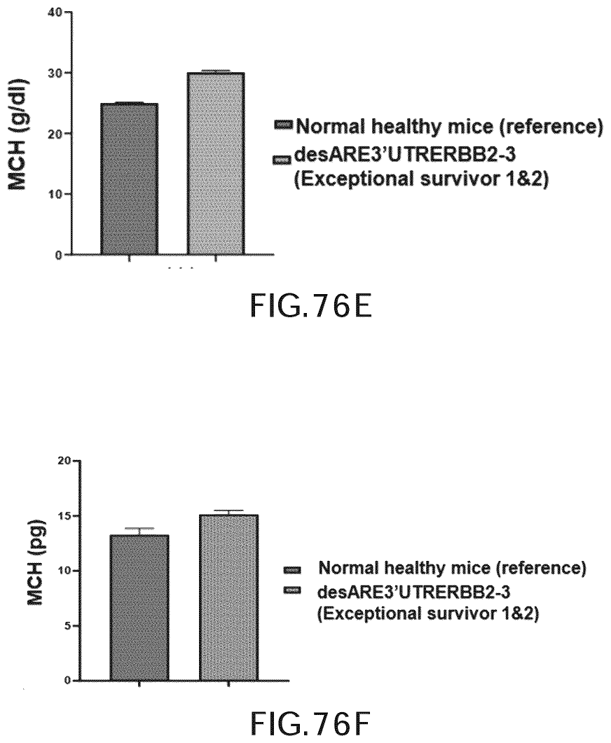

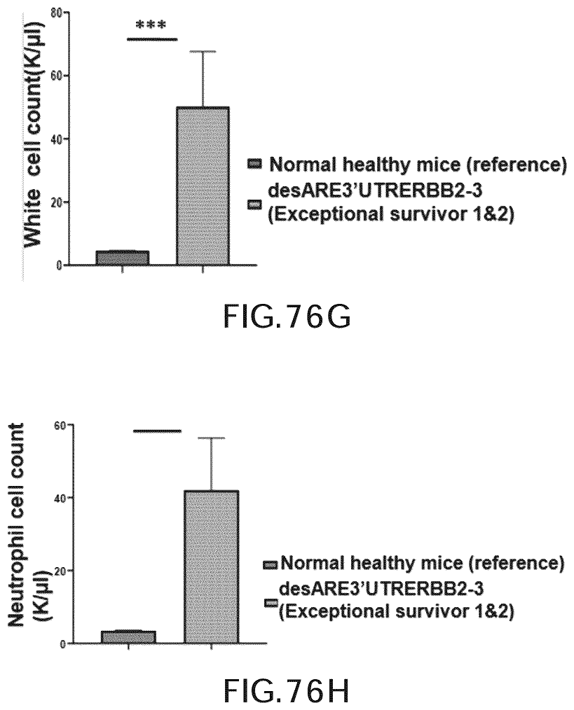

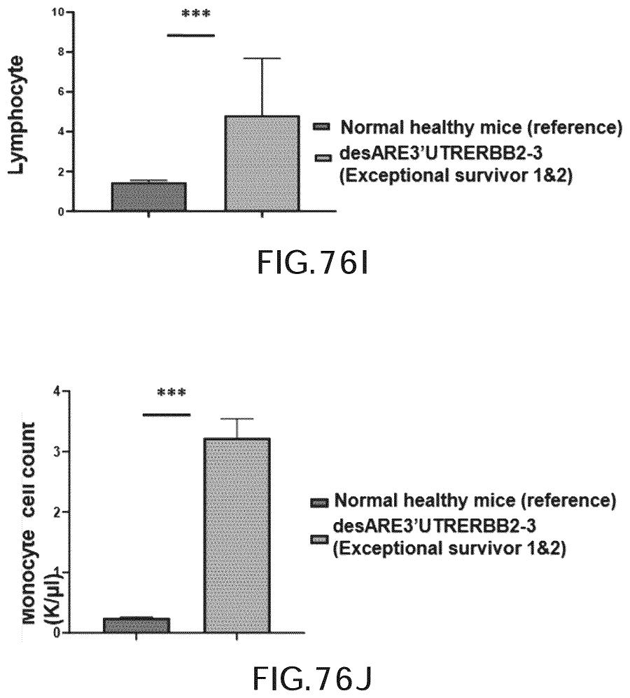

The short term (9 days) the constructs were safe. Next, to assess the safety of the constructs for long term 57 days treatment. A toxicity assay profile was performed examining the vital organ functions of the blood, electrolyte, liver, kidney, gall bladder and pancreatic function by an independent core pathology lab, the Memorial Sloan Kettering Cancer Center Core Pathology laboratory. There is mild reduction in red blood cell count and hematocrit level and hemoglobin levels (FIG. 76A, FIG. 76B, FIG. 76C) which indicates mild anemia. No change in the mean corpuscular volume, mean corpuscular hemoglobin concentration, and mean corpuscular hemoglobin was found (FIG. 76D, FIG. 76E, FIG. 76F), indicating there is no iron deficiency anemia or macrocytic anemia. Elevated levels of white blood cells, neutrophils, lymphocytes, and monocytes were found (FIG. 76G, FIG. 76H, FIG. 76I, FIG. 76J) indicating the immune cells are actively attacking the cancers.

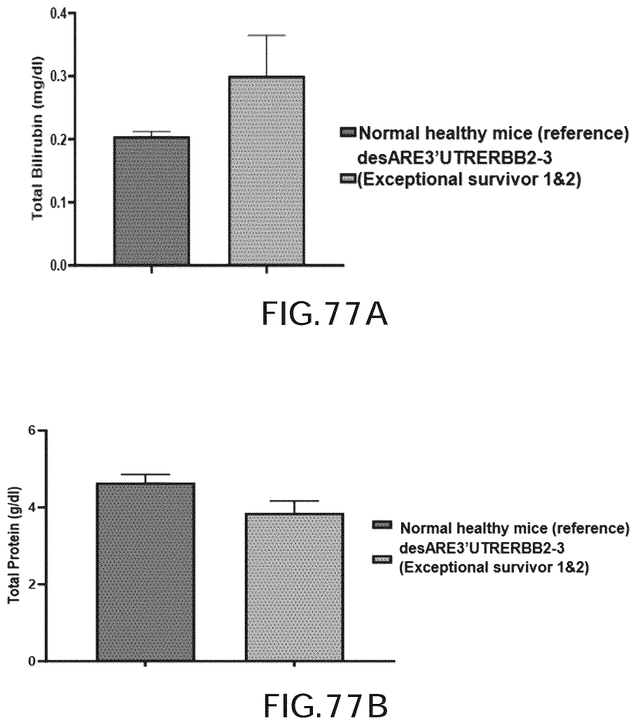

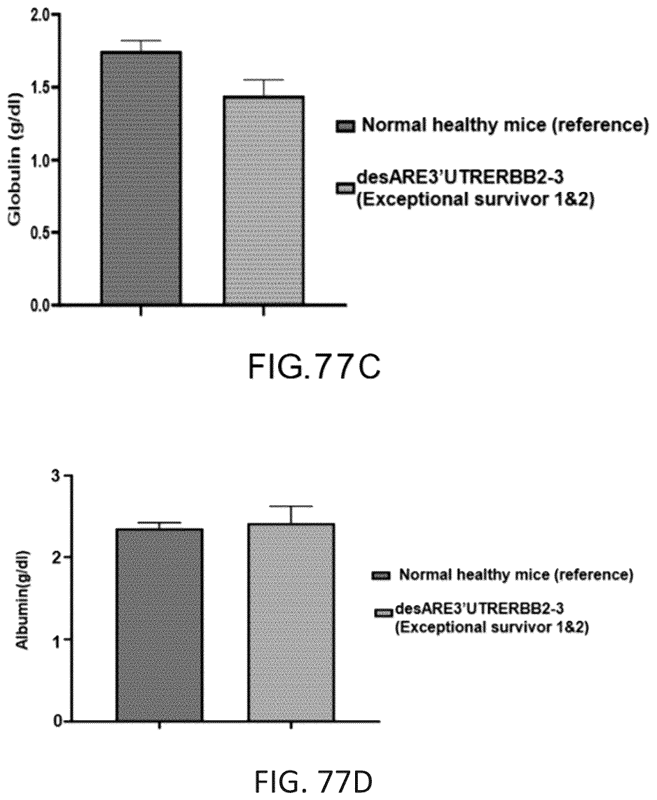

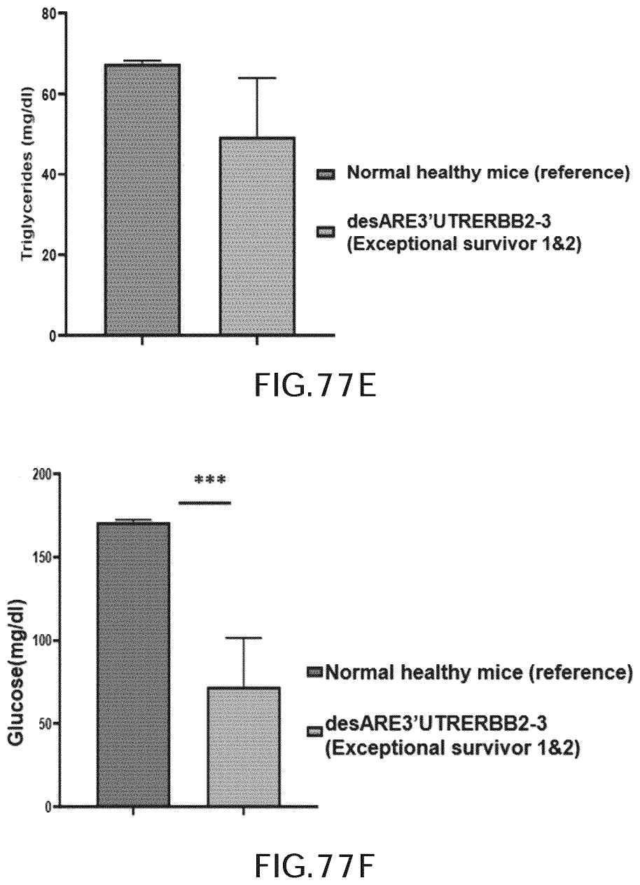

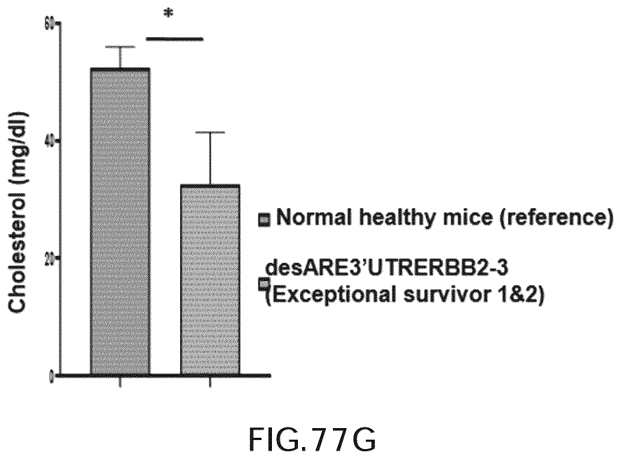

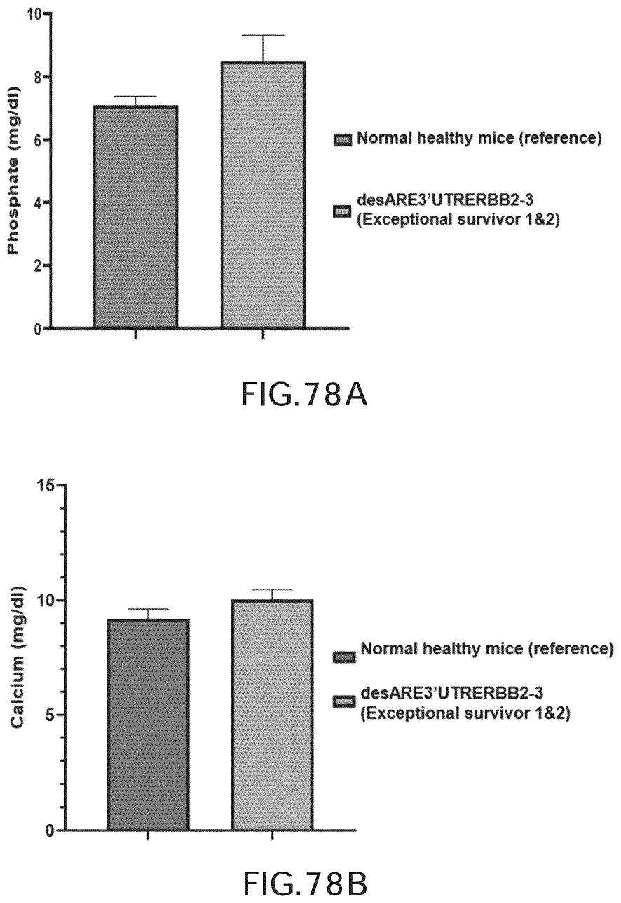

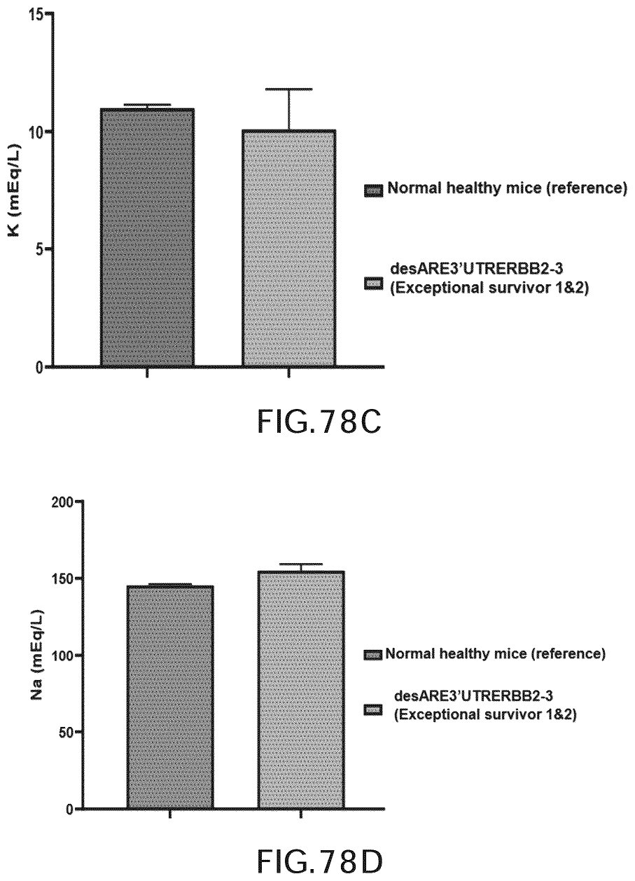

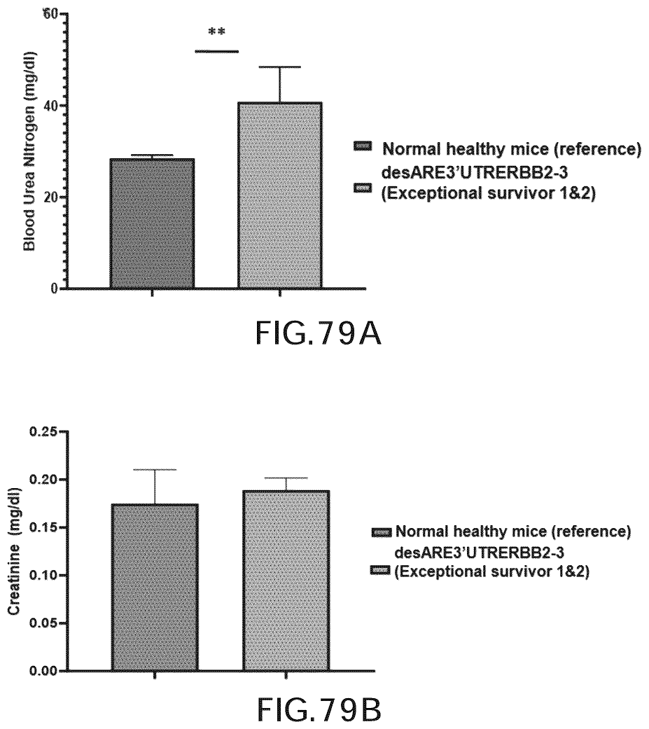

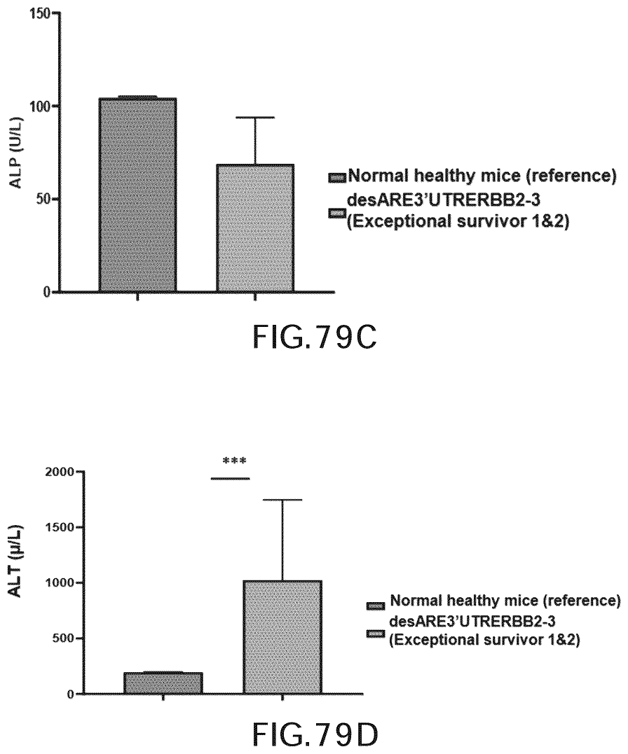

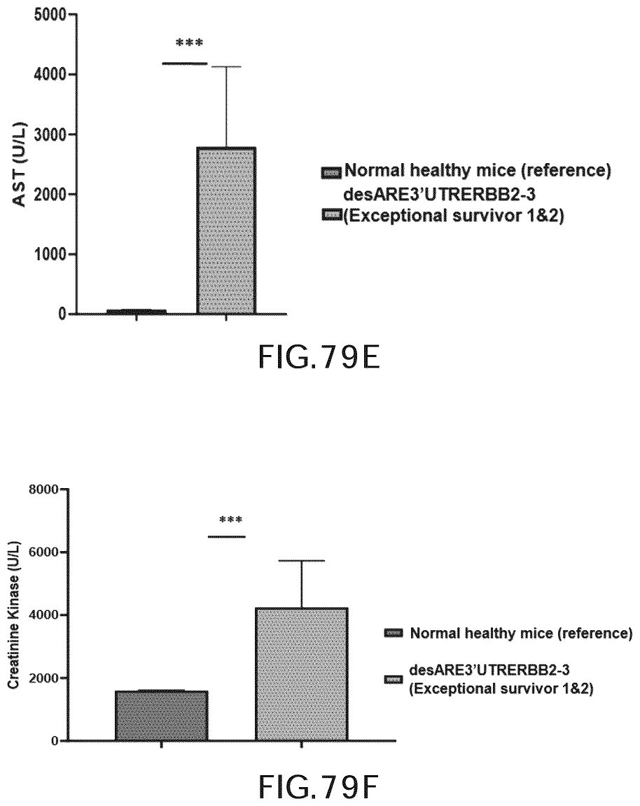

A liver function test found slightly elevated total bilirubin, slight decrease in total protein and globulin and no changes in albumin (FIG. 77A, FIG. 77B, FIG. 77C, FIG. 77D). A slight decrease in the levels of triglycerides and cholesterol were found (FIG. 77E, FIG. 77G) and severe decrease in the levels of glucose (FIG. 77F). This data indicates that there was no severe liver function damage, but this therapy might cause hypoglycemia which can be treated with 5% glucose saline infusion as done in standard medical practice. Furthermore, we assessed the impact of therapy on electrolytes. No changes in the phosphate, calcium, potassium, and sodium levels were found (FIG. 78A, FIG. 78B, FIG. 78C, FIG. 78D). This indicates that there are no abnormal changes in the blood chemistry by this therapy. Lastly, the levels of liver enzymes were assessed. Slightly elevated blood urea nitrogen was found along with no changes in creatinine levels, slight decrease in alkaline phosphatase and elevated levels of alanine transaminase, aspartate transferase and one exceptional survivor had elevated creatinine kinase (FIG. 79A, FIG. 79B, FIG. 79C, FIG. 79D, FIG. 79E, FIG. 79F). This data indicates there is ongoing kidney abnormality, no cachexic muscle loss as seen in chemotherapy, no liver, bone damage or cirrhosis. Elevated ALT, AST indicates inflammation of the liver, this could be explained by very elevated levels of the white blood cells, neutrophils and lymphocytes and monocytes seen in (FIG. 76A-J).

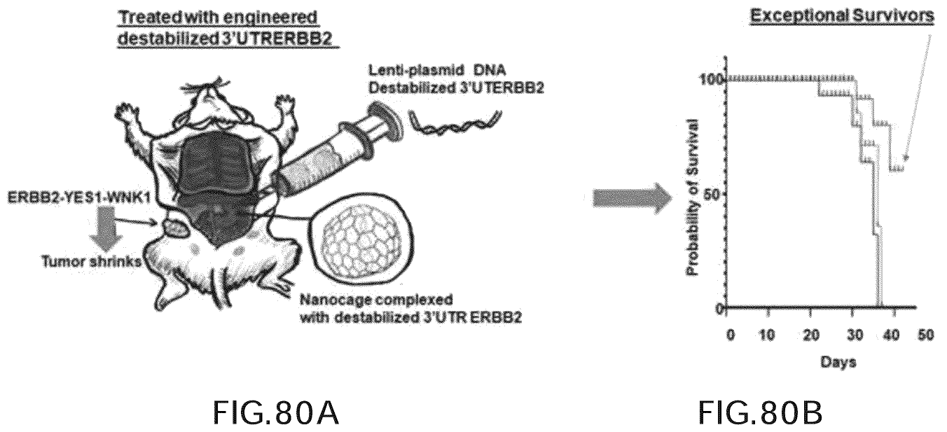

FIG. 80A is a depiction of mice bearing tumor treated with the engineered destabilized 3′UTRERBB2 either as lenti-plasmid DNA or packaged into iron oxide nanocage shrinks the tumors with the downregulation of the ERBB2-YES1-WNK1 kinases. FIG. 80B is a survival graph showing the therapeutic effects of exceptional survivors in the treated animals.

Taken together, this demonstrates the therapeutic efficacy of the constructs and the iron oxide nanocage delivery system and their safety levels in-vivo on a long-term basis. In summary, the data indicates that constructs are on target with ERBB2 inhibition, and it is safe in-vivo.

### Discussion

The engineered destabilized 3′UTR of ERBB2 achieved therapeutic degradation of ERBB2 as well as WNK1 and YES1 kinases that mediate drug resistance. This led to complete tumor lysis in the two independent experiments. About 40% of the mice bearing tumor that received this therapy became exceptional survivors of the deadly metastatic drug resistant breast cancer. The constructs were well tolerated and overall impact on the blood chemistry, liver, pancreatic and kidney functions were moderate, and they caused no blood dyscrasia, pointing that they were safe. Moreso, there was no cachexia which indicates that this therapy might be superior to the current chemotherapies which are known to be toxic to all the cells of the body and lacks selectivity. This study was done in two independent experiments involving a total of 30 female NSG mice.

### Materials and Methods

Cell Culture: The BT474 clone 5 cells obtained from ATCC were cultured in RPMI supplemented with the 10% FBS and 5% antibiotics. Cells were grown until they are 80% confluent before use.

Development of the destabilized 3′UTR ERBB2 constructs: The design and engineering of the destabilized ERBB2 3′UTR constructs is described in detail elsewhere in this disclosure. In brief, the poly U mRNA stabilizing sequences on the 3′UTR of ERBB2 were identified across different ERBB2 driven cancer. Then, an engineered synthetic construct was designed where the stable elements were changed to unstable forms on the 3′UTR driven by DCP1A minimal promoter, this triggered nonsense mediated decay mediated by CNOT1 and XRN1 exonucleases. The synthetic construct was cloned either by sticky end205 ligation using BamH1 and BstB1 site or by Gibson assembly to the receiving vector (pLenti-206CMVSP6-nEGFP-SV40-PURO, Addgene #138364). They were used to transform competent E. coli and the clones picked were miniprepped and confirmed by Sanger Sequencing. The Zymo Research (Cat no: D4203) maxi prep kit was used for the generation of high yield ultrapure plasmid DNA containing the constructs for animal experiments.

Animal Study: 30 mice NSG female mice were ordered from the Jackson Laboratory. The mice were received and allowed to acclimatize according to institutional protocol. The BT474 clone 5 were cultured in RPM1, when at 80% confluency, the cells were harvested and washed in PBS and counted. 5 million cells in 0.1 ml were implanted in the mammary fat pad of animals. After 14 days, huge tumors engrafted and on the 15 days, the animals were randomized into 5 mice per cage into 5 cages with equal distribution of tumor size (FIG. 75C, FIG. 75D, FIG. 76C). On day 16, intraperitoneal administration of the iron oxide complexed with the engineered destabilized construct was started at 20 μg 12 hrly 2× per week for 41 days with daily measurement of tumor volume and animal weight and body condition score. At 57 days, the experiment ended. Complete necropsy was performed and the organs were sent to the core pathology group of the Memorial Sloan Kettering Cancer Institute for histological analysis and H& E staining. The expert pathologist was blinded to the experimental details.

Packaging of the engineered destabilized construct into iron oxide nanocage: 3,4-dihydroxyhydrocinnamic acid (DHCA) by Alfa Aesar, Manganese (II) acetate, oleylamine, oleic acid, and iron (II) perchlorate) were purchased from Sigma-Aldrich. p-Xylene, 1-ethyl-3-[3-(dimethylamino) propyl]carbodiimide (EDC), and N-hydroxysuccinimide (NHS) were purchased from Thermo Scientific. Tetrahydrofuran (THF), dimethylsulfoxide (DMSO), ferric chloride hexahydrate, and phosphate buffer saline (PBS) was purchased from Fisher Scientific. Cy7.5 dye was purchased from Lumiprobe.

Generation and structure analysis of DNA plasmid-IO-nanocage complexes: The IO-nanocages were synthesized by a galvanic reaction as manganese ions of template Mn₃O₄ nanocubes are replaced by iron ions to form a cage-structure with a hollow center, as shown in previous publications (Rampersaud et al. Nanocages. Nano Lett., 2016, 16, 7357-7363; Liu et al. J. Am. Chem. Soc., 2014, 136, 12552-5; Raghubir et al. Oncol. Rep., 2020, 43, 169-176). Water-soluble IO-nanocages were engineered by capping with 3-(3,4-Dihydroxyphenyl) propionic acid (DHCA) before loading the DNA plasmids containing the destabilized ERBB2 constructs as well as vector controls only. DNA plasmids were mixed with IO-nanocages with the mass ratio of 1:1 for the complexation based on the concentration of DNA plasmids needed for the sufficient efficacy with respect to the optimized dosage of IO-nanocage concentration for in vivo experiments.

Haematoxylin and Eosin (H&E) Staining and Immunohistochemistry (IHC): BB performed ERBB2, WNK1 and YES1 IHC and primary tumor H&E according to standard protocol. After sacrifice, tumor, spleen, liver, kidney, lung, hearth and brain samples obtained from mice via dissection were fixed in formalin and embedded in paraffin. Sections of 4 μm thickness was stained with hematoxylin & eosin (HE) and IHC antibodies.

Briefly, samples of the tumors, livers, brains, spleens, kidneys, and lungs were fixed, stained with HE, and visualized under an optical microscope. HE staining enabled determination of the morphological features of the tumors. For IHC staining, slides were deparaffinized, dehydrated, blocked. The slides were blocked with mouse serum for 30 min and incubated with HER 2 antibody (1:500) (Proteintech, 18299-1-AP), YES 1 antibody (1:500) (Proteintech, 20243-1-AP), WNK 1 (1:500) (Proteintech, 28357-1-AP) were performed at 4 C overnight. All the buffer and reagents used in IHC was used from IHC Prep & Detect Kit for Mouse Primary Antibody (Proteintech, PK10018) according to manufacturer's instructions.

Next, the slides were incubated with the secondary antibody, stained with diaminobenzidine substrate and counterstained with hematoxylin. Lastly, all samples were analyzed via optical microscope.

Pathological scoring: Reporting of cancer cell lysis, mitotic counts, histological subtyping and ERBB2, WNK1 and YES1 grading were done according to the following protocols. Cell lysis was reported using high resolution microscopy as well as observing the appearance of disrupted cell membranes and naked nuclei under high power resolution. Nottingham's prognostic index was used to grade the breast cancer. While histological typing was done according to the degree of differentiation, with poorly differentiated cells forming no glands and growing in sheets.

ERBB2 grading was considered positive if at least 30% of tumor cells exhibited 3+ cell membrane staining, and a borderline result was given when at least 10% of cells showed 2+ cytoplasmic membrane staining.

Toxicity profile analysis: For the analysis of the blood cell count, electrolyte, liver function, kidney, pancreatic and gall bladder function. The exceptional survivor animals were submitted to the Memorial Sloan Kettering Core pathology lab, and they performed this analysis. They are blinded to the details of the experiment.

Statistical analysis: All the experiments done were performed in replicates. Starting with the animal experiment. A total of 30 NSG female mice were used at 5 mice per group per cage. The groups were IO-nanocage+vector, desARE3′UTRERBB2-3+IO-nanocage, desARE3′UTRERBB2-30+Nanocage and vector only, desARE3′UTRERBB2-3 and 30. Two-tailed t-test were used to determine statistically significant between the controls and the different treatment groups. Log rank mantel test were used to determine statistically significant survival differences.

c-MYC

c-MYC is a master transcription factor overexpressed in more than 70% of human cancers, including triple negative breast cancer (TNBC), yet there is no clinically approved drug that directly targets it. Herein, the mRNA stabilizing poly U sequences were engineered within the 3′UTR of c-MYC to specifically destabilize and promote the degradation of c-MYC transcripts. Interestingly, the engineered derivative outcompetes with the endogenous overexpressed c-MYC mRNA, leading to reduced c-MYC mRNA and protein levels. Delivering iron oxide nanocages (IO-nanocages) complexed with MYC destabilizing constructs into mice bearing TNBC inhibits primary tumors and metastases and significantly prolongs survival by degrading the c-MYC-STAT5A/B-PDL-1 complexes that drives c-MYC positive TNBC. This disclosure provides a therapy for c-MYC-driven TNBC and uncovered c-MYC-STAT5A/B-PDL-1 interaction as the target. Resistance to this approach appears to be driven by STAT5A/5B-independent c-MYC program.

c-MYC is a basic helix loop helix (b-HLH) master transcription factor that binds the E-box sequences and is overexpressed in >74% of human cancers. This includes breast cancers, lung cancers, brain tumors, prostate cancers, lymphomas, pediatrics cancers, pancreatic cancers, colorectal cancers, and ovarian cancers. Currently, there is no approved direct clinical inhibitors of c-MYC, which is a significant biological and clinical challenge that needs to be overcome for more effective cancer treatment.

Triple negative breast cancer (TNBC), a very aggressive tumor with limited treatment option, 61.27% of TNBC is driven by c-MYC signal. In the USA, TNBC accounts for 10.34% of breast cancers. In China, triple negative breast cancer represents 14.01% cases of invasive breast cancer. In Africa, very high rates of TNBC have been reported. About of 36-79% of breast cancer in African women has been reported to be estrogen receptor (ER) negative, 30-87% is progesterone receptor (PR) negative and fewer studies have reported on HER2 positive status in African women with the majority largely been classified as HER2 negative.

Various approaches have been attempted to control MYC, but they all have limitations. The RNAi approach has high off target effects, the anti-sense oligonucleotides (ASO) approach has low cellular penetrance and needs phosphorothioate modification, G-quadruplex inhibitors are unspecific as they block POLII, the BRD4 and CDK inhibitors are indirect inhibitors of MYC. Only OmoMyc, a b-HLH domain MYC mutant with enhanced leucine zipper dimerization is in phase 1 clinical study.

To address this grand challenge of direct and specific targeting of the pervasive c-MYC expression in human cancers, this disclosure provide a method based on engineering the genetic codes on the 3′UTR of the MYC gene. The mRNA stability sequences in the 3′UTR of c-MYC were engineered to unstable forms and their expression to be driven by the mRNA de-capping promoters, and achieved direct spatial and temporal control of c-MYC transcripts and protein and degraded them by overwriting the endogenous MYC message. Three c-MYC destabilizing constructs specifically destabilized MYC transcript, degraded MYC transcripts and proteins as well as kinases and other transcription factor STAT5A/B and PDL-1 under MYC controls in TNBC cells and killed these cancer cells within 1 week, the cells ruptured from the nucleus and expressed active caspase 3/7 leading to the cancer cell death. In vivo, delivering IO-nanocage complexed with the destabilizing constructs into mice bearing TNBC reduced primary tumor volume by 60-80% and inhibited metastasis. Mechanistically, destabilization of c-MYC degraded MYC and its complex interacting partner STAT5A/B and PDL-1 in primary tumors and metastases that were controlled. However, in uncontrolled metastatic tumors, MYC was downregulated, but STAT5A/B was expressed, suggesting that MYC-dependent STAT5A/B-PDL1 was controlled in the primary and metastatic tumors where the disclosed approach worked whereas in the nonresponsive metastatic lesions were governed by MYC-independent STAT5A/B interaction.

### Materials and Methods

Cell culture of MDAMB231, BT474, BT474 clone 5, MCF7, T47D, RWPE1: MDA MB231 was grown in DMEM supplemented with 10% FBS and 1% penicillin streptomycin. BT474, BT474 clone5, MCF7 and T47D was grown in DMEM supplemented with 10% FBS and 1% penicillin-streptomycin. C4-2B were grown in DMEM/F12 with 10% FBS, 1% penicillin streptomycin, supplemented as described in ATCC. RWPE1 were grown in Gibco keratinocyte serum free medium, supplemented with bovine pituitary extract (BPE) and human recombinant epidermal growth factor (EGF) with 1% penicillin streptomycin. Cells were grown to 80% confluency before use.

Cell line authentication: The cell lines used in this experiment, which are MDAMB231, BT474, BT474 clone 5, MCF7, T47D, C4-2B and RWPE1, were authenticated by STR (short tandem repeats sequencing) and frequent testing for mycoplasma were conducted regularly on these cells.

RNA extraction from multiple cancers: Total RNA was extracted from the MDAMB231, BT474, T47D, MCF7, RWPE1 and using the RNA Easy kit from the Qiagen (cat no: 74104). The RNA was stored at −80C until use.

QPCR with c-MYC 3′UTR primer: To determine the stabilizing poly U rich elements on the 3′UTR of c-MYC, cDNA was made from the total RNA using the Qiagen reverse transcription kit (Catalog no: 205311). RT-PCR was performed using the cDNA according to the Qiagen manufacturers protocol using the primer sequences in Table 10.

Sanger sequencing of 3′UTR of c-MYC cDNA amplicon: To confirm the c-MYC stabilizing poly U 3′UTR sequences. The amplified bands were excised under UV light and then extracted with Qiagen gel extraction kit (Catalog no: 28706X4) according to manufacturer's protocol. The amplicon was then sequenced with the c-MYC 3′UTR PCR primers by Sanger Sequencing method by the commercial company Psomagen Inc. Brooklyn New York USA. Subsequently, an online software DNA to mRNA translator was used to identify the stable poly U motifs of c-MYC 3′UTR.

Design of destabilized 3′UTR of c-MYC: To design the destabilized 3′UTR of c-MYC, we replaced the consensus stabilized sequences with the AU rich elements. Next, some residues on the 3′ end of the 3′UTR were modified to increase stability. Torabi S. F. et al Science 2021, used structural biology and biochemical assays to delineate residues of helices of the nucleic acids on the PolyA tail of 3′UTR mRNA that determines their strong, moderate stability as well as rapid degradation. With this information, the loop structures of the stabilized and destabilized 3′UTR of c-MYC were analyzed. Changes in these residues have a remarkable impact on the stability of transcript up to 120 mins. Having established the structure of stabilized c-MYC 3′UTR and mutated residue of destabilized c-MYC 3′UTR that increased their stability. These destabilized constructs were incorporated with an upstream 5′ BstB1 restriction site followed by a polyA sequence to stop the RFP transcription of the vector and then followed by a DCP1A promoter. At the 3′ end a polyA sequence was added followed by BamH1 restriction site.

Synthesis of destabilized 3′UTR ARE of c-MYC as gblock: The destabilized 3′UTR of c-MYC was synthesized as gblock from Integrated DNA Technologies. (IDT Inc USA).

Vector: The destabilized 3′UTR of c-MYC was cloned into pLenti-CMVSP6-nEGFP-SV40-PURO (Addgene: #138364).

Design of Gibson Assembly Primers: c-MYC and Vector Gibson Assembly primers design and Assembly: The sequences of the synthetic gblock of destabilized 3′UTR c-MYC and vector (Sp6 vector) were loaded into the NEB builder which generated the following primers (Table 10) to amplify the c-MYC and Sp6 vector primers for Gibson Assembly, and NEB HIFI DNA assembly kit were used (NEB cat no: E2621S).

Transformation Recombination deficient E. coli (NEB Cat no C3019H): Deficient competent E-coli were transformed with the DNA assembly using SOC media. 25 μl of each cell was incubated on ice with 5 μl of the ligation mix for 30 mins. After 30 mins was heat shocked in water bath at 42° C. for 30 sec. Tubes were placed back on ice for 2 mins and 900 ul of SOC media was added and then incubated at 37 C shaking for 1 hr at 250 rpm. After which they were plated on LB Agar plate containing ampicillin and the plate was incubated overnight at 37 C.

Colony picking and Miniprep: Colonies were picked with pipette tips and inoculated into 5 ml LB media containing Ampicillin and grown overnight at 37 C shaking at 250 rpm. The pellets were spurned down and gDNA was extracted using the Qiagen Midikit (Cat no: 12943). And using a nanodrop machine the gDNA were quantified.

Colony PCR with c-MYC Gibson primers: PCR was performed with c-MYC Gibson primers by using the PCR cycle described above.

Gel extraction: The colony PCR products of c-MYC 3′UTR Gibson assembled was gel extracted using Qiagen gel extraction kit (Cat no: 28704).

Sanger sequencing of the cloned destabilized 3′UTR of c-MYC amplicon: The colony PCR product of Gibson assembled c-MYC 3′UTR was sequenced using the Gibson primers described above by Sanger Sequencing with Psomagen Inc, Brooklyn New York. Clones 3′UTR MYC2-3, 3′UTR MYC1-18, 3′UTR MYC 1-14 were obtained.

Sequence alignment of wildtype cDNA, RNA versus the destabilized 3′UTR c-MYC cDNA and RNA sequences: To confirm the engineered changes of the destabilized ARE 3′UTR of c-MYC versus the wildtype c-MYC cDNA and RNA, the wildtype and the engineered destabilized and cloned sequences were aligned, both on the DNA and RNA level, with their control wildtype using the software Clustalw Omega.

Transfection/electroporation of destabilized 3′UTR of c-MYC into MDAMB231 cancer cells and normal epithelial cells RWPE1: To introduce the destabilized 3′UTR into the cancer cells, constructs were electroporated into 200,000 to 500,000 cells with 50 ng of plasmid containing the constructs using the BioRad electroporation system. The preset mammalian protocol set was used for 293T cells and pulsed the cells in a cuvette 2×. Cells were then seeded into 6 well plates and viewed for morphology and red fluorescent protein (RFP) expression in 24 hrs. After 24 hrs the cells expressed RFP indicating that the constructs were successfully integrated into the cells.

Cell Microscopy: The cells were observed regularly under the light microscope. Within day 4, the MDAMB231 wildtype containing the destabilized elements showed ruptured nuclei compared to the control.

Cellular Immunofluorescence: To determine the c-MYC protein expression changes in the destabilized breast cancers, osteosarcoma compared to the wildtype. The cells were seeded on a tissue culture slide or 6 well plate at 2,000-3,000 per well. The cells were allowed to grow for 1 day and after they were fixed with 200 μl of 4% paraformaldehyde added to the cell media. The cells were placed at 4 C for 5 mins, after which the paraformaldehyde was decanted. Anti-c-MYC (1:1000) in BSA were added to the slide wells and sealed with aluminum foil and kept at 4 C overnight. After which the antibody is removed and secondary antibody Alexa 488 (green) or Alexa 610 (red) were added at 1:10,000 and covered with aluminum foil and kept at room temperature for 1 hr. After 1 hr, the secondary dye was washed with PBS (phosphate buffer saline) 3× and then with a Kim wipes the edges were wiped off water. And then a drop of DAPI (Nuceloblue-nuclear stain) was added and then sealed with cover slips and the slides were viewed under a Nikon confocal microscope.

Western blot: To quantify the c-MYC protein expression changes in wildtype cells compared to the cells containing the destabilized 3′UTR. The cells were harvested and lysed in a cocktail of protease inhibitor in MPER buffer. The protein extract was stored in −80C until use. The protein was separated in 12% stacked SDS page gels and separated by initial run at 75 volts and after 15 mins at 120 V until complete separation. The proteins were transferred unto a PVDF membrane already charged with methanol at 25 V for 1 hr. Membrane was blocked with BSA and subsequently incubated with anti-c-MYC (Cat no: 67447-1-1g protein tech) and control GAPDH/Actin overnight in BSA. Primary antibody was removed and secondary anti-mouse IR 800CW dye (Li-COR) was added at 1:1000 incubated with BSA covered from light for 2 hrs. After incubation, membrane was washed with TBST 3× and image was taken on Li-COR Oddysey chemiluminescent imager.

Cell Viability: To quantify if the destabilized constructs affect the cell survival. Cell viability was tested comparing the wildtype cells with the cells carrying destabilized constructs with cell titre glo (Promega cat: G7570).

Phosphorylation kinase array: To determine how the kinases are affected by the destabilization of the c-MYC protein. The human phospho-kinase array kit (Cat no: ARY003C) was used according to the protocol.

RNA Seq: To confirm the loss of c-MYC on genome scale following the destabilization of the 3′UTR as well as to ascertain to what level the c-MYC interactome is affected by loss of c-MYC and its specificity compared to the wild type controls. RNA Seq was performed on the MDAMB231 RNA from 3′UTR MYC 1-18 compared to the wildtype and vector controls.

Quantitative Reverse Transcript PCR: To quantify the endogenous c-MYC expression changes upon destabilization of the c-MYC comparing the wildtype and the vector controls, quantitative PCR was performed using primers that target the endogenous c-MYC exon.

IO-nanocage packaging of the engineered destabilized 3′UTRMYC: 3,4-dihydroxyhydrocinnamic acid (DHCA) by Alfa Aesar, Manganese (II) acetate, oleylamine, oleic acid, and iron (II) perchlorate) were purchased from Sigma-Aldrich. p-Xylene, 1-ethyl-3-[3-(dimethylamino) propyl]carbodiimide (EDC), and N-hydroxysuccinimide (NHS) were purchased from Thermo Scientific. Tetrahydrofuran (THF), dimethylsulfoxide (DMSO), ferric chloride hexahydrate, and phosphate buffer saline (PBS) was purchased from Fisher Scientific. Cy7.5 dye was purchased from Lumiprobe.

Generation and structure analysis of DNA plasmid-IO-nanocage complexes: The IO-nanocages were constructed through a galvanic reaction in which manganese ions of template Mn 3 04 nanocubes are replaced by iron ions to form a lattice-structure with a hollow center, as shown in previous publications (Nano lett. 2016, 16, 7357-7363, (Nano Lett., 2016, 16, 7357-7363; J. Am. Chem. Soc., 2014, 136, 12552-5; Oncol. Rep., 2020, 43, 169-176; Nat. Commun., 2021, 12, 2903). Water-soluble IO-nanocages were engineered by capping with 3-(3,4-Dihydroxyphenyl) propionic acid (DHCA) before loading with the DNA plasmids containing the engineered destabilized c-MYC constructs as well as vector control only. DNA plasmids were mixed with IO-nanocages with the mass ratio of 1:1 for the complexation based on the concentration of DNA plasmids needed for sufficient efficacy with respect to the optimized IO-nanocage concentration for in vivo experiments.

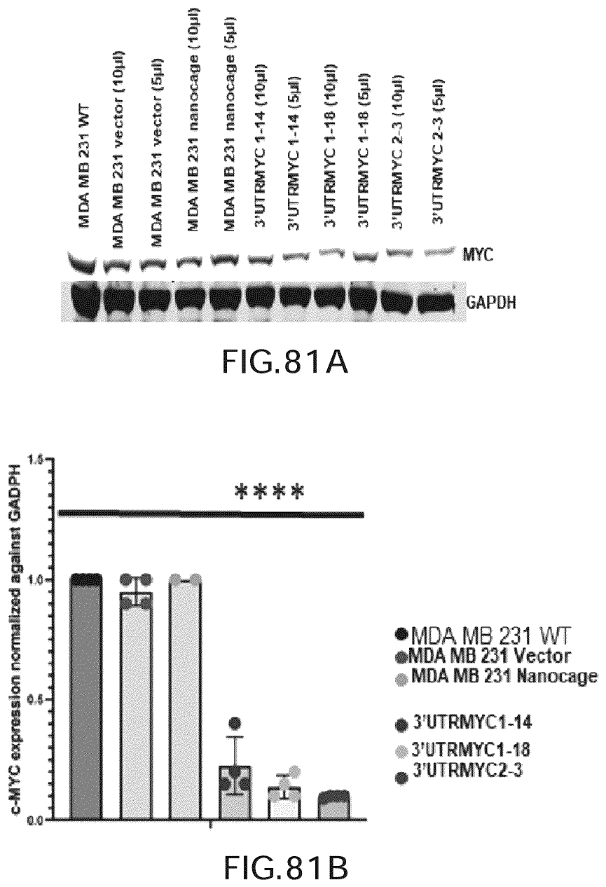

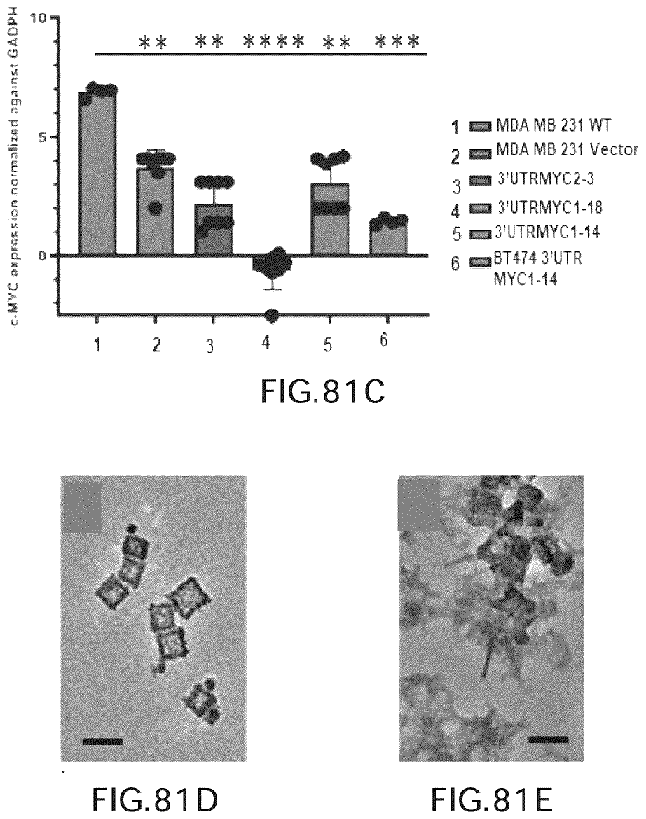

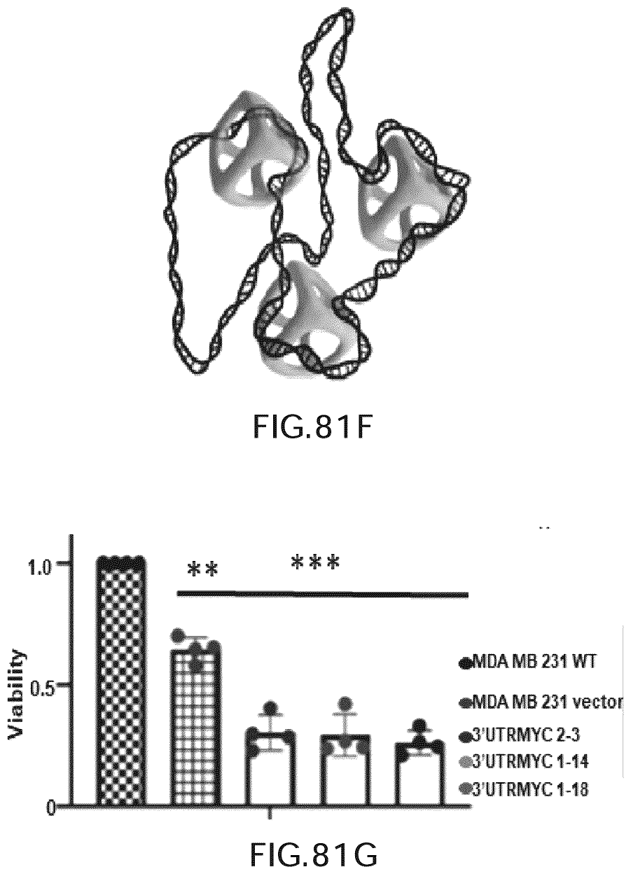

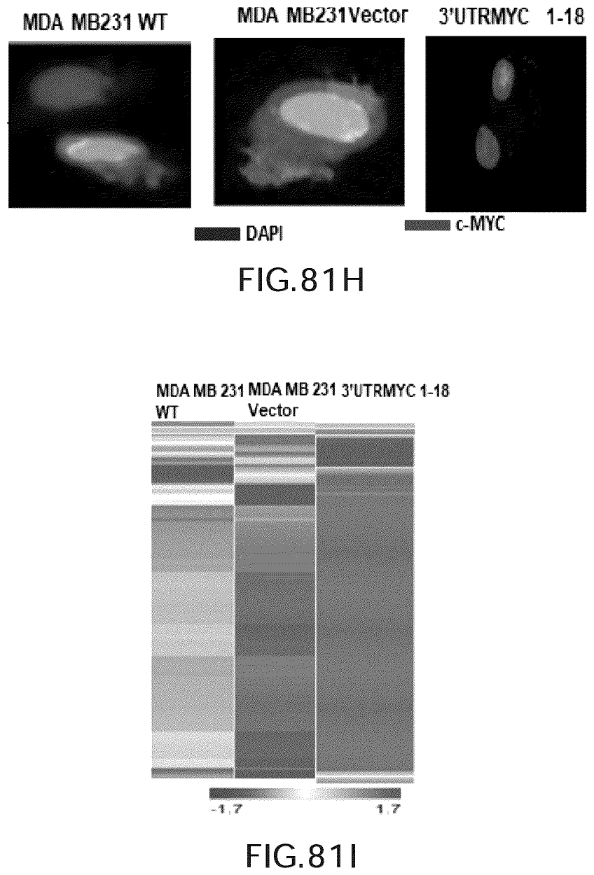

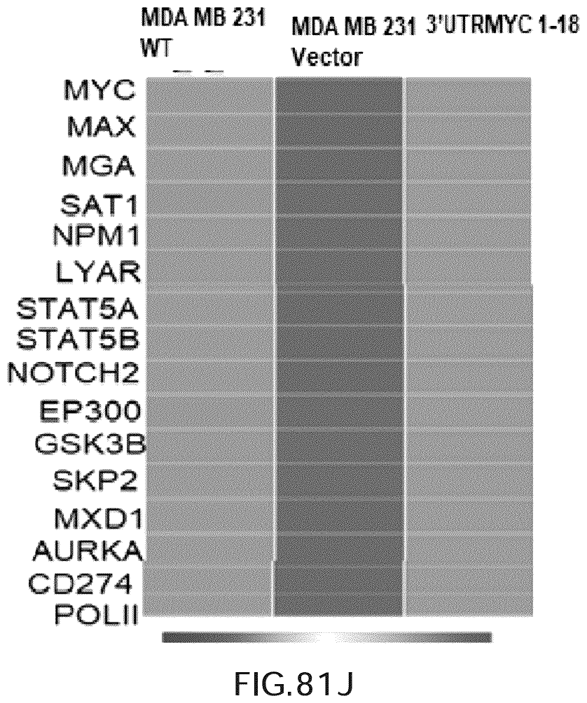

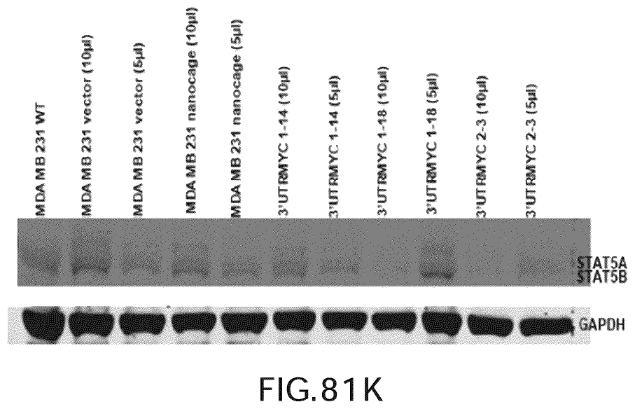

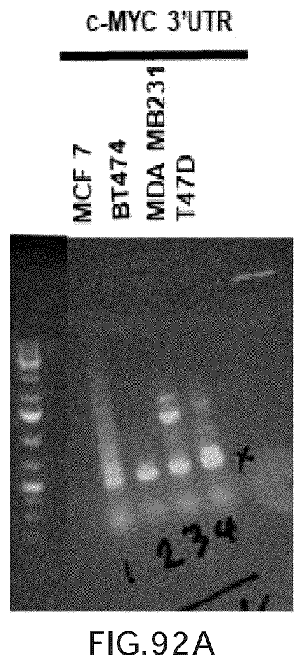

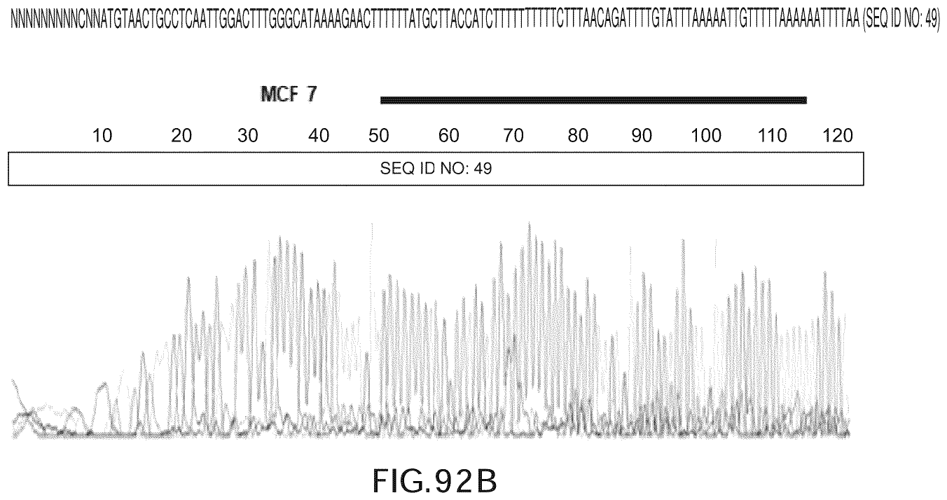

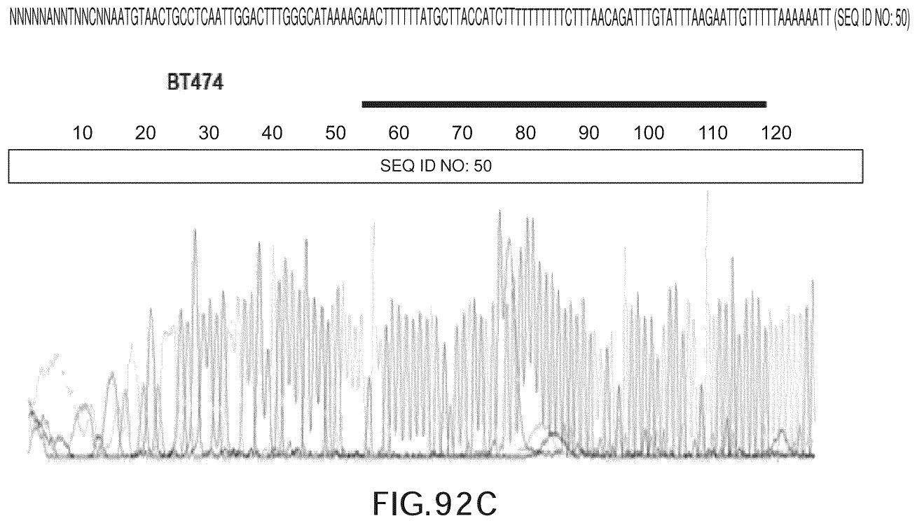

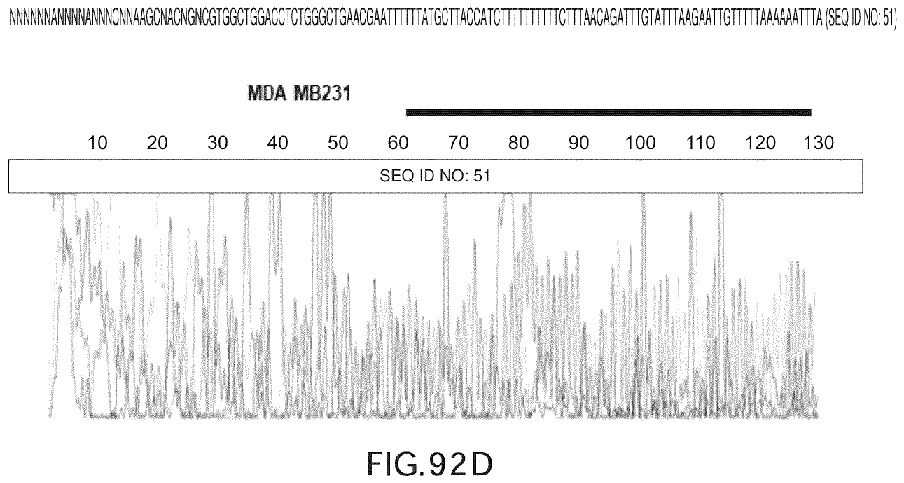

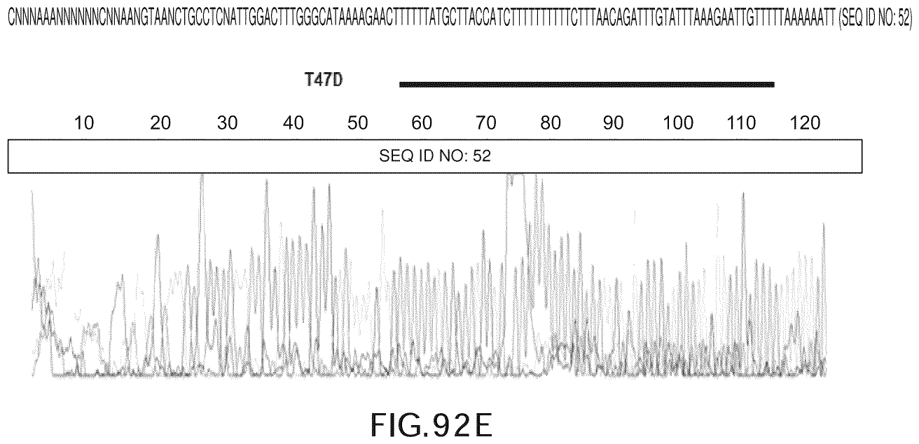

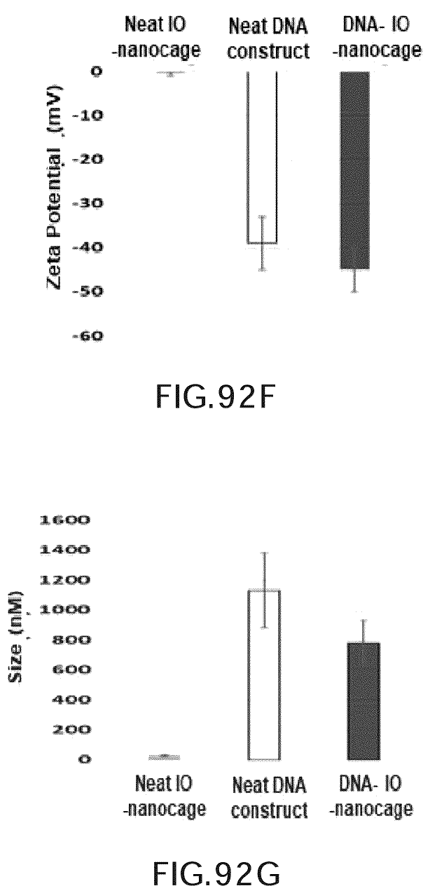

The structures of IO-nanocage and the DNA-loaded IO-nanocages generated by the protocol above were analyzed by transmission electron microscopy (TEM), dynamic light scattering (DLS), and zeta potential. The surface charge of the neat IO-nanocage was determined to be −0.5±0.4 mV with zeta potential measurement. When the IO-nanocage was loaded with DNA plasmids, the surface charge was decreased significantly to −45±5.0 mV, matching with the charge of neat DNA plasmid determined as −39±6.0 mV by zeta potential (FIG. 92F). Thus, the surface charge analysis indicates that DNA plasmid is certainly complexed with IO-nanocages. Due to existence of negatively charged carboxyl capping groups of DHCA on the exterior of the coated IO-nanocages with respect to the more neutral core, the charge gradient on IO-nanocages created by this configuration could also be attributed for the stable complexation between IO-nanocages and DNA plasmids. Furthermore, while the TEM image in (FIG. 81D) displays IO-nanocages with consistent cuboidal structure and well-defined lattice structure with little to no aggregation, the TEM image of the loaded IO-nanocages in (FIG. 81E, FIG. 81F) displays DNA plasmids wrapped around the surface of IO-nanocages, supporting the driving force of charge gradient for the stable complexation discussed above. According to DLS (FIG. 92G), the hydrodynamic diameter of the IO-nanocage was 28±5.0 nm with a polydispersity index (PDI) of 0.26, which is consistent with the size observed by TEM and the low PDI can be constituted as monodisperse. After loading with DNA plasmids, the size of IO-nanocage increased to 780±150 nm. As the size of neat DNA plasmids is much larger that the size of IO-nanocages, which is 1.306±0.25 μm by DLS, the size increase of IO-nanocages after the complexation with DNA plasmids is plausible.

Then, the IO-nanocages were capped with DHCA and transferred to the aqueous phase. First, 100 mg of DHCA was dissolved in 5 mL of THE in a three-neck flask (25 mL). The resulting solution was heated to 50° C. after bubbling for 30 seconds with flowing nitrogen gas. Then, 20 mg of IO-nanocages capped by oleic acid were dispersed in 1 mL of the THF which was added to the solution. The solution was heated to 50° C. for 3 hrs, and then cooled to room temperature, and 500 μL NaOH (0.5 M) was introduced to precipitate water-soluble IO-nanocages. The precipitate was collected by centrifugation and redispersed in 2 mL water, then dialyzed overnight with 10K MWCO membrane.

Complexation of DNA plasmids with IO-nanocages: To conjugate the DNA plasmids with IO-nanocages, these IO-nanocages were vortexed for even distribution of particles. The conjugation was completed with a 1:1 mass ratio between the DNA plasmid and IO-nanocage and the samples were incubated overnight at 4 C. The DNA plasmid complexation with IO-nanocages were then confirmed by TEM imaging, zeta potential, and DLS.

Structural Analysis of DNA plasmid-loaded IO-nanocages by TEM: The DNA-loaded IO-nanocages were then further processed for imaging using transmission electron microscope as described previously. Uranyl acetate, filtered using a 0.2 M filter with a 3 ml syringe, was used for staining DNA plasmids for TEM. After 10 l of the loaded IO-nanocage sample was placed on TEM grids for 10 minutes, 5 l of filtered uranyl acetate was added to these grids. After 45 seconds, the excess uranyl acetate was removed and added again on the grid for two more cycles. Finally, these samples were imaged by a JEOL JEM-2100 TEM at 200 kV with a LaB6 gun. Images were captured using 4 GB Gatan ultrascan camera model 994 US 1000XP and Digital Micrograph V.2.1.

Migration assay: Wounds were made on the cell monolayer using a sterile 200 μL pipet tip. Cells were washed with 1×PBS and fresh media were added to each well following the wound. Images of scratched areas were taken at 10× magnification using an AE30 inverted microscope (Motic, Richmond, BC, Canada).

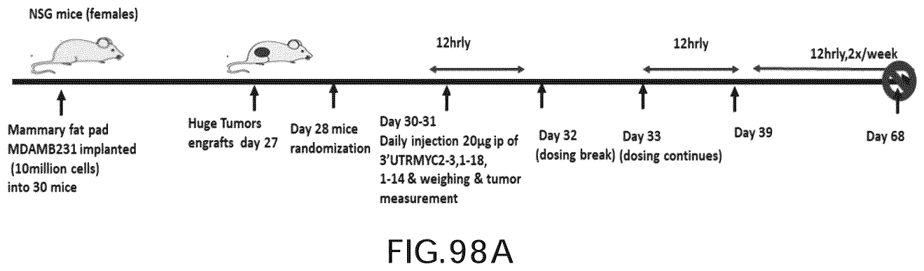

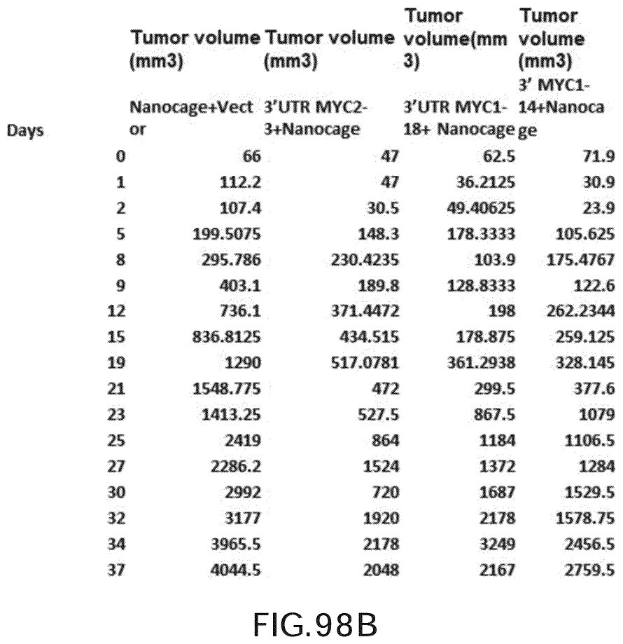

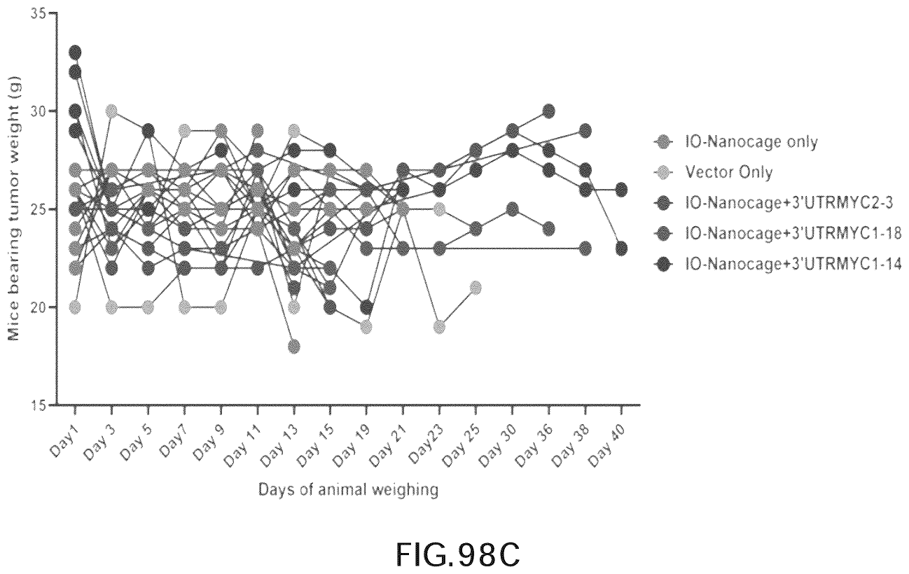

Mouse Animal Study: 30 female NSG mice were purchased from the Jackson laboratory. Animals were received and allowed to acclimate according to institutional protocol. MDA MB231 were expanded as described above and on the day of implantation, cells were harvested, washed, and resuspended in phosphate buffer saline (PBS). The confluency of the cells was 80%. Ten million cells were implanted in the matrigel in the mammary fat pad of each mouse. After 27 days, tumors engrafted and on 28 days, the mice were randomized into 5 mice per cage into 6 groups and equal distribution of tumor size within each group was ensured. The untreated, IO-nanocage only, vector in IO-nanocage and 3′UTRMYC2-3 in IO-nanocage, 3′UTRMYC1-14 in IO-nanocage and 3′UTRMYC1-18 on IO-nanocage were administered at 20 μg per mice intraperitoneally 12 hourly for 3 days with a one-day dosing break and dosing continued 12 hourly for another 6 days. Then dosing continued 29 days 12 hourly 2×/week till the end of the experiment on day 68 (FIG. 98A). Measurement of tumor size (length and width), weight and body condition score were obtained and recorded. Upon death of an animal, organs were harvested and stored in formalin. Pathological analyses were performed by experts from the Memorial Sloan Kettering Hospital, New York. The pathologists were blinded to the experimental details.

Animal subject demographics: Age of mice used: 7-8 weeks old mice were used. Weight of mice: mice weighed 18-22 g without tumors but with tumor in the mammary fat pad, mice weighed 20-34 g.

Power analysis: A total of 30 mice were randomized into 6 groups, at 5 mice per group (WT, IO-nanocage only, 3′UTRMYC2-3+IO-nanocage, 3′UTRMYC1-18+IO-nanocage, 3′UTRMYC1-14+IO-nanocage) of equal size. Based on pilot studies c-MYC driven prostate cancer, the tumor specific growth rate in mice is estimated to be 7.0±0.9 (%/day). A difference of at least 4 (%/day) between intervention and the empty vector control groups is of interest. With sample size 10 and α=0.05 significance level, one has 0.996 power to detect this difference with a one-sided two-sample t-test. To be conservative, a was adjusted using Bonferroni procedure to control the family-wise type I error at 0.05 for all of the potential comparisons, and still have a power of 0.98 to detect a difference of 2 (%/day) or more in tumor specific growth rate.

Exclusion and Inclusion criteria: All 30 mice implanted with tumors engrafted, no mice were excluded.

Tissue Immunofluorescence: Tissues slides were stained with anti-c-MYC and STAT5A/B. Briefly, slides were washed with PBS (1×) and primary antibody was added (c-MYC and STAT5A/B) at 1:1000 and dropped on the tissue to incubate overnight after which it was washed off and the secondary was added after 1 hr, it was washed and DAPI was dropped, and cover slip mounted, and the image was captured.

PDL-1 Immunohistochemistry: PDL-1 staining of the tumors and tissue were done by experts pathologist of the Memorial Sloan Kettering Cancer Center, Core pathology laboratory New York.

Prussian blue staining and detection of IO-nanocage in tissues: The Prussian Blue Staining on the tissue slides with and without the treatment of MYC-incorporated IO-nanocages was performed as follows. First, the tissue slides were deparaffinized with xylene for 10 min, 100% ethanol, for 9 min, 95% ethanol for 6 min, 70% ethanol for 3 min, and lastly hydrated with MilliQ water 5 min. Then, the tissue slides were placed in the Working Iron Staining Solution (Sigma Aldrich) for 30 min as the Working Iron Stain Solution was prepared by mixing equal volumes (100 mL) of potassium ferrocyanide solution and hydrochloric acid solution. The tissue slides were then collected and rinsed thoroughly in MilliQ water. The tissue slides were then placed in the Working pararosamiline solution (Sigma Aldrich) for 3-5 min as the Working Pararosamiline solution was prepared by mixing 1 mL of Pararosamiline Solution with 50 mL of MilliQ water. After the tissue slides were collected and rinsed thoroughly in MilliQ water, the tissue slides were then rapidly dehydrated through a series of alcohol treatment and cleaning with xylene as stated above before mounting. One drop of Permount Mounting Media (Fisher Scientific) was placed on the tissue slide and a cover slip was placed on top of the tissue slide for imaging.

4SU mRNA labeling pulse chase experiments: One million cells of the MDAMB231 WT, vector, 3′UTRMYC2-3, 3′UTRMYC1-18 and 3′UTRMYC1-14 were seeded in a 6 well plate and treated with 4SU at lug/ml and collected cells at 0 hr, 30 mins, 1 hr, 3 hr, 6 hr and 24 hr. RNA was collected, made into cDNA, and RT-qPCR was performed with primers targeting the endogenous c-MYC as well as primers targeting the DCP1A promoter of the vector and the cloned destabilized c-MYC, and control primer is ACTB. Delta Ct (Ct target endoc-MYC or exodestabilized c-MYC-CtActB) and fold enrichment was calculated as 0.5\hat{ }Ct.

Targeted sequencing to detect genomic sites of constructs integration: To determine the sites of the integration in the genome, the gDNA was extracted from tumors and tissues of the animals and then used the primers that can detect both the vector and destabilized constructs. This approach allows detection of the vector and the constructs together. The extracted gDNA was sent to Psomagen Inc. New York and the amplified reads were mapped back to the human genome to detect sites where they are integrated.

Statistical Analysis: All experiments were done in multiple replicates. The cell-based experiments were done more than 3× at the minimum as indicated on the legends. All animal data presented were performed as 5 mice in a cage per group. This includes the pathology work, H&E, PDL1 stain, c-MYC and STAT5A/5B stain. Spearman correlation was performed to establish correlation analysis. Two-tailed t-test was used to determine statistical significance. Log rank (Mantel-cox) test were used to determine statistical significance from the Kaplan Meier survival curve between treatment groups and control.

### Results

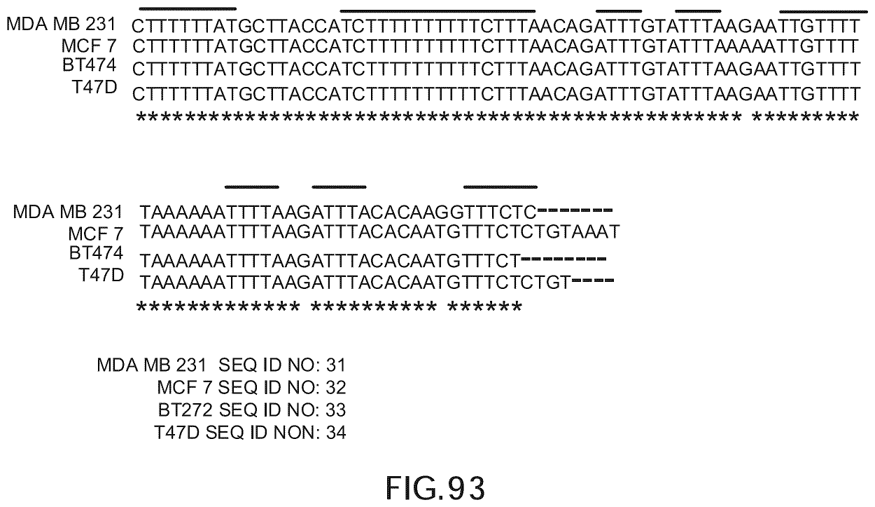

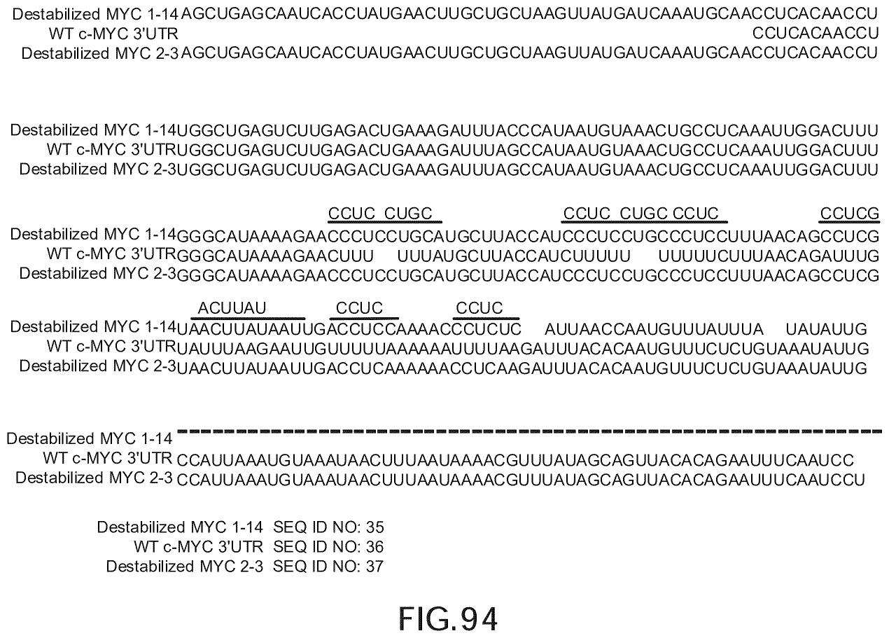

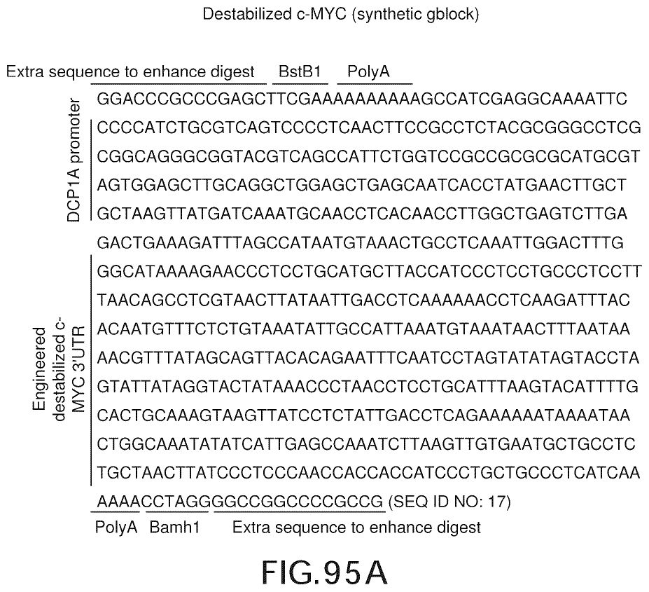

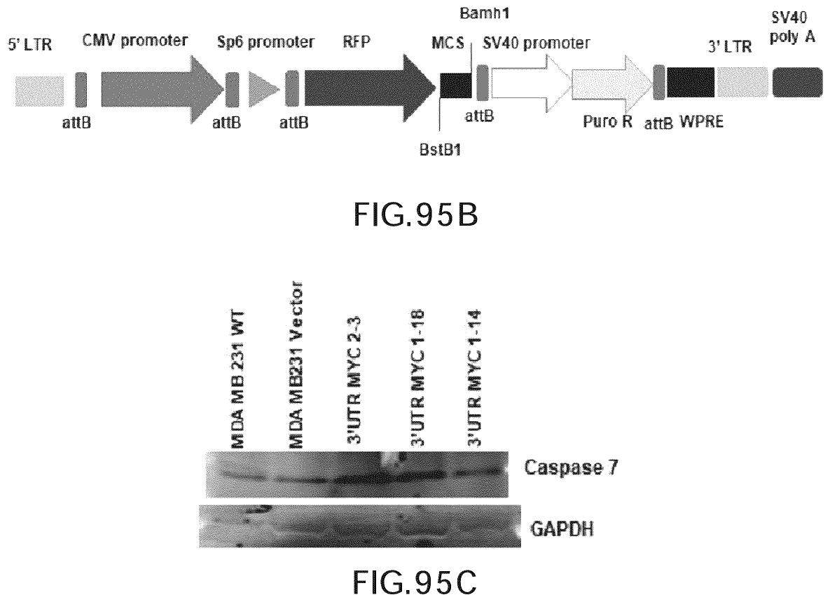

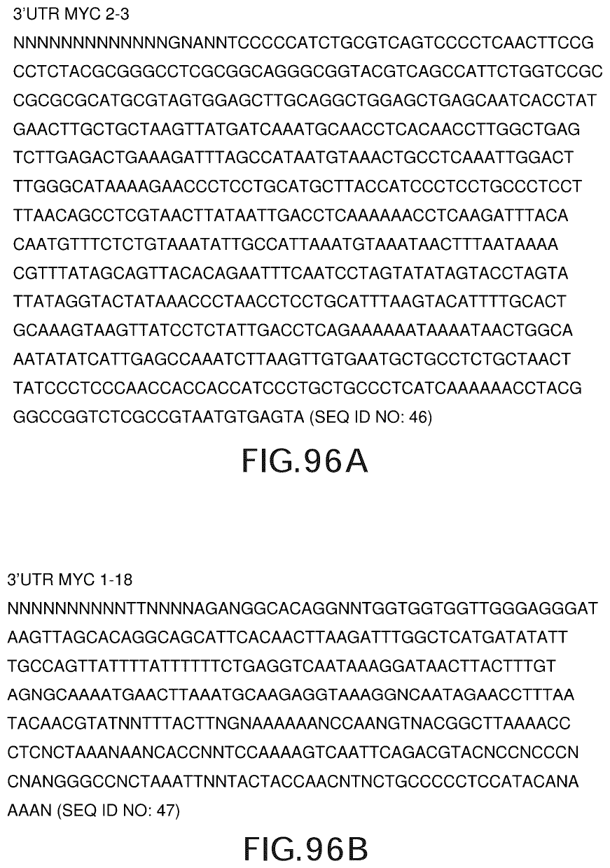

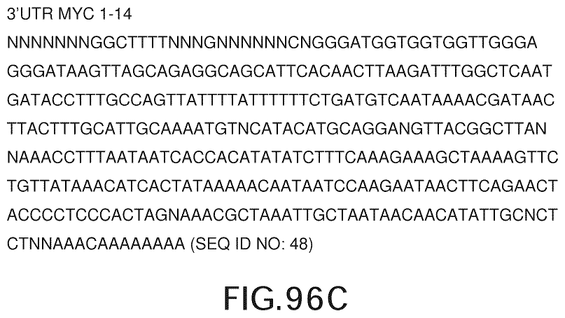

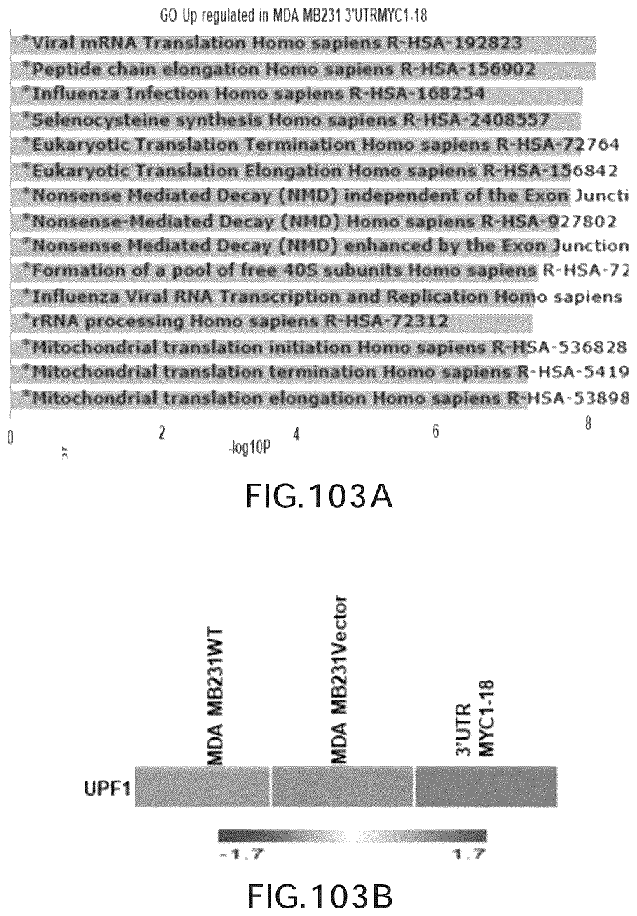

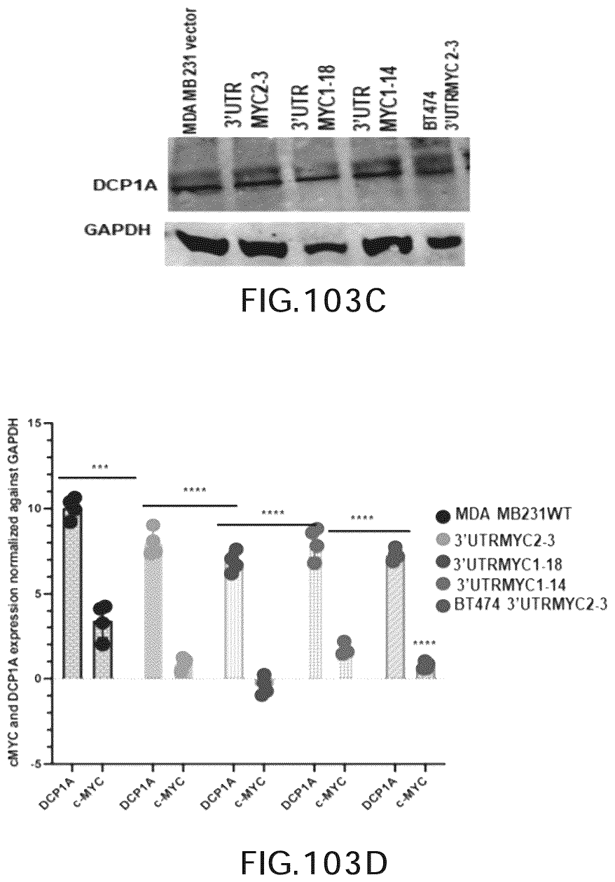

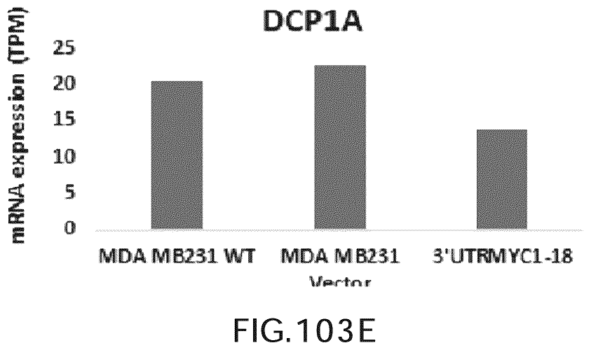

Identification of mRNA poly U sequences of c-MYC 3′UTR and design of the engineered destabilized 3′UTR of c-MYC constructs: Sanger sequencing identified poly U sequences of mRNA stabilizing elements in 3′UTR of the c-MYC gene that were 99% conserved across many breast cancer cell lines (FIG. 92A, FIG. 92B, FIG. 92C, FIG. 92D, FIG. 92E, FIG. 93). To generate the destabilized c-MYC 3′UTR, we replaced all the stabilizing elements with destabilizing elements, which are CCUC, CCUGC, ACUUAU (FIG. 94). These were then engineered to have these cDNAs under the control of the mRNA decapping enzyme gene (DCP1A) promoter (FIG. 95A, FIG. 95B) and created three c-MYC destabilizing constructs (3′UTRMYC2-3,1-18 and 1-14) (FIG. 96A, FIG. 96B, FIG. 96C). The logic behind the design was that if the MYC-3′UTR were destabilized and its expression were driven, that the MYC transcript and protein could be degraded specifically through nonsense-mediated decay by overwriting the endogenous messages with infidel exogenous destabilized transcript that was made at such a high copious amount. As designed, these constructs triggered nonsense-mediated decay (FIG. 103A, FIG. 103B, FIG. 103C, FIG. 103D, FIG. 103E).

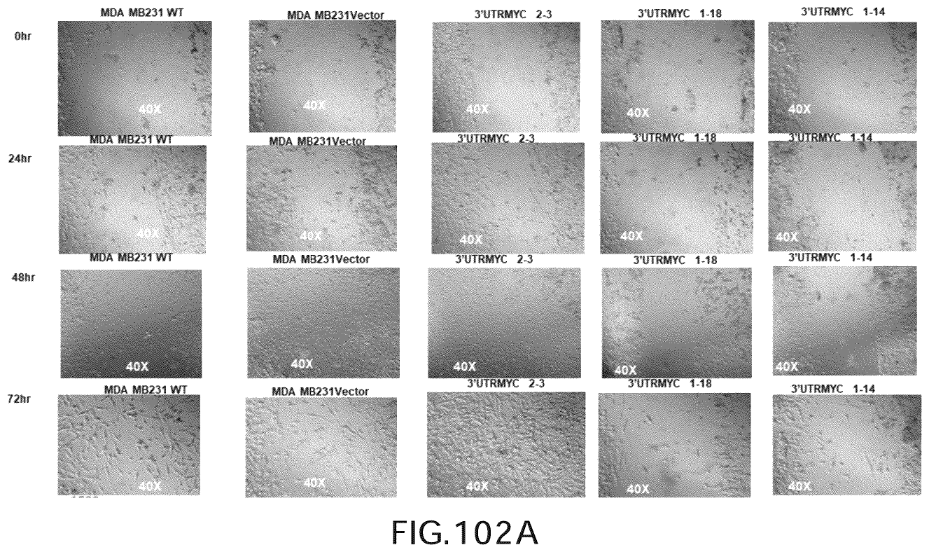

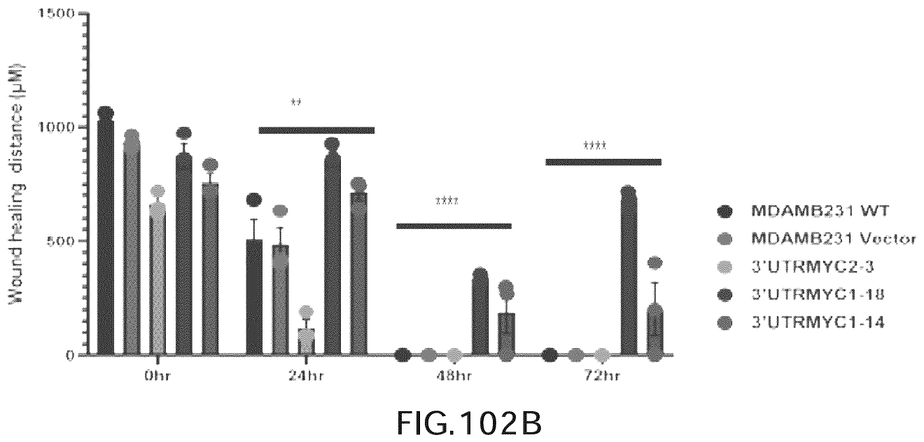

Engineered destabilized 3′UTR of c-MYC degrades c-MYC transcript and proteins in c-MYC driven TNBC: To validate whether the destabilized 3′UTR of c-MYC degrades c-MYC transcript and proteins in TNBC, after 4 days of incubation, c-MYC expression was assayed on these cells and found that all three constructs of 3′UTR (MYC 2-3,1-18,1-14) degraded c-MYC protein and transcript in MDA MB231 cells via IO-nanocage delivery vehicles as compared to their controls (FIG. 81A, FIG. 81B, FIG. 81C). IO-nanocages were complexed with the constructs and the vector respectively. Transmission electron microscopy (TEM) showed that DNA constructs and vectors were wrapped around IO-nanocages (FIG. 81D, FIG. 81E, FIG. 81F), and zeta potential of DNA construct-IO-nanocage complex shifted to highly negative value as compared to neat IO-nanocage due to the wrapping of negatively charged DNA constructs, consistent with the observation in TEM image (FIG. 92F). Dynamic light scattering (DLS) also revealed that the hydrodynamic size of DNA construct-IO-nanocage complex increased to 800 nm due to wrapping of DNA construct, while this hydrodynamic diameter is smaller than the one for neat DNA construct, indicating the compacting of construct on IO-nanocage (FIG. 92G). This degradation of c-MYC led to the significant loss of viability of these cells which received the constructs compared to controls (FIG. 81G). By immunofluorescence (FIG. 81H), destabilized 3′UTR of c-MYC construct degraded c-MYC (stained red and DAPI in blue, respectively) in the MDA MB231 cells after the treatment as compared to the controls. The loss of c-MYC increased active caspase 7 (FIG. 95C), leading to the impaired migration of these cancer cells (FIG. 102A, FIG. 102B).

To confirm on the genome scale the destabilization of c-MYC degraded the c-MYC transcript and its interactome, we performed RNA Seq of the wildtype c-MYC-driven cancers MDAMB231, vector and the 3′UTR MYC1-18. The destabilized c-MYC constructs degraded c-MYC mRNA expression (FIG. 81I) as well as c-MYC interacting partners with known high MYC binding site, MAX, MGA, SAT1, NPM1, LYAR, STAT5A/B, NOTCH2, NOTCH3, EP300, GSK3B, SKP2, MXD1, AURKA, CD274, POLII, WNT11, ASCL1, JUNB, NAT16, VEGFA, TLL1, TAL2, JUN, PDGFB, EGR1 in the cancer cells carrying the destabilized ARE 3′UTR MYC 1-18 (FIG. 81J). These findings strongly suggest that the disclosed technology reliably controlled c-MYC transcript, protein, and interactome.

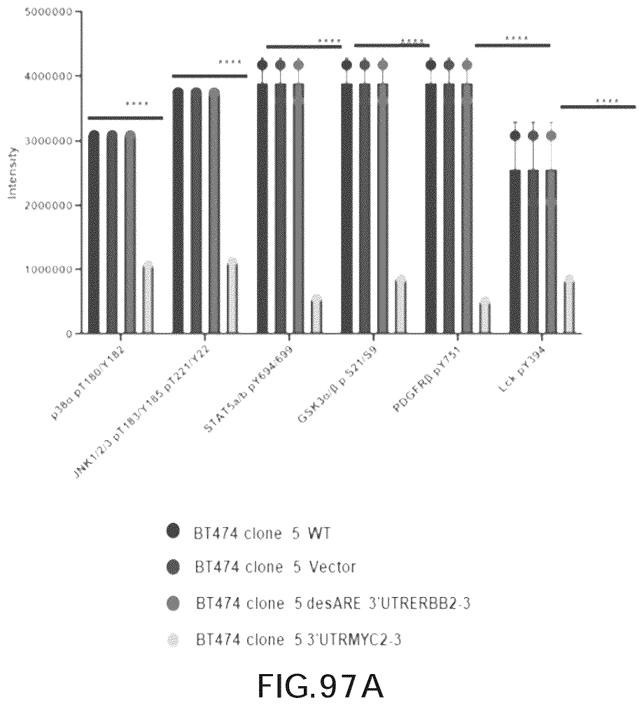

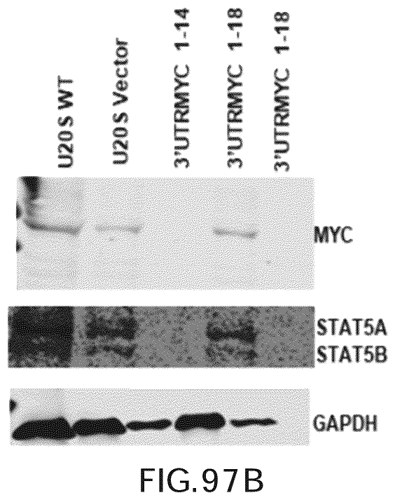

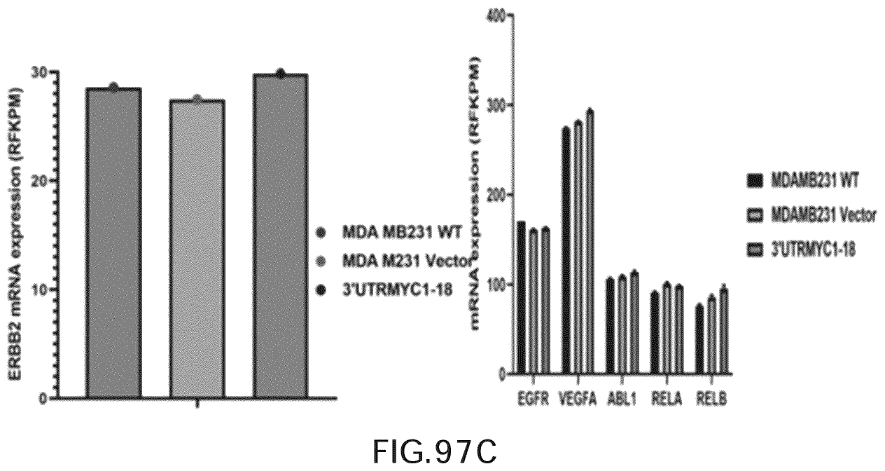

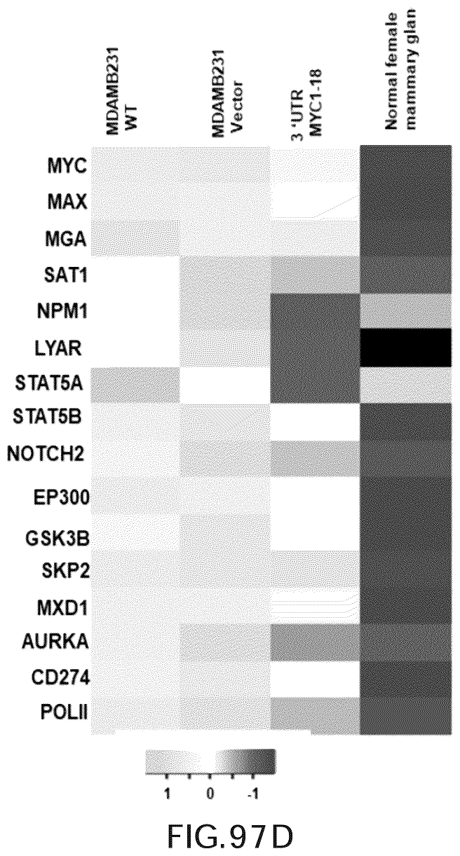

Certain kinases are known to be downregulated only upon the degradation of the c-MYC. These kinases are p38α pT180/Y182, JNK1/2/3 pT183/pY185 (18), pT221/pY22, GSK3α/β pS21/pS9, PDGFRβ pY751, LckpY394, STAT5A/B pY694/p699 are significantly downregulated compared to their controls (FIG. 97A). STAT5A/5B was chosen as there is paucity of information on c-MYC-STAT5A/5B interaction in cancers. To validate the STAT5A/B lost upon c-MYC destabilization, w MDA MB231 cells fed with nanocages carrying c-MYC destabilizing constructs were probed. The results showed that 3′UTRMYC1-18, and 3′UTRMYC2-3 show loss of STAT5A and STAT5B upon loss of c-MYC at day 4 (FIG. 81K). A trend emerges in that where there is loss or reduction of c-MYC upon destabilization, also, there is loss/reduction of STAT5A/B (FIG. 81J, FIG. 81K, FIG. 97A, FIG. 97B).

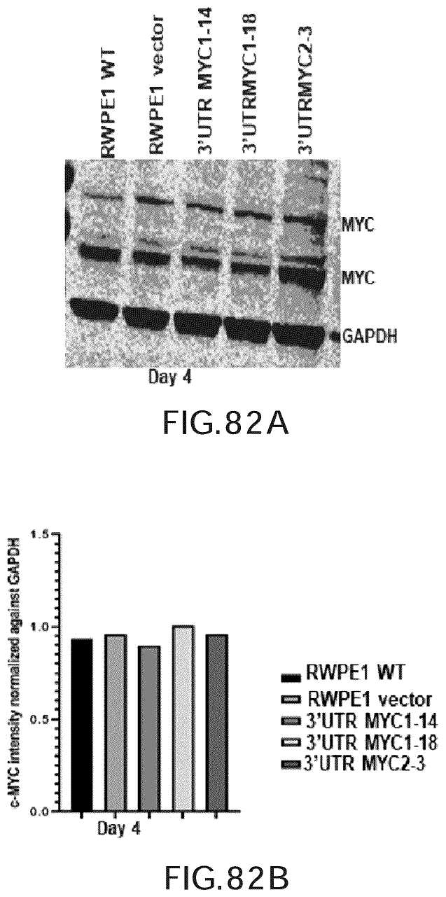

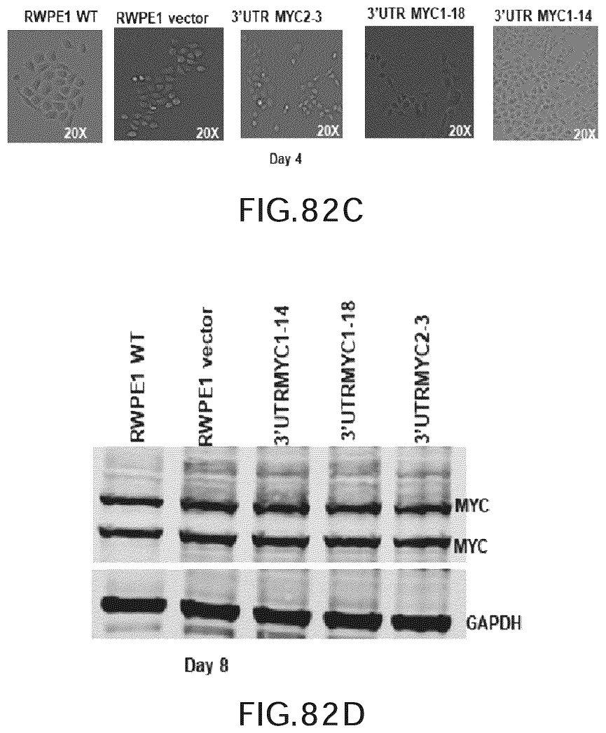

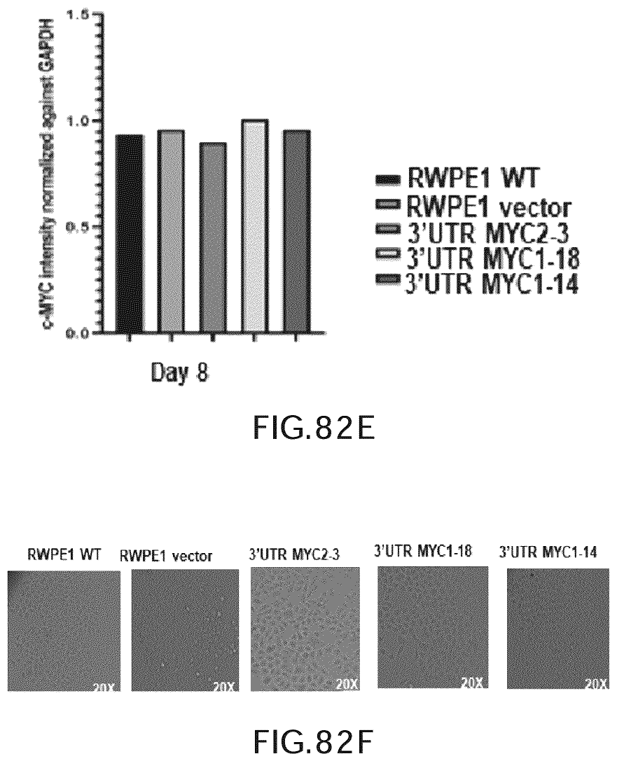

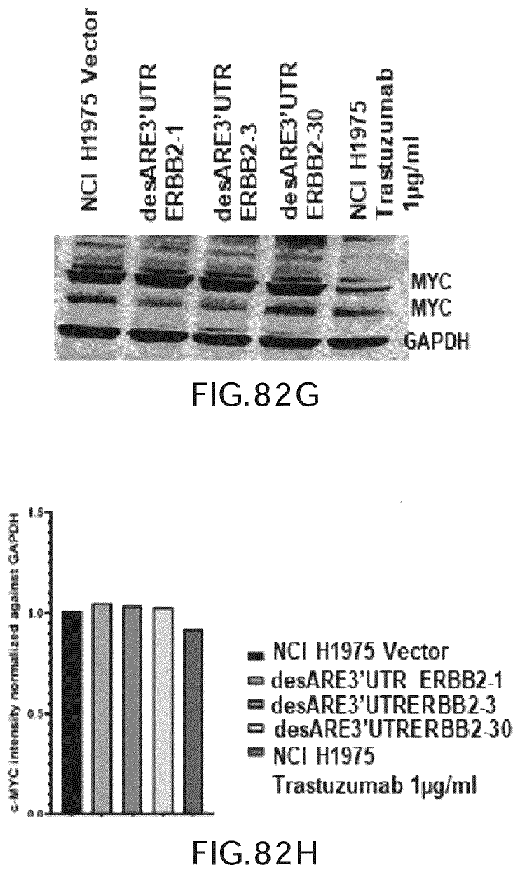

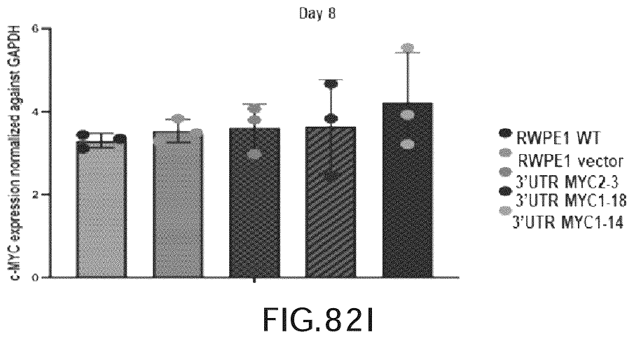

The engineered destabilized 3′UTR of c-MYC do not degrade MYC in normal cells: To prove that the engineered destabilized 3′UTR of c-MYC does not affect normal epithelial cells, the normal epithelial cells RWPE1 were transduced with the engineered destabilized ARE 3′UTR of c-MYC constructs. After 4- and 8-days independent experiments, western blot was performed and mRNA transcript analysis by RT-qPCR on the cells containing the constructs compared to the wildtype cells and vector control. No loss of c-MYC was found, both on transcript and protein level in the RWPE1 containing the constructs compared to the wildtype and vector control (FIG. 82A, FIG. 82B, FIG. 82C, FIG. 82D, FIG. 82E, FIG. 82F, FIG. 82I). Taken together, these results show that the engineered destabilized 3′UTR does not affect normal healthy cells. To confirm the specificity of engineered destabilized c-MYC 3′UTR to MYC and show that it does not affect other oncogenes, a test probed for loss c-MYC in NCI H1975 destabilized with engineered 3′UTR of ERBB2 that targets ERBB2. No loss of c-MYC in the desARE 3′UTR ERBB2 cells was found (FIG. 82G, FIG. 82H). Conversely, no loss of ERBB2, EGFR, ABL1, RELA and RELB transcript in c-MYC destabilized cells was found compared to controls (FIG. 97C) and the 3′UTRMYC1-18 treated cells tend to restore MYC expression towards the normal female mammary epithelial expression levels (FIG. 97D).

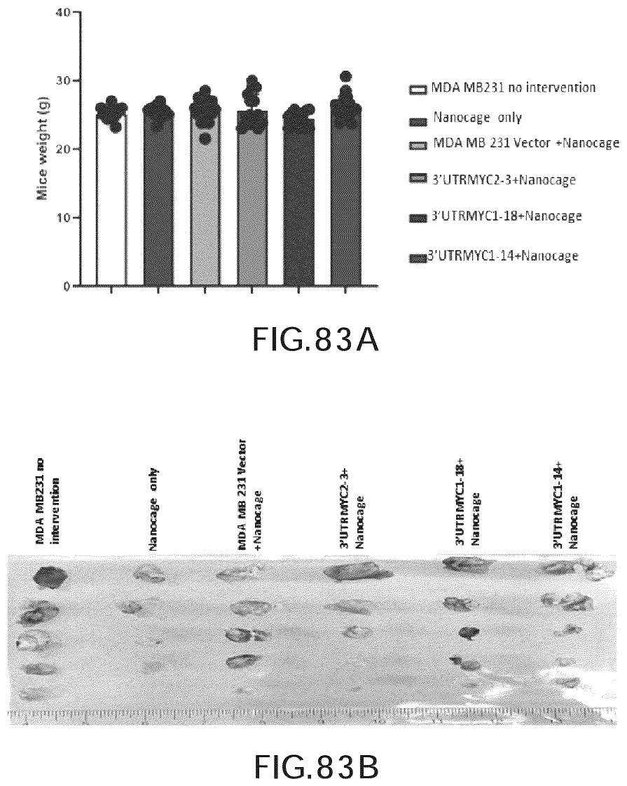

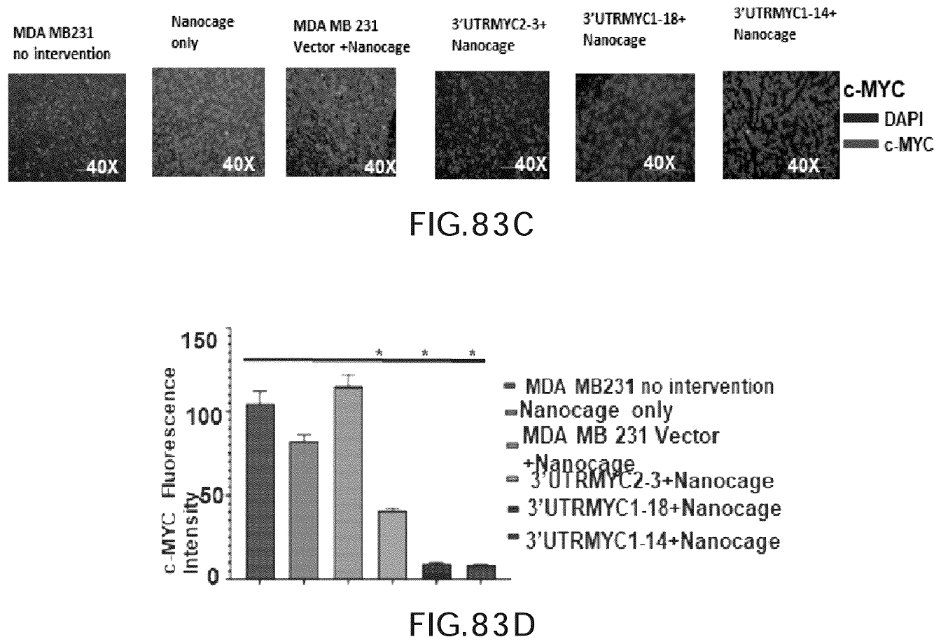

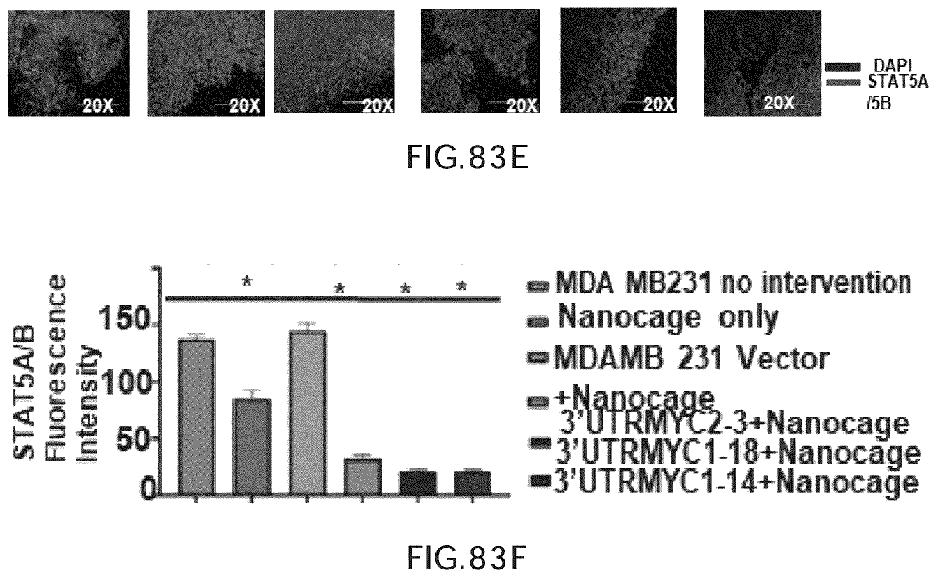

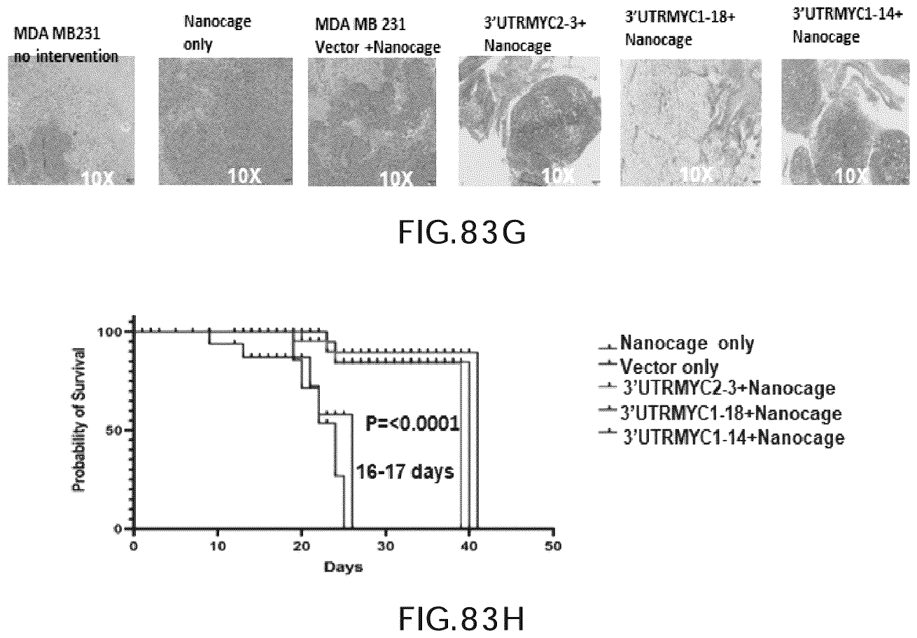

Engineered destabilized 3′UTR ARE degradation of c-MYC shows significant survival outcome and inhibition of primary and metastatic tumors in c-MYC driven TNBC in vivo: To prove in vivo that the constructs indeed inhibited tumor growth, 30 female mice were implanted with TNBC in their mammary fat pads. On the 27th day, the tumor engrafted, and they were randomized into 5 mice per IO-nanocage in 6 groups (FIG. 83A), namely MDA MB231 no intervention only, IO-nanocage only, IO-nanocage+vector, 3′UTR MYC2-3+IO-nanocage, 3′UTRMYC1-18+nanocage and 3′UTRMYC1-14+IO-nanocage. 20 μg of the constructs packaged in nanocages was administered intraperitoneally into the mice, first 12 hrly daily from day 30-39, then from day 40 12 hrly 2× per week till day 68 (FIG. 98A, FIG. 98B, FIG. 98C). No change in body weight was found comparing the control and treatment group (FIG. 83A, FIG. 98C). Tumors were collected and the treatment with the 3′UTRMYC1-18+nanocage caused 80% reduction of the tumor volume, the 3′UTRMYC2-3+nanocage and 3′UTRMYC1-14+nanocage reduced the tumor volume to 60% (FIG. 83B, FIG. 97B) compared to the no intervention and vector group. Survival analysis comparing the animals that received the constructs (3′UTRMYC2-3, 1-18 and 1-14) with the no intervention group (IO-nanocage and vector only groups) showed that the treated groups survived very significantly (p<0.0001), with 17 days more than the control groups (FIG. 83H). This finding strongly shows the therapeutic benefit of directly drugging MYC by this approach.

Loss of c-MYC in vivo leads to loss of STAT5A/5B in the responsive c-MYC driven TNBC: To confirm the finding in vitro that the destabilization and degradation of c-MYC leads to loss of c-MYC and its interacting partner STAT5A/5B in vivo, tumor tissues were stained from FIG. 83B with antibodies against c-MYC and STAT5A/5B respectively. The tumors that responded to the treatment with the constructs 3′UTRMYC2-3, 1-18 and 1-14 show loss of c-MYC and STAT5A/B (FIG. 83C, FIG. 83D, FIG. 83E, FIG. 83F) compared to the untreated controls. The loss of c-MYC was most profound in the tumors that received the constructs 3′UTRMYC1-18 and 1-14. Next, to ascertain to what extent the constructs controlled the TNBC tumors, a H& E (Haematoxylin and Eosin) stain was performed on the tumors that showed constructs 3′UTRMYC1-18 almost completely eradicated the tumors histologically compared to any group (FIG. 83G). Taken together, this demonstrates that the loss of c-MYC led to the loss STAT5A/5B in vivo validating the mRNA data and the construct were effective, especially that the 3′UTRMYC1-18 completely histologically inhibited the deadly c-MYC driven TNBC.

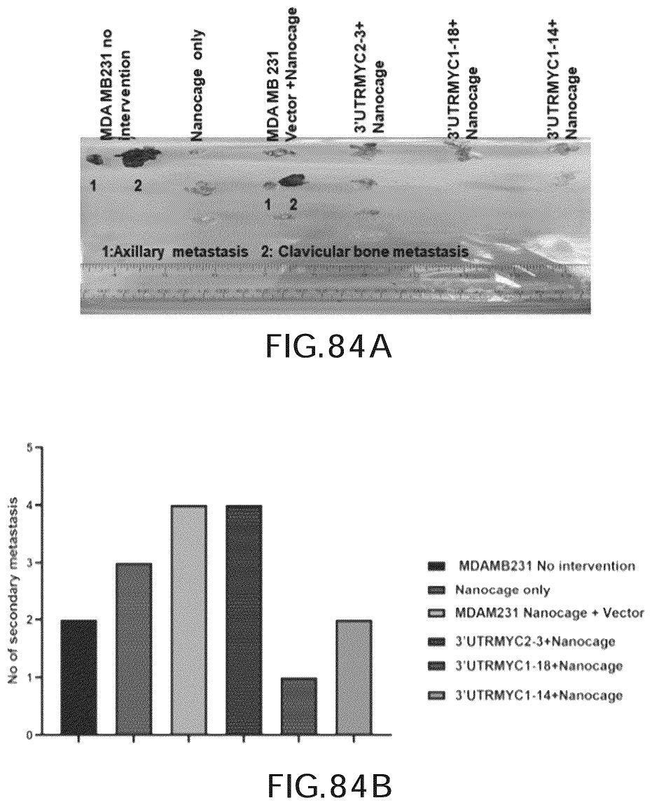

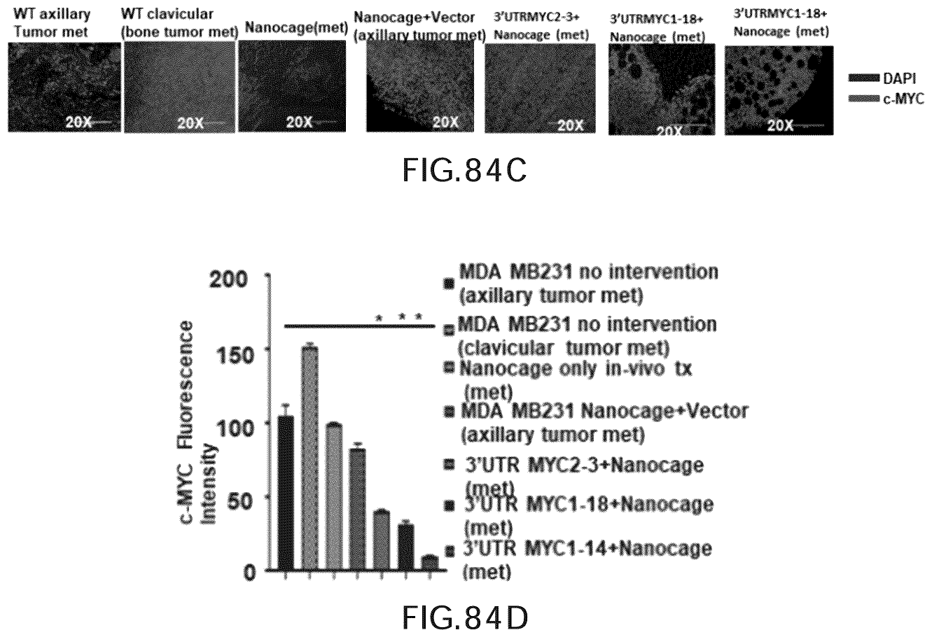

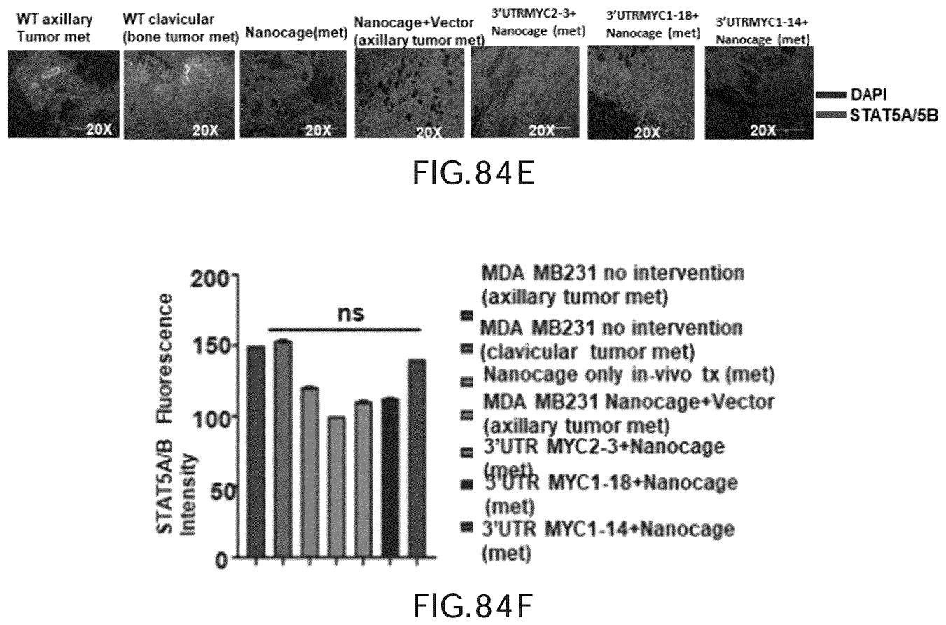

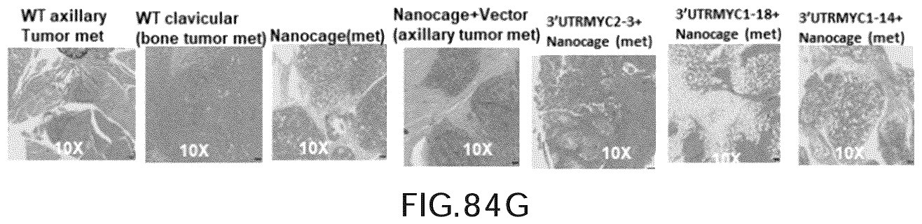

Constructs 3′UTRMYC1-18 and 14 inhibited 80% and 60% metastatic TNBC by downregulating c-MYC but the unresponsive tumors are driven by c-MYC independent STAT5A/5B expression: Metastatic TNBC is deadly, and the engineered destabilized c-MYC constructs inhibited metastasis. In the 3′UTRMYC1-18 and 1-14 treated tumor bearing animals that 80% and 60% of metastatic tumors were inhibited respectively (FIG. 84A, FIG. 84B). Next, to understand why these constructs inhibited metastasis but the other groups and controls did not. The tissues were stained with antibodies against c-MYC and STAT5A/5B and the results showed that, in tissues where metastasis was significantly inhibited, they lost c-MYC compared to the controls (FIG. 84C, FIG. 84D). However, when stained for the STAT5A/5B, they have robust expression of the protein (FIG. 84E, FIG. 84F). This led to the conclusion that the tumors with inhibited metastasis were those in which c-MYC-STAT5A/B interaction were controlled. But, for 20% and 40% of the tumors (FIG. 84G) that were not responsive to the 3′UTRMYC1-18 and 1-14 treatment respectively, they are exclusively under STAT5A/5B expression that is independent of the c-MYC. This result agrees with the findings of c-MYC and STAT5A/B super enhancer interaction in tumorigenesis and implicates STAT5A/B as driver of metastasis in TNBC for 20-40% tumors that escaped our c-MYC dependent treatment.

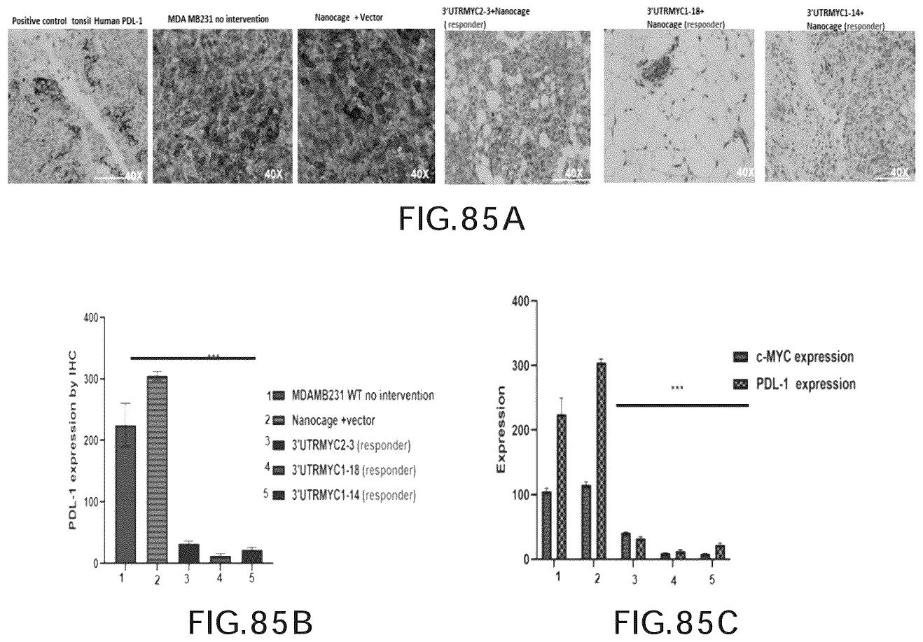

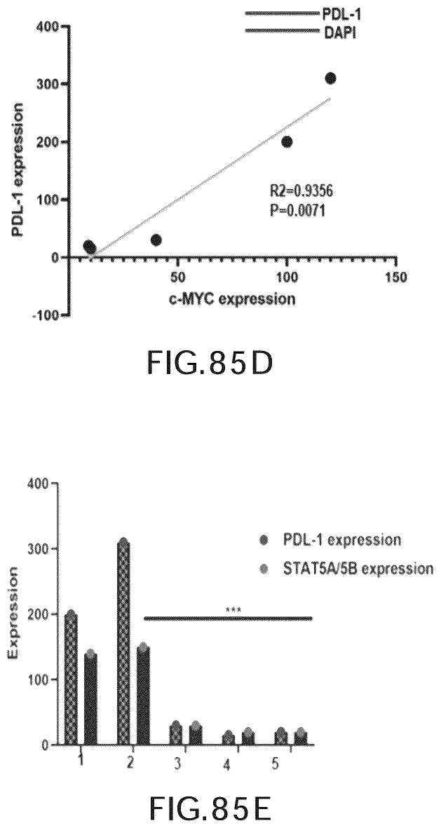

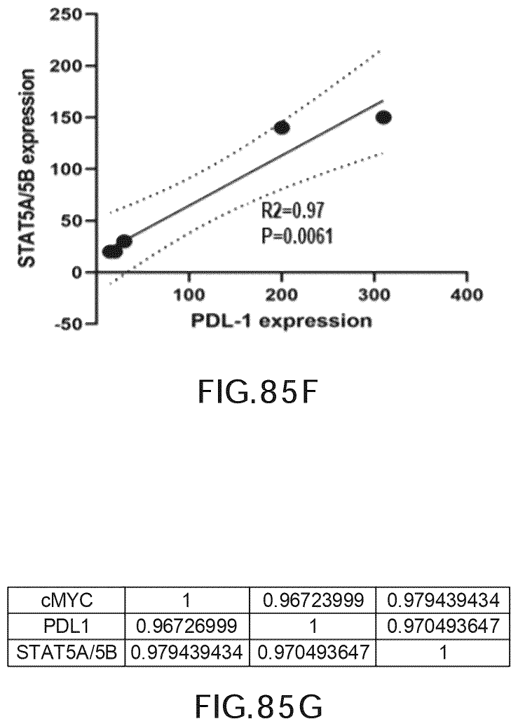

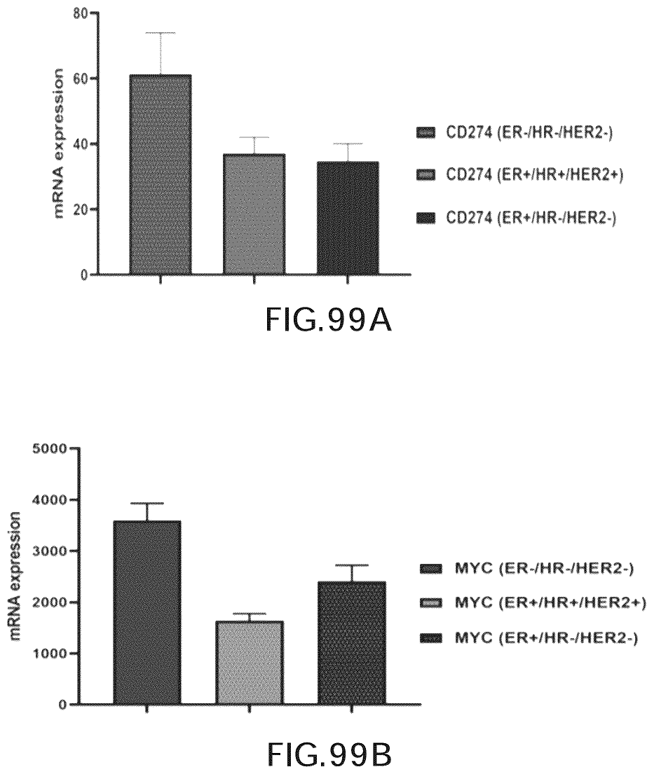

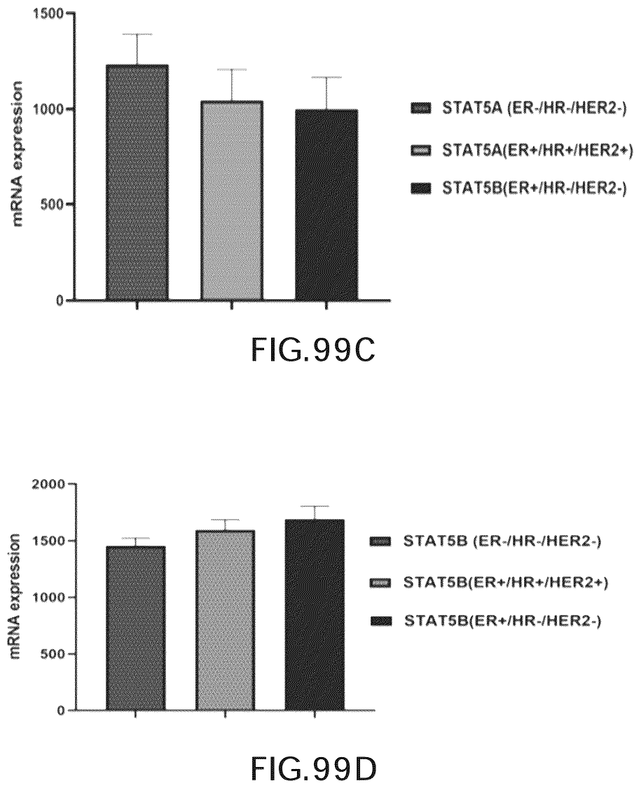

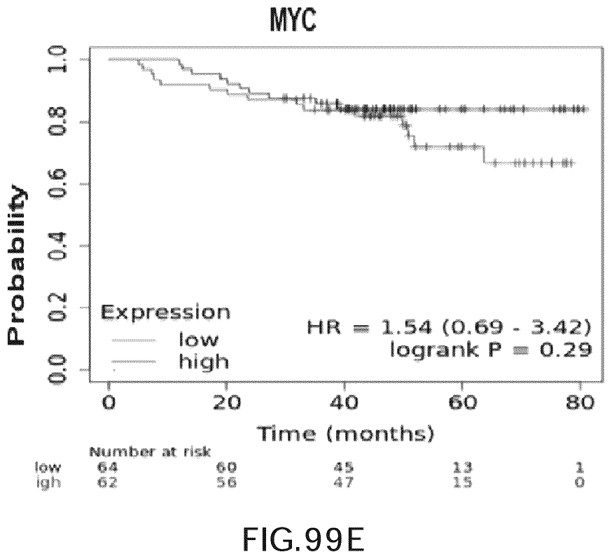

The destabilization and degradation of c-MYC led to the loss of PDL-1 in c-MYC driven TNBC: The PDL-1 is a “don't find me marker” that the tumor cells use to evade immune cells. The PDL-1 expression inhibition in tumors is an intense area of interest and immune checkpoint blockade therapy. There is significant interest into understanding genes that regulates PDL-1 expression as a target for cancer therapy. The Cancer Genome Atlas (TCGA) data was analyzed and could show that TNBC have a high level of c-MYC, PDL-1 and STAT5A/5B expression (FIG. 99A, FIG. 99B, FIG. 99C, FIG. 99D, FIG. 99E). Upon the destabilization of c-MYC transcript by the construct 3′UTRMYC1-18 (FIG. 81I, FIG. 81J) loss of CD274 (PDL-1) was found. The primary TNBC tumors were treated and controls were stained with anti-PDL-1 by immunohistochemistry, the positive PDL-1 control were normal human tonsil. Near total loss of PDL-1 was found in the tumor bearing animals that received 3′UTRMYC1-18 (FIG. 85A) and mild levels of PDL-1 in 3′UTRMYC2-3 and 1-14 treated tumors (FIG. 85A) whereas the untreated controls have very high level of PDL-1 (FIG. 85A). Quantification of the PDL-1 in the treated versus untreated controls show that the PDL-1 is significantly reduced in the treated group compared to the controls (FIG. 85B). Further, c-MYC and PDL-1 expression was quantified in head-to-head comparison between the treated and untreated group. Loss of PDL-1 in the treated follows the loss c-MYC in the same tumors in the treated group and, in the untreated groups with high c-MYC expression, also have high PDL-1 expression (FIG. 85C). Pearson correlation analysis shows a linear relation between c-MYC and PDL-1 expression at high (P-val=0.0071, Pearson correlation test, R²=0.925, FIG. 85D). This finding clearly demonstrates that the loss of c-MYC leads to loss PDL-1 following treatment, suggesting that c-MYC may control PDL-1 expression. It is already established that c-MYC binds on the promoter of PDL-1 to control it. The findings herein agree with that and demonstrate that the direct destabilization and degradation of c-MYC both at the transcript and protein levels lead to loss of PDL-1 transcript and protein.

While c-MYC is known to regulate PDL-1, it is of interest to know other regulators of PDL-1. This disclosure established that c-MYC-STAT5A/B interaction is lost in tumors that responded to this therapy. There may also be a correlation between STAT5A/B and PDL-1. The expression STAT5A/5B and PDL-1 expression was quantified in a head-to-head comparison between the treated and untreated tumors. The loss of STAT5A/5B in the treated responsive tumors follows loss of PDL-1 (FIG. 85E), and again in the untreated controls STAT5A/5B and PDL-1 expression were concomitantly high (FIG. 85E). A Pearson correlation analysis was performed between STAT5A/5B and PDL-1 expression and found high (P-val=0.0061, Pearson correlation test, R²=0.97, FIG. 85F). This finding could be interpreted as a strong functional relationship between PDL-1 and STAT5A/5B and could support that STAT5A/5B might be a regulator of PDL-1. The analysis was extended to ascertain if there is a functional correlation between c-MYC-STAT5A/5B-PDL-1 in the responsive tumors to our therapy. Multivariate correlation analysis indeed supports a functional relationship between the three molecules at (R²=0.97, FIG. 85G), in which t a higher complex of c-MYC-STAT5A/5B might be regulating PDL-1 expression by binding on its promoter.

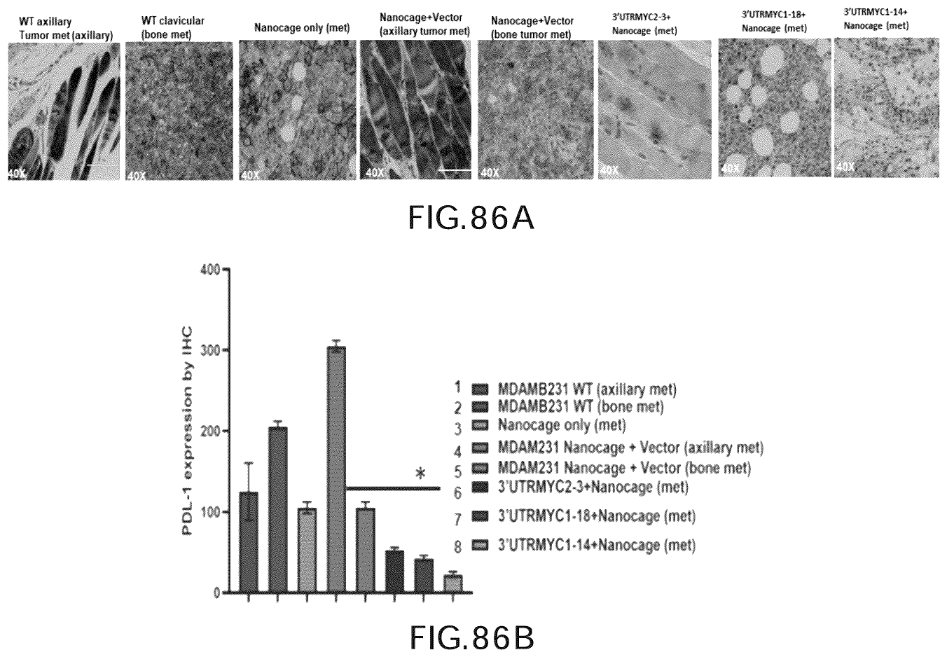

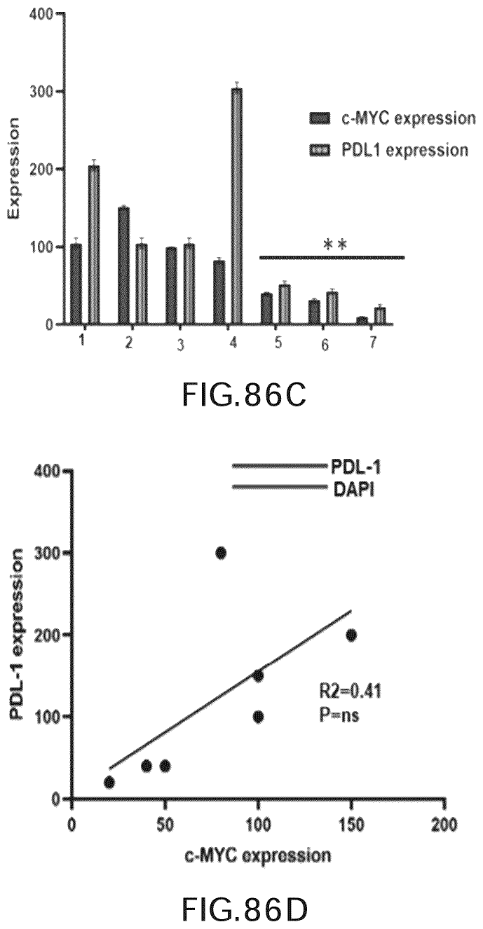

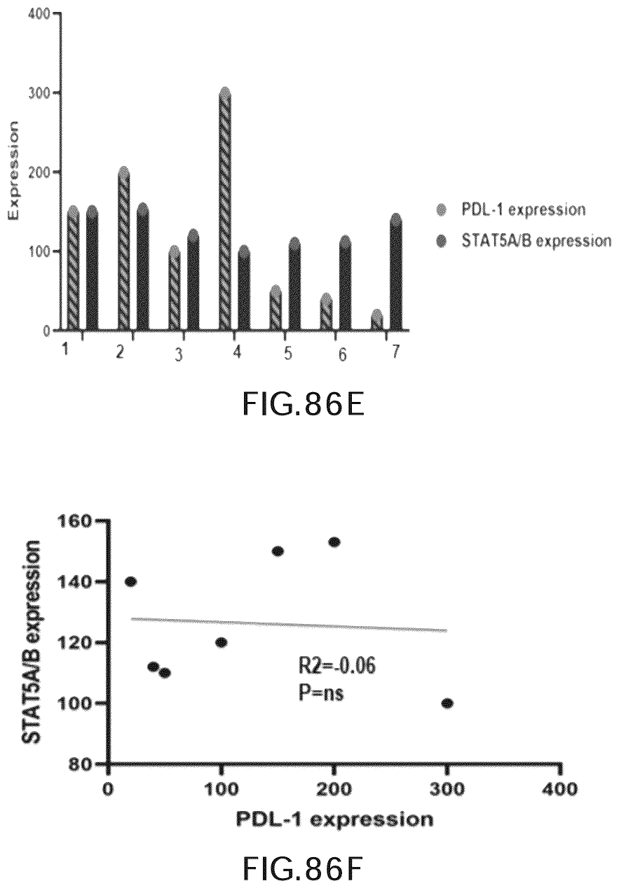

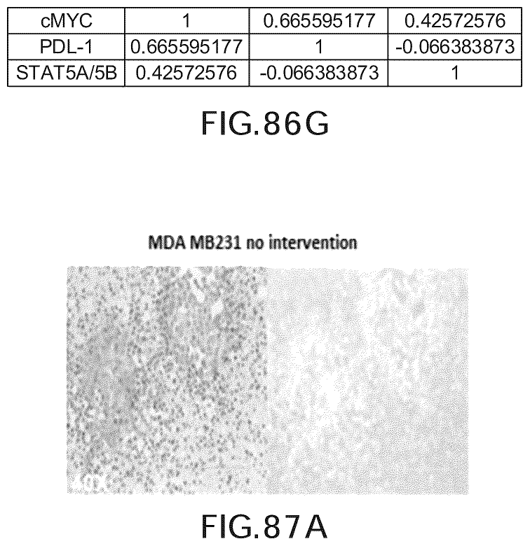

Non-responsive Metastatic TNBC are driven by c-MYC independent STAT5A/5B and PDL-1 expression: Having established the potential molecular interaction between c-MYC-STAT5A/5B-PDL-1 interaction on the c-MYC driven TNBC that responded to the disclosed therapy. Next, the molecular pathways that are active on the non-responsive metastatic TNBC were investigated. The metastatic tumors were stained with anti-PDL-1 and the metastatic tumors from the untreated groups had very high to moderate levels of PDL-1 expression, while those from the treated groups had mild to low levels of PDL-1 expression (FIG. 86A, FIG. 86B). Subsequently, to understand the correlation between c-MYC and PDL-1 in these metastatic tissues, the expression of c-MYC and PDL-1 was compared. There is no correlation between these molecules in the metastatic tissues (FIG. 86C, FIG. 86D). There is no functional correlation between STAT5A/5B and PDL-1 expressions in the metastatic tissues (FIG. 86E, FIG. 86F). Lastly, a less strong correlation was found between c-MYC-STAT5A/5B and PDL-1 expressions in the metastatic tissues than the responsive primary tumor (FIG. 86G). These results suggest that the metastatic tissues are not driven by the c-MYC-STAT5A/5B-PDL-1 interaction found in the primary tumors, but rather by independent STAT5A/5B+. It is worthwhile to mention that the metastatic tissue from the treated group showed mild to low PDL-1 expression, which agreed with the earlier results that c-MYC has been effectively targeted in them via the reduction of PDL-1 levels, and these tissues were metastatic due to STAT5A/5B independent signal for which our therapy was not designed for.

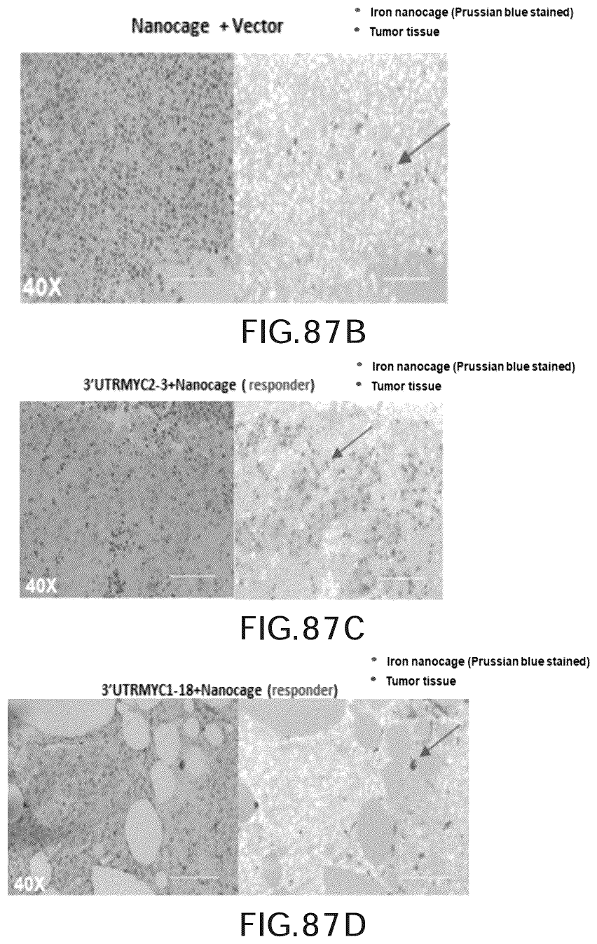

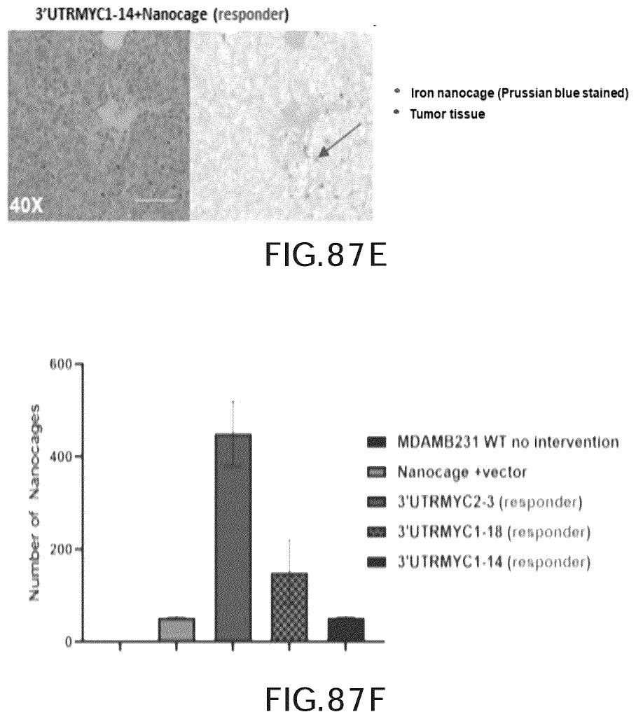

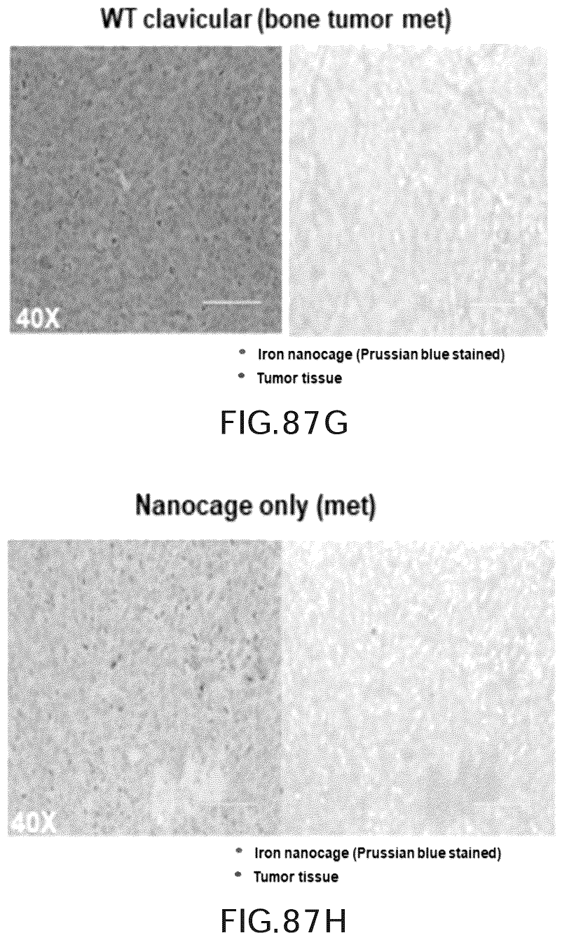

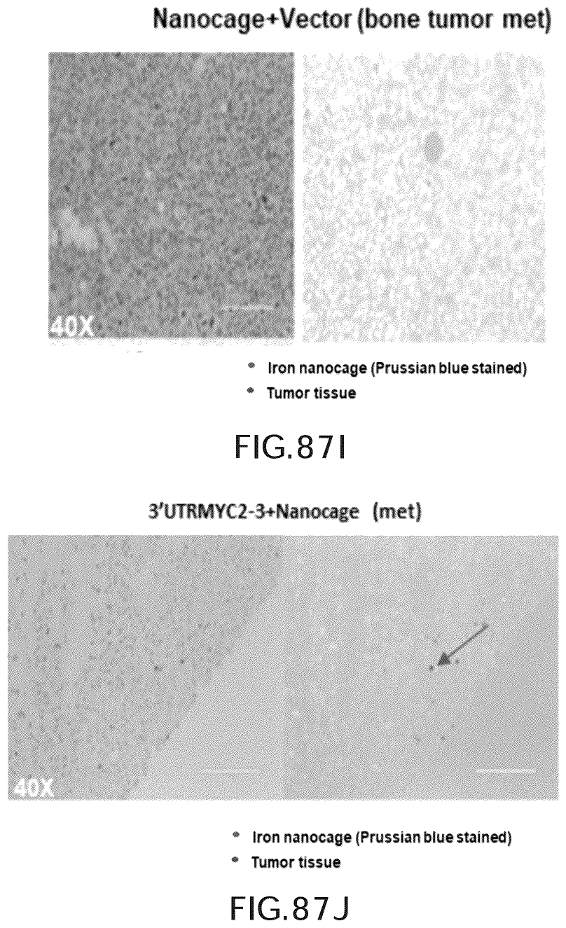

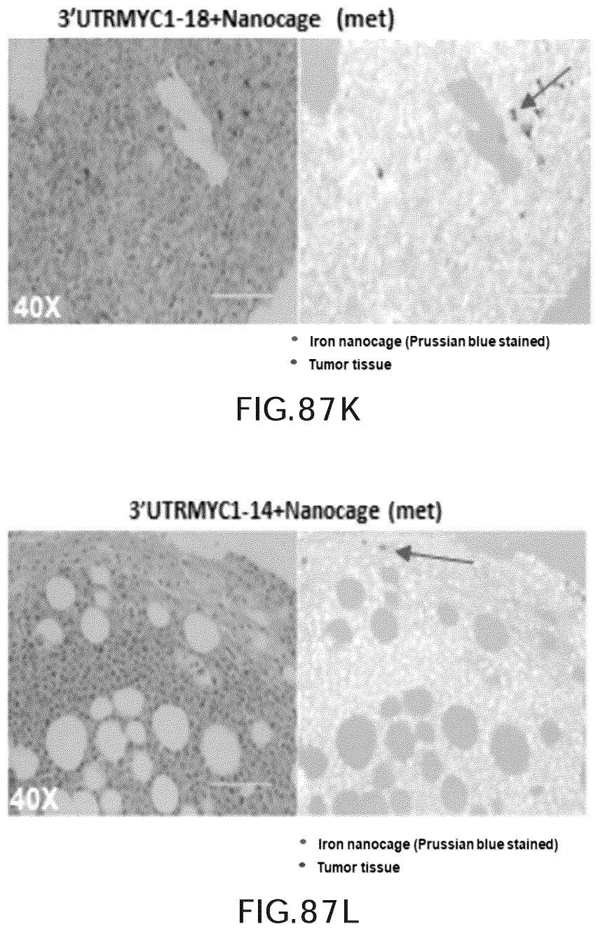

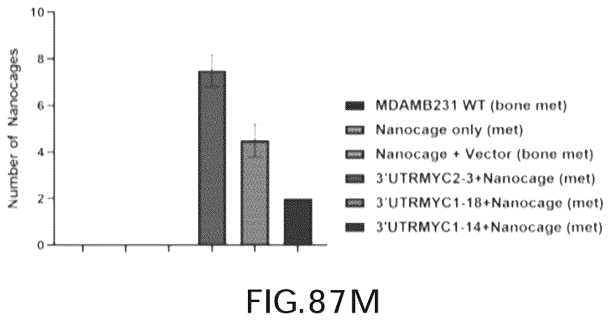

IO-nanocages delivered destabilized c-MYC constructs to tumors leading to effective targeting of c-MYC in the responsive TNBC: Many excellent therapies are limited by the ineffectiveness of delivery vehicles. IO-nanocages have effectively delivered therapeutic molecules and RNAs to tumor sites. In this report, the c-MYC constructs were packaged in IO-nanocages and achieved therapeutic destabilization and degradation of c-MYC (FIGS. 81A to 6G), and this IO-nanocage-destabilizing construct complex were used in animal studies in FIGS. 83A to 84F). First, to determine whether these nanocages delivered the constructs to tumors, the tumor tissues were stained with Prussian blue which labeled Fe elements. By comparing the positive control primary tumors treated by IO-nanocages with vectors (FIG. 87B) with the negative control (non-treated tumors) (FIG. 87A), the constructs 3′UTRMYC2-3, 1-18 and 1-14 were found to be robustly delivered into the tumors and responded to the treatment (FIG. 87C, FIG. 87D, FIG. 87E) with relative abundance (FIG. 87F). When the analysis was extended to the metastatic tumors between treated and untreated groups, no IO-nanocages in the metastatic tissues from the control groups including WT clavicular bone met (FIG. 87G), the met treated by only IO-nanocages (FIG. 87H), bone tumor met treated by IO-nanocages with vectors (FIG. 87I). In the metastatic tumors from the treated group, very little amount of IO-nanocages was found in these tissues (FIG. 87J, FIG. 87K, FIG. 87L), also quantified in FIG. 87M. These outcomes are consistent with the outcome that IO-nanocages could successfully deliver the therapeutic cargo and show the inhibition of robust tumor growth, and these delivered constructs could target and reduce tumor and metastasis sites.

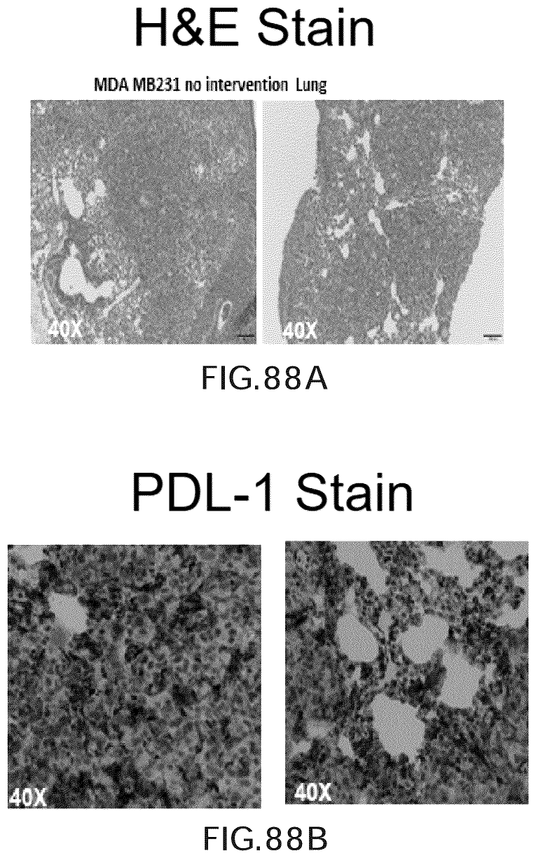

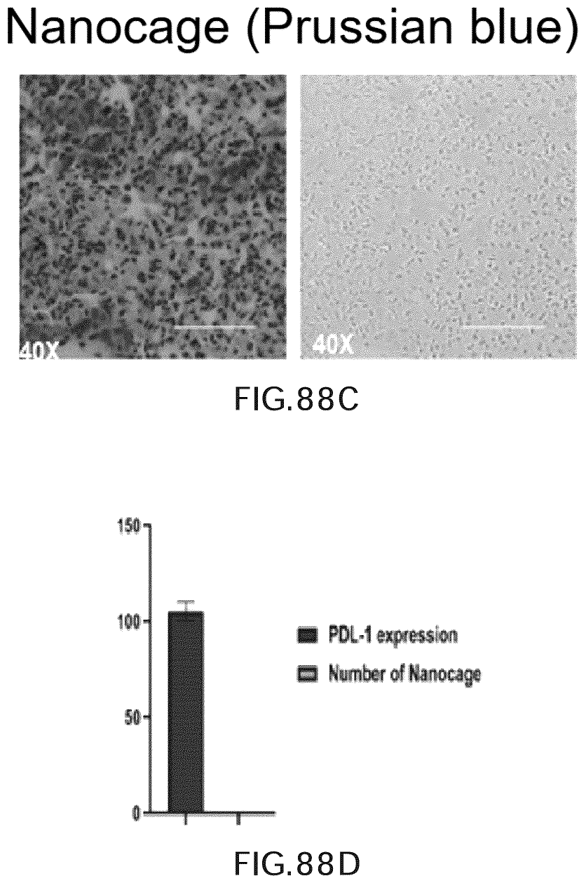

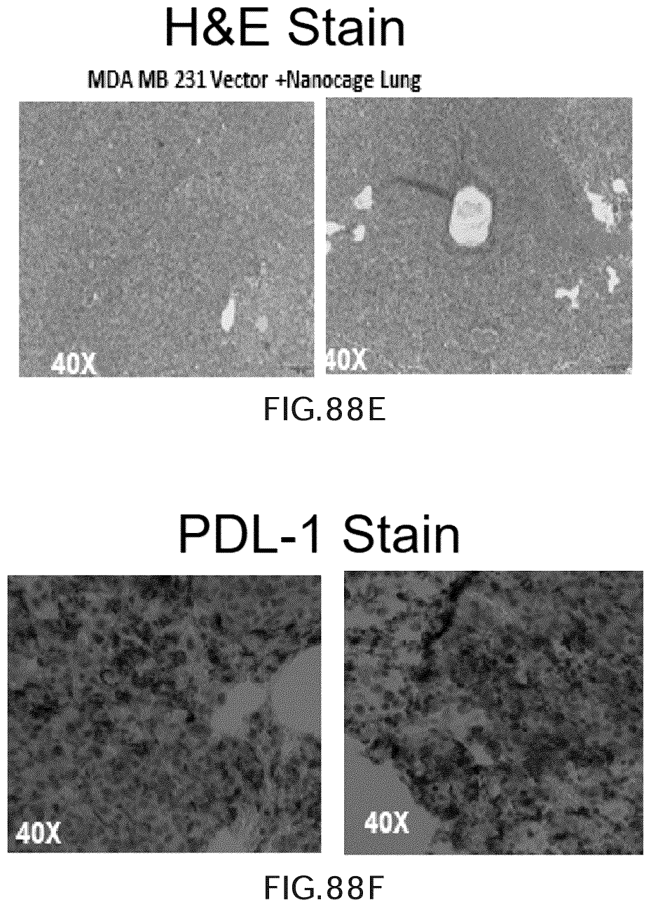

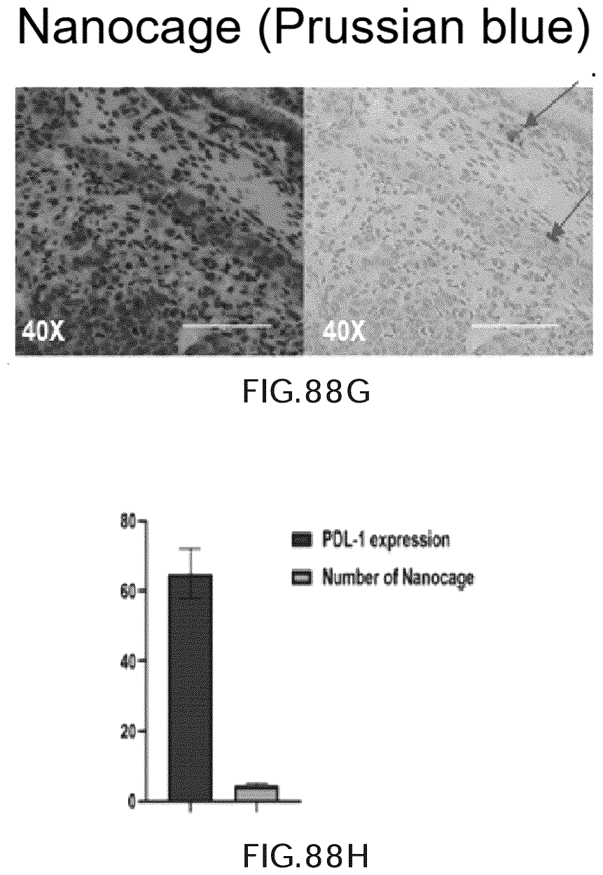

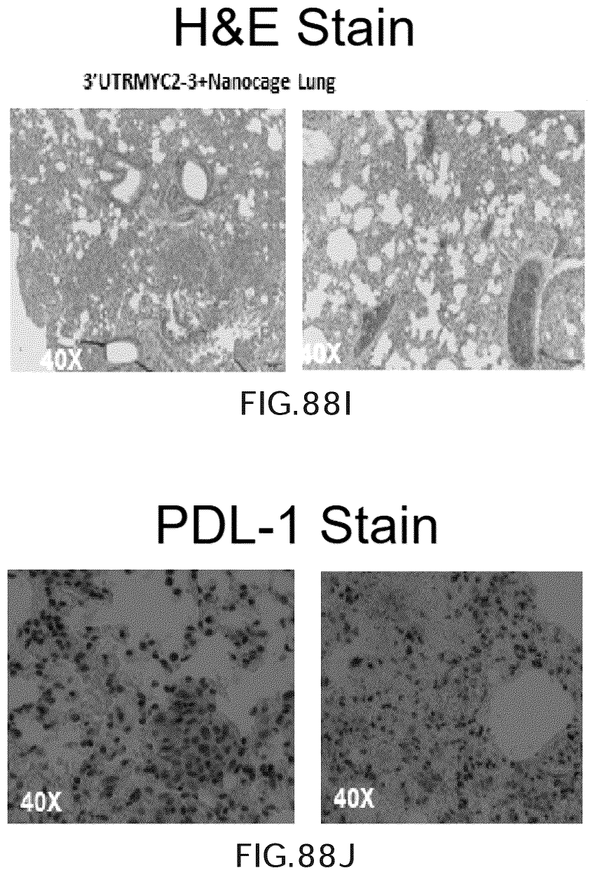

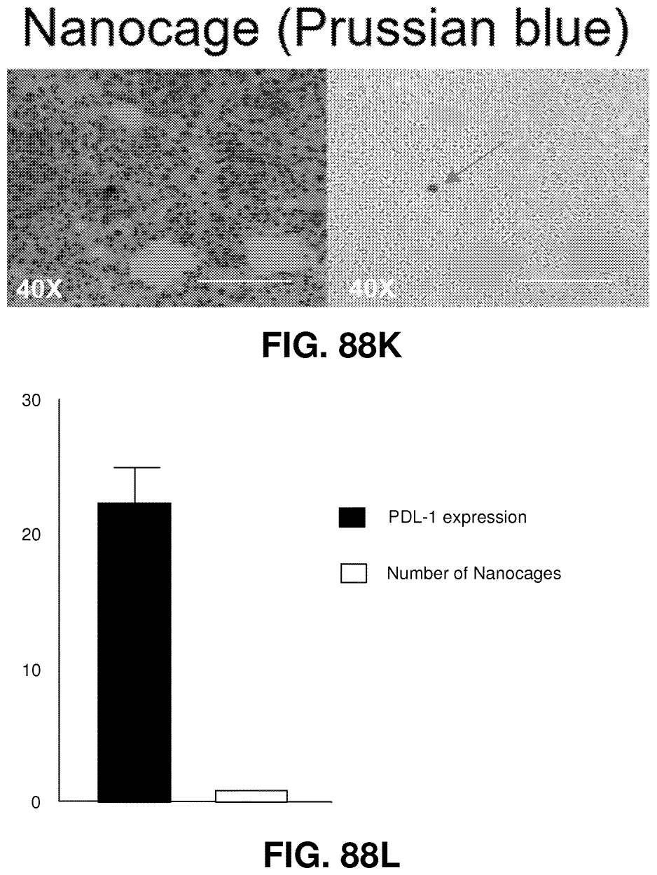

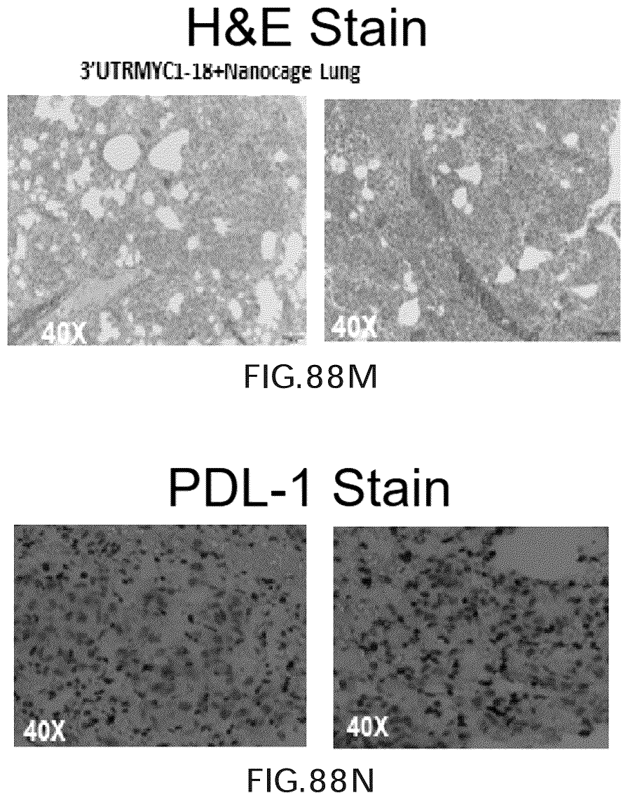

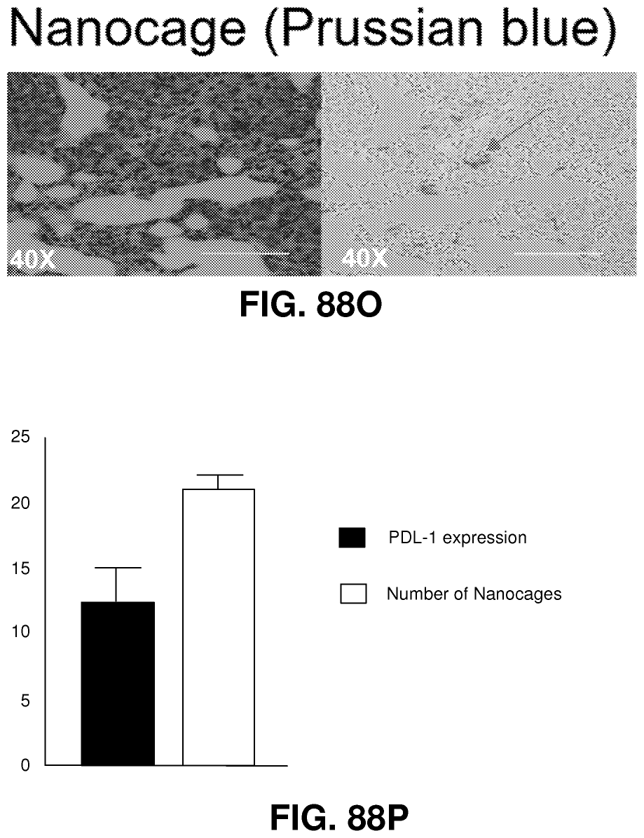

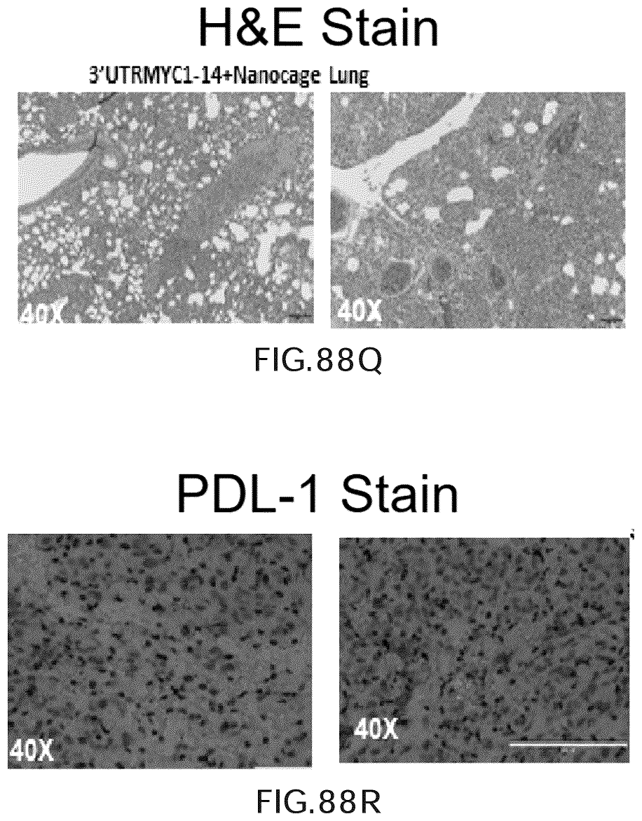

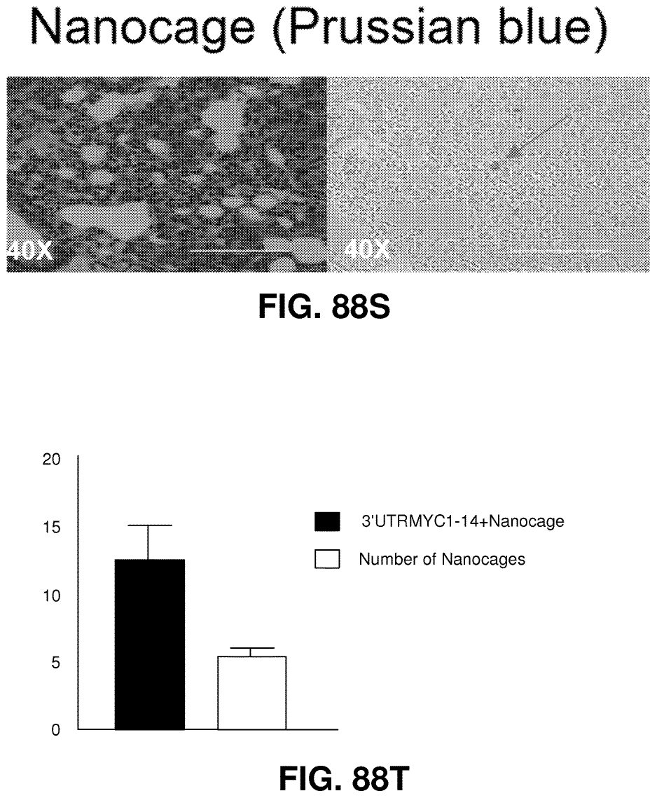

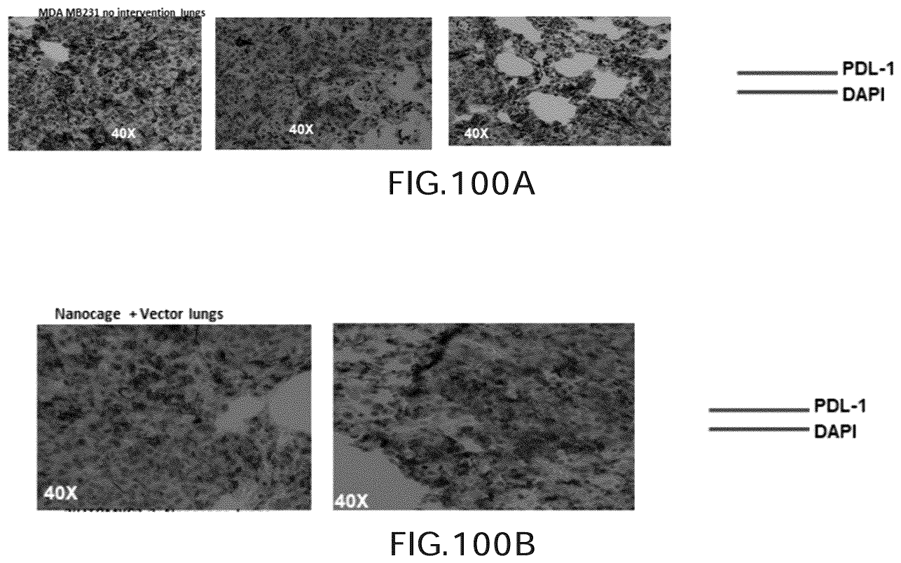

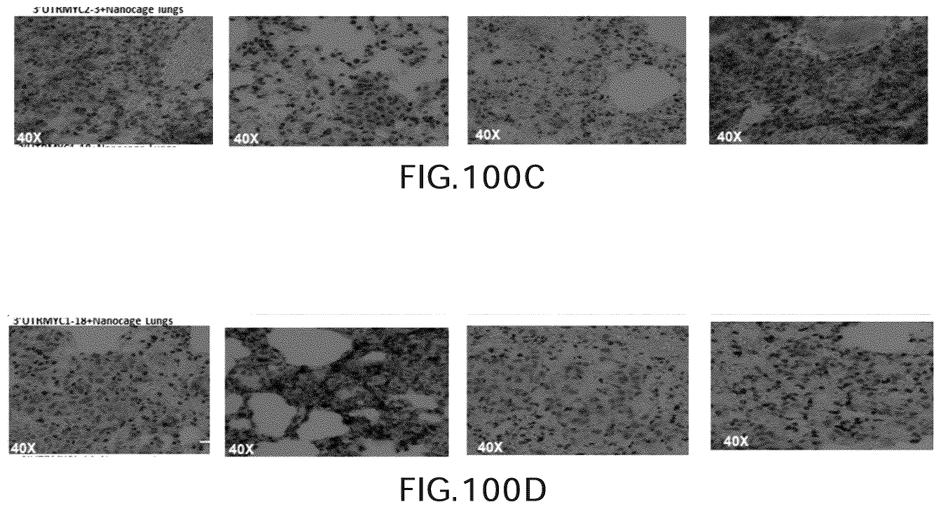

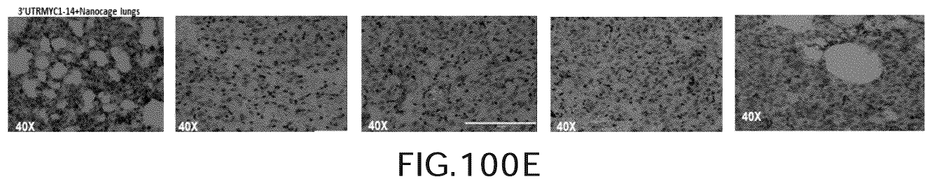

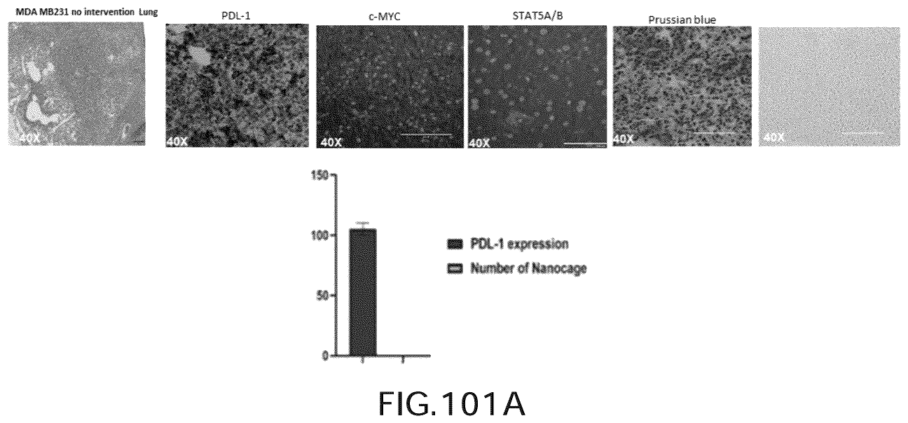

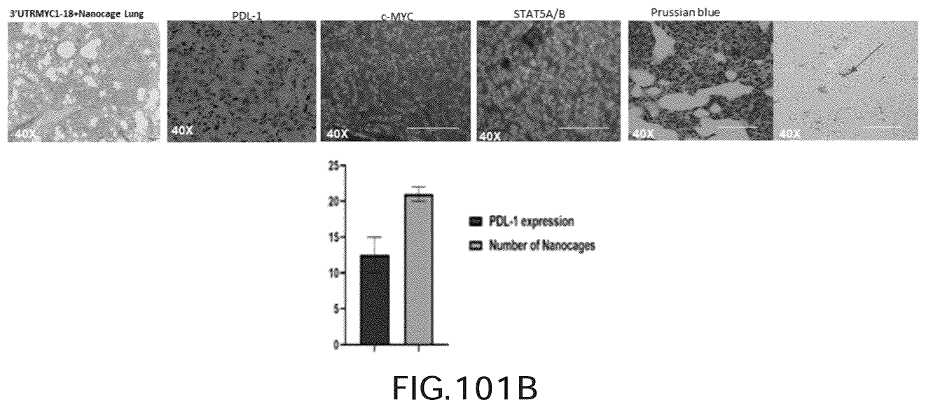

IO-nanocage packaged delivered destabilized c-MYC constructs effectively targeted c-MYC-STAT5A/5B-PDL-1 in the lungs and inhibit distant organ lung metastasis: Distant organ metastasis is a major killer of cancer patients and TNBC has preponderance to metastasize to the lungs, liver, brain, and bone. There is little to no therapeutics that effectively targets metastasis. In this disclosure, engineered destabilized c-MYC 3′UTRMYC1-18 and 3′UTRMYC1-14 were found to inhibit 80% and 60% secondary tumor metastasis, respectively (FIGS. 84A-84F), through downregulation of c-MYC-STAT5A/5B-PDL-1 in the responsive tumors (FIGS. 84A-85G). In addition, this treatment was effective in targeting the distant organ lungs and inhibited metastasis. For this evaluation, H&E, anti-c-MYC, PDL-1, STAT5A/5B and IO-nanocage staining of lung tissues was performed from the treated and untreated groups. The lungs from the no intervention group have lost its parenchymal architecture due to metastasis (FIG. 88A), with very high level of PDL-1 expression (FIG. 88B, FIG. 100A, FIG. 100B, FIG. 100C, FIG. 100D, FIG. 100E) and there was no presence of IO-nanocage as a negative control (FIG. 88C), which was also quantified in FIG. 88D. The loss of lung parenchyma in the control of IO-nanocages with vectors (FIG. 88E), with high levels of PDL-1 (FIG. 88F) and IO-nanocages were found in the tumors as a positive control (FIG. 88G, FIG. 88H). The lungs from the treated groups still have some of their lung parenchyma intact (FIG. 88I, FIG. 88M, FIG. 88Q) with low level of PDL-1 expression (FIG. 88J, FIG. 88N, FIG. 88R) and IO-nanocages were found in their lung parenchyma (FIG. 88K, FIG. 88L, FIG. 88O, FIG. 88P, FIG. 88S, FIG. 88T). The most effective construct 3′UTRMYC1-18 group has the most IO-nanocages in the lung parenchyma, which is consistent with the reduced level of c-MYC and STAT5A/5B in the lungs of mice treated with 3′UTRMYC1-18 (FIG. 101A, FIG. 101B). Taken together, we have demonstrated our therapy reached distant organ lungs and reduced c-MYC, STAT5A/5B and PDL-1 expression which inhibited metastasis and preserved the lung parenchyma in the treated group.

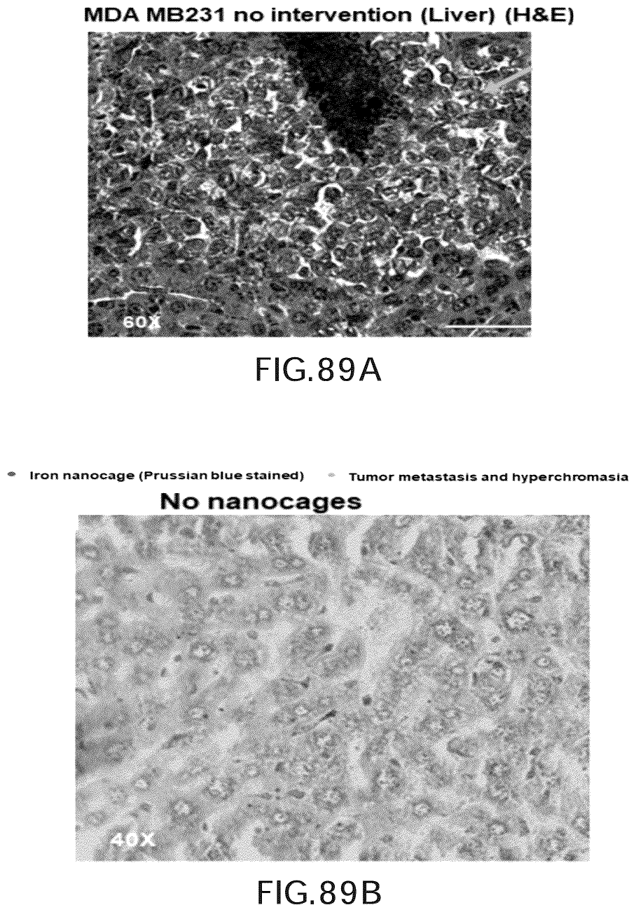

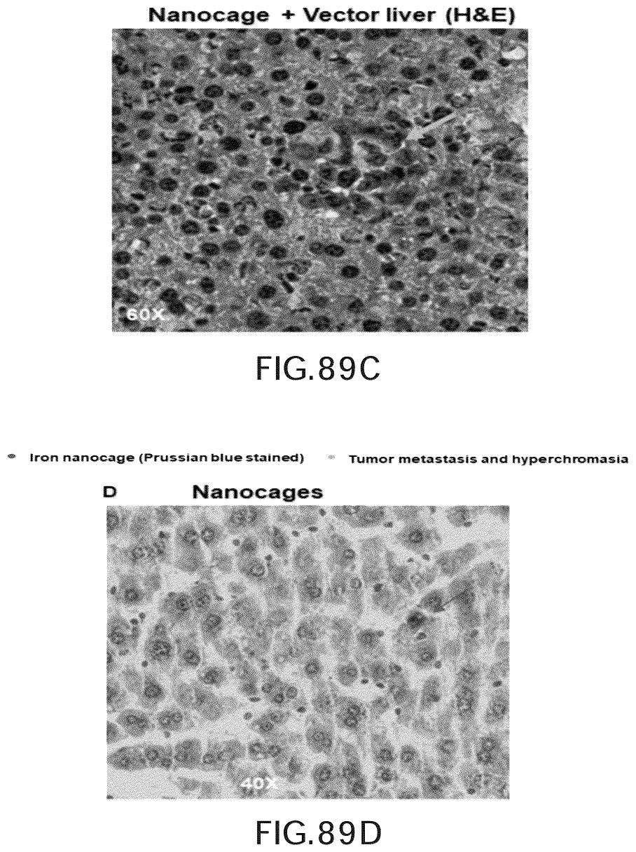

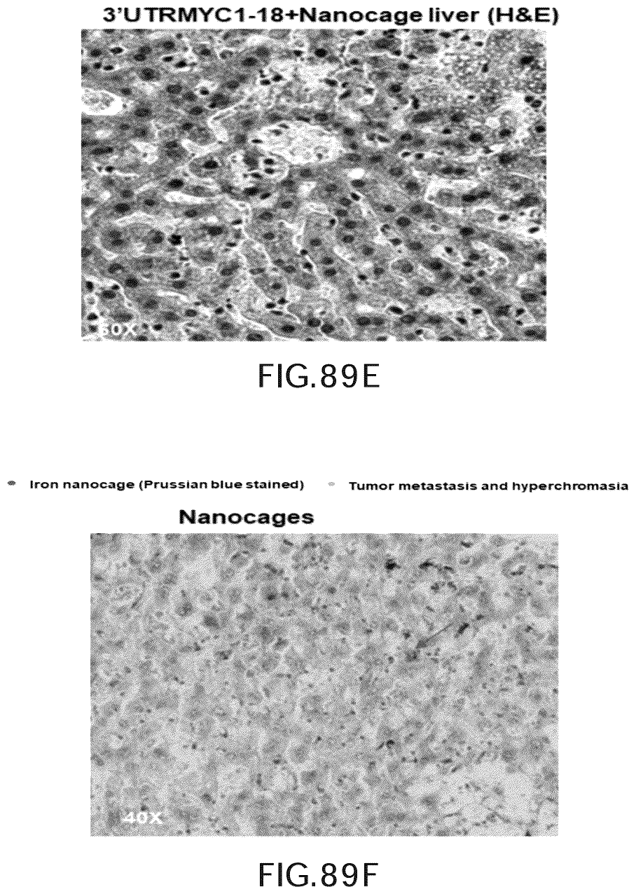

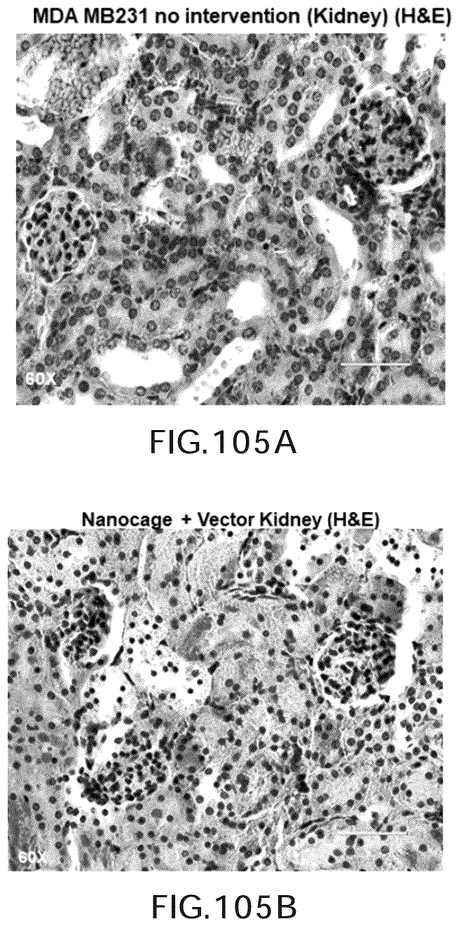

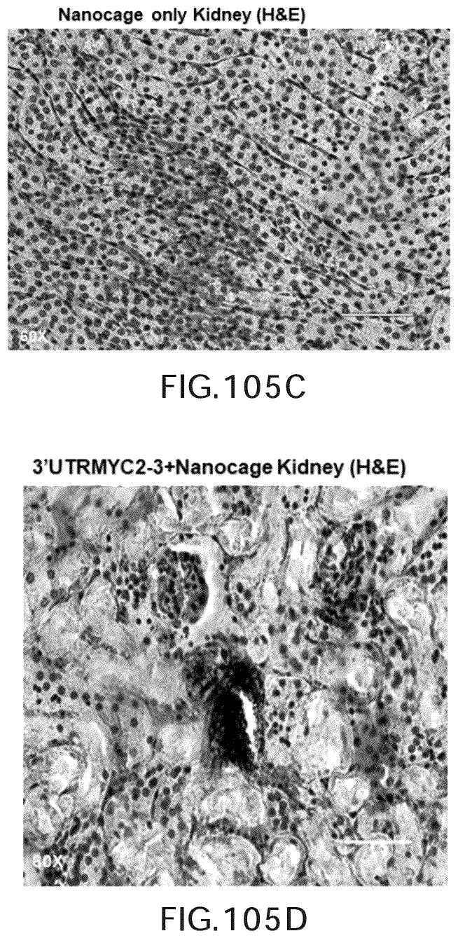

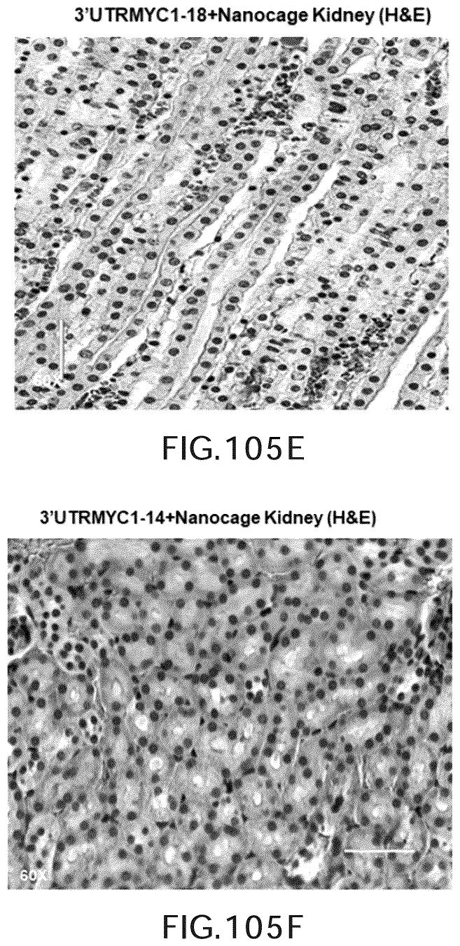

IO-nanocage robustly delivered destabilized c-MYC constructs to the liver and inhibited liver metastasis: Liver is a major site for tumor metastasis, and thus the constructs were examined for inhibition of liver metastasis and if the IO-nanocage was robustly delivered there. The 3′UTRMYC1-18 inhibited liver metastasis and preserved the liver parenchyma compared to untreated control liver and vector treated liver groups (FIG. 89A, FIG. 89C, FIG. 89E). IO-nanocages were robustly delivered in the liver of the 3′UTRMYC1-18 treated mice compared to the control (FIG. 89B, FIG. 89C, FIG. 89F). This was extended into kidneys and H&E staining of the treated and control kidneys was performed. 3′UTRMYC1-18 preserved kidney tubule cellular architecture compared to controls (FIG. 105A, FIG. 105B, FIG. 105C). This data validates the findings on the inhibition of primary tumor and metastasis to distant organs. Also see FIG. 105D, FIG. 105E, and FIG. 105F.

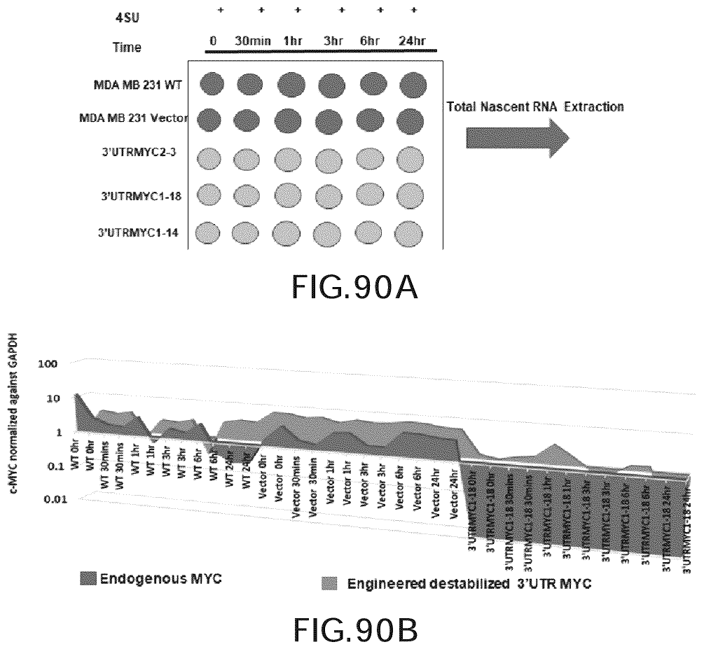

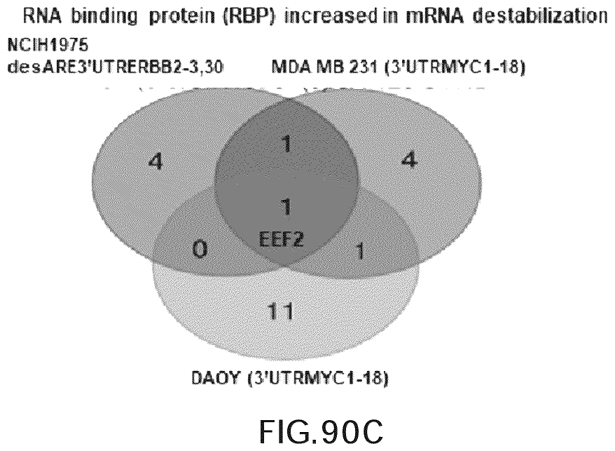

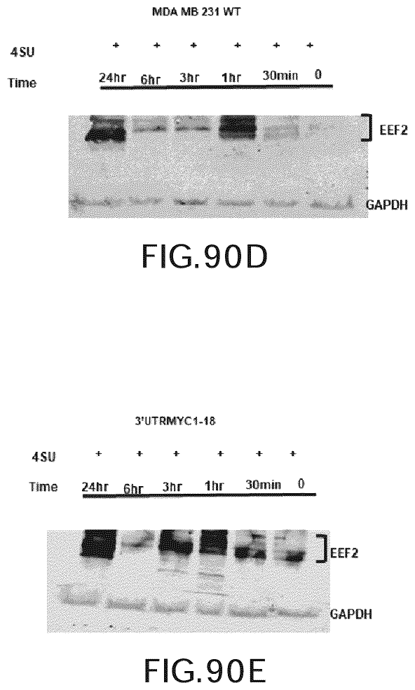

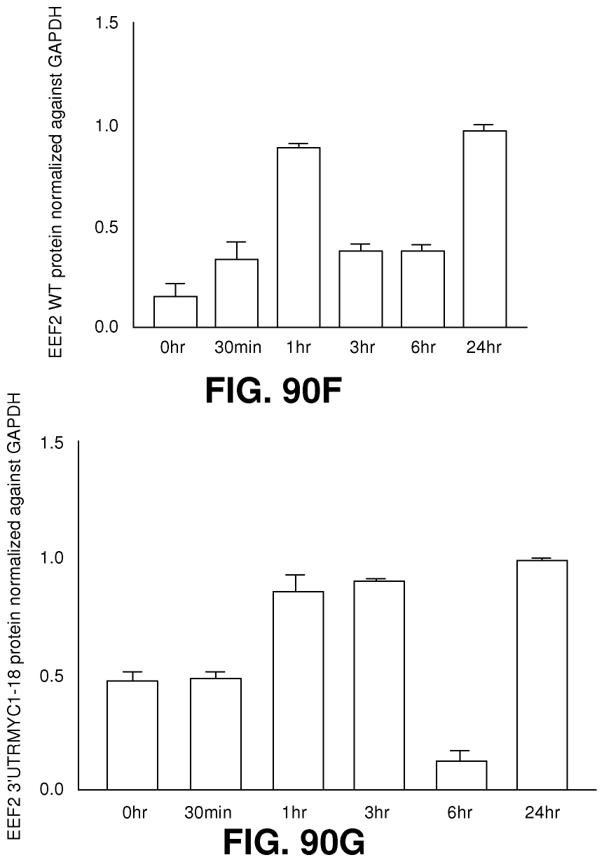

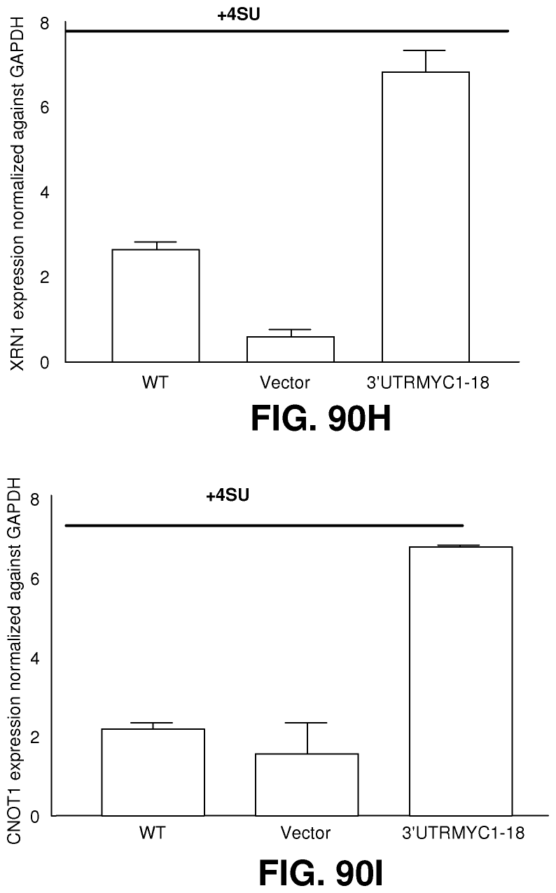

The engineered destabilized c-MYC degrades the endogenous MYC by overwriting its mRNA message through EEF2 upregulation and increased XRN1 and CNOT1 expression: To understand mechanistically how the engineered destabilized constructs overwrote the endogenous c-MYC encoded messages, control MDAMB231 and destabilized cells were labeled with 4SU (thiouridine) and collected RNAs at 0 hr, 30 mins, 1 hr, 3 hrs, 6 hrs and 24 hrs to assay the expression of the endogenous and destabilized c-MYC in the same cells and at the same time point (FIG. 90A). The 3′UTRMYC1-18 outcompeted the mRNA of endogenous MYC (FIG. 90B) by a factor of 4-fold in the transcript production, and this production of infidel destabilizing transcript triggers nonsense mediated decay through the upregulation of UPF1 (FIG. 103A, FIG. 103B, FIG. 103C, FIG. 103D, FIG. 103E). To find the ribosomal machinery and how RNA binding proteins (RBPs) orchestrating the 3′UTRMYC 1-18 outcompeted the endogenous MYC. RNAseq data of the destabilized cells and controls was searched for RBPS and EEF2 was found to be upregulated in the 3′UTRMYC1-18 treated cells or cells destabilized by the desARE3′UTRERBB2-3 and 30 constructs (FIG. 90C). To validate this, we performed western blot on the 4SU time treated samples both in the wildtype and 3′UTRMYC1-18 cells. The EEF2 protein expression was relatively higher in 3′UTRMYC1-18 at time points 30 mins to 3 hrs. compared to the wildtype (FIG. 90D, FIG. 90E, FIG. 90F, FIG. 90G). An assay was conducted for the expression of 5′-3′ and 3′-5′ exonucleases that degrades the destabilized transcript, and both XRN1 and CNOT1 were markedly upregulated in the 3′UTRMYC1-18 treated cells as compared to the controls (FIG. 90I, FIG. 90J).

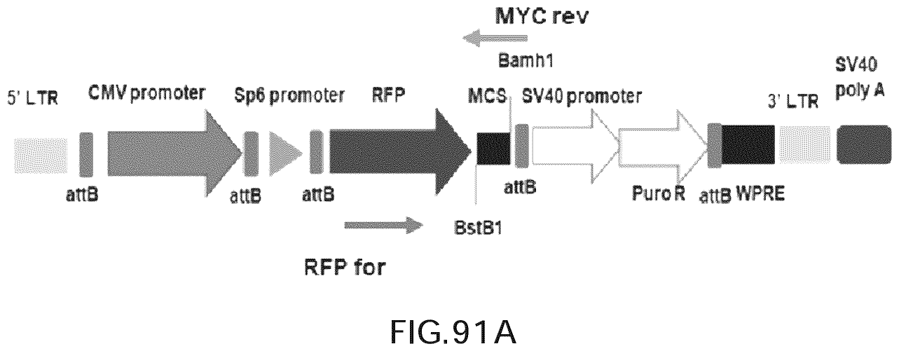

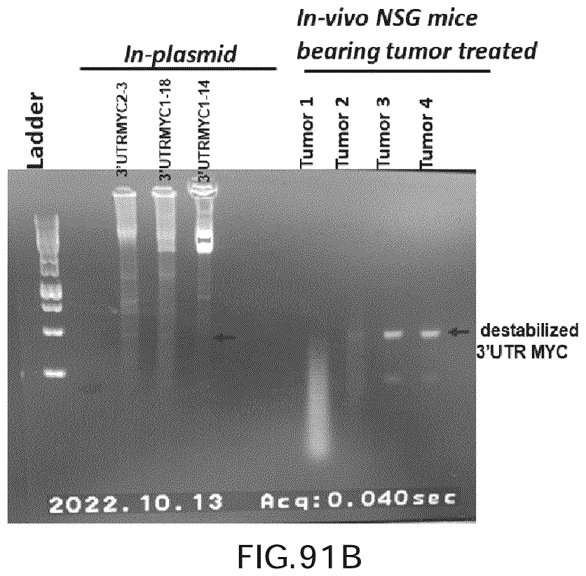

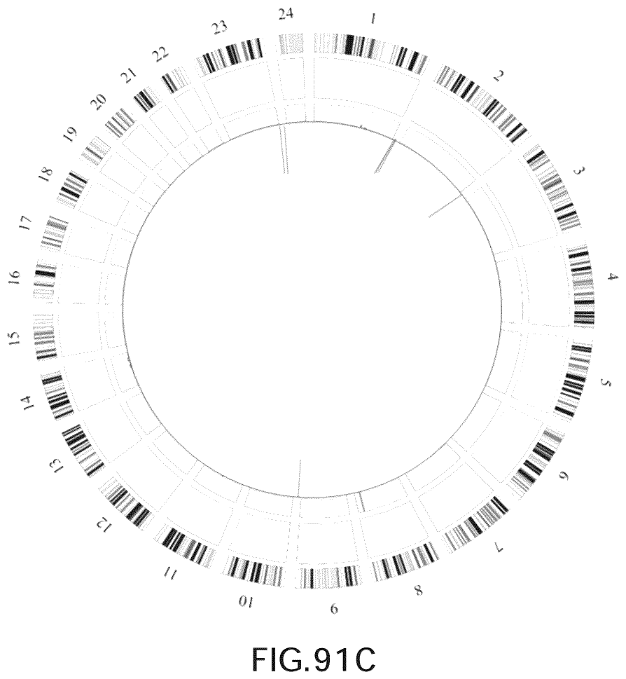

Engineered destabilized c-MYC constructs safely were integrated to the non-coding region of the genome: One of the major concerns of gene therapy, is unwanted integration of constructs into hotspots or near or into oncogenic loci, etc. This disclosure demonstrates the disclosed constructs are safe by performing optical genome mapping. To validate the sites of integration of the constructs in the genome, targeted sequencing of the genomic DNA of tumors was performed that received the constructs using primers that recognize the component part of the constructs (FIG. 91A, FIG. 91B). The constructs integrated into the non-coding regions of the chromosome 1, 2, 9, X (FIG. 91C), and in the intron of the genes PPARGC1B and EFR3B. All of these results indicate that the constructs are integrated into safe harbors and are not within a hot spot or oncogenic loci. This supports the safety profile observed in the therapy.

### Discussion

c-MYC overexpression is a major target to treat many difficult cancers. This disclosure shows the development of the engineered destabilized 3′UTR c-MYC constructs which reliably destabilized and degraded the c-MYC transcript and protein, and they inhibited c-MYC driven TNBC and metastasis by downregulating c-MYC-STAT5A/5B-PDL-1 interaction. This breakthrough was achieved by overwriting the endogenous oncogenic mRNA message with a c-MYC destabilizing mRNA message. The administration of the constructs complexed with IO-nanocage to mice-bearing TNBC caused no adverse reaction, and it did not kill normal epithelial cells expressing c-MYC. The IO-nanocages were well distributed to the tumors, liver and lungs of the animals that received the constructs to assist the effectiveness of treatment. The constructs were integrated safely into the non-coding region of genome and not in an oncogenic hotspots.

The primary tumor responsive to this therapy lost c-MYC-STAT5A/5B-PDL 1 interaction upon the treatment, while the metastatic tumors which responded to the therapy have lost c-MYC and PDL-1, yet they retain the robust expression of STAT5A/5B. This finding suggests that one can stratify patients that can receive this therapy based on the presence of c-MYC-STAT5A/5B and PDL1 positivity for the primary TNBC. However, for the treatment of metastatic tumors, the combination of construct and a STAT5A/5B inhibitor is needed or the combination of destabilizing c-MYC and STAT5A/5B constructs is more appropriate.

This disclosure provides a new paradigm to combine constructs with anti-PDL-1 and anti STAT5A/5B inhibitor or with a newly designed destabilizing STAT5A/5B construct, which is critical for the improvement of cancer treatment since PDL-1 inhibitor (atezolizumab) alone in many clinical trials was not able to control metastatic TNBC in many clinical trials. Since PDL-1 negative metastatic TNBC is more prevalent (NCT03164993), it is plausible that they might be the same STAT5A/5B only TNBC, and thus this strategy might be applicable to target the PDL-1 negative-STAT5A/5B only positive metastatic TNBC.

These constructs offer survival benefits of 16-17 days corresponding to 680 days (22 months) of survival for human, given that one mice day is equivalent to 40 human days. Atezolizumab plus paclitaxel offered 7.5 months survival benefit versus placebo plus paclitaxel, while atezolizumab plus chemotherapy achieved 20.6 months survival benefit and placebo plus chemotherapy gave 19.8 months survival benefit. (Impassion 031, Impassion 130, NCT02489448, MK-3475-119/KEYNOTE-119). Therefore, the 22 months survival benefit of these construct/anti-PDL-1/STAT5A/5B combinations could be superior to the standard of cancer care.

This disclosure also shows the generalizability of this approach by targeting and degrading c-MYC in c-MYC driven osteosarcoma, medulloblastoma, prostate cancer, and Burkitt's lymphoma. This technology may be used as a platform to safely engineer various transcripts as well as targeting therapeutic genes similar to previously shown ERBB2-driven cancer treatments.

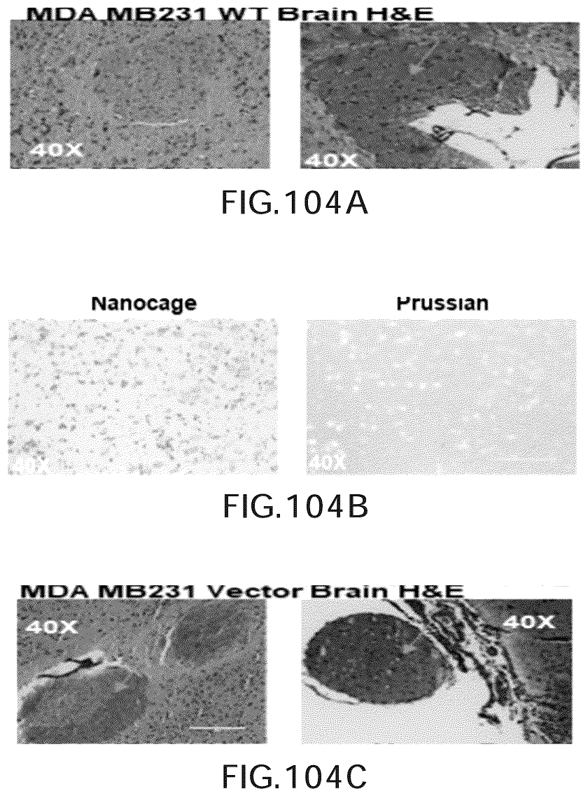

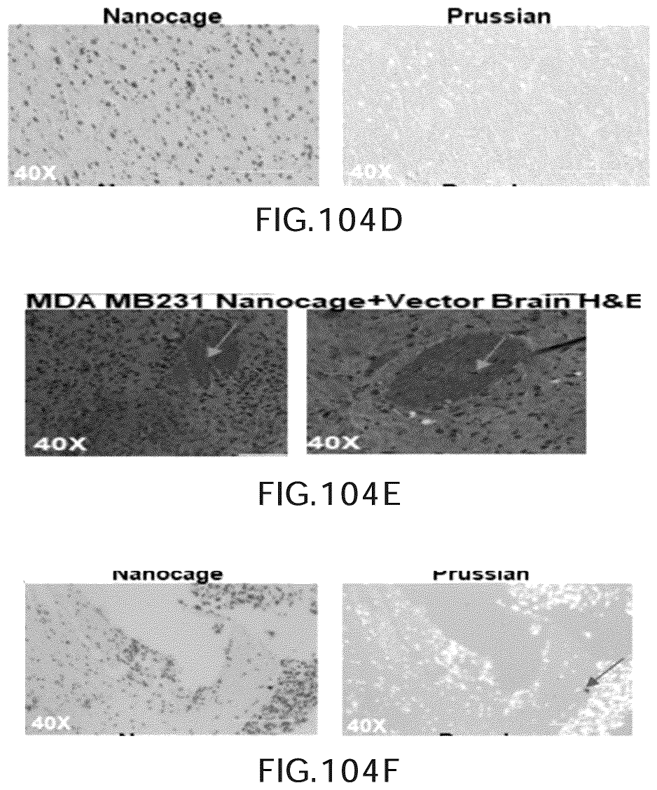

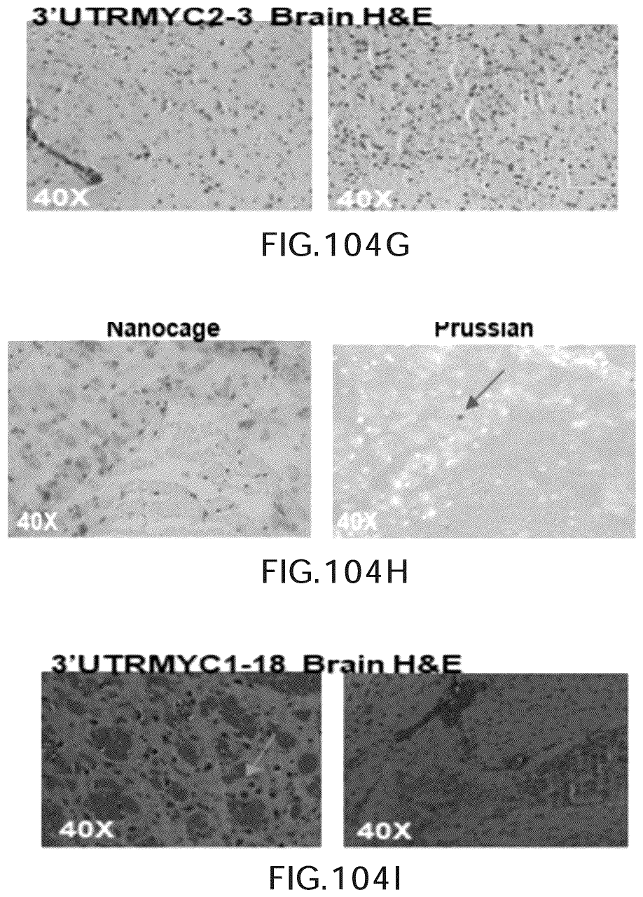

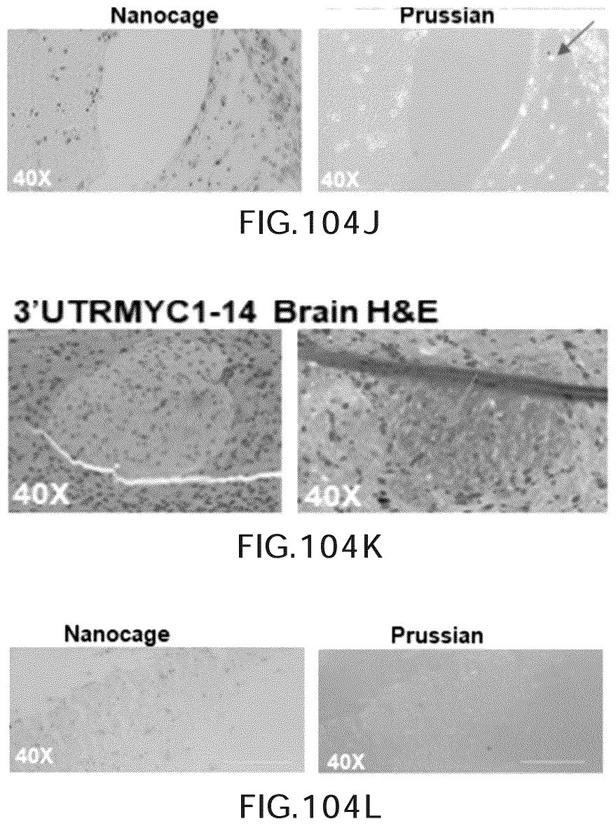

Brain cancer: 3′UTRMYC2-3 inhibited brain metastasis (FIG. 104A-L) and very few nanocages were detected.

Prostate Cancer: In one embodiment, the constructs are used to treat prostate cancer. For such cancers the modes of delivery include transuretheral, transperineal, and trans rectal delivery modes.

TABLE 10
Primers

c-MYC forward (Gibson Assembly primer)
gcaacgcaatGGACCCGCCCGAGCTTCGAAAAAAAAAAGCCATCG
(SEQ ID NO: 53)
c-MYC reverse (Gibson Assembly Primer)
actcacattaCGGCGGGGCCGGCCCCTA (SEQ ID NO: 54)
pLenti CMVSp6 Vector forward
(Gibson Assembly Primer)
ggccccgccgTAATGTGAGTTAGCTCACTCATTAGG
(SEQ ID NO: 55)
pLentiCMVSp6 Vector reverse
(Gibson Assembly primer)
gggcgggtccATTGCGTTGCGCTCA CTG (SEQ ID NO: 56)
DCP1A promoter forward (Gibson Assembly primer)
CCCTCAACTTCCGCCTCTAC (SEQ ID NO: 24)
DCP1A promoter reverse (Gibson Assembly primer)
CAGCCTGCAAGCTCCACTAC (SEQ ID NO: 25)
c-MYC3′UTR Forward primers (RTPCR)
CCTCACAACCTTGGCTGAGT (SEQ ID NO: 22)
c-MYC3′UTR Reverse primer (RTPCR)
GGATTGAAATTCTGTGTAACTGC (SEQ ID NO: 23)
c-MYC forward primers (qPCR)
GTCACACCCTTCTCCCTTCG (SEQ ID NO: 57)
c-MYC reverse primers (qPCR)
CAGGTACAAGCTGGAGGTGG (SEQ ID NO: 58)
EEF2 Forward primer (qPCR)
ACGTTCACGTTCTCCACGAT (SEQ ID NO: 59)
EEF2 Reverse primer (qPCR)
TGTCAGGTAAGCAGTGGGTC (SEQ ID NO: 60)
CNOT1 Forward primer (qPCR)
TCTAGTGCAGCAATCCCGAA (SEQ ID NO: 61)
CNOT1 Reverse primer (qPCR)
GGACTGGTCACTTACATGGC (SEQ ID NO: 62)
XRN1 Forward primer (qPCR)
TCCATAAAGAAGAGGCAGTGC (SEQ ID NO: 63)
XRN1 Reverse Primer (qPCR)
AGATGATTGGATTTTGATGGGGT (SEQ ID NO: 64)

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A method of treating a cancer in a subject having a metastatic tumor, the method comprising administering to the subject a pharmaceutical composition comprising a plasmid vector comprising a therapeutically effective amount of a DNA molecule, wherein the DNA molecule comprises in the 5′→3′ direction of transcription relative to synthesis of a messenger RNA (mRNA) molecule comprising a 3′ UTR of an mRNA encoding a MYC protein:
(a) a promoter, operatively linked to
(b) a nucleic acid sequence encoding a 3′ UTR of a MYC gene in the mRNA molecule in which one, or more ARE poly(U) stabilizing motifs of the 3′ UTR of the mRNA are substituted with a nucleic acid sequence selected from AUUUU, CCUC, CUGC, UAAGUUAU, UAACUUAU, and GUAAAUAG, wherein the 3′ UTR of the mRNA is at least 20% identical to SEQ ID NO: 12.

2. The method of claim 1, wherein the 3′ UTR of the mRNA is at least 80% identical to SEQ ID NO: 12.

3. The method of claim 1, wherein the 3′ UTR of the mRNA is at least 85% identical to SEQ ID NO: 12.

4. The method of claim 1, wherein the 3′ UTR of the mRNA is at least 90% identical to SEQ ID NO: 12.

5. The method of claim 1, wherein the 3′ UTR of the mRNA is at least 95% identical to SEQ ID NO: 12.

6. The method of claim 1, wherein the 3′ UTR of the mRNA is at least 99% identical to SEQ ID NO: 12.

7. The method of claim 1, wherein the promoter sequence is selected from a DCP1A promoter, a DCP2 promoter, and a ZFP36 promoter.

8. The method of claim 1, wherein the nucleic acid sequence encoding the 3′ UTR of the MYC gene is selected from SEQ ID NO: 46; SEQ ID NO: 47, or SEQ ID NO: 48.

9. The method of claim 8, wherein the nucleic acid sequence encoding the 3′ UTR of the MYC gene is at least 20% identical to SEQ ID NO: 46; SEQ ID NO: 47, or SEQ ID NO: 48.

10. The method of claim 8, wherein the nucleic acid sequence encoding the 3′ UTR of the MYC gene is at least 80% identical to SEQ ID NO: 46; SEQ ID NO: 47, or SEQ ID NO: 48.

11. The method of claim 8, wherein the nucleic acid sequence encoding the 3′ UTR of the MYC gene is at least 85% identical to SEQ ID NO: 46; SEQ ID NO: 47, or SEQ ID NO: 48.

12. The method of claim 8, wherein the nucleic acid sequence encoding the 3′ UTR of the MYC gene is at least 90% identical to SEQ ID NO: 46; SEQ ID NO: 47, or SEQ ID NO: 48.

13. The method of claim 8, wherein the nucleic acid sequence encoding the 3′ UTR of the MYC gene is at least 95% identical to SEQ ID NO: 46; SEQ ID NO: 47, or SEQ ID NO: 48.

14. The method of claim 8, wherein the nucleic acid sequence encoding the 3′ UTR of the MYC gene is at least 99% identical to SEQ ID NO: 46; SEQ ID NO: 47, or SEQ ID NO: 48.

15. The method of claim 8, wherein the DNA molecule is at least 20% identical to SEQ ID NO: 81; SEQ ID NO: 82, or SEQ ID NO: 83.

16. The method of claim 8, wherein the DNA molecule is at least 80% identical to SEQ ID NO: 81; SEQ ID NO: 82, or SEQ ID NO: 83.

17. The method of claim 8, wherein the DNA molecule is at least 90% identical to SEQ ID NO: 81; SEQ ID NO: 82, or SEQ ID NO: 83.

18. The method of claim 8, wherein the DNA molecule is at least 95% identical to SEQ ID NO: 81; SEQ ID NO: 82, or SEQ ID NO: 83.

19. The method of claim 8, wherein the DNA molecule is at least 99% identical to SEQ ID NO: 81; SEQ ID NO: 82, or SEQ ID NO: 83.

20. A deoxyribonucleic acid (DNA) molecule comprising in the 5′→3′ direction of transcription relative to synthesis of a messenger RNA (mRNA) molecule comprising a 3′ UTR of an mRNA encoding a MYC protein:
(c) a promoter, operatively linked to
(d) a nucleic acid sequence encoding a 3′ UTR of a MYC gene in the mRNA molecule in which one, or more ARE poly(U) stabilizing motifs of the 3′ UTR of the mRNA are substituted with a nucleic acid sequence selected from AUUUU, CCUC, CUGC, UAAGUUAU, UAACUUAU, and GUAAAUAG, wherein the 3′ UTR of the mRNA is at least 20% identical to SEQ ID NO: 12.
